SEQUENCE LISTING (3 sequences)

SEQ ID NO: 1 - DNA, 19 bp, Artificial Sequence
  PCR primer
ctgtgccttgggtggcttt

SEQ ID NO: 2 - DNA, 24 bp, Artificial Sequence
  PCR primer
aaggaaagaagtcagaaggcaaaa

SEQ ID NO: 3 - DNA, 30 bp, Artificial Sequence
  Fluorescent probe
  misc_feature (1)..(1): FAM
  misc_feature (9)..(9): ZEN quencher
  misc_feature (30)..(30): 3IABkFQ quencher
agctccaaattctttataagggtcgatgtc

(19) United States
(12) Patent Application Publication
(10) Pub. No.: US 2023/0295096 A1
(43) Pub. Date: Sep. 21, 2023

(54) PYRAZOLE CARBOXAMIDE COMPOUNDS FOR TREATMENT OF HBV

(71) Applicant: ASSEMBLY BIOSCIENCES, INC., South San Francisco, CA (US)

(72) Inventors: Simon Nicolas HAYDAR, South San Francisco, CA (US); Thilo HECKRODT, South San Francisco, CA (US); Michael WALKER, South San Francisco, CA (US); Min ZHONG, South San Francisco, CA (US)

(21) Appl. No.: 17/920,546

(22) PCT Filed: Apr. 21, 2021
(86) PCT No.: PCT/US2021/028305

§ 371 Date: Oct. 21, 2022

Related U.S. Application Data
(60) Provisional application No. 63/013,996, filed on Apr. 22, 2020.

(51) Int. Cl. C07D 231/38; C07D 401/08; C07D 417/08; C07D 403/08; C07D 413/08; C07D 405/12; C07D 401/12; C07D 453/02; C07D 409/12; C07D 413/04; C07D 417/14; C07D 403/04; C07D 417/04; C07D 405/14; C07D 401/14; C07D 403/14; A61P 31/20

(52) U.S. Cl. CPC C07D 231/38; A61P 31/20; C07D 401/08; C07D 401/12; C07D 401/14; C07D 403/04; C07D 403/08; C07D 403/14; C07D 405/12; C07D 405/14; C07D 409/12; C07D 413/04; C07D 413/08; C07D 417/04; C07D 417/08; C07D 417/14; C07D 453/02

(57) ABSTRACT

The present disclosure provides, in part, pyrazole carboxamide compounds, and pharmaceutical compositions thereof, useful for disruption of HBV core protein assembly, and methods of treating Hepatitis B (HBV) infection.

### BACKGROUND

Hepatitis B (HBV) causes viral hepatitis that can further lead to chronic liver disease and increase the risk of liver cirrhosis and liver cancer (hepatocellular carcinoma). Worldwide, about 2 billion people have been infected with HBV, around 360 million people are chronically infected, and every year HBV infection causes more than one half million deaths. HBV can be spread by body fluids: from mother to child, by sex, and via blood products. Children born to HBV-positive mothers may also be infected, unless vaccinated at birth.

The hepatitis virus particle is composed of a lipid envelope studded with surface protein (HBsAg) that surrounds the viral core. The core is composed of a protein shell, or capsid, built of 120 core protein (Cp) dimers, which in turn contains the relaxed circular DNA (rcDNA) viral genome as well as viral and host proteins. In an infected cell, the genome is found as a covalently closed circular DNA (cccDNA) in the host cell nucleus. The cccDNA is the template for viral RNAs and thus viral proteins. In the cytoplasm, Cp assembles around a complex of full-length viral RNA (the so-called pregenomic RNA or pgRNA and viral polymerase (P). After assembly, P reverse transcribes the pgRNA to rcDNA within the confines of the capsid to generate the DNA-filled viral core.

At present, chronic HBV is primarily treated with nucleos(t)ide analogs (e.g., entecavir) that suppress the virus while the patient remains on treatment, but do not eliminate the infection, even after many years of treatment. Once a patient starts taking nucleos(t)ide analogs, most must continue taking them or risk the possibility of a life-threatening immune response due to viral rebound. Further, nucleotide therapy may lead to the emergence of antiviral drug resistance.

The only FDA approved alternative to nucleos(t)ide analogs is treatment with interferon α or pegylated interferon α. Unfortunately, the adverse event incidence and profile of interferon α can result in poor tolerability, and many patients are unable to complete therapy. Moreover, only a small percentage of patients are considered appropriate for interferon therapy, as only a small subset of patients is likely to have a sustained clinical response to a course of interferon therapy. As a result, interferon-based therapies are used in only a small percentage of all diagnosed patients who elect treatment.

Thus, current HBV treatments can range from palliative to watchful waiting. Nucleotide analogs suppress virus production, treating the symptom, but leave the infection intact. Interferon α has severe side effects and less tolerability among patients and is successful as a finite treatment strategy in only a small minority of patients. There is a clear on-going need for more effective treatments for HBV infections.

### SUMMARY

The present disclosure provides, in part, pyrazole carboxamide compounds and pharmaceutical compositions thereof, useful for disruption of HBV core protein assembly, and methods of treating HBV infections.

In one aspect, the disclosure provides a compound of Formula I:

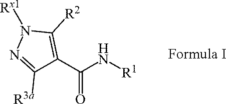

or a pharmaceutically acceptable salt thereof, where the variables are described in the detailed description.

In another aspect, the disclosure provides a compound of Formula II:

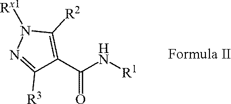

or a pharmaceutically acceptable salt thereof, where the variables are described in the detailed description.

In another aspect, the disclosure provides pharmaceutical compositions comprising a compound of Formula I or II, or a pharmaceutically acceptable salt thereof, and a pharmaceutically acceptable excipient.

In another aspect, the disclosure provides a method of treating an HBV infection in a subject in need thereof, comprising: administering to the subject a therapeutically effective amount of compound of Formula I or II, or a pharmaceutically acceptable salt thereof.

In another aspect, the disclosure provides a method of treating an HBV infection in a subject in need thereof, comprising: administering to the subject a pharmaceutical composition comprising a therapeutically effective amount of a compound of Formula I or II, or a pharmaceutically acceptable salt thereof, and a pharmaceutically acceptable excipient.

### BRIEF DESCRIPTION OF DRAWINGS

FIG. 1 shows the ORTEP plot for compound CP-AIA-227-2.

FIG. 2 shows the relative stereochemistry scheme of compound CP-AIA-227-2.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art.

### I. Definitions

The term “alkenyl” as used herein refers to an unsaturated straight or branched hydrocarbon having at least one carbon-carbon double bond. Exemplary alkenyl groups include, but are not limited to, a straight or branched group of 2-6 carbon atoms, referred to herein as C₂₋₆alkenyl. Exemplary alkenyl groups include, but are not limited to, vinyl, allyl, butenyl, pentenyl, etc....

The term “alkoxy” as used herein refers to a straight or branched alkyl group attached to oxygen (i.e., alkyl—O—). Exemplary alkoxy groups include, but are not limited to, alkoxy groups of 1-6 or 1-4 carbon atoms, referred to herein as C₁₋₆alkoxy and C₁₋₄alkoxy, respectively. Exemplary alkoxy groups include, but are not limited to methoxy, ethoxy, and isopropoxy, etc.

The term “alkoxyalkyl” as used herein refers to an alkyl group substituted with an alkoxy group. Examples include, but are not limited to, CH₃CH₂OCH₂—, CH₃OCH₂CH₂— and CH₃OCH₂-, etc.

The term “alkyl” as used herein refers to a saturated straight or branched hydrocarbon. Exemplary alkyl groups include, but are not limited to, straight or branched hydrocarbons of 1-6 or 1-4 carbon atoms, referred to herein as C₁₋₆ alkyl and C₁₋₄ alkyl, respectively. Exemplary alkyl groups include, but are not limited to, methyl, ethyl, n-propyl, isopropyl, 2-methyl-1-butyl, 3-methyl-2-butyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl, 2-ethyl-1-butyl, n-butyl, isobutyl, t-butyl, n-pentyl, isopentyl, neopentyl, and n-hexyl, etc.

The term “alkylene” as used herein refers to a biradical alkyl group.

The term “alkynyl” as used herein refers to an unsaturated straight or branched hydrocarbon having at least one carbon-carbon triple bond. Exemplary alkynyl groups include, but are not limited to, straight or branched groups of 2-6 carbon atoms, referred to herein as C₂₋₆alkynyl. Exemplary alkynyl groups include, but are not limited to, ethynyl, propynyl, butynyl, pentynyl, hexynyl, and methylpropynyl, etc.

The term “carbonyl” as used herein refers to the biradical —C(O)—.

The term “cyano” as used herein refers to the radical —CN.

The term “cycloalkyl” as used herein refers to a saturated monocyclic hydrocarbon group of, for example, 3-6 carbons, referred to herein as C₃₋₆ monocycloalkyl, or bicyclic hydrocarbon ring structure of, for example, 8-12 carbons, referred to herein as C₈₋₁₂bicycloalkyl. For bicyclic cycloalkyl groups, the two rings may be attached through the same or different carbons. Exemplary monocyclic cycloalkyl groups include, but are not limited to, cyclohexyl, cyclopentyl, cyclopentenyl, cyclobutyl and cyclopropyl. Exemplary bicyclic cycloalkyl groups include, but are not limited to, spiro[2.5]octanyl, spiro[3.5]nonanyl, bicyclo[2.2.2]octanyl, bicyclo[4.1.0]heptanyl, octahydropentalenyl, bicyclo[4.2.0]octanyl, bicyclo[1.1.1]pentanyl, bicyclo[2.2.1]heptanyl, and bicyclo[2.2.2]octanyl.

The term “cycloalkenyl” as used herein refers to a partially unsaturated monocyclic hydrocarbon group of, for example, 4-6 carbons, referred to herein as C₄₋₆monocycloalkenyl, or bicyclic hydrocarbon ring structure of, for example, 8-12 carbons, referred to herein as C₈₋₁₂bicycloalkenyl. For bicyclic cycloalkenyl groups: 1) either one or both rings may contain one or more double bonds and 2) the two rings may be attached through the same or different ring carbons. Exemplary monocyclic cycloalkenyl groups include, but are not limited to, cyclopropenyl, cyclobutenyl, cyclopentenyl, cyclohexenyl and cycloheptenyl. Exemplary bicyclic cycloalkenyl groups include, but are not limited to, spiro[2.5]oct-5-enyl, spiro[2.5]oct-4-enyl, spiro[3.5]non-5-enyl, spiro[3.5]non-6-enyl, bicyclo[4.1.0]hept-3-enyl, bicyclo[4.1.0]hept-2-enyl, and bicyclo[2.2.2]oct-2-enyl.

The term “carbocyclyl” as used herein refers to a bicyclic ring system formed by fusing a phenyl ring to a C₃₋₆monocycloalkyl or C₄₋₆monocycloalkenyl ring. Examples of carbocyclyls include, but are not limited to, 2,3-dihydro-1H-indenyl, 1,2,3,4-tetrahydronaphthalenyl and 1H-indenyl.

The terms “halo” or “halogen” as used herein refer to F, Cl, Br or I.

The term “haloalkyl” as used herein refers to an alkyl group substituted with one or more halogen atoms. For example, haloC₁₋₆alkyl refers to a straight or branched alkyl group of 1-6 carbon atoms substituted with one or more halogen atoms. Examples include, but are not limited to, CH₂F—, CHCl₂—, —CHF₂, CF₃—, CF₃CH₂—, CH₃CF₂, CF₃CCl₂— and CF₃CF₂—.

The term “haloalkoxy” as used herein refers to an alkoxy group substituted with one or more halogen atoms. Examples include, but are not limited to, CCl₃O—, CF₃O—, CHF₂O—CF₃CH₂O—, and CF₃CF₂O—.

The terms “heteroaryl” as used herein refers to a 5-6 membered monocyclic or 8-12 membered bicyclic aromatic ring system containing one to four independently selected heteroatoms, such as nitrogen, oxygen and sulfur. Where possible, the heteroaryl ring may be linked to the adjacent radical though carbon or nitrogen. Examples of 5-6 membered monocyclic heteroaryl groups include, but are not limited to, furanyl, thiophenyl (also referred to as thienyl), pyrrolyl, thiazolyl, oxazolyl, isothiazolyl, isoxazolyl, imidazolyl, pyrazolyl, 1H-1,2,3-triazolyl, 2H-1,2,3-triazolyl, 1,2,4-triazolyl, pyridinyl (also referred to as pyridyl), pyridazinyl, pyrimidinyl, pyrazinyl, 1,3,5-triazinyl, 1,2,4-triazinyl, 1,2,3-triazinyl, 1,2,4-oxadiazolyl, 1,3,4-oxadiazolyl, 1,2,5-oxadiazolyl, 1,2,4-thiadiazolyl, 1,3,4-thiadiazolyl, 1,2,5-thiadiazolyl and tetrazolyl. Examples of 8-12 membered bicyclic heteroaryl groups include, but are not limited to, benzofuranyl, isobenzofuranyl, benzo[b]thiophenyl, benzo[c]thiophenyl, indolyl, isoindolyl, benzo[d]isoxazolyl, benzo[c]isoxazolyl, benzo[d]oxazolyl, benzo[d]isothiazolyl, benzo[c]isothiazolyl, benzo[d]thiazolyl, indazolyl, benzo[d]imidazolyl, benzo[d]imidazolyl, and benzo[d][1,2,3]triazolyl.

The term “heterocycloalkyl” refers to a saturated 3-6 membered monocyclic or 8-12 membered bicyclic ring system, referred to herein as C₃₋₆monoheterocycloalkyl and C₈₋₁₂biheterocycloalkyl, containing one to four independently selected heteroatoms, such as nitrogen, oxygen, and sulfur (including its oxidation states: S(O) and SO₂). Where possible, heterocycloalkyl rings may be linked to the adjacent radical through carbon or nitrogen. Examples of C₃₋₆monoheterocycloalkyl groups include, but are not limited to, aziridinyl, oxiranyl, thiiranyl 1,1-dioxide, oxetanyl, azetidinyl, thietanyl 1,1-dioxide, pyrrolidinyl, tetrahydrofuranyl, piperidinyl, tetrahydro-2H-pyranyl, morpholinyl, thiomorpholinyl, and piperazinyl. Examples of C₈₋₁₂biheterocycloalkyl groups include, but are not limited to, 1,4-dioxaspiro[4.5]decanyl and 1,5-dioxaspiro[5.5]undecanyl.

The term “heterocycloalkenyl” refers to a partially unsaturated 3-6 membered monocyclic or 8-12 membered bicyclic ring system, referred to herein as C₃₋₆monoheterocycloalkenyl and C₈₋₁₂biheterocycloalkenyl, containing one to four independently selected heteroatoms, such as nitrogen, oxygen, and sulfur (including its oxidation states: S(O) or S(O)₂). Where possible, heterocycloalkenyl rings may be linked to the adjacent radical through carbon or nitrogen. For bicyclic heterocycloalkenyl groups: 1) either one or both rings may contain one or more double bonds and 2) the two rings may be attached through the same or different ring atoms. Examples of C₃₋₆monoheterocycloalkenyl groups include, but are not limited to, 2,3-dihydro-1H-pyrrolyl, 2,5-dihydro-1H-pyrrolyl, 4,5-dihydro-1H-pyrazolyl, 2,3-dihydro-1H-pyrazolyl, 4,5-dihydro-1H-imidazolyl, 2,3-dihydro-1H-imidazolyl, 2,3-dihydrothiophenyl, 2,5-dihydrothiophenyl, 4,5-dihydrothiazolyl, 2,3-dihydrothiazolyl, 4,5-dihydroisothiazolyl, 2,3-dihydroisothiazolyl, 2,3-dihydrofuranyl, 2,5-dihydrofuranyl, 4,5-dihydrooxazolyl, 2,3-dihydrooxazolyl, 4,5-dihydroisoxazolyl, 2,3-dihydroisoxazolyl, 3,4-dihydropyridinyl, 2,3-dihydropyridinyl, 2,3,4,5-tetrahydropyridinyl, 1,6-dihydropyridazinyl, 4,5-dihydropyridazinyl, 3,4,5,6-tetrahydropyridazinyl, 4,5-dihydropyrimidinyl, 1,2,5,6-tetrahydropyrimidinyl, 1,2-dihydropyrimidinyl, 1,2-dihydropyrazinyl, 2,3-dihydropyrazinyl, 1,2,3,6-tetrahydropyrazinyl, 4H-1,4-oxazinyl, 3,4-dihydro-2H-1,4-oxazinyl, 4H-1,4-thiazinyl, and 3,4-dihydro-2H-1,4-thiazinyl. Examples of C₈₋₁₂biheterocycloalkenyl groups include, but are not limited to, 6,7-dihydroindolyl, 4,5-dihydroindolyl, 7,8-dihydroimidazo[1,2-a]pyridinyl, 5,6-dihydroimidazo[1,2-a]pyridinyl, 4,5-dihydrobenzo[d]imidazolyl, 6,7-dihydro-1H-indazolyl, 4,5-dihydro-1H-indazolyl, 4,5-dihydropyrazolo[1,5-a]pyridinyl, and 6,7-dihydropyrazolo[1,5-a]pyridinyl.

The term “heterocyclyl” as used herein refers to a bicyclic ring system formed by either (1) fusing a phenyl ring to a 3-6 membered monocyclic heterocycloalkyl or 4-7 membered monocyclic heterocycloalkenyl ring, or (2) fusing a 5-6 membered monocyclic heteroaryl ring to a C₃₋₆ cycloalkyl, C₄₋₇ cycloalkenyl, 3-6 membered monocyclic heterocycloalkyl or 4-6 membered monocyclic heterocycloalkenyl ring. Where possible, the rings may be linked to the adjacent radical though carbon or nitrogen. Examples of heterocyclyls include, but are not limited to isochromanyl, 2H-quinolinyl, 6,7,8,9-tetrahydro-5H-[1,2,4]triazolo[4,3-a]azepine, 5,6,8,9-tetrahydro-[1,2,4]triazolo[4,3-d][1,4]oxazepane, 6,7-dihydro-5H,9H-[1,2,4]triazolo[3,4-c][1,4]oxazepane, 5,6,8,9-tetrahydro-712-[1,2,4]triazolo[4,3-d][1,4]diazepine, 8,9-dihydro-5H-[1,2,4]triazolo[4,3-a]azepine, 6,9-dihydro-5H-[1,2,4]triazolo[4,3-a]azepine, 5,6,7,8-tetrahydro-[1,2,4]triazolo[4,3-a]pyridine, 5,6-dihydro-8H-[1,2,4]triazolo[3,4-c][1,4]oxazine, 5,6,7,8-tetrahydroimidazo[1,2-a]pyridine, and 5H,9H-[1,2,4]triazolo[3,4-c][1,4]oxazepine.

The terms “hydroxy” and “hydroxyl” as used herein refers to the radical —OH.

The term “hydroxyalkyl” as used herein refers to an alkyl group substituted with one or more hydroxy groups. Examples include, but are not limited to, HOCH₂—, HOCH₂CH₂—, CH₃CH(OH)CH₂— and HOCH₂CH(OH)CH₂—.

The term “hydroxyalkoxy” as used herein refers to an alkoxy group substituted with one or more hydroxy groups. Examples include but are not limited to HOCH₂O—, HOCH₂CH₂O—, CH₃CH(OH)CH₂O— and HOCH₂CH(OH)CH₂O—.

The term “R^{a}R^{b}NC₁₋₆alkyl-,” as used herein refers to an alkyl group substituted with a R^{a}R^{b}N— group, as defined herein. Examples include but are not limited to NH₂CH₂—, NH(CH₃)CH₂—, N(CH₃)₂CH₂CH₂— and CH₃CH(NH₂)CH₂—.

The term “R^{a}R^{b}NC₁₋₆alkoxy,” as used herein refers to an alkoxy group substituted with a R^{a}R^{b}N— groups, as defined herein. Examples include but are not limited to NH₂CH₂—, NH(CH₃)CH₂O—, N(CH₃)₂CH₂CH₂O— and CH₃CH(NH₂)CH₂O—.

The term “oxo” as used herein refers to the radical =O.

As used herein, when a bicyclic ring is shown with a floating point of attachment and/or floating substituents, for example as in

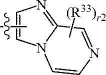

it signifies that the bicyclic ring can be attached via a carbon atom on either ring, and that the substituents (e.g., the R³³ group(s)) can be independently attached to either or both rings.

The terms “Individual,” “patient,” or “subject” are used interchangeably and include any animal, including mammals, preferably mice, rats, other rodents, rabbits, dogs, cats, swine, cattle, sheep, horses, or primates, and most preferably humans. The compounds or pharmaceutical compositions of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, dogs, primates, and the like). The mammal treated in the methods of the disclosure is desirably a mammal in which treatment of HBV infection is desired.

The term “modulation” includes antagonism (e.g., inhibition), agonism, partial antagonism and/or partial agonism.

The term “Pharmaceutically acceptable” include molecular entities and compositions that do not produce an adverse, allergic or other untoward reaction when administered to an animal, or a human, as appropriate. For human administration, preparations should meet sterility, pyrogenicity, and general safety and purity standards as required by FDA Office of Biologics standards.

The term “pharmaceutically acceptable carrier” or “pharmaceutically acceptable excipient” as used herein refers to any and all solvents, dispersion media, coatings, isotonic and absorption delaying agents, fillers, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable excipients.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, including, but not limited to, malate, oxalate, chloride, bromide, iodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming base salts with various pharmacologically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

The term “therapeutically effective amount” or “effective amount” as used herein refers to the amount of the subject compound that will elicit the biological or medical response of a tissue, system or animal, (e.g., mammal or human) that is being sought by the researcher, veterinarian, medical doctor or other clinician. The compounds or pharmaceutical compositions of the disclosure are administered in therapeutically effective amounts to treat a disease. Alternatively, a therapeutically effective amount of a compound is the quantity required to achieve a desired therapeutic and/or prophylactic effect.

The term “treating” includes any effect, e.g., lessening, reducing, modulating, or eliminating, via disruption of HBV core protein assembly, that results in the improvement of the disease. “Disruption” includes inhibition of HBV viral assembly and infection.

The compounds of the disclosure may contain one or more chiral centers and, therefore, exist as stereoisomers. The term “stereoisomers” when used herein consist of all enantiomers or diastereomers. These compounds may be designated by the symbols “(+),” “(-),” “R” or “S,” depending on the configuration of substituents around the stereogenic carbon atom, but the skilled artisan will recognize that a structure may denote a chiral center implicitly. The present disclosure encompasses various stereoisomers of these compounds and mixtures thereof. Mixtures of enantiomers or diastereomers may be designated “(±)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol

$$\overline{- - -}$$

denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic ring may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantiomeric and diastereoselective transformations and may involve the use of chiral auxiliaries. For examples, see Carreira and Kvaemo, Classics in Stereoselective Synthesis, Wiley-VCH: Weinheim, 2009.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively. For example, a compound of the disclosure may have one or more H atom replaced with deuterium.

Certain isotopically-labeled disclosed compounds (e.g., those labeled with ³H and ¹⁴C) are useful in compound and/or substrate tissue distribution assays. Tritiated (i.e., ³H) and carbon-14 (i.e., ¹⁴C) isotopes are particularly preferred for their ease of preparation and detectability. Further, substitution with heavier isotopes such as deuterium (i.e., ²H) may afford certain therapeutic advantages resulting from greater metabolic stability (e.g., increased in vivo half-life or reduced dosage requirements) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

The term “prodrug” refers to compounds that are transformed in vivo to yield a disclosed compound or a pharmaceutically acceptable salt, hydrate or solvate of the compound. The transformation may occur by various mechanisms (such as by esterase, amidase, phosphatase, oxidative and or reductive metabolism) in various locations (such as in the intestinal lumen or upon transit of the intestine, blood or liver). Prodrugs are well known in the art (for example, see Rautio, Kumpulainen, et al., Nature Reviews Drug Discovery 2008, 7, 255).

### II. 5-Membered Heteroaryl Carboxamide Compounds

In one aspect, the present disclosure provides a compound of Formula I

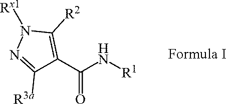

or a pharmaceutically acceptable salt thereof, wherein:
L is C₁₋₄alkylene or haloC₁₋₄alkylene;
L¹ is a bond, C₁₋₆alkylene, O, NR^{c}, C(O), C(O)O, C(O)NR^{c}, S(O)t or S(O)ₜNR^{c};
X³ is NR⁴ or CR⁴R⁸;
X⁴ is O or S;
X⁵ is O, S or NR⁰;
R^{a}, R^{b} and R^{c} are independently selected for each occurrence from the group consisting of hydrogen, C₁₋₆ alkyl, haloC₁₋₆ alkyl and C₃₋₆ monocycloalkyl;
R^{d} is hydrogen, OH, C₁₋₆ alkyl or C₁₋₆ alkoxy;
R^{x1} is hydrogen, C₁₋₄ alkyl, C₁₋₄ alkenyl, C₁₋₄ alkynyl, haloC₁₋₄ alkyl, or C₃₋₆ monocycloalkyl; or R^{x1} and R² together form a —CH₂CH₂CH₂—, —CH₂CH₂CH₂CH₂—, —CH₂CH₂O—, —CH₂OCH₂—, —CH₂CH₂CH₂O— —CH₂CH₂OCH₂—, —CH₂CH₂—NH— —CH₂NHCH₂—, —CH₂CH₂CH₂NH— or —CH₂CH₂NHCH₂— group;
R^{0a} is independently selected for each occurrence from the group consisting of halogen, OH, CN, NO₂, R^{a}R^{b}N—, C₁₋₄alkyl and haloC₁₋₅alkyl;
R^{4a} and R^{6b} are independently hydrogen or C₁₋₄ alkyl;
R⁰, R⁶ and R¹¹ are independently selected for each occurrence from the group consisting of halogen, OH, CN, NO₂, oxo, R^{d}N═, hydrazino, formyl, azido, silyl, siloxy, HOC(O)—, R^{a}R^{b}N—, R^{a}R^{b}NS(O)ₜ—, C₁₋₆alkyl, C₂₋₆alkenyl, C₂₋₆alkynyl, haloC₁₋₆alkyl, hydroxyC₁₋₆alkyl-, R^{a}R^{b}NC₁₋₆alkyl-, HOC(O)C₁₋₆alkyl-, R^{a}R^{b}NC₁₋₆alkylNR^{c}-, C₁₋₆alkylNR^{a}C₁₋₆alkylNR^{c}-, C₁₋₆alkoxy, haloC₁₋₆alkoxy, hydroxyC₁₋₆alkoxy-, R^{a}R^{b}NC₁₋₆alkoxy-, C₁₋₆alkoxyC₁₋₆alkyl-, haloC₁₋₆alkoxyC₁₋₆alkyl-, R^{a}R^{b}NC(O)—, C₁₋₆alkylC(O)-, C₁₋₆alkoxyC(O)-, C₁₋₆alkylC(O)O-, C₁₋₆alkylS(O)_{q}-, C₁₋₆alkylS(O)ₜNR^{c}-, C₁₋₆alkylS(O)ₜC₁₋₆alkyl-C₁₋₆alkylS(O)ₜNR^{a}C₁₋₆alkyl-, C₃₋₆cycloalkylS(O)ₜC₁₋₆alkyl-, C₁₋₆alkylC(O)C₁₋₆alkyl-, and C₁₋₆alkylC(O)OC₁₋₆alkyl-;
R¹ is a phenyl or 5-6 membered monocyclic heteroaryl, wherein the phenyl or 5-6 membered monocyclic heteroaryl is optionally substituted with one, two, or three independently selected R¹¹ groups;
R² and R⁸ are independently selected from the group consisting of hydrogen, halo, CN, OH, R^{a}R^{b}N, C₁₋₄alkyl, haloC₁₋₄alkyl, C₃₋₅monocycloalkyl, C₁₋₄alkoxy, and haloC₁₋₄alkoxy;
R^{3a} is

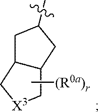

R⁴ is R⁵—L¹— or R⁹; or R⁴ and R⁸ together with the carbon atom to which they are attached form a

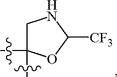

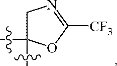

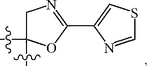

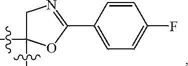

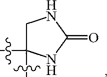

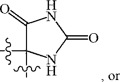

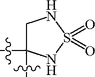

group;
R⁵ is

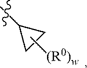

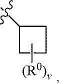

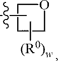

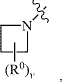

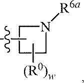

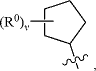

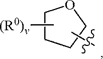

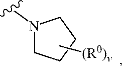

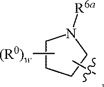

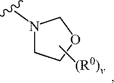

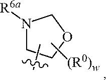

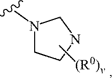

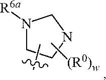

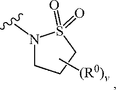

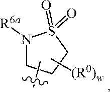

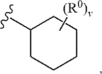

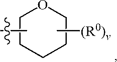

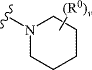

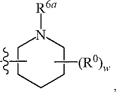

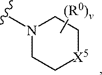

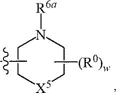

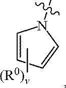

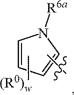

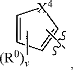

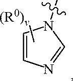

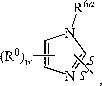

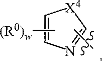

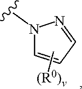

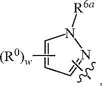

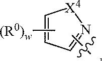

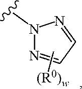

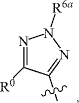

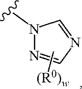

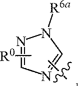

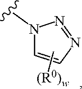

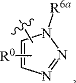

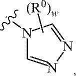

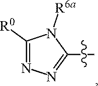

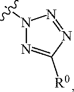

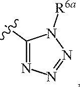

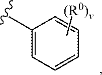

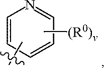

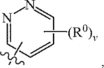

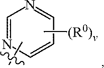

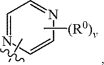

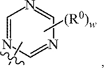

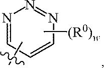

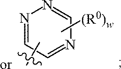

R⁹ is R¹⁴S(O)_{q}—L—, R¹⁴S(O)_{q}NH—L—, or R¹⁴C(O)NH—L—;
R¹⁴ is R^{a}R^{b}N—, C₁₋₆alkyl, C₂₋₆alkenyl, C₂₋₆alkynyl, C₁₋₆haloalkyl, C₁₋₆alkoxy, C₁₋₆haloalkyl, C₁₋₆haloalkoxy, or R⁵—L¹—;
q, r, t, and w are independently selected for each occurrence from 0, 1 and 2; and
v is independently selected for each occurrence from 0, 1, 2 and 3.

In another aspect, the present disclosure provides a compound of Formula Ia

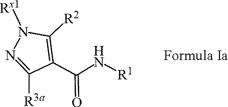

, or a pharmaceutically acceptable salt thereof, wherein:
L is C₁₋₄alkylene or haloC₁₋₄alkylene;
L¹ is a bond, C₁₋₆alkylene, O, NR^{c}, C(O), C(O)O, C(O)NR^{c}, S(O)t or S(O)ₜNR^{c};
X³ is NR⁴ or CR⁴R⁸;
X⁴ is O or S;
X⁵ is O, S or NR⁰;
R^{a}, R^{b} and R^{c} are independently selected for each occurrence from the group consisting of hydrogen, C₁₋₆ alkyl, haloC₁₋₆ alkyl and C₃₋₆ monocycloalkyl;
R^{d} is hydrogen, OH, C₁₋₆ alkyl or C₁₋₆ alkoxy;
R^{x1} is hydrogen, C₁₋₄ alkyl, C₁₋₄ alkenyl, C₁₋₄ alkynyl, haloC₁₋₄ alkyl, or C₃₋₆ monocycloalkyl; or R^{x1} and R² together form a —CH₂CH₂CH₂—, —CH₂CH₂CH₂CH₂—, —CH₂CH₂O—, —CH₂OCH₂—, —CH₂CH₂CH₂O— —CH₂CH₂OCH₂—, —CH₂CH₂—NH— —CH₂NHCH₂—, —CH₂CH₂CH₂NH— or —CH₂CH₂NHCH₂— group;
R^{0a} is independently selected for each occurrence from the group consisting of halogen, OH, CN, NO₂, R^{a}R^{b}N—, C₁₋₄alkyl and haloC₁₋₅alkyl;
R^{4a} and R^{6b} are independently hydrogen or C₁₋₄ alkyl;
R⁰, R⁶ and R¹¹ are independently selected for each occurrence from the group consisting of halogen, OH, CN, NO₂, oxo, R^{d}N═, hydrazino, formyl, azido, silyl, siloxy, HOC(O)—, R^{a}R^{b}N—, R^{a}R^{b}NS(O)ₜ—, C₁₋₆alkyl, C₂₋₆alkenyl, C₂₋₆alkynyl, haloC₁₋₆alkyl, hydroxyC₁₋₆alkyl-, R^{a}R^{b}NC₁₋₆alkyl-, HOC(O)C₁₋₆alkyl-, R^{a}R^{b}NC₁₋₆alkylNR^{c}-, C₁₋₆alkylNR^{a}C₁₋₆alkylNR^{c}-, C₁₋₆alkoxy, haloC₁₋₆alkoxy, hydroxyC₁₋₆alkoxy-, R^{a}R^{b}NC₁₋₆alkoxy-, C₁₋₆alkoxyC₁₋₆alkyl-, haloC₁₋₆alkoxyC₁₋₆alkyl-, R^{a}R^{b}NC(O)—, C₁₋₆alkylC(O)-, C₁₋₆alkoxyC(O)-, C₁₋₆alkylC(O)O-, C₁₋₆alkylS(O)_{q}-, C₁₋₆alkylS(O)ₜNR^{c}-, C₁₋₆alkylS(O)ₜC₁₋₆alkyl-C₁₋₆alkylS(O)ₜNR^{a}C₁₋₆alkyl-, C₃₋₆cycloalkylS(O)ₜC₁₋₆alkyl-, C₁₋₆alkylC(O)C₁₋₆alkyl-, and C₁₋₆alkylC(O)OC₁₋₆alkyl-;
R¹ is a phenyl or 5-6 membered monocyclic heteroaryl, wherein the phenyl or 5-6 membered monocyclic heteroaryl is optionally substituted with one, two, or three independently selected R¹¹ groups;
R² and R⁸ are independently selected from the group consisting of hydrogen, halo, CN, OH, R^{a}R^{b}N, C₁₋₄alkyl, haloC₁₋₄alkyl, C₃₋₅monocycloalkyl, C₁₋₄alkoxy, and haloC₁₋₄alkoxy;
R^{3a} is

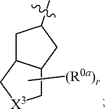

R⁴ is R⁵—L¹—, R⁶, or R⁹; or R⁴ and R⁸ together with the carbon atom to which they are attached form a

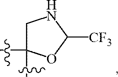

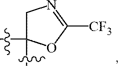

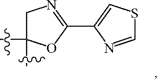

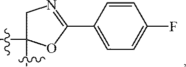

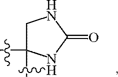

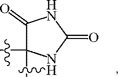

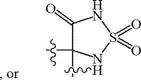

group;

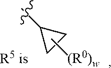

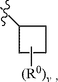

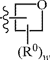

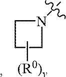

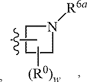

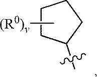

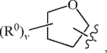

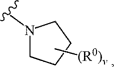

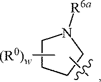

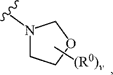

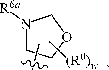

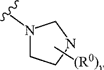

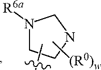

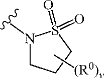

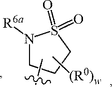

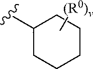

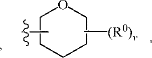

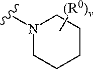

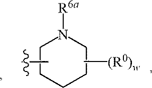

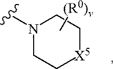

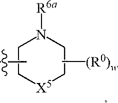

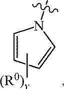

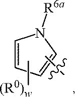

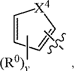

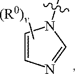

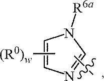

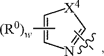

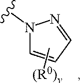

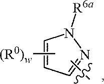

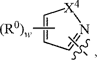

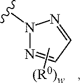

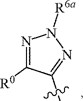

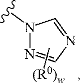

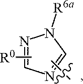

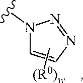

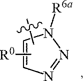

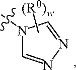

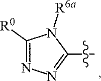

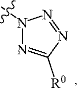

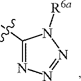

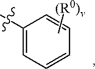

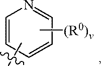

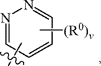

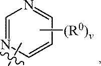

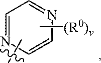

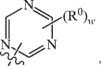

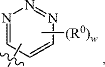

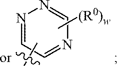

R⁹ is R¹⁴S(O)_{q}—L—, R¹⁴S(O)_{q}NH—L—, or R¹⁴C(O)NH—L—;
R¹⁴ is R^{a}R^{b}N—, C₁₋₆alkyl, C₂₋₆alkenyl, C₂₋₆alkynyl, C₁₋₆haloalkyl, C₁₋₆alkoxy, C₁₋₆haloalkyl, C₁₋₆haloalkoxy, or R⁵—L¹—;
q, r, t, and w are independently selected for each occurrence from 0, 1 and 2; and
v is independently selected for each occurrence from 0, 1, 2 and 3.

The following embodiments further describe a compound of Formula I or Formula Ia, or a pharmaceutically acceptable salt thereof. It will be appreciated that all chemically allowable combinations of the embodiments described herein are envisioned as further embodiments of the invention.

In certain embodiments, R^{x1} is hydrogen of methyl.

In certain embodiments, R^{x1} is methyl.

In certain embodiments, L¹ is a bond.

In certain embodiments, L¹ is a C₁₋₆alkylene.

In certain embodiments, X³ is NR⁴.

In certain embodiments, X³ is CR⁴R⁸.

In certain embodiments, r is 0.

In certain embodiments, R¹ is

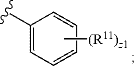

; R¹¹ is independently selected for each occurrence from the group consisting of halogen, CN, C₁₋₆alkyl and haloC₁₋₆alkyl; and z1 is 0, 1, 2 or 3.

In certain embodiments, R¹¹ is independently selected for each occurrence from the group consisting of halogen and CN.

In certain embodiments, R¹¹ is independently selected for each occurrence from the group consisting of F, Cl, Br and I.

In certain embodiments, R¹ is selected from the group consisting of:

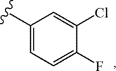

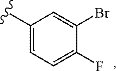

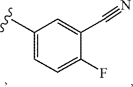

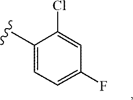

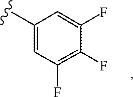

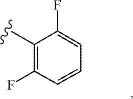

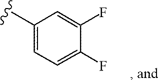

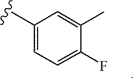

In certain embodiments, R¹ is

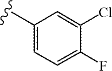

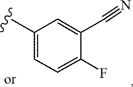

In certain embodiments, R¹ is

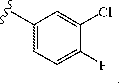

In certain embodiments, R^{x1} is hydrogen or methyl and R¹ is

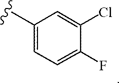

In certain embodiments, R¹ is a 5-6 membered monocyclic heteroaryl optionally substituted with one, two, or three substituents independently selected from the group consisting of halogen, CN, C₁₋₆alkyl, and haloC₁₋₆alkyl.

In certain embodiments, R¹ is

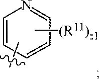

R¹¹ is independently selected for each occurrence from the group consisting of halogen, CN, C₁₋₆alkyl and haloC₁₋₆alkyl; and z1 is 0, 1, 2 or 3.

In certain embodiments, R² is R^{a}R^{b}N.

In certain embodiments, R² is R^{a}R^{b}N, and R^{a} and R^{b} are independently selected the group consisting of hydrogen and C₁₋₆alkyl.

In certain embodiments, R² is NH₂.

In certain embodiments, R^{x1} is hydrogen or methyl, R¹ is

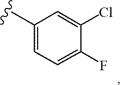

and R² is NH₂.

In certain embodiments, R^{x1} is hydrogen or methyl, R¹ is

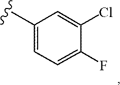

R² is NH₂; and r is 0.

In certain embodiments, R^{3a} is

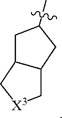

In certain embodiments, R^{3a} is

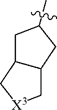

and X3 is NR⁴.

In certain embodiments, R^{3a} is

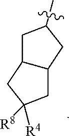

In certain embodiments, R^{3a} is

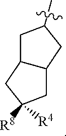

In certain embodiments, R^{3a} is

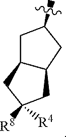

In certain embodiments, R⁴ is R⁵—L¹—.

In certain embodiments, R⁴ is R⁵.

In certain embodiments, R⁴ is R⁶.

In certain embodiments, R⁴ is R⁹.

In certain embodiments, or R⁴ and R⁸ together with the carbon atom to which they are attached form a

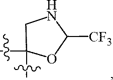

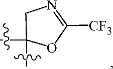

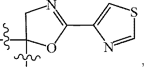

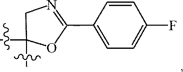

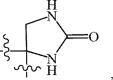

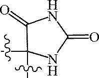

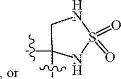

group;

In certain embodiments, R⁵ is

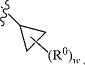

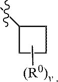

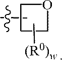

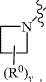

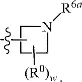

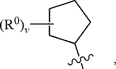

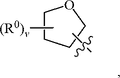

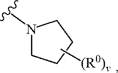

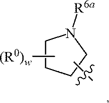

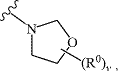

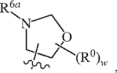

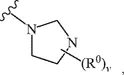

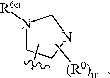

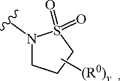

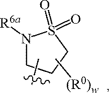

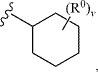

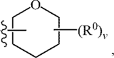

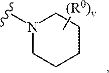

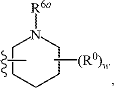

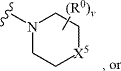

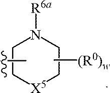

In certain embodiments, R⁵ is

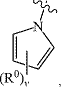

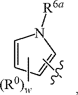

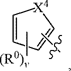

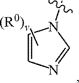

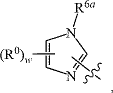

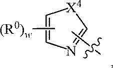

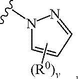

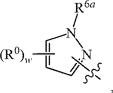

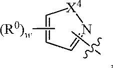

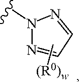

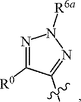

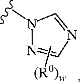

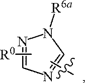

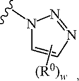

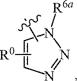

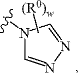

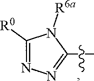

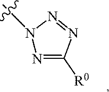

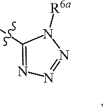

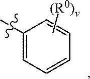

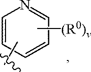

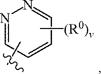

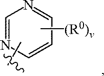

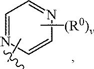

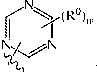

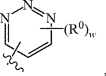

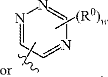

In certain embodiments, R⁵ is

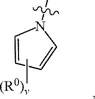

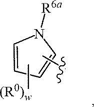

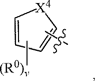

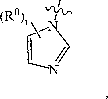

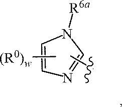

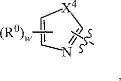

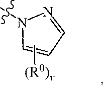

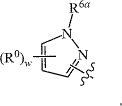

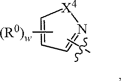

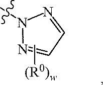

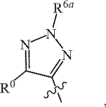

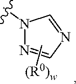

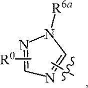

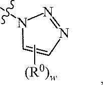

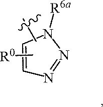

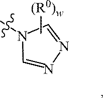

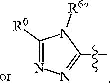

In certain embodiments, R⁵ is

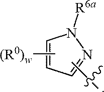

In certain embodiments, R⁸ is hydrogen, OH or C₁₋₆alkoxy.

In certain embodiments, and R⁸ is hydrogen.

In certain embodiments, and R⁸ is OH.

In certain embodiments, R¹⁴ is R^{a}R^{b}N—, C₁₋₆alkyl, C₂₋₆alkenyl, C₂₋₆alkynyl, C₁₋₆haloalkyl, C₁₋₆alkoxy, C₁₋₆haloalkyl, or C₁₋₆haloalkoxy.

In certain embodiments, R¹⁴ is R⁵—L¹—.

In certain embodiments, R¹⁴ is R⁵.

In certain embodiments, R^{x1} is hydrogen or methyl; R¹ is

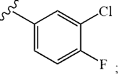

R² is NH₂; X³ is CR⁴R⁸; and R⁸ is hydrogen, OH or C₁₋₆alkoxy.

In certain embodiments, R^{x1} is hydrogen or methyl, R¹ is

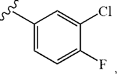

R² is NH₂, X³ is CR⁴R⁸, and R⁸ is OH.

In certain embodiments, R^{x1} is hydrogen or methyl; R¹ is

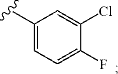

; R² is NH₂; X³ is CR⁴R⁸; R⁸ is hydrogen, OH or C₁₋₆alkoxy; and r is 0.

In certain embodiments, R^{x1} is hydrogen or methyl, R¹ is

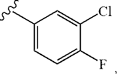

R² is NH₂, X³ is CR⁴R⁸, R⁸ is OH, and r is 0.

In certain embodiments, R^{x1} is hydrogen or methyl; R¹ is

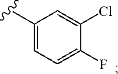

R² is NH₂; and X³ is NR⁴.

In certain embodiments, R^{x1} is hydrogen or methyl; R¹ is

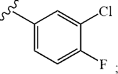

; R² is NH₂; X³ is NR⁴; and r is 0.

In another aspect, the present disclosure provides a compound of Formula II

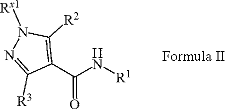

, or a pharmaceutically acceptable salt thereof, wherein:
L is C₁₋₄alkylene or haloC₁₋₄alkylene;
L¹ is a bond, C₁₋₆alkylene, O, NR^{c}, C(O), C(O)O, C(O)NR^{c}, S(O)t or S(O)ₜNR^{c};
X³ is O, NR⁴, CR⁴R⁸, C(O) or S(O)t;
X⁴ is O or S;
X⁵ is O, S or NR⁰;
R^{x1} is hydrogen, C₁₋₄ alkyl, C₁₋₄ alkenyl, C₁₋₄ alkynyl, haloC₁₋₄ alkyl, or C₃₋₆ monocycloalkyl; or R^{x1} and R² together form a —CH₂CH₂CH₂—, —CH₂CH₂CH₂CH₂—, —CH₂CH₂O—, —CH₂OCH₂—, —CH₂CH₂CH₂O— —CH₂CH₂OCH₂—, —CH₂CH₂—NH— —CH₂NHCH₂—, —CH₂CH₂CH₂NH— or —CH₂CH₂NHCH₂— group;
R^{a}, R^{b} and R^{c} are independently selected for each occurrence from the group consisting of hydrogen, C₁₋₆ alkyl, haloC₁₋₆ alkyl and C₃₋₆ monocycloalkyl;
R^{d} is hydrogen, OH, C₁₋₆ alkyl or C₁₋₆ alkoxy;
R^{0a} is independently selected for each occurrence from the group consisting of hydrogen, halogen, OH, CN, NO₂, R^{a}R^{b}N—, C₁₋₄alkyl and haloC₁₋₄alkyl;
R^{4a} and R^{6a} are independently hydrogen or C₁₋₄ alkyl;
R⁰, R⁶ and R¹¹ are independently selected for each occurrence from the group consisting of hydrogen, halogen, OH, CN, NO₂, oxo, R^{d}N═, hydrazino, formyl, azido, silyl, siloxy, HOC(O)—, R^{a}R^{b}N—, R^{a}R^{b}NS(O)ₜ—, C₁₋₆alkyl, C₂₋₆alkenyl, C₂₋₆alkynyl, haloC₁₋₆alkyl, hydroxyC₁₋₆alkyl-, R^{a}R^{b}NC₁₋₆alkyl-, HOC(O)C₁₋₆alkyl-, R^{a}R^{b}NC₁₋₆alkylNR^{c}-, C₁₋₆alkylNR^{a}C₁₋₆alkylNR^{c}-, C₁₋₆alkoxy, haloC₁₋₆alkoxy, hydroxyC₁₋₆alkoxy-, R^{a}R^{b}NC₁₋₆alkoxy-, C₁₋₆alkoxyC₁₋₆alkyl-, haloC₁₋₆alkoxyC₁₋₆alkyl-, R^{a}R^{b}NC(O)—, C₁₋₆alkylC(O)-, C₁₋₆alkoxyC(O)-, C₁₋₆alkylC(O)O-, C₁₋₆alkylS(O)_{q}-, C₁₋₆alkylS(O)ₜNR^{c}-, C₁₋₆alkylS(O)ₜC₁₋₆alkyl-C₁₋₆alkylS(O)ₜNR^{a}C₁₋₆alkyl-, C₃₋₆cycloalkylS(O)ₜC₁₋₆alkyl-, C₁₋₆alkylC(O)C₁₋₆alkyl-, and C₁₋₆alkylC(O)OC₁₋₆alkyl-;
R¹ is a phenyl or 5-6 membered monocyclic heteroaryl, wherein the phenyl or 5-6 membered monocyclic heteroaryl is optionally substituted with one, two, or three independently selected R¹¹ groups;
R² and R⁸ are independently selected from the group consisting of hydrogen, halo, CN, OH, R^{a}R^{b}N, C₁₋₄alkyl, haloC₁-₄alkyl, C₃₋₅monocycloalkyl, C₁-₄alkoxy, and haloC₁₋₄alkoxy;

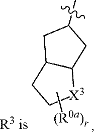

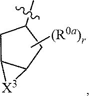

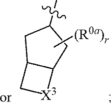

R⁴ is R⁵—L¹— or R⁹; or R⁴ and R⁸ together with the carbon atom to which they are attached form a

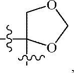

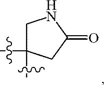

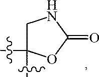

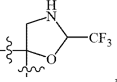

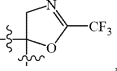

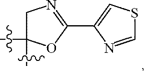

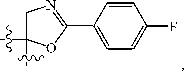

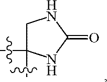

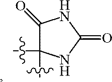

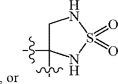

group;

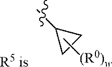

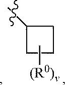

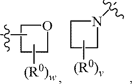

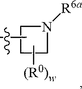

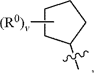

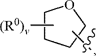

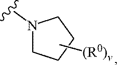

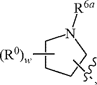

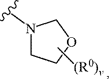

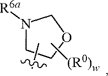

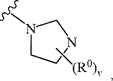

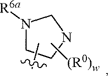

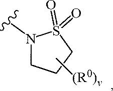

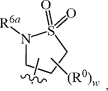

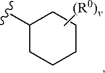

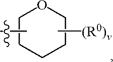

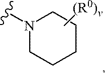

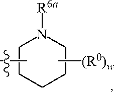

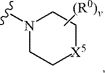

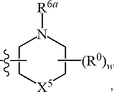

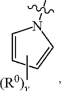

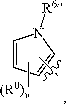

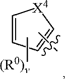

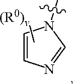

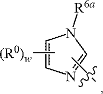

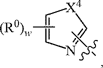

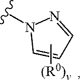

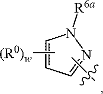

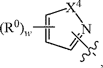

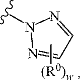

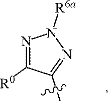

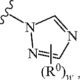

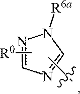

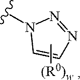

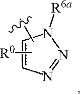

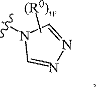

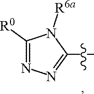

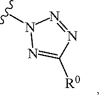

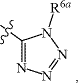

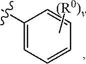

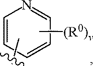

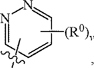

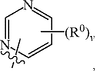

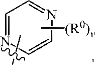

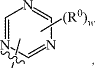

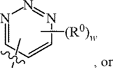

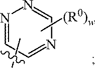

R⁹ is R¹⁴S(O)_{q}—L—, R¹⁴S(O)_{q}NH—L—, or R¹⁴C(O)NH—L—;
R¹⁴ is R^{a}R^{b}N—, C₁₋₆alkyl, C₂₋₆alkenyl, C₂₋₆alkynyl, C₁-₆haloalkyl, C₁₋₆alkoxy, C₁₋₆haloalkyl, C₁₋₆haloalkoxy, or R⁵—L¹—;
q, r, t, and w are independently selected for each occurrence from 0, 1 and 2; and
v is independently selected for each occurrence from 0, 1, 2 and 3.

In another aspect, the present disclosure provides a compound of Formula IIa

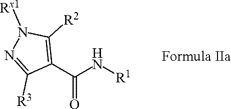

, or a pharmaceutically acceptable salt thereof, wherein:
L is C₁₋₄alkylene or haloC₁₋₄alkylene;
L¹ is a bond, C₁₋₆alkylene, O, NR^{c}, C(O), C(O)O, C(O)NR^{c}, S(O)t or S(O)ₜNR^{c};
X³ is O, NR⁴, CR⁴R⁸, C(O) or S(O)t;
X⁴ is O or S;
X⁵ is O, S or NR⁰;
R^{x1} is hydrogen, C₁-₄ alkyl, C₁-₄ alkenyl, C₁-₄ alkynyl, haloC₁₋₄ alkyl, or C₃₋₆ monocycloalkyl; or R^{X1} and R² together form a —CH₂CH₂CH₂—, —CH₂CH₂CH₂CH₂—, —CH₂CH₂O—, —CH₂OCH₂—, —CH₂CH₂CH₂O— —CH₂CH₂OCH₂—, —CH₂CH₂—NH— —CH₂NHCH₂—, —CH₂CH₂CH₂NH— or —CH₂CH₂NHCH₂— group;
R^{a}, R^{b} and R^{c} are independently selected for each occurrence from the group consisting of hydrogen, C₁₋₆ alkyl, haloC₁₋₆ alkyl and C₃₋₆ monocycloalkyl;
R^{d} is hydrogen, OH, C₁₋₆ alkyl or C₁₋₆ alkoxy;
R^{0a} is independently selected for each occurrence from the group consisting of hydrogen, halogen, OH, CN, NO₂, R^{a}R^{b}N—, C₁₋₄alkyl and haloC₁-₄alkyl;
R^{4a} and R^{6a} are independently hydrogen or C₁₋₄ alkyl;
R⁰, R⁶ and R¹¹ are independently selected for each occurrence from the group consisting of hydrogen, halogen, OH, CN, NO₂, oxo, R^{d}N═, hydrazino, formyl, azido, silyl, siloxy, HOC(O)—, R^{a}R^{b}N—, R^{a}R^{b}NS(O)t—, C₁₋₆alkyl, C₂₋₆alkenyl, C₂₋₆alkynyl, haloC₁₋₆alkyl, hydroxyC₁₋₆alkyl-, R^{a}R^{b}NC₁₋₆alkyl-, HOC(O)C₁₋₆alkyl-, RaRbNC₁₋₆alkylNR^{c}-, C₁₋₆alkylNR^{a}C₁₋₆alkylNR^{c}-, C₁-₆alkoxy, haloC₁₋₆alkoxy, hydroxyC₁₋₆alkoxy-, R^{a}R^{b}NC₁₋₆alkoxy-, C₁₋₆alkoxyC₁₋₆alkyl-, haloC₁₋₆alkoxyC₁₋₆alkyl-, R^{a}R^{b}NC(O)—, C₁₋₆alkylC(O)-, C₁₋₆alkoxyC(O)-, C₁₋₆alkylC(O)O-, C₁₋₆alkylS(O)_{q}-, C₁₋₆alkylS(O)tNR^{c}-, C₁₋₆alkylS(O)ₜC₁₋₆alkyl-C₁₋₆alkylS(O)ₜNR^{a}C₁₋₆alkyl-, C₃₋₆cycloalkylS(O)tC₁₋₆alkyl-, C₁₋₆alkylC(O)C₁₋₆alkyl-, and C₁-₆alkylC(O)OC₁₋₆alkyl-;
R¹ is a phenyl or 5-6 membered monocyclic heteroaryl, wherein the phenyl or 5-6 membered monocyclic heteroaryl is optionally substituted with one, two, or three independently selected R¹¹ groups;
R² and R⁸ are independently selected from the group consisting of hydrogen, halo, CN, OH, R^{a}R^{b}N, C₁₋₄alkyl, haloC₁-₄alkyl, C₃₋₅monocycloalkyl, C₁-₄alkoxy, and haloC₁₋₄alkoxy;

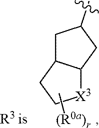

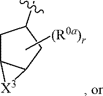

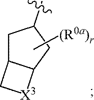

R⁴ is R⁵—L¹—, R⁶, or R⁹; or R⁴ and R⁸ together with the carbon atom to which they are attached form a

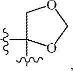

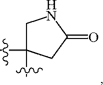

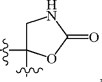

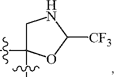

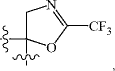

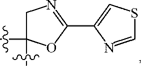

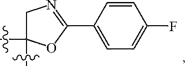

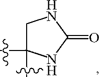

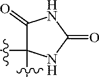

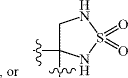

group;

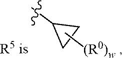

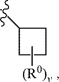

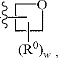

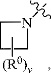

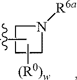

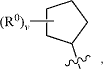

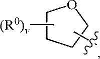

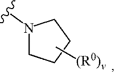

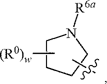

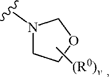

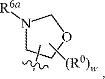

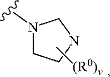

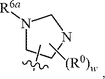

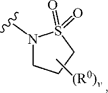

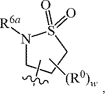

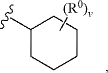

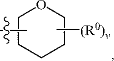

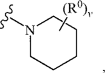

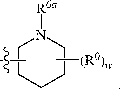

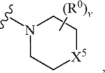

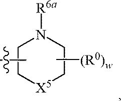

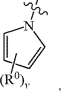

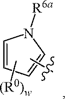

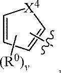

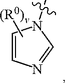

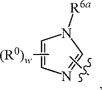

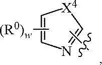

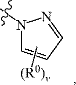

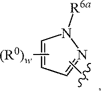

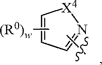

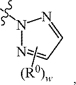

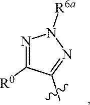

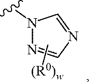

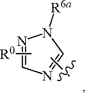

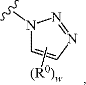

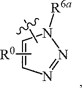

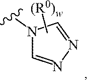

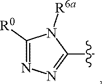

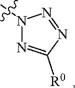

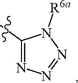

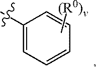

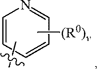

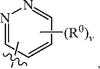

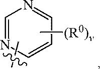

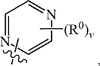

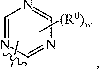

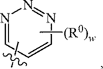

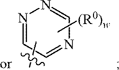

R⁹is R¹⁴S(O)_{q}—L—, R¹⁴S(O)_{q}NH—L—, or R¹⁴C(O)NH—L—;
R¹⁴ is R^{a}R^{b}N—, C₁₋₆alkyl, C₂₋₆alkenyl, C₂₋₆alkynyl, C₁₋₆haloalkyl, C₁₋₆alkoxy, C₁₋₆haloalkyl, C₁-₆haloalkoxy, or R⁵—L¹—;
q, r, t, and w are independently selected for each occurrence from 0, 1 and 2; and
v is independently selected for each occurrence from 0, 1, 2 and 3.

The following embodiments further describe a compound of Formula II of IIa, or a pharmaceutically acceptable salt thereof. It will be appreciated that all chemically allowable combinations of the embodiments described herein are envisioned as further embodiments of the invention.

In certain embodiments, R^{X1} is hydrogen of methyl.

In certain embodiments, R^{X1} is methyl.

In certain embodiments, L¹ is a bond.

In certain embodiments, L¹ is a C₁₋₆alkylene.

In certain embodiments, X³ is CR⁴R⁸.

In certain embodiments, r is 0.

In certain embodiments, R¹ is

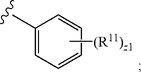

R¹¹ is independently selected for each occurrence from the group consisting of halogen, CN, C₁₋₆alkyl and haloC₁₋₆alkyl; and z1 is 0, 1, 2 or 3.

In certain embodiments, R¹¹ is independently selected for each occurrence from the group consisting of halogen and CN.

In certain embodiments, R¹¹ is independently selected for each occurrence from the group consisting of F, Cl, Br and I.

In certain embodiments, R¹ is selected from the group consisting of:

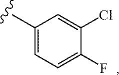

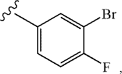

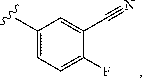

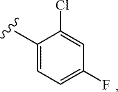

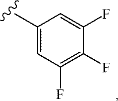

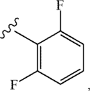

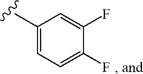

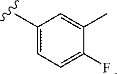

In certain embodiments, R¹ is

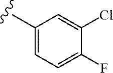

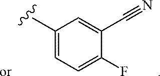

In certain embodiments, R¹ is

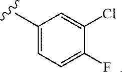

In certain embodiments, R^{X1} is hydrogen or methyl and R¹ is

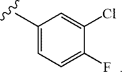

In certain embodiments, R¹ is a 5-6 membered monocyclic heteroaryl optionally substituted with one, two, or three substituents independently selected from the group consisting of halogen, CN, C₁₋₆alkyl, and haloC₁₋₆alkyl.

In certain embodiments, R¹ is

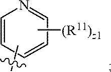

; R¹¹ is independently selected for each occurrence from the group consisting of halogen, CN, C₁₋₆alkyl and haloC₁₋₆alkyl; and z1 is 0, 1, 2 or 3.

In certain embodiments, R² is R^{a}R^{b}N;

In certain embodiments, R² is R^{a}R^{b}N, and R^{a} and R^{b} are independently selected the group consisting of hydrogen and C₁₋₆alkyl.

In certain embodiments, R² is NH₂.

In certain embodiments, R^{x1} is hydrogen or methyl, R¹ is

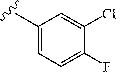

, and R² is NH₂.

In certain embodiments, R³ is

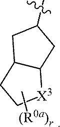

In certain embodiments, R³ is

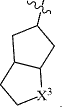

In certain embodiments, R³ is

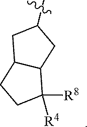

In certain embodiments, R³ is

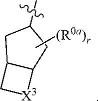

In certain embodiments, R³ is

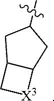

In certain embodiments, R³ is

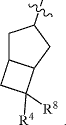

In certain embodiments, R⁴ is R⁵—L¹—.

In certain embodiments, and R⁴ is R⁵.

In certain embodiments, R⁴ is R⁶.

In certain embodiments, R⁴ is R⁹.

In certain embodiments, R⁴ and R⁸ together with the carbon atom to which they are attached form a

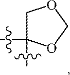

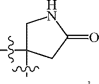

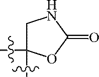

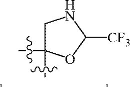

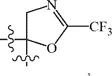

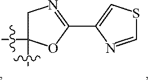

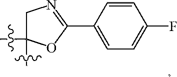

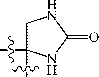

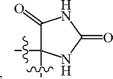

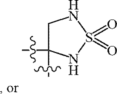

group;

In certain embodiments, R⁵ is

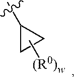

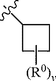

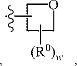

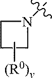

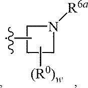

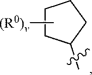

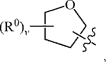

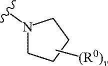

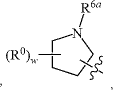

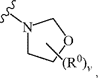

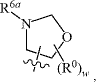

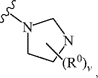

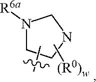

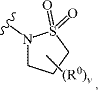

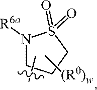

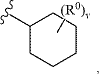

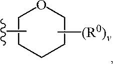

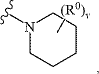

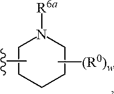

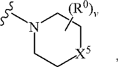

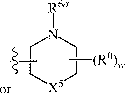

In certain embodiments, R⁵ is

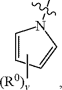

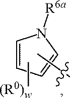

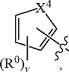

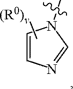

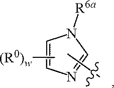

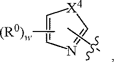

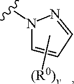

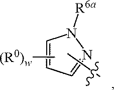

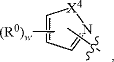

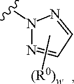

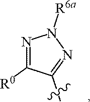

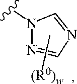

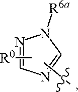

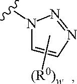

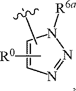

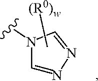

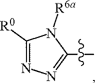

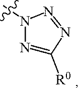

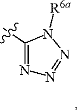

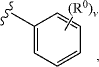

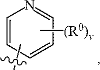

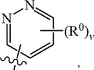

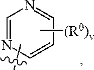

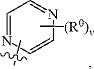

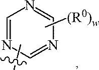

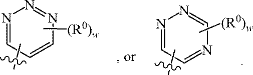

In certain embodiments, R⁵ is

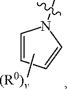

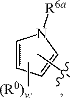

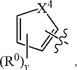

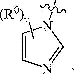

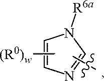

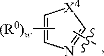

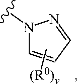

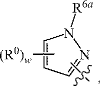

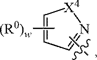

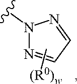

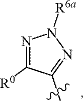

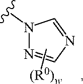

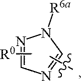

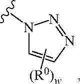

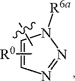

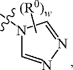

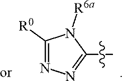

In certain embodiments, R⁵ is

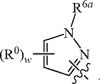

In certain embodiments, R⁶ is C₁₋₆alkylS(O)ₜC₁₋₆alkyl- or C₁₋₆alkylS(O)ₜNR^{a}C₁₋₆alkyl-.

In certain embodiments, R⁸ is hydrogen, OH or C₁₋₆alkoxy.

In certain embodiments, R⁸ is hydrogen.

In certain embodiments, R⁸ is OH.

In certain embodiments, R¹⁴ is R^{a}R^{b}N—, C₁₋₆alkyl, C₂₋₆alkenyl, C₂₋₆alkynyl, C₁₋₆haloalkyl, C₁₋₆alkoxy, C₁₋₆haloalkyl, or C₁₋₆haloalkoxy.

In certain embodiments, R¹⁴ is R⁵—L¹—.

In certain embodiments, R¹⁴ is R⁵.

In certain embodiments, R^{x1} is hydrogen or methyl; R¹ is

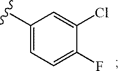

R² is NH₂;

R³ is

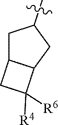

; R⁶ is C₁₋₆alkylS(O)ₜC₁₋₆alkyl- or C₁₋₆alkylS(O)ₜNR^{a}C₁₋₆alkyl-; R⁸ is hydrogen, OH or C₁₋₆alkoxy.

In certain embodiments, R^{x1} is hydrogen or methyl; R¹ is

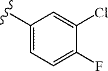

; R² is NH₂;

R³ is

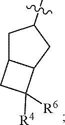

R⁶ is C₁₋₆alkylS(O)ₜC₁₋₆alkyl- or C₁₋₆alkylS(O)ₜNR^{a}C₁₋₆alkyl-; and R⁸ is OH.

### IV. Pharmaceutical Compositions and Kits

In another aspect, the disclosure provides pharmaceutical compositions comprising a compound of Formula I or II, or a pharmaceutically acceptable salt thereof, and a pharmaceutically acceptable excipient. In particular, the present disclosure provides pharmaceutical compositions comprising compounds as disclosed herein formulated together with one or more pharmaceutically acceptable carriers. These formulations include those suitable for oral, rectal, topical, buccal, parenteral (e.g., subcutaneous, intramuscular, intradermal, or intravenous), rectal, vaginal, or aerosol administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

In another aspect, the disclosure provides a pharmaceutical composition comprises a compound of Table 1 or 2, or a pharmaceutically acceptable salt and/or stereoisomer thereof.

Exemplary pharmaceutical compositions of this disclosure may be used in the form of a pharmaceutical preparation, for example, in solid, semisolid or liquid form, which contains one or more compounds of the disclosure, as an active ingredient, in admixture with an organic or inorganic carrier or excipient suitable for external, enteral or parenteral applications. The active ingredient may be compounded, for example, with the usual nontoxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a nontoxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable nonirritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants.

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methyacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Advantageously, the disclosure also provides kits for use by e.g., a consumer in need of HBV infection treatment. Such kits include a suitable dosage form such as those described above and instructions describing the method of using such dosage form tomediate, reduce or prevent HBV infection. The instructions would direct the consumer or medical personnel to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, ... etc.... Second Week, Monday, Tuesday, ...” etc. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

### IV. Methods

In a further aspect, a method for treating a hepatitis B infection in a patient in need thereof is provided, comprising administering to a subject or patient an effective amount of a disclosed compound, and/or administering a first disclosed compound and optionally, an additional, different disclosed compound(s). In another embodiment, a method for treating a hepatitis B infection in a patient in need thereof is provided, comprising administering to a subject or patient a therapeutically effective amount of a disclosed pharmaceutical composition or a pharmaceutical composition comprising a disclosed compound, or two or more disclosed compounds, and a pharmaceutically acceptable excipient.

For use in accordance with this aspect, the appropriate dosage is expected to vary depending on, for example, a particular compound employed, the mode of administration, and the nature and severity of the infection to be treated as well as the specific infection to be treated and is within the purview of the treating physician. Usually, an indicated administration dose may be in the range between about 0.1 to about 1000 µg/kg body weight. In some cases, the administration dose of the compound may be less than 400 µg/kg body weight. In other cases, the administration dose may be less than 200 µg/kg body weight. In yet other cases, the administration dose may be in the range between about 0.1 to about 100 µg/kg body weight. The dose may be conveniently administered once daily, or in divided doses up to, for example, four times a day or in sustained release form.

A compound of the present disclosure may be administered by any conventional route, in particular: enterally, topically, orally, nasally, e.g., in the form of tablets or capsules, via suppositories, or parenterally, e.g., in the form of injectable solutions or suspensions, for intravenous, intra-muscular, sub-cutaneous, or intra-peritoneal injection. Suitable formulations and pharmaceutical compositions will include those formulated in a conventional manner using one or more physiologically acceptable carriers or excipients, and any of those known and commercially available and currently employed in the clinical setting. Thus, the compounds may be formulated for oral, buccal, topical, parenteral, rectal or transdermal administration or in a form suitable for administration by inhalation or insufflation (either orally or nasally).

For oral administration, pharmaceutical compositions may take the form of, for example, tablets or capsules prepared by conventional means with pharmaceutically acceptable excipients such as binding agents (e.g., 44ecarbonate44ed maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycollate); or wetting agents (e.g., sodium lauryl sulphate). Tablets may be coated by methods well known in the art. Liquid preparations for oral administration may take the form of, for example, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol or fractionated vegetable oils); and preservatives (e.g., methyl or propyl-p-hydroxybenzoates or sorbic acid). Preparations may also contain buffer salts, flavoring, coloring and sweetening agents as appropriate.

Preparations for oral administration may also be suitably formulated to give controlled-release or sustained release of the active compound(s) over an extended period. For buccal administration the compositions may take the form of tablets or lozenges formulated in a conventional manner known to the skilled artisan.

A disclosed compound may also be formulated for parenteral administration by injection e.g., by bolus injection or continuous infusion. Formulations for injection may be presented in unit dosage form e.g., in ampoules or in multi-dose containers, with an added preservative. The compositions may take such forms as suspensions, solutions or emulsions in oily or aqueous vehicles, and may contain additives such as suspending, stabilizing and/or dispersing agents. Alternatively, the compound may be in powder form for constitution with a suitable vehicle, e.g., sterile pyrogen-free water, before use. Compounds may also be formulated for rectal administration as suppositories or retention enemas, e.g., containing conventional suppository bases such as cocoa butter or other glycerides.

Also contemplated herein are methods and compositions that include a second active agent or administering a second active agent. For example, in addition to being infected with HBV, a subject or patient can further have HBV infection-related co-morbidities, i.e., diseases and other adverse health conditions associated with, exacerbated by, or precipitated by being infected with HBV. Contemplated herein are disclosed compounds in combination with at least one other agent that has previously been shown to treat these HBV-infection-related conditions.

In some cases, a disclosed compound may be administered as part of a combination therapy in conjunction with one or more antivirals. Example antivirals include nucleoside analogs, interferon α, and other assembly effectors, for instance
heteroaryldihydropyrimidines (HAPs) such as methyl 4-(2-chloro-4-fluorophenyl)-6-methyl-2-(46ecarbon-2-yl)-1,4-dihydropyrimidine-5-carboxylate (HAP-1). For example, provided herein is a method of treating a patient suffering from hepatitis B infection comprising administering to the patient a first amount of a disclosed compound and a second amount of an antiviral, or other anti HBV agent, for example a second amount of a second compound selected from the group consisting of: a HBV capsid assembly promoter (for example, GLS4, BAY 41-4109, AT-130, DVR-23 (e.g., as depicted below),

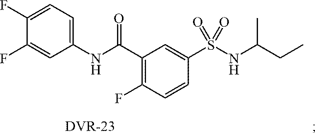

NVR 3-778, NVR1221 (by code); and N890 (as depicted below):

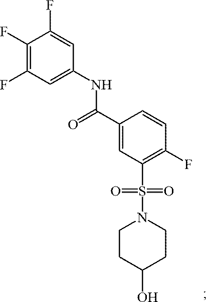

other capsid inhibitors such as those disclosed in the following patent applications hereby incorporated by reference: WO2014037480, WO2014184328, WO2013006394, WO2014089296, WO2014106019, WO2013102655, WO2014184350, WO2014184365, WO2014161888, WO2014131847, WO2014033176, WO2014033167, and WO2014033170; Nucleos(t)ide analogs interfering with viral polymerase, such as entecavir (Baraclude), Lamivudine, (Epivir-HBV), Telbivudine (Tyzeka, Sebivo), Adefovir dipivoxil (Hepsera), Tenofovir (Viread), Tenofovir alafenamide fumarate (TAF), prodrugs of tenofavir (e.g. AGX-1009), L-FMAU (Clevudine), LB80380 (Besifovir) and:

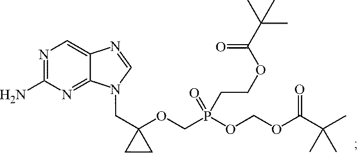

viral entry inhibitors such as Myrcludex B and related lipopeptide derivatives; HbsAg secretion inhibitors such as REP 9AC′ and related nucleic acid-based amphipathic polymers, HBF-0529 (PBHBV-001), PBHBV-2-15 as depicted below:

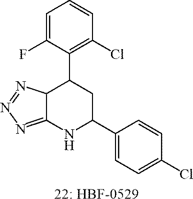

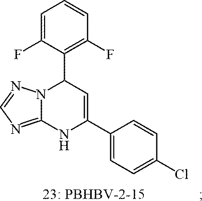

and BM601 as depicted below:

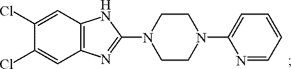

disruptors of nucleocapsid formation or integrity such as NZ-4/W28F:

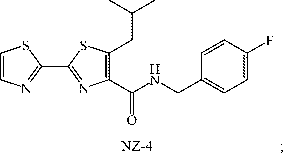

cccDNA formation inhibitors such as BSBI-25, CCC-0346, CCC-0975 (as depicted below):

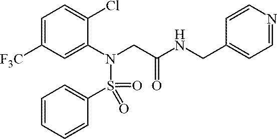 [SMILES: O=C(CN(c1cc(C(F)(F)F)ccc1Cl)S(=O)(=O)c1ccccc1)NCc1ccncc1]

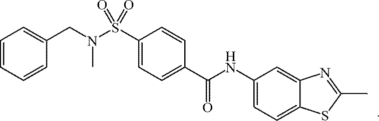

HBc directed transbodies such as those described in Wang Y., et al, Transbody against hepatitis B virus core protein inhibits hepatitis B virus replication in vitro, Int. Immunopharmacol (2014), located at //dx.doi.org/10.1016/j.intimp.2015.01.028; antiviral core protein mutant (such as Cp183-V124W and related mutations as described in WO/2013/010069, WO2014/074906, each incorporated by reference); inhibitors of HBx-interactions such as RNAi, antisense and nucleic acid based polymers targeting HBV RNA;, e.g., RNAi (for example ALN-HBV, ARC-520, TKM-HBV, ddRNAi), antisense (ISIS-HBV), or nucleic acid based polymer: (REP 2139-Ca); immunostimulants such as Interferon alpha 2a (Roferon), Intron A (interferon alpha 2b), Pegasys (peginterferon alpha 2a), Pegylated IFN 2b, IFN lambda 1a and PEG IFN lambda 1a, Wellferon, Roferon, Infergen, lymphotoxin beta agonists such as CBE11 and BS1); Non-Interferon Immune enhancers such as Thymosin alpha-1 (Zadaxin) and Interleukin-7 (CYT107); TLR-7/9 agonists such as GS-9620, CYT003, Resiquimod; Cyclophilin inhibitors such as NVP018; OCB-030; SCY-635; Alisporivir; NIM811 and related cyclosporine analogs; vaccines such as GS-4774, TG1050, Core antigen vaccine; SMAC mimetics such as birinapant and other IAP-antagonists; Epigenetic modulators such as KMT inhibitors (EZH½, G9a, SETD7, Suv39 inhibitors), PRMT inhibitors, HDAC inhibitors, SIRT agonists, HAT inhibitors, WD antagonists (e.g., OICR-9429), PARP inhibitors, APE inhibitors, DNMT inhibitors, LSD1 inhibitors, JMJD HDM inhibitors, and Bromodomain antagonists; kinase inhibitors such as TKB1 antagonists, PLK1 inhibitors, SRPK inhibitors, CDK2 inhibitors, ATM & ATR kinase inhibitors; STING Agonists; Ribavirin; N-acetyl cysteine ; NOV-205 (BAM205); Nitazoxanide (Alinia), Tizoxanide; SB 9200 Small Molecule Nucleic Acid Hybrid (SMNH); DV-601; Arbidol; FXR agonists (such as GW 4064 and Fexaramin); antibodies, therapeutic proteins, gene therapy, and biologics directed against viral components or interacting host proteins.

In some embodiments, the disclosure provides a method of treating a hepatitis B infection in a patient in need thereof, comprising administering a first compound selected from any one of the disclosed compounds, and one or more other HBV agents each selected from the group consisting of HBV capsid assembly promoters, HBF viral polymerase interfering nucleosides, viral entry inhibitors, HbsAg secretion inhibitors, disruptors of nucleocapsid formation, cccDNA formation inhibitors, antiviral core protein mutant, HBc directed transbodies, RNAi targeting HBV RNA, immunostimulants, TLR-7/9 agonists, cyclophilin inhibitors, HBV vaccines, SMAC mimetics, epigenetic modulators, kinase inhibitors, and STING agonists. In some embodiments, the disclosure provides a method of treating a hepatitis B infection in a patient in need thereof, comprising administering an amount of a disclosed compound, and administering another HBV capsid assembly promoter.

In some embodiments, the first and second amounts together comprise a pharmaceutically effective amount. The first amount, the second amount, or both may be the same, more, or less than effective amounts of each compound administered as monotherapies. Therapeutically effective amounts of a disclosed compound and antiviral may be co-administered to the subject, i.e., administered to the subject simultaneously or separately, in any given order and by the same or different routes of administration. In some instances, it may be advantageous to initiate administration of a disclosed compound first, for example one or more days or weeks prior to initiation of administration of the antiviral. Moreover, additional drugs may be given in conjunction with the above combination therapy.

In another embodiment, a disclosed compound may be conjugated (e.g., covalently bound directly or through molecular linker to a free carbon, nitrogen (e.g., an amino group), or oxygen (e.g., an active ester) of a disclosed compound), with a detection moiety, for e.g., a fluorophore moiety (such a moiety may for example re-emit a certain light frequency upon binding to a virus and/or upon photon excitation). Contemplated fluorophores include AlexaFluor® 488 (Invitrogen) and BODIPY FL (Invitrogen), as well as fluorescein, rhodamine, cyanine, indocarbocyanine, anthraquinones, fluorescent proteins, aminocoumarin, methoxycoumarin, hydroxycoumarin, Cy2, Cy3, and the like. Such disclosed compounds conjugated to a detection moiety may be used in e.g. a method for detecting HBV or biological pathways of HBV infection, e.g., in vitro or in vivo; and/or methods of assessing new compounds for biological activity.

### V. EXAMPLES

The compounds described herein can be prepared in a number of ways based on the teachings contained herein and synthetic procedures known in the art. In the description of the synthetic methods described below, it is to be understood that all proposed reaction conditions, including choice of solvent, reaction atmosphere, reaction temperature, duration of the experiment and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “intermediates” herein are contemplated as compounds of the disclosure.

Abbreviations:

| | |
|---|---|
| AcOH | Acetic acid |
| Boc₂O | Di-tert-butyl dicarbonate |
| nBuLi | n-Butyllithium |
| DCM | Dichloromethane |
| DIAD | Diisopropyl azodicarboxylate |
| DIEA | Diisopropyl ethylamine |
| DMF | N,N-Dimethylformamide |
| DMSO | Dimethyl sulfoxide |
| DPPF | 1,1′ -Bis(diphenylphosphino)ferrocene |
| EtOAc | Ethyl acetate |
| Et₃N | Triethylamine |
| HATU | Hexafluorophosphate Azabenzotriazole Tetramethyl Uronium |
| h, hr | Hour(s) |
| HPLC | High performance liquid chromatography |
| LCMS | Liquid chromatography-mass spectrometry |
| MeOH | Methanol |
| MeCN | Acetonitrile |
| NBS | N-Bromosuccinimide |
| NMO | N-Methylmorpholine-N-Oxide |
| PE | Petroleum ether |
| iPrOH | Isopropanol |
| rt | Room temperature |
| SFC | Supercritical Fluid Chromatography |
| TEA | Triethylamine |
| TFA | Trifluoroacetic acid |
| THF | Tetrahydrofuran |
| TLC | Thin-layer chromatography |
| XPhos | 2-Dicyclohexylphosphino-2′,4′,6′ -triisopropylbiphenyl |

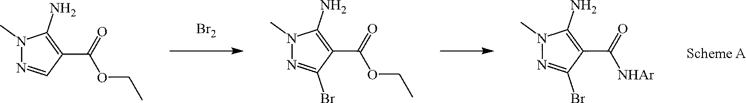

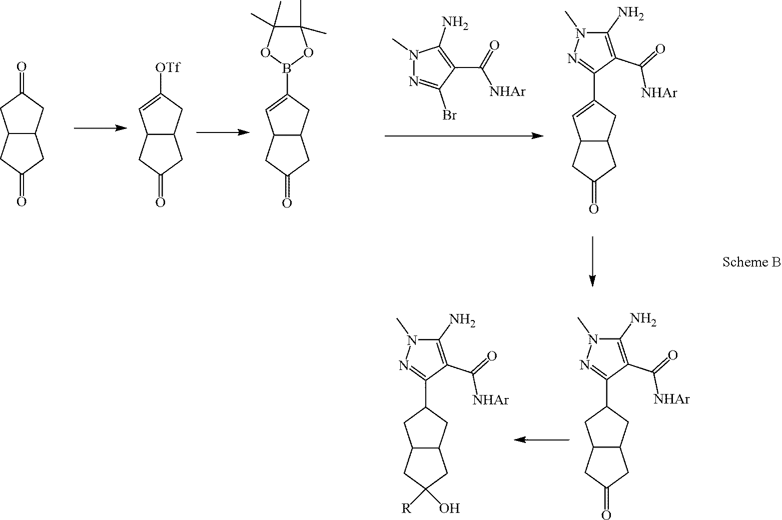

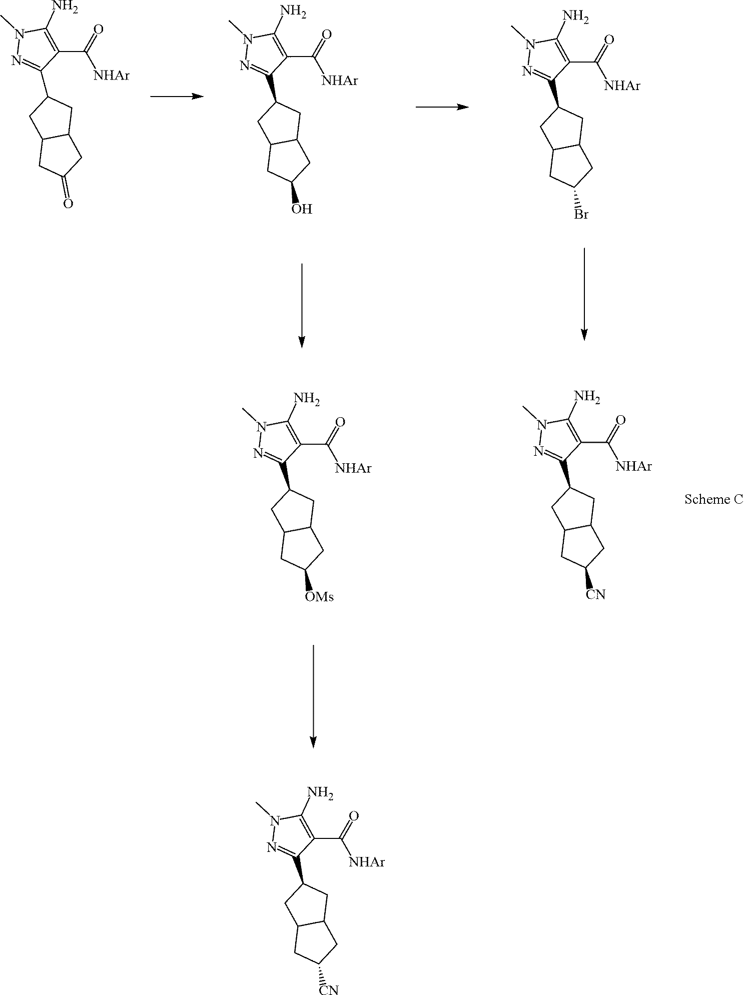

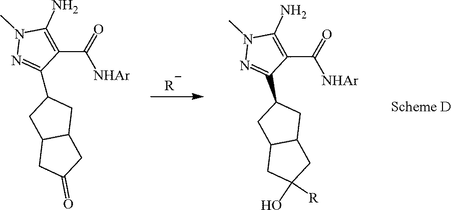

### General Procedure for Amidation

Method A (amide coupling using EDC.HCl): To a stirred solution of corresponding acid compound (1 eq.) in 1,4-dioxane (5.84 mL/mmol) were added EDC·HCl (1.1 eq.), HOBt (1.1 eq.) and corresponding amine (1 eq.) at 0° C. and stirred for 5 min. To this solution, DIPEA (3 eq.) was added and the reaction resulting reaction mixture was stirred at 90° C. for overnight. After completion, the reaction mixture was diluted with ice water and extracted with ethyl acetate. The organic layer was washed with sat. NaHCO₃ solution, water, dried over sodium sulfate, filtered and concentrated in vacuo to afford crude compound which was purified by silica gel column chromatography/prep. HPLC to afford the desired compound.

Method B (amide coupling using HATU): To a stirred solution of acid compound (1.1-1.2 eq.) in DMF/DCM (1.01 mL/mmol) at 0° C., DIPEA (2-3 eq.) and HATU (1.5-2.5 eq.) were added and stirred for 5 min. To this solution, corresponding amine (1 eq.) was added. The resulting reaction mixture was stirred at room temperature for 12-16 hr. After completion, the reaction mixture was diluted with ice cold water and extracted with ethyl acetate. The organic layer was collected; washed with brine; dried over anhydrous sodium sulphate and concentrated under reduced pressure to afford a crude compound. The crude compound was purified by either prep-HPLC or combiflash column chromatography to afford the desired compound.

Method C (AlMe₃ mediated amidation): To a stirred solution of corresponding anilines (1.1 eq.) in DCM/Toluene(3 mL/mmol) at 0° C. under Argon atmosphere, AlMe₃ (2 M in toluene, 2.5 eq.) was added and the reaction mixture was stirred at 0° C. for 10 min and continued stirring at room temperature for 1 h. To this solution, corresponding ester compound (1 eq.) was added at 0° C. under Argon atmosphere and resulting reaction mixture was refluxed at 100° C. for 16 hr. After completion, the reaction mixture was cooled to 0° C.; quenched with aqueous 1N HCl solution slowly and extracted with ethyl acetate. The combined organic layers were collected, dried over anhydrous sodium sulphate and concentrated in vacuo. The crude compound was purified by washing with methanol to afford the desired compound.

Method D (amide coupling using acid chloride/derivatives): To a stirred solution of amine compound (1 eq.) in DCM (1.01 mL/mmol) was added TEA (1.5-3 eq.) at 0° C. and stirred for 5 min. To this solution, corresponding acid chloride/carbamic chloride/chloroformate (1.1-1.5 eq.) was added slowly at 0° C. and the reaction mixture was allowed to stir at room temperature till completion. After completion, the reaction mixture was diluted with ice cold water and extracted with ethyl acetate/DCM. The organic layer was collected; washed with brine; dried over anhydrous sodium sulphate and concentrated under reduced pressure to afford a crude compound. The crude compound was purified by either prep-HPLC or combiflash column chromatography to afford the desired compound.

### General Procedure for Nucleophilic Addition of Keto Compound

Method A (at lower temperature): To a stirred solution of keto compound (1 eq.) in dry THF (0.2 mL/mmol) in an inert atmosphere was added a metallic reagent (e.g., Grignard reagent RMgX, RLi, R₂Zn, or R₃Al etc.) (10 eq.) slowly via glass syringe at -78° C. and stirred the reaction mixture for 4 hr at same temperature & then at room temperature for 2h. After completion, the reaction mixture was diluted with sat. aq. solution of ammonium chloride and extracted with ethyl acetate/DCM. The organic layer was collected; washed with brine; dried over anhydrous sodium sulphate and concentrated on rota vapor to afford a crude compound. The crude compound was purified by either by combiflash column chromatography or prep-HPLC to afford the desired compound.

### General Method for Suzuki Coupling

Method A: To a mixture of halo compound (1 eq.) and corresponding boronic acid/boronate ester (1.2-1.5 eq.) in 1, 4-dioxane:water (4:1) (2.17 mL/mmol), Na₂CO₃ (2-3 eq.) was added and purged with Argon for 15 min. To this solution, Pd(dppf)Cl2 (0.1 eq.) was added and purged with Argon for another 10 min. The resulting reaction mixture was stirred at 100° C. for 12-16 h. After completion of the reaction, the reaction mixture was filtered through Celite®545and evaporated to dryness. The residue was taken in ethyl acetate, washed with water, followed by brine, dried over anhydrous sodium sulphate and evaporated under reduced pressure. The crude product was purified by either combiflash column chromatography or prep-HPLC to afford the desired compound.

### General Procedure for Hydrogenation

Method A: To a stirred solution of olefinic compound (1 eq.) in EtOAc (2.67 mL/mmol) under nitrogen atomsphere, 10% Pd/C (20% by w/w of olefinic compound) was added. The reaction mixture was stirred under hydrogen atmosphere (100 psi) at 40-50° C. for 4-7 hr. After completion, the reaction mixture was filtered through a pad of Celite®545and washed with EtOAc/methanol. The filtrate was concentrated under reduced pressure to compound which was purified by silica gel column chromatography or prep-HPLC to give the desired compound.

### General Procedure for Keto-Reduction

Method A: To a stirred solution of keto compound (1 eq.) in EtOH/MeOH (5 vol) (4.7 mL/mmol), at 0° C. under Argon atmosphere, NaBH₄ (1-2 eq.) was added and stirred at room temperature for 2-6 hr. After completion, the reaction mixture was concentrated in vacuo, the residue obtained was diluted with water and extracted using ethyl acetate. The combined organic layers were collected, dried over anhydrous sodium sulphate, filtered, concentrated in vacuo and purified by silica gel column chromatography/prep. HPLC to give the desired compound. Note: THF was also (1 vol) added as a co-solvent for substrates which are having poor solubility in alcoholic solvents.

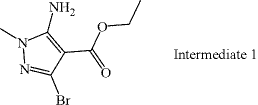

Ethyl 5-amino-3-bromo-1-methyl-1H-pyrazole-4-carboxylate. To a yellow solution of ethyl 5-amino-1-methyl-pyrazole-4-carboxylate (0.206 g, 1.22 mmol, 1 eq) in EtOH (5 mL) was added a solution of sodium acetate (929.89 mg, 11.34 mmol, 9.28 eq) in H₂O (8 mL), followed by dropwise addition of Br₂ (1.12 g, 7.04 mmol, 362.82 uL, 5.78 eq). The orange suspension was stirred at 15° C. for 3 hr. The reaction mixture was poured into H₂O (15 mL). The mixture was extracted with ethyl acetate (3 × 20 mL). The organic layers were combined and washed with saturated aqueous sodium thiosulfate solution (2 × 5 mL), dried (Na₂SO₄), filtered and concentrated under reduced pressure. The solid was triturated with a solution of methyl t-butyl ether: petroleum ether (1:10) (10 mL) for 5 min. Ethyl 5-amino-3-bromo-1-methyl-pyrazole-4-carboxylate was obtained as a yellow solid. ¹H NMR (400 MHz, CDCl3): 8 5.14 (br s, 2 H), 4.32 (q, J = 7.13 Hz, 2 H), 3.61 (s, 3 H), 1.38 (t, J = 7.15 Hz, 3 H) ppm.

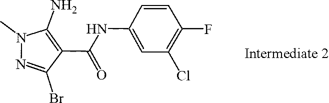 [SMILES: Cn1nc(Br)c(C(=O)Nc2ccc(F)c(Cl)c2)c1N]

5-Aminobromo-N-(3-chloro-4-fluorophenyl)-1-methyl-1H-pyrazole-4-carboxamide. To a colorless solution of 3-chloro-4-fluoro-aniline (281.65 mg, 1.93 mmol, 2 eq) in toluene (6 mL) was added Me₃Al (2 M in toluene) (2 M, 1.45 mL, 3 eq) at 0° C. The light brown solution was allowed to warm to 15° C. and stirred for 0.5 hr. To the solution was added ethyl 5-amino-3-bromo-1-methyl-pyrazole-4-carboxylate (0.24 g, 967.44 umol, 1 eq). The brown solution was stirred at 80° C. for 16 hr. Dark brown suspension was observed. The mixture was cooled to 0° C. and quenched with 1 N HCl (2 mL). A brown suspension was observed. The mixture was filtered. The filtrate was diluted with water (10 mL), extracted with EtOAc (15 mL x 3). The organic layers were combined, dried over MgSOa, filtered and concentrated under vacuum to give a residue as a yellow solid. The residue was triturated with methyl t-butyl ether (3 mL) for 5 min. 5-amino-3-bromo-N-(3-chloro-4-fluoro-phenyl)-1-methylpyrazole-4-carboxamide was obtained as a light yellow solid. ¹H NMR (400 MHz, CDCl3): δ 8.34 (br s, 1 H), 7.80 (dd, J = 6.54, 2.63 Hz, 1 H), 7.29 - 7.41 (m, 1 H), 7.12 (t, J = 8.74 Hz, 1 H), 5.53 (br s, 2 H), 3.64 (s, 3 H), 1.57 (s, 3 H) ppm.

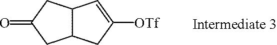 [SMILES: O=C1CC2C=C(OS(=O)(=O)C(F)(F)F)CC2C1]

5-Oxo-1,3a,4,5,6,6a-hexahydropentalen-2-yl trifluoromethanesulfonate. To a solution of 1,3,3a,4,6,6a-hexahydropentalene-2,5-dione (40.0 g, 289.5 mmol) and pyridine (24.0 g, 304.0 mmol) in DCM (600 ml) was added Tf₂O (89.8 g, 318.5 mmol) dropwise at room temperature. The mixture was stirred at room temperature for 3 h. Brine (300 mL) was added and the aqueous layer extracted with DCM (200 mL x 3). The organic layer was separated, dried over Na₂SO₄ and concentrated to give the crude product which was purified by silica gel column chromatography using 8:1 petroleum ether/ethyl acetate to afford 5-oxo-1,3a,4,5,6,6a-hexahydropentalen-2-yl trifluoromethanesulfonate as a yellow oil. ¹H NMR (400 MHz, CDCl3): 8 5.63 (q, J = 1.92 Hz, 1 H), 3.57 - 3.50 (m, 1 H), 3.14 - 3.00 (m, 2 H), 2.67 - 2.58 (m, 1 H), 2.56 -2.40 (m, 2 H), 2.34 - 2.26 (m, 1 H), 2.17 (ddd, J = 19.14, 7.34, 1.63 Hz, 1 H) ppm.

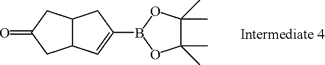 [SMILES: CC1(C)OB(C2=CC3CC(=O)CC3C2)OC1(C)C]

5-(4,4,5,5-Tetramethyl-1,3,2-dioxaborolan-2-yl)-3,3a,6,6a-tetrahydropentalen-2(1H)-one. A mixture of 5-oxo-1,3a,4,5,6,6a-hexahydropentalen-2-yl trifluoromethanesulfonate (110.0 g, 407.0 mmol), 4,4,5,5-tetramethyl-2-(4, 4,5,5-tetramethyl-1,3,2-dioxaborolan-2-yl)-1,3,2-dioxaborolane (108.5 g, 427.4 mmol), Pd(dppf)Cl2 (8.9 g, 12.2 mmol) and potassium acetate (119.7 g, 1221.0 mmol) in dioxane (1000 ml) was stirred at 80° C. under an N₂ atmosphere for 2 h. The reaction mixture was filtered through a pad of Celite® and the filter cake washed with EtOAc (250 mL × 3). The filtrate was concentrated under vacuum and the residue was purified by silica gel column chromatography using 8:1 petroleum ether/ethyl acetate to afford 5-(4,4,5,5-tetramethyl-1,3,2-dioxaborolan-2-yl)-3,3a,6,6a-tetrahydropentalen-2(1H)-one as a yellow oil. ¹H NMR (400 MHz, CDCl3): 8 6.37 (q, J = 2.08 Hz, 1 H), 3.54 - 3.41 (m, 1 H), 3.05 - 2.93 (m, 1 H), 2.79 (ddt, J = 16.48, 7.58, 2.64, 2.64 Hz, 1 H), 2.55 - 2.24 (m, 4 H), 2.07 - 1.95 (m, 1 H), 1.28 (s, 13 H) ppm.

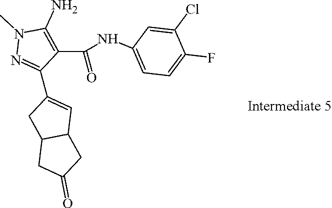 [SMILES: Cn1nc(C2=CC3CC(=O)CC3C2)c(C(=O)Nc2ccc(F)c(Cl)c2)c1N]

5-Amino-N-(3-chloro-4-fluorophenyl)-1-methyl-3-(5-oxo-1,3a,4,5,6,6a-hexahydropentalen-2-yl)-1H-pyrazole-4-carboxamide. A mixture of 5-amino-3-bromo-N-(3-chloro-4-fluorophenyl)-1-methyl-1H-pyrazole-4-carboxamide (68.6 g, 197.5 mmol), 5-(4,4,5,5-tetramethyl-1,3,2-dioxaborolan-2-yl)-3,3a,6,6a-tetrahydropentalen-2(1H)-one (70.0 g, 282.1 mmol), Pd(dppf)Ch (10.1 g, 13.8 mmol) and Na₂CO₃ (41.9 g, 395.0 mmol) in dioxane (1200 mL) and H₂O (150 mL) was stirred at 80° C. overnight under N₂. The mixture was a brown suspension. The solvent was evaporated under vacuum. The residue was purified by silica gel column chromatography using 1:2 petroleum ether/ethyl acetate to afford 5-amino-N-(3-chloro-4-fluorophenyl)-1-methyl-3-(5-oxo-1,3,4,5,6,6a-hexahydropentalen-2-yl)-1H-pyrazole-4-carboxamide as a yellow solid. MS (m/z): calcd.: 388.1, Found: 389.0 [M+1]; ¹H NMR (400 MHz, CDCl3): 8 2.19 (dd, J=19.20, 5.26 Hz, 1 H), 2.33 (br d, J=18.83 Hz, 1 H), 2.55 - 2.79 (m, 3 H), 3.15 - 3.28 (m, 2 H), 3.63 (s, 3 H), 3.66 (s, 1 H), 3.68 - 3.77 (m, 1 H), 5.24 - 5.45 (m, 2 H), 6.05 (d, J=1.71 Hz, 1 H), 6.95 - 7.20 (m, 2 H), 7.47 - 7.58 (m, 1 H), 7.68 - 7.86 (m, 2 H) ppm.

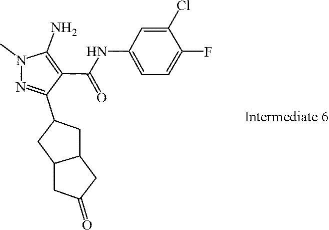 [SMILES: Cn1nc(C2CC3CC(=O)CC3C2)c(C(=O)Nc2ccc(F)c(Cl)c2)c1N]

5-Amino-N-(3-chloro-4-fluorophenyl)-1-methyl-3-(5-oxooctahydropentalen-2-yl)-1H-pyrazole-4-carboxamide. To a solution of 5-(4,4,5,5-tetramethyl-1,3,2-dioxaborolan-2-yl)-3,3a,6,6a-tetrahydropentalen-2(1H)-one (5.0 g, 12.9 mmol) in EtOAc (500 ml) was added Pd/C (2.5 g, 10% w/w Pd). The mixture was stirred at 40° C. for 2 h under H₂. The mixture was filtered, and solvent removed under vacuum to give the target compound as a white solid. The crude product was used directly without any further purification. MS (m/z): Calcd.: 390.1, Found: 391.0 [M+1]⁺ ; ¹H NMR (400 MHz, CDCl3): δ 1.85 - 2.01 (m, 2 H), 2.07 - 2.29 (m, 2 H), 2.41 - 2.67 (m, 4 H), 2.83 - 3.06 (m, 2 H), 3.32 - 3.50 (m, 1 H), 3.54 - 3.61 (m, 3 H), 5.15 - 5.32 (m, 2 H), 7.12 (t, J=8.74 Hz, 1 H), 7.27 - 7.35 (m, 2 H), 7.65 - 7.83 (m, 1 H) ppm.

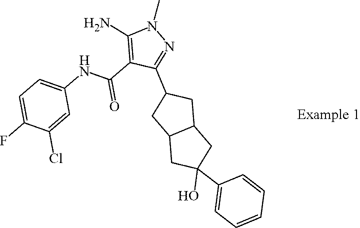 [SMILES: Cn1nc(C2CC3CC(O)(c4ccccc4)CC3C2)c(C(=O)Nc2ccc(F)c(Cl)c2)c1N]

5-Amino-N-(3-chloro-4-fluorophenyl)-3-(5-hydroxy-5-phenyl-octahydropentalen-2-yl)-1-methyl-1H-pyrazole-4-carboxamide. 2 M PhMgBr in THF (6.4 mL, 12.8 mmol) was added to a stirred solution of 5-amino-N-(3-chloro-4-fluorophenyl)-1-methyl-3-(5-oxooctahydropentalen-2-yl)-1H-pyrazole-4-carboxamide (0.5 g, 1.28 mmol) in THF (10 mL) at -40° C., and the mixture stirred at room temperature for 3 h. After completion, the reaction mixture was diluted with saturated aqueous NH₄Cl and extracted with ethyl acetate. The combined organic layer was dried over anhydrous sodium sulphate, filtered, and concentrated under reduced pressure. The crude compound was purified by trituration with DCM and filtered to afford 5-amino-N-(3-chloro-4-fluorophenyl)-3-(5-hydroxy-5-phenyl-octahydropentalen-2-yl)-1-methyl-1H-pyrazole-4-carboxamide as a white solid. MS (m/z): Calcd. for C₂₅H₂₆CIFN₄O₂— 468.17, Found; 491.15 [ M + Na]⁺. ¹H NMR (400 MHz, DMSO-d₆): δ 8.91 (s, 1H), 7.92 (dd, J = 7.0, 2.4 Hz, 1H), 7.56 - 7.48 (m, 1H), 7.42 (d, J = 8.0 Hz, 2H), 7.34 (t, J = 8.8 Hz, 1H), 7.27 (t, J = 7.6 Hz, 2H), 7.20 - 7.14 (m, 1H), 5.98 (s, 2H), 4.75 (s, 1H), 3.50 (s, 3H), 3.46 - 3.38 (m, 1H, merged), 2.68 - 2.58 (m, 2H), 2.20 - 2.02 (m, 4H), 2.00 - 1.88 (m, 2H), 1.86 - 1.76 (m, 2H) ppm.

| | |
|---|---|
| Example 2 | 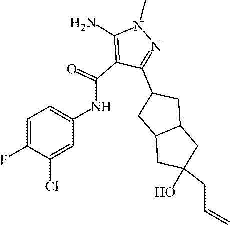 [SMILES: C=CCC1(O)CC2CC(c3nn(C)c(N)c3C(=O)Nc3ccc(F)c(Cl)c3)CC2C1] |
| | 3-(5-Allyl-5-hydroxyoctahydropentalen-2-yl)-5-amino-N-(3-chloro-4-fluorophenyl)-1-methyl-1H-pyrazole-4-carboxamide. MS (m/z): Calcd. for C₂₂H₂₆CIFN₄O₂— 432.17, Found: 433.16 [M+1]+; ¹H NMR (400 MHz, DMSO-d₆): δ 8.91 (s, 1H), 7.90 (dd, J = 6.8, 2.4 Hz, 1H), 7.54 - 7.46 (m, 1H), 7.34 (t, J = 9.2 Hz, 1H), 5.96 (s, 2H), 5.92 - 5.80 (m, 1H), 5.00 (s, 1H), 4.98 - 4.96 (m, 1H), 4.24 (s, 1H), 3.48 (s, 3H), 3.48 - 3.38 (m, 1H), 2.48 -2.32 (m, 2H), 2.20 - 2.10 (m, 4H), 1.76 - 1.58 (m, 4H), 1.42 - 1.36 (m, 2H) ppm. |
| Example 3 | 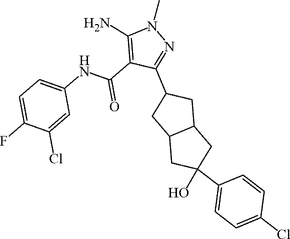 [SMILES: Cn1nc(C2CC3CC(O)(c4ccc(Cl)cc4)CC3C2)c(C(=O)Nc2ccc(F)c(Cl)c2)c1N] |
| | 5-Amino-N-(3-chloro-4-fluorophenyl)-3-(5-(4-chlorophenyl)-5-hydroxyoctahydropentalen-2-yl)-1-methyl-1H-pyrazole-4-carboxamide. MS (m/z): Calcd. for C₂₅H₂₅CI2FN₄O₂— 502.13, Found: 501.15 [M - 1]⁻. ¹H NMR (400 MHz, DMSO-d₆): δ 8.91 (s, 1H), 7.91 (dd, J = 6.8, 2.4 Hz, 1H), 7.54 - 7.49 (m, 1H), 7.43 (d, J = 8.8 Hz, 2H), 7.37 - 7.31 (m, 3H), 5.98 (m, 2H), 4.87 (s, 1H), 3.49 (s, 3H), 3.45 - 3.30 (m, 1H), 2.67 - 2.60 (m, 2H), 2.15 - 1.78 (m, 8H) ppm. |
| Example 4 | 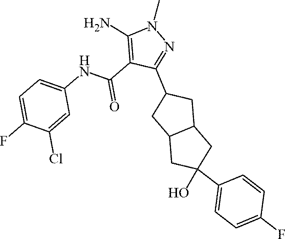 [SMILES: Cn1nc(C2CC3CC(O)(c4ccc(F)cc4)CC3C2)c(C(=O)Nc2ccc(F)c(Cl)c2)c1N] |
| | 5-Amino-N-(3-chloro-4-fluorophenyl)-3-(5-(4-fluorophenyl)-5-hydroxyoctahydropentalen-2-yl)-1-methyl-1H-pyrazole-4-carboxamide. MS (m/z): Calcd. for C₂₅H₂₅CIF₂N₄O₂— 486.16, Found: 486.90 [M + 1]⁺; ¹H NMR (400 MHz, DMSO-d₆): δ 8.91 (s, 1H), 7.91 (dd, J = 7.2, 2.4 Hz, 1H), 7.54 - 7.51 (m, 1H), 7.50 - 7.42 (m, 2H), 7.34 (t, J = 9.6 Hz, 1H), 5.98 (m, 2H), 7.08 (d, J = 8.8 Hz, 2H), 4.82 (s, 1H), 3.49 (s, 3H), 3.43 - 3.36 (m, 1H), 2.67 - 2.58 (m, 2H), 2.16 - 1.91 (m, 6H), 1.82 - 1.78 (m, 2H) ppm. |
| Example 5 | 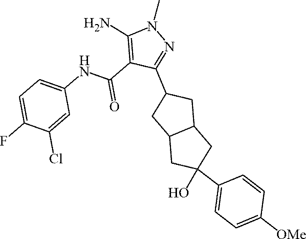 [SMILES: COc1ccc(C2(O)CC3CC(c4nn(C)c(N)c4C(=O)Nc4ccc(F)c(Cl)c4)CC3C2)cc1] |
| | 5-Amino-N-(3-chloro-4-fluorophenyl)-3-(5-hydroxy-5-(4-methoxyphenyl)octahydropentalen-2-yl)-1-methyl-1H-pyrazole-4-carboxamide. MS (m/z): Calcd. for C₂₆H₂₈CIFN₄O₃— 498.18, Found: 481.00 [M-18]; ¹H NMR (400 MHz, DMSO-d₆): δ 8.91 (s, 1H), 7.91 (dd, J = 6.8, 2.4 Hz, 1H), 7.52 - 7.50 (m, 1H), 7.37 - 7.32 (m, 3H), 6.81 (d, J = 8.8 Hz, 1H), 5.98 (s, 3H), 4.66 (s, 1H), 3.71 (s, 3H), 3.49 (s, 3H), 3.48 - 3.35 (m, 1H), 2.58 - 2.56 (m, 2H), 2.14 - 1.75 (m, 8H) ppm. |
| Example 6 | 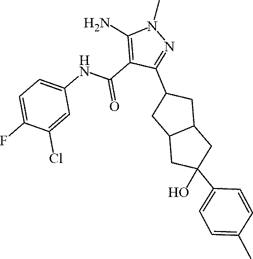 [SMILES: Cc1ccc(C2(O)CC3CC(c4nn(C)c(N)c4C(=O)Nc4ccc(F)c(Cl)c4)CC3C2)cc1] |
| | 5-Amino-N-(3-chloro-4-fluorophenyl)-3-(5-hydroxy-5-(p-tolyl)octahydropentalen-2-yl)-1-methyl-1H-pyrazole-4-carboxamide. MS (m/z): Calcd. for C₂₆H₂₈CIFN₄O₂— 482.19, Found: 465.10 [M - 18]; ¹H NMR (400 MHz, DMSO-d₆): δ 8.90 (s, 1H), 7.92 (dd, J = 6.8, 2.4 Hz, 1H), 7.54 - 7.50 (m, 1H), 7.67 - 7.29 (m, 3H), 7.06 (d, J = 8.0 Hz, 2H), 5.98 (s, 2H), 4.68 (s, 1H), 3.50 (s, 3H), 3.45 - 3.37 (s, 1H), 2.59 - 2.53 (m, 2H), 2.25 (s, 3H), 2.15 - 1.76 (m, 8H) ppm. |
| Example 7 | 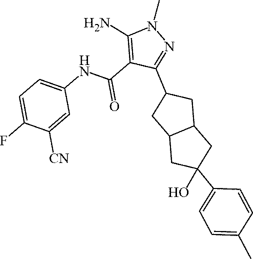 [SMILES: Cc1ccc(C2(O)CC3CC(c4nn(C)c(N)c4C(=O)Nc4ccc(F)c(C#N)c4)CC3C2)cc1] |
| | 5-Amino-N-(3-cyano-4-fluorophenyl)-3-(5-hydroxy-5-(p-tolyl)octahydropentalen-2-yl)-1-methyl-1H-pyrazole-4-carboxamide. MS (m/z): Calcd. for C₂₇H₂₈FN₅O₂: 473.22, Found: 456.03 [M-18]; ¹H NMR (400 MHz, DMSO-d₆): δ 9.04 (s, 1H), 8.12 (dd, J = 6.0, 3.3 Hz, 1H), 7.92 - 7.88 (m, 1H), 7.47 (t, J = 9.2 Hz, 1H), 7.29 (d, J = 8.0 Hz, 2H), 7.06 (d, J = 8.0 Hz, 2H), 6.02 (s, 2H), 4.69 (s, 1H), 3.50 (s, 3H), 3.45 - 3.40 (m, 1H), 2.60 - 2.59 (m, 2H), 2.25 (s, 3H), 2.18 - 1.2.05 (m, 4H), 1.97 - 1.88 (m, 2H), 1.80 - 1.76 (m, 2H) ppm. |
| Example 8 | 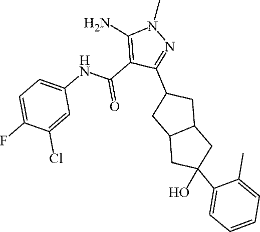 [SMILES: Cc1ccccc1C1(O)CC2CC(c3nn(C)c(N)c3C(=O)Nc3ccc(F)c(Cl)c3)CC2C1] |
| | 5-Amino-N-(3-chloro-4-fluorophenyl)-3-(5-hydroxy-5-(o-tolyl)octahydropentalen-2-yl)-1-methyl-1H-pyrazole-4-carboxamide. MS (m/z): Calcd. for C₂₆H₂₈CIFN₄O₂— 482.19, Found: 465.10 [M-18]; ¹H NMR (400 MHz, DMSO-d₆): δ 8.91(s, 1H), 7.93 - 7.90 (m, 1H), 7.52 - 7.49 (m, 1H), 7.36 - 7.27 (m, 2H), 7.10 - 7.07 (m, 3H), 5.99 (s, 2H), 4.75 (s, 1H), 3.50 (s, 3H), 3.47 - 3.40 (m, 1H), 2.50 - 2.47 (s, 3H, merged), 2.46 - 2.40 (m, 2H), 2.30 - 2.25 (m, 2H), 2.17 - 2.14 (m, 2H), 1.91 - 1.75 (m, 4H) ppm. |
| Example 9 | 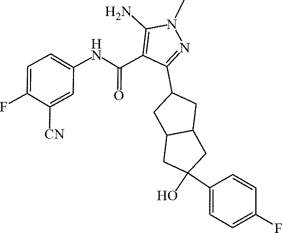 [SMILES: Cn1nc(C2CC3CC(O)(c4ccc(F)cc4)CC3C2)c(C(=O)Nc2ccc(F)c(C#N)c2)c1N] |
| | 5-Amino-N-(3-cyano-4-fluorophenyl)-3-(5-(4-fluorophenyl)-5-hydroxyoctahydropentalen-2-yl)-1-methyl-1H-pyrazole-4-carboxamide. MS (m/z): Calcd. for C₂₆H₂₅F₂N₅O₂: 477.20, Found: 460.45 [M-18]; ¹H NMR (400 MHz, DMSO-d₆): δ 9.04 (s, 1H), 8.12-8.10 (m, 1H), 7.90 - 7.85 (m, 1H), 7.47 - 7.40 (m, 3H), 7.07 (t, J = 8.8 Hz, 2H), 6.01 (s, 2H), 4.82 (s, 1H), 3.49 (s, 3H), 3.48 - 3.35 (m, 1H, merged), 2.65 - 2.55 (m, 2H), 2.15 -2.02 (m, 4H), 1.93 - 1.89 (m, 2H), 1.82 - 1.75 (m, 2H) ppm. |
| Example 10 | 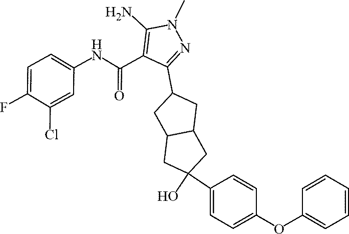 [SMILES: Cn1nc(C2CC3CC(O)(c4ccc(Oc5ccccc5)cc4)CC3C2)c(C(=O)Nc2ccc(F)c(Cl)c2)c1N] |
| | 5-Amino-N-(3-chloro-4-fluorophenyl)-3-(5-hydroxy-5-(4-phenoxyphenyl)octahydropentalen-2-yl)-1-methyl-1H-pyrazole-4-carboxamide. MS (m/z): Calcd. for C₃₁H₃₀CIFN₄O₃— 560.20, Found: 543.05 [M-18]; ¹H NMR (400 MHz, DMSO-d₆): δ 8.92 (s, 1H), 7.94 - 7.90 (m, 1H), 7.53 - 7.50 (m, 1H), 7.45 - 7.32 (m, 5H), 7.11 (t, J = 7.2 Hz, 1H), 6.98 - 6.90 (m, 4H), 5.99 (s, 2H), 4.79 (s, 1H), 3.50 (s, 3H), 3.48 - 3.32 (m, 1H), 2.60 - 2.55 (m, 2H), 2.15 - 2.04 (m, 4H), 1.95 - 1.91 (m, 2H), 1.83 - 1.79 (m, 2H) ppm. |
| Example 11 | 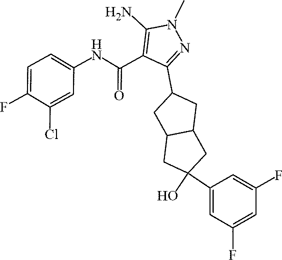 [SMILES: Cn1nc(C2CC3CC(O)(c4cc(F)cc(F)c4)CC3C2)c(C(=O)Nc2ccc(F)c(Cl)c2)c1N] |
| | 5-Amino-N-(3-chloro-4-fluorophenyl)-3-(5-(3,5-difluorophenyl)-5-hydroxyoctahydropentalen-2-yl)-1-methyl-1H-pyrazole-4-carboxamide. MS (m/z): Calcd. for C₂₅H₂₄CIF₃N₄O₂— 504.15, Found: 505.45 [M+1]⁺; ¹H NMR (400 MHz, DMSO-d₆): δ 8.92 (s, 1H), 7.94-7.90 (m, 1H), 7.54-7.50 (m, 1H), 7.35 (t, J = 8.8 Hz, 1H), 7.10 - 7.08 (m, 2H), 7.04 - 6.99 (m, 1H), 5.96 (s, 2H), 5.03 (s, 1H), 3.50 (s, 3H), 3.42 - 3.36 (m, 1H), 2.70 - 2.64 (m, 2H), 2.15 - 2.04 (m, 4H), 1.99 - 1.90 (m, 2H), 1.81 - 1.76 (m, 2H) ppm. |
| Example 12 | 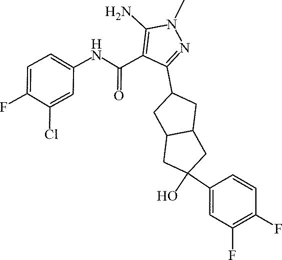 [SMILES: Cn1nc(C2CC3CC(O)(c4ccc(F)c(F)c4)CC3C2)c(C(=O)Nc2ccc(F)c(Cl)c2)c1N] |
| | 5-Amino-N-(3-chloro-4-fluorophenyl)-3-(5-(3,4-difluorophenyl)-5-hydroxyoctahydropentalen-2-yl)-1-methyl-1H-pyrazole-4-carboxamide. MS (m/z): Calcd. for C₂₅H₂₄CIF₃N₄O₂— 504.15, Found: 487.15 [M-18]; ¹H NMR (400 MHz, DMSO-d₆): δ 8.92 (s, 1H), 7.94 - 7.90 (m, 1H), 7.53 -7.50 (m, 1H), 7.45 - 7.25 (m, 4H), 5.98 (s, 2H), 4.96 (s, 1H), 3.50 (s, 3H), 3.45 - 3.35 (m, 1H), 2.65 - 2.60 (m, 2H), 2.14 - 2.02 (m, 4H), 1.97 - 1.92 (m, 2H), 1.82 - 1.75 (m, 2H) ppm. |
| Example 13 | 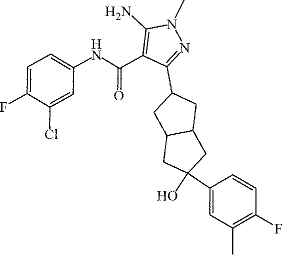 [SMILES: Cc1cc(C2(O)CC3CC(c4nn(C)c(N)c4C(=O)Nc4ccc(F)c(Cl)c4)CC3C2)ccc1F] |
| | 5-Amino-N-(3-chloro-4-fluorophenyl)-3-(5-(4-fluoro-3-methylphenyl)-5-hydroxyoctahydropentalen-2-yl)-1-methyl-1H-pyrazole-4-carboxamide. MS (m/z): Calcd. for C₂₆H₂₇CIF₂N₄O₂— 500.18, Found: 483.25 [M-18]; ¹H NMR (400 MHz, DMSO-d₆): δ 8.91 (s, 1H), 7.93 - 7.89 (m, 1H), 7.52 - 7.48 (m, 1H), 7.36 - 7.25 (m, 3H), 7.00 (t, J = 8.8 Hz, 1H), 5.98 (s, 2H), 4.77 (s, 1H), 3.49 (s, 3H), 3.42-3.39 (m, 1H), 2.65 - 2.60 (m, 2H), 2.20 (s, 3H), 2.13 - 2.04 (m, 4H), 1.95 - 1.88 (m, 2H), 1.79 - 1.75 (m, 2H) ppm. |
| Example 14 | 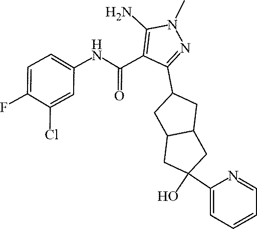 [SMILES: Cn1nc(C2CC3CC(O)(c4ccccn4)CC3C2)c(C(=O)Nc2ccc(F)c(Cl)c2)c1N] |
| | 5-Amino-N-(3-chloro-4-fluorophenyl)-3-(5-hydroxy-5-(pyridin-2-yl)octahydropentalen-2-yl)-1-methyl-1H-pyrazole-4-carboxamide. MS (m/z): Calcd. for C₂₄H₂₅CIFN₅O₂— 469.17, Found: 470 [M+1]+; ¹H NMR (400 MHz, DMSO-d₆): δ 8.92 (s, 1H), 8.47-8.44 (m, 1H), 7.93 (dd, J = 6.8, 2.4 Hz, 1H), 7.76 - 7.70 (m, 1H), 7.65 - 7.62 (m, 1H), 7.54 - 7.50 (m, 1H), 7.35 (t, J = 8.8 Hz, 1H), 7.20 - 7.17 (m, 1H), 5.99 (s, 2H), 5.02 (s, 1H), 3.51 (s, 3H), 3.43 - 3.38 (m, 1H), 2.70 - 2.65 (m, 2H), 2.34 - 2.29 (m, 2H), 2.17 -2.13 (m, 2H), 2.00 - 1.94 (m, 2H), 1.73 - 1.69 (m, 2H) ppm. |
| Example 15 | 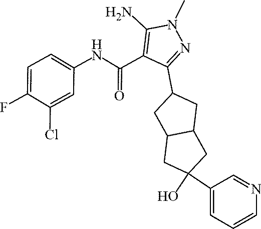 [SMILES: Cn1nc(C2CC3CC(O)(c4cccnc4)CC3C2)c(C(=O)Nc2ccc(F)c(Cl)c2)c1N] |
| | 5-Amino-N-(3-chloro-4-fluorophenyl)-3-(5-hydroxy-5-(pyridin-3-yl)octahydropentalen-2-yl)-1-methyl-1H-pyrazole-4-carboxamide MS (m/z): Calcd. for C₂₄H₂₅CIFN₅O₂— 469.17, Found: 470.15 [M + 1]⁺; ¹H NMR (400 MHz, DMSO-d₆): δ 8.92 (s, 1H), 8.64 (s, 1H), 8.39-8.38 (m, 1H),7.93 - 7.91 (m, 1H), 7.80 - 7.78 (m, 1H), 7.52 - 7.51 (m, 1H), 7.37 -7.28 (m, 2H), 5.98 (s, 2H), 4.98 (s, 1H), 3.50 (s, 3H), 2.46 - 2.42 (m, 1H), 2.63 (br.s, 2H), 2.15 - 2.07 (m, 4H), 1.95 - 1.93 (m, 2H), 1.86 - 1.83 (m, 2H) ppm. |
| Example 16 | 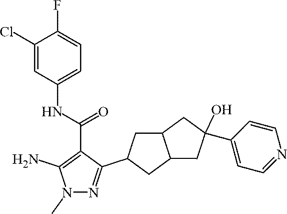 [SMILES: Cn1nc(C2CC3CC(O)(c4ccncc4)CC3C2)c(C(=O)Nc2ccc(F)c(Cl)c2)c1N] |
| | 5-Amino-N-(3-chloro-4-fluorophenyl)-3-(5-hydroxy-5-(pyridin-4-yl)octahydropentalen-2-yl)-1-methyl-1H-pyrazole-4-carboxamide MS (m/z): Calcd.: 469.17, Found: 470.10 [M + 1]⁺. |
| Example 17 | 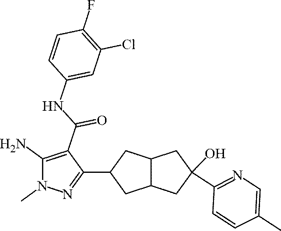 |
| | 5-Amino-N-(3-chloro-4-fluorophenyl)-3-(5-hydroxy-5-(5-methylpyridin-2-yl)octahydropentalen-2-yl)-1-methyl-1H-pyrazole-4-carboxamide. ¹H NMR (400 MHz, DMSO-d₆): δ 8.92 (s, 1H), 8.29 (s, 1H), 7.92 (dd, J = 6.8, 2.4 Hz, 1H), 7.54 - 7.50 (m, 1H), 7.35 (t, J = 9.2 Hz, 1H), 5.99 (s, 2H), 4.96 (s, 1H), 3.50 (s, 3H), 3.43 - 3.31 (m, 1H), 2.66 - 2.65 (m, 2H), 2.31 - 2.25 (m, 5H), 2.14 - 2.07 (m, 2H), 1.95 - 1.92 (m, 2H), 1.69 -1.66 (m, 2H) ppm |

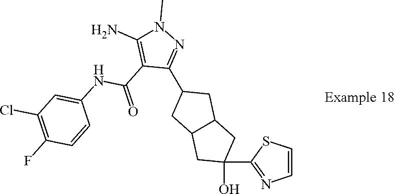 [SMILES: Cn1nc(C2CC3CC(O)(c4nccs4)CC3C2)c(C(=O)Nc2ccc(F)c(Cl)c2)c1N]

5-Amino-N-(3-chloro-4-fluorophenyl)-3-(5-hydroxy-5-(thiazol-2-yl)octahydropentalen-2-yl)-1-methyl-1H-pyrazole-4-carboxamide. To a solution of thiazole (87 mg, 1.0 mmol) in THF (2 mL) was added n-BuLi (0.4 mL, 2.5 M, 1.0 mmol) at -78° C. under N₂ and the mixture stirred at -78° C. for 1 h. To this was added 5-amino-N-(3-chloro-4-fluorophenyl)-1-methyl-3-(5-oxooctahydropentalen-2-yl)-1H-pyrazole-4-carboxamide (50 mg, 0.13 mmol) in one portion. The resulting mixture was kept -78° C. and stirred for 1 h. The reaction was quenched with sat. aqueous NH₄Cl and extracted with ethyl acetate. The organic layer was dried and concentrated. The crude residue was purified by prep-HPLC to afford 5-amino-N-(3-chloro-4-fluorophenyl)-3-(5-hydroxy-5-(thiazol-2-yl)octahydropentalen-2-yl)-1-methyl-1H-pyrazole-4-carboxamide as a white solid. TLC 100% ethyl acetate (R_{f,} 0.3). MS calcd.: 475.1; MS Found: 476.2 [M + 1]⁺. ¹H-NMR (400 MHz, DMSO-d₆-): δ 8.93 (s, 1H), 7.94-7.92 (m, 1H), 7.67 (d, J = 3.2 Hz, 1H), 7.55-7.50 (m, 2H), 7.35 (t, J = 9.2 Hz, 1H), 6.00 (s, 2H), 5.86 (s, 1H), 3.50 (s, 3H), 3.45-3.38 (m, 1H), 2.70-2.69 (m, 2H), 2.28-2.23 (m, 2H), 2.19-2.15 (m, 2H), 1.90-1.83 (m, 4H) ppm.

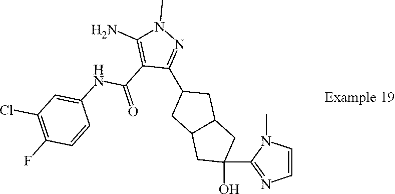 [SMILES: Cn1ccnc1C1(O)CC2CC(c3nn(C)c(N)c3C(=O)Nc3ccc(F)c(Cl)c3)CC2C1]

5-Amino-N-(3-chloro-4-fluorophenyl)-3-(5-hydroxy-5-(1-methyl-1H-imidazol-2-yl)octahydropentalen-2-yl)-1-methyl-1H-pyrazole-4-carboxamide. To a solution of 1-methyl-1H-imidazole (410 mg, 5.0 mmol) in THF (5 mL) was added n-BuLi (2.5 M in hexanes, 2.0 mL) at - 20° C. and stirred 0.5 h. To this was added 5-amino-N-(3-chloro-4-fluorophenyl)-1-methyl-3-(5-oxooctahydropentalen-2-yl)-1H-pyrazole-4-carboxamide (200 mg, 0.5 mmol) in THF (1 mL) and stirring continued for another 3 hr at - 20° C. The reaction was quenched with sat. aqueous NH₄Cl (10 mL) and extracted with EtOAc (10 mL x 3). The combined organic layers were dried over anhydrous Na₂SOa, concentrated under reduced pressure and purified by reversed column chromatography to afford 5-amino-N-(3-chloro-4-fluorophenyl)-3-(5-hydroxy-5-(1-methyl-1H-imidazol-2-yl)octahydropentalen-2-yl)-1-methyl-1H-pyrazole-4-carboxamide as a yellow solid. MS calcd.: 472.2. Found 473.3. ¹H NMR (400 MHz, DMSO-d₆): δ 8.90 (s, 1H), 7.93 (dd, J = 7.2 Hz, 2.8 Hz, 1H), 7.53 - 7.49 (m, 1H), 7.35 (t, J = 8.8 Hz, 1H), 7.00 (s, 1H), 6.66 (s, 1H), 6.01 (s, 2H), 5.19 (s, 1H), 3.73 (s, 3H), 3.50 (s, 3H), 3.47 - 3.40 (m, 1H), 2.51-2.50 (m, 2H), 2.43 - 2.38 (m, 2H), 2.18 - 2.13 (m, 2H), 1.81 - 1.70 (m, 4H) ppm.

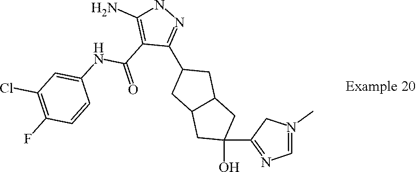

3-Amino-N-(3-chloro-4-fluorophenyl)-5-(5-hydroxy-5-(1-methyl-1H-imidazol-4-yl)octahydropentalen-2-yl)-2λ²-pyrazole-4-carboxamide. To a solution of 4-iodo-1-methyl-1H-imidazole (166 mg, 0.8 mmol) in DCM (2 mL) was added EtMgBr (1.0 M, 0.8 mL, 0.8 mmol) at room temperature under nitrogen atmosphere. After stirring 1 h, a suspension of 5-amino-N-(3-chloro-4-fluorophenyl)-1-methyl-3-(5-oxooctahydropentalen-2-yl)-1H-pyrazole-4-carboxamide (39 mg, 0.1 mmol) in THF (1 mL) was added. The reaction was stirred for 2 h at room temperature, quenched with sat. aqueous NH₄Cl and extracted with ethyl acetate (30 mL × 3). The organic layer was dried over anhydrous Na₂SO₄ and concentrated to give the crude compound which was purified by prep-HPLC to afford 3-amino-N-(3-chloro-4-fluorophenyl)-5-(5-hydroxy-5-(1-methyl-1H-imidazol-4-yl)octahydropentalen-2-yl)-2λ²-pyrazole-4-carboxamide as a white solid. TLC; 50% ethyl acetate/petroleum ether (R_{f}— 0.3). MS calcd.: 472.2, MS Found: 473.2 [M+1]⁺. ¹H-NMR (400 MHz, DMSO-d₆): δ 8.90 (brs, 1H), 7.92 (dd, J = 2.4 Hz, 6.4 Hz, 1H), 7.52-7.46 (m, 1H), 7.40 (s, 1H), 7.34 (t, J = 9.0 Hz, 1H), 6.85 (d, J = 1.6 Hz, 1H), 5.99 (s, 2H), 4.49 (s, 1H), 3.57 (6H, s), 3.49 (s, 1H), 2.50 (t, J = 1.8 Hz, 2H), 2.21-2.10 (m, 4H), 1.83-1.78 (m, 2H), 1.62-1.57 (m, 2H) ppm.

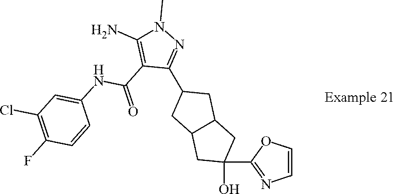 [SMILES: Cn1nc(C2CC3CC(O)(c4ncco4)CC3C2)c(C(=O)Nc2ccc(F)c(Cl)c2)c1N]

5-Amino-N-(3-chloro-4-fluorophenyl)-3-(5-hydroxy-5-(oxazol-2-yl)octahydropentalen-2-yl)-1-methyl-1H-pyrazole-4-carboxamide. A solution of oxazole (142 mg, 2.0 mmol) and BH₃-THF (2.0 mL, 1.0 M, 2.0 mmol) was stirred for 30 min. The mixture was cooled to -78° C., and n-BuLi (0.8 mL, 2.5 M, 2.0 mmol) added. The reaction was stirred at -78° C. for 1 h. Following this 5-amino-N-(3-chloro-4-fluorophenyl)-1-methyl-3-(5-oxooctahydropentalen-2-yl)-1H-pyrazole-4-carboxamide (100 mg, 0.26 mmol) was added in one portion. The resulting mixture was kept -78° C. and stirred for 1 h. The reaction was quenched with sat. aqueous NH₄Cl and extracted with ethyl acetate. The organic layer was dried and concentrated. The residue was purified by prep-HPLC to afford 5-amino-N-(3-chloro-4-fluorophenyl)-3-(5-hydroxy-5-(oxazol-2-yl)octahydropentalen-2-yl)-1-methyl-1H-pyrazole-4-carboxamide as a white solid. TLC: 100% ethyl acetate (R_{f}: 0.4). MS calcd.: 459.1. Found: 460.2 [M + 1]⁺. ¹H-NMR (400 MHz, DMSO-d₆): δ 8.92 (s, 1H), 8.01 (s, 1H), 7.94-7.91 (m, 1H), 7.54-7.50 (m, 1H), 7.35 (t, J = 9.2 Hz, 1H), 7.11 (s, 1H), 6.00 (s, 2H), 5.51 (s, 1H), 3.50 (s, 3H), 3.46-3.40 (m, 1H), 2.51-2.50 (m, 2H), 2.35-2.30 (m, 2H), 2.19-2.15 (m, 2H), 1.83-1.72 (m, 4H) ppm.

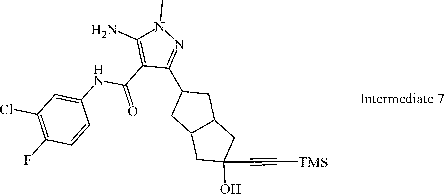 [SMILES: Cn1nc(C2CC3CC(O)(C#C[Si](C)(C)C)CC3C2)c(C(=O)Nc2ccc(F)c(Cl)c2)c1N]

5-Amino-N-(3-chloro-4-fluorophenyl)-3-(5-hydroxy-5-((trimethylsilyl)ethynyl)octahydropentalen-2-yl)-1-methyl-1H-pyrazole-4-carboxamide A solution of ethynyltrimethylsilane (2.0 g, 20.4 mmol) in THF (20 mL) was added n-BuLi (8.2 mL, 2.5 M, 20.5 mmol) at 0° C. under N₂, the mixture was stirred at 0° C. for 1 h. 5-amino-N-(3-chloro-4-fluorophenyl)-1-methyl-3-(5-oxooctahydropentalen-2-yl)-1H-pyrazole-4-carboxamide (1.0 g, 2.56 mmol) was then added in one portion. The resulting mixture was kept at 0° C. and stirred for 1 h. The reaction was quenched with NH₄Cl solution and extracted with ethyl acetate. The organic layer was dried and concentrated. The residue was purified through silica gel column chromatography (petroleum ether/ethyl acetate=1:50) to afford 5-amino-N-(3-chloro-4-fluorophenyl)-3-(5-hydroxy-5-((trimethylsilyl)ethynyl)octahydropentalen-2-yl)-1 -methyl- 1H-pyrazole-4-carboxamide as a white solid. TLC; 100% ethyl acetate (R_{f}: 0.4). MS calcd.: 488.2. Found: 489.3 [M+1]⁺. ¹H-NMR (400 MHz, DMSO-d₆): δ 8.79 (s, 1H), 7.79 (dd, J = 7.2, 2.4 Hz, 1H), 7.41-7.37 (m, 1H), 7.23 (t, J = 9.2 Hz, 1H), 5.87 (s, 2H), 5.16 (s, 1H), 3.37 (s, 3H), 3.30-3.26 (m, 1H), 2.43-2.38 (m, 2H), 2.01-1.98 (m, 2H), 1.89-1.84 (m, 2H), 1.59-1.94 (m, 4H), 0.00 (s, 9H) ppm.

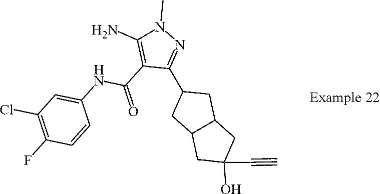 [SMILES: C#CC1(O)CC2CC(c3nn(C)c(N)c3C(=O)Nc3ccc(F)c(Cl)c3)CC2C1]

5-Amino-N-(3-chloro-4-fluorophenyl)-3-(5-ethynyl-5-hydroxyoctahydropentalen-2-yl)-1-methyl-1H-pyrazole-4-carboxamide. To a solution of 5-amino-N-(3-chloro-4-fluorophenyl)-3-(5-hydroxy-5-((trimethylsilyl)ethynyl)octahydropentalen-2-yl)-1-methyl-1H-pyrazole-4-carboxamide ( 400 mg, 0.82 mmol) in MeOH (5 ml) was added K₂CO₃ (170 mg, 1.23 mmol). The mixture was stirred at room temperature for 2 h. Water (50 ml) was added and the solution extracted with ethyl acetate. The organic layer was dried and concentrated. The crude was purified through prep-HPLC to afford 5-amino-N-(3-chloro-4-fluorophenyl)-3-(5-ethynyl-5-hydroxyoctahydropentalen-2-yl)-1-methyl-1H-pyrazole-4-carboxamide as a white solid. TLC: 100% ethyl acetate (R_{f}— 0.2). MS; calcd; 416.1. Found; 417.3 [M+ 1]⁺. ¹H-NMR (400 MHz, DMSO-d₆): δ 8.92 (s, 1H), 7.91 (dd, J = 6.8, 2.4 Hz, 1H), 7.53-7.49 (m, 1H), 7.35 (t, J = 8.8 Hz, 1H), 5.98 (s, 2H), 5.29 (s, 1H), 3.49 (s, 3H), 3.46-3.37 (m, 1H), 3.23 (s, 1H), 2.55-2.50 (m, 2H), 2.15-2.08 (m, 2H), 2.02-1.97 (m, 2H), 1.70-1.62 (m, 4H) ppm.

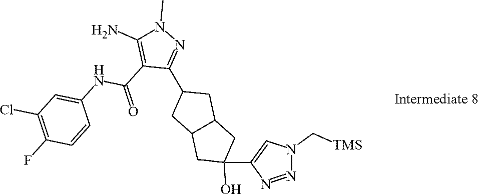 [SMILES: Cn1nc(C2CC3CC(O)(c4cn(C[Si](C)(C)C)nn4)CC3C2)c(C(=O)Nc2ccc(F)c(Cl)c2)c1N]

5-Amino-N-(3-chloro-4-fluorophenyl)-3-(5-hydroxy-5-(1-((trimethylsilyl)methyl)-1H-1.2,3-triazol-4-yl)octahydropentalen-2-yl)-1-methyl-1H-pyrazole-4-carboxamide. To a solution of 5-amino-N-(3-chloro-4-fluorophenyl)-3-(5-ethynyl-5-hydroxyoctahydropentalen-2-yl)-1-methyl-1H-pyrazole-4-carboxamide ( 150 mg, 0.36 mmol) and (azidomethyl)trimethylsilane (56 mg, 0.43 mmol) in THF (2 ml) and H₂O(2 ml) was added CuSO₄ -5H₂O (15 mg, 0.058 mmol) and Na ascorbate (15 mg, 0.076 mmol). The mixture was stirred at room temperature for 6 h. Water (20 ml) was added and the solution extracted with ethyl acetate. The organic layer was dried and concentrated. The crude product was purified by silica gel column chromatography using DCM/MeOH=20:1 to afford 5-amino-N-(3-chloro-4-fluorophenyl)-3-(5-hydroxy-5-(1-((trimethylsilyl)methyl)-1H-1.2,3-triazol-4-yl)octahydropentalen-2-yl)-1-methyl-1H-pyrazole-4-carboxamide as a white solid. TLC: 5% MeOH/DCM (R_{f}: 0.5). MS Calcd.: 545.2. Found: 546.3 [M+ 1]⁺.

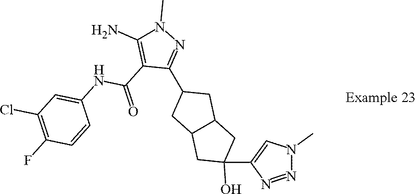 [SMILES: Cn1cc(C2(O)CC3CC(c4nn(C)c(N)c4C(=O)Nc4ccc(F)c(Cl)c4)CC3C2)nn1]

5-Amino-N-(3-chloro-4-fluorophenyl)-3-(5-hydroxy-5-(1-methyl-1H-1,2,3-triazol-4-yl)octahydropentalen-2-yl)-1-methyl-1H-pyrazole-4-carboxamide To a solution of 5-amino-N-(3-chloro-4-fluorophenyl)-3-(5-hydroxy-5-(1-((trimethylsilyl)methyl)-1H-1,2,3-triazol-4-yl)octahydropentalen-2-yl)-1-methyl-1H-pyrazole-4-carboxamide ( 180 mg, 0.33 mmol) in THF(3 ml) was added Bu₄NF (0.5 mL, 1.0 M, 0.5 mmol). The mixture was stirred at room temperature overnight. Water(50 ml) was added and extracted with ethyl acetate. The organic layer was dried and concentrated. The crude product was purified by prep-HPLC to afford 5-amino-N-(3-chloro-4-fluorophenyl)-3-(5-hydroxy-5-(1-methyl-1H-1,2,3-triazol-4-yl)octahydropentalen-2-yl)-1-methyl-1H-pyrazole-4-carboxamide (30 mg, 19%) as a white solid. TLC: 5% MeOH/DCM (R_{f}: 0.3). MS Calcd.: 473.2. Found: 474.2 [M+ 1]. ¹H-NMR (400 MHz, DMSO-d₆): δ 8.91 (s, 1H), 7.92 (dd, J = 6.8, 2.4 Hz, 1H), 7.81 (s, 1H), 7.53-7.49 (m, 1H), 7.35 (t, J = 9.2 Hz, 1H), 6.00 (s, 2H), 4.99 (s, 1H), 3.98 (s, 3H), 3.50 (s, 3H), 3.43-3.40 (m, 1H), 2.54-2.50 (m, 2H), 2.26-2.21 (m, 2H), 2.18-2.13 (m, 2H), 1.84-1.72 (m, 4H) ppm.

| | |
|---|---|
| Example | Structure and analysis |
| Example 24 | 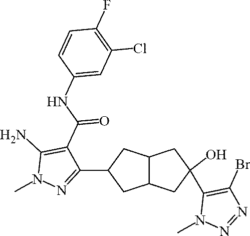 [SMILES: Cn1nc(C2CC3CC(O)(c4c(Br)nnn4C)CC3C2)c(C(=O)Nc2ccc(F)c(Cl)c2)c1N] |
| | 5-Amino-3-(5-(4-bromo-1-methyl-1H-1,2,3-triazol-5-yl)-5-hydroxyoctahydropentalen-2-yl)-N-(3-chloro-4-fluorophenyl)-1-methyl-1H-pyrazole-4-carboxamide ¹H-NMR (400 MHz, DMSO-d6): δ 8.94 (s, 1H), 7.91 (dd, J = 6.8, 2.4 Hz, 1H), 7.50-7.53 (m, 1H), 7.35 (t, J = 9.2 Hz, 1H), 5.98 (s, 2H), 5.48 (s, 1H), 4.15 (s, 3H), 3.49 (s, 3H), 3.38-3.44 (m, 1H), 2.63-2.67 (m, 2H), 2.29-2.35 (m, 2H), 2.05-2.18 (m, 4H), 1.85-1.93 (m, 2H) ppm |
| Example 25 | 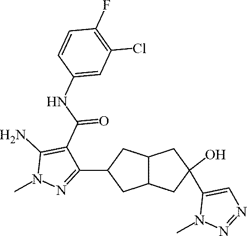 [SMILES: Cn1nncc1C1(O)CC2CC(c3nn(C)c(N)c3C(=O)Nc3ccc(F)c(Cl)c3)CC2C1] |
| | 5-Amino-N-(3-chloro-4-fluorophenyl)-3-(5-hydroxy-5-(1-methyl-1H-1,2,3-triazol-5-yl)octahydropentalen-2-yl)-1-methyl-1H-pyrazole-4-carboxamide ¹H-NMR (400 MHz, CD₃OD): 7.80 (dd, J = 6.8, 2.4 Hz, 1H), 7.57 (s, 1H), 7.39-7.42( m, 1H), 7.18 (t, J = 8.8 Hz, 1H), 4.17 (s, 3H), 3.58 (s, 3H), 3.43-3.47 (m, 1H), 2.66-2.67 (m, 2H), 2.32-2.37 (m, 2H), 2.01-2.05 (m, 2H), 1.85-1.93 (m, 2H) ppm |
| Example 26 | 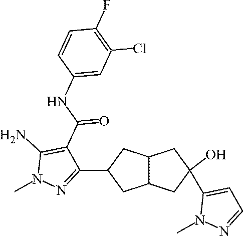 [SMILES: Cn1nccc1C1(O)CC2CC(c3nn(C)c(N)c3C(=O)Nc3ccc(F)c(Cl)c3)CC2C1] |
| | 5-Amino-N-(3-chloro-4-fluorophenyl)-3-(5-hydroxy-5-(1-methyl-1H-pyrazol-5-yl)octahydropentalen-2-yl)-1-methyl-1H-pyrazole-4-carboxamide ¹H-NMR (400 MHz, DMSO-d6): δ 8.93 (s, 1H), 7.91 (dd, J = 6.8, 2.4 Hz, 1H), 7.53-7.49 (m, 1H), 7.34 (t, J = 9.2 Hz, 1H), 7.21 (d, J = 1.6 Hz, 1H), 6.08 (d, J = 2 Hz, 1H), 5.99 (s, 2H), 5.17 (s, 1H), 3.90 (s, 3H), 3.50 (s, 3H), 3.46-3.40 (m, 1H), 2.51-2.49 (m, 2H), 2.20-2.16 (m, 4H), 1.90-1.85 (m, 2H), 1.81-1.73 (m, 2H) ppm |

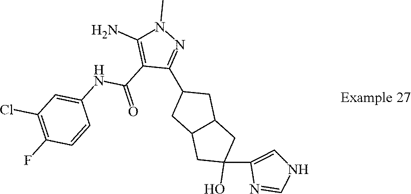 [SMILES: Cn1nc(C2CC3CC(O)(c4c[nH]cn4)CC3C2)c(C(=O)Nc2ccc(F)c(Cl)c2)c1N]

5-Amino-N-(3-chloro-4-fluorophenyl)-3-(5-hydroxy-5-(1H-imidazol-4-yl)octahydropentalen-2-yl)-1-methyl-1H-pyrazole-4-carboxamide To a solution of 4-bromo-1H-imidazole (200.0 mg, 1.4 mmol) in THF (15 mL) was added n-Butyllithium (1.4 mL, 3.4 mmol, 2.5 M) dropwise at -78° C. The resulting solution was allowed to warm to room temperature and stirred for 1 hour. The reaction was then cooled to -78° C. and a solution of 5-amino-N-(3-chloro-4-fluorophenyl)-1-methyl-3-(5-oxooctahydropentalen-2-yl)-1H-pyrazole-4-carboxamide (200.0 mg, 0.5 mmol) in anhydrous tetrahydrofuran (4 mL) added over 5 min. The mixture was allowed to warm to room temperature and stirred overnight. The reaction was quenched with saturated aqueous ammonium chloride solution (1 mL). The solvent was removed and the residue diluted with water, extracted with ethyl acetate (3 × 20 mL), dried over Na₂SO₄, filtered and concentrated to give the crude product which was purified by column chromatography (0-10% methanol in DCM) and basic prep-HPLC to afford 5-amino-N-(3-chloro-4-fluorophenyl)-3-(5-hydroxy-5-(1H-imidazol-4-yl)octahydropentalen-2-yl)-1-methyl-1H-pyrazole-4-carboxamide as a white solid. TLC: 10% MeOH/DCM (R_{f}: 0.2). MS Calcd.: 458.2. Found: 441.0 [M - 18 + 1]. ¹H NMR (400 MHz, CD₃OD) δ 7.80 (dd, J = 6.8, 2.8 Hz, 1H), 7.59 (s, 1H), 7.42-7.38 (m, 1H), 7.18 (t, J = 8.8 Hz, 1H), 6.95 (s, 1H), 3.56 (s, 3H), 3.45-3.39 (m, 1H), 2.63-2.62 (m, 2H), 2.42-2.37 (m, 2H), 1.90-1.82 (m, 4H) ppm.

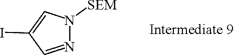 [SMILES: C[Si](C)(C)CCOCn1cc(I)cn1]

4-Iodo((2-(trimethylsilyl)ethoxy)methyl)-1H-pyrazole. A mixture of 4-iodo-1H-pyrazole (3.0 g, 15.5 mmol) and NaH (744.0 mg, 60%) in THF (30 mL) was stirred at 0° C. for 30 min. SEMC1 (2.84 g, 17.1 mmol) was then added dropwise over 5 min and the reaction mixture stirred for 2 hours. The mixture was quenched with saturated aqueous ammonium chloride solution (1 mL). The solvent was removed and the residue diluted with water, extracted with ethyl acetate (3 × 40 mL), dried over Na₂SO₄, filtered and concentrated to give the crude product which was purified by column chromatography using 0-25% petroleum ether in ethyl acetate to afford 4-iodo-1-((2-(trimethylsilyl)ethoxy)methyl)-1H-pyrazole (4.5 g, 90.0%) as a colorless oil. TLC: 25% PE/EA (R_{f}: 0.6). MS Calcd.: 324.0. MS Found: 325.1 [M + 1]⁺.

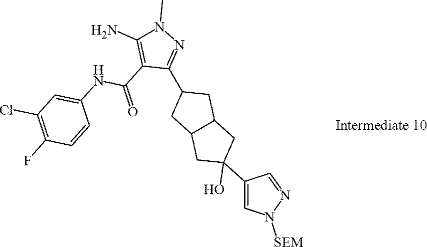 [SMILES: Cn1nc(C2CC3CC(O)(c4cnn(COCC[Si](C)(C)C)c4)CC3C2)c(C(=O)Nc2ccc(F)c(Cl)c2)c1N]

5-Amino-N-(3-chloro-4-fluorophenyl)-3-(5-hydroxy-5-(1-((2-(trimethylsilyl)-ethoxy)methyl)-1H-pyrazol-4-yl)octahydropentalen-2-yl)-1-methyl-1H-pyrazole-4-carboxamide. A mixture of 4-iodo-1-((2-(trimethylsilyl)ethoxy)methyl)-1H-pyrazole (2.1 g, 6.4 mmol) and iPrMgCl (2.5 mL, 2 M in THF) in THF (20 mL) was stirred at room temperature for 1 hour, then a solution of 5-amino-N-(3-chloro-4-fluorophenyl)-1-methyl-3-(5-oxooctahydropentalen-2-yl)-1H-pyrazole-4-carboxamide (500.0 mg, 1.3 mmol) in THF (4 mL) was added dropwise over 5 min. The reaction mixture was stirred overnight. The reaction was quenched with saturated aqueous ammonium chloride solution (1 mL). The solvent was removed and the residue diluted with water, extracted with ethyl acetate (3 × 20 mL), dried over Na₂SO₄, filtered and concentrated to give the crude product, which was purified by column chromatography using 0-15% methanol in DCM to afford 5-amino-N-(3-chloro-4-fluorophenyl)-3-(5-hydroxy-5-(1-((2-(trimethylsilyl)ethoxy)methyl)-1H-pyrazol-4-yl)octahydropentalen-2-yl)-1-methyl-1H-pyrazole-4-carboxamide as a brown solid. TLC: 10% MeOH/DCM (R_{f}: 0.3). MS Calcd.: 588.2. Found: 589.3 [M + 1]⁺.

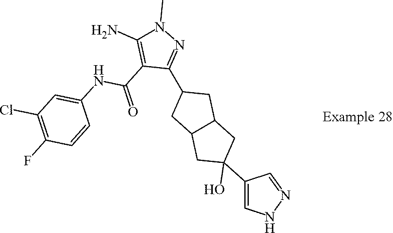 [SMILES: Cn1nc(C2CC3CC(O)(c4cn[nH]c4)CC3C2)c(C(=O)Nc2ccc(F)c(Cl)c2)c1N]

5-Amino-N-(3-chloro-4-fluorophenyl)-3-(5-hydroxy-5-(1H-pyrazol-4-yl)octahydropentalen-2-yl)-1-methyl-1H-pyrazole-4-carboxamide A mixture of afford 5-amino-N-(3-chloro-4-fluorophenyl)-3-(5-hydroxy-5-(1-((2-(trimethylsilyl)ethoxy)methyl)-1H-pyrazol-4-yl)octahydropentalen-2-yl)-1-methyl-1H-pyrazole-4-carboxamide (100.0 mg, 0.17 mmol) and TFA (1 mL) in DCM (5 mL) was stirred at room temperature for 1 hour. The reaction mixture was quenched with saturated aqueous ammonium chloride solution (1 mL) and the solvent removed. The mixture was diluted with water, basified by Sat. NaHCO₃ (aq.) (pH > 7) then extracted with ethyl acetate (3 × 20 mL), dried over Na₂SO₄, filtered and concentrated to give the crude product which was purified by column chromatography using 0-20% methanol in DCM and basic prep-HPLC to afford 5-amino-N-(3-chloro-4-fluorophenyl)-3-(5-hydroxy-5-(1H-pyrazol-4-yl)octahydropentalen-2-yl)-1-methyl-1H-pyrazole-4-carboxamide as a white solid. TLC: 15% MeOH/DCM (R_{f}: 0.3). MS Calcd.: 458.2. MS Found: 441.0 [M- 18]. ¹H NMR (400 MHz, DMSO-d₆,): δ 12.68 (brs, 1H), 9.00 (s, 1H), 7.92 (dd, J = 7.2, 2.8 Hz, 1H), 7.65 (brs, 2H), 7.55-7.51 (m, 1H), 7.36 (t, J = 8.8 Hz, 1H), 6.95 (s, 2H), 5.76 (s, 1H), 3.46 (s, 3H), 3.45-3.40 (m, 1H), 3.33-3.23 (m, 2H), 2.74-2.69 (m, 2H), 2.33-2.17 (m, 2H), 1.47-1.35 (m, 4H) ppm.

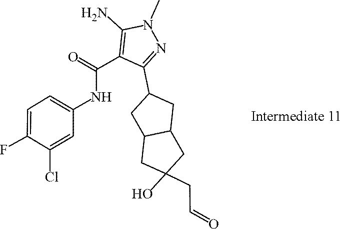 [SMILES: Cn1nc(C2CC3CC(O)(CC=O)CC3C2)c(C(=O)Nc2ccc(F)c(Cl)c2)c1N]

5-Amino-N-(3-chloro-4-fluorophenyl)-3-(5-hydroxy-5-(2-oxoethyl)octahydropentalen-2-yl)-1-methyl-1H-pyrazole-4-carboxamide. OsO₄ (15 mg, 0.0578 mmol) in tBuOH (5 mL) and NaIO₄ (0.74 g, 3.47 mmol) were added to a stirred solution of 3-(5-allyl-5-hydroxyoctahydropentalen-2-yl)-5-amino-N-(3-chloro-4-fluorophenyl)-1-methyl-1H-pyrazole-4-carboxamide (0.5 g, 1.15 mmol) in 10 mL 1:1 ether/H₂O and the reaction stirred at room temperature for 2 hr. After completion, the reaction mixture was diluted with water and extracted with ethyl acetate. The combined organic layers were dried over anhydrous sodium sulphate, filtered and concentrated under reduced pressure to afford 0.3 g of 5-amino-N-(3-chloro-4-fluorophenyl)-3-(5-hydroxy-5-(2-oxoethyl)octahydropentalen-2-yl)-1-methyl-1H-pyrazole-4-carboxamide which used for the next step without further purification.

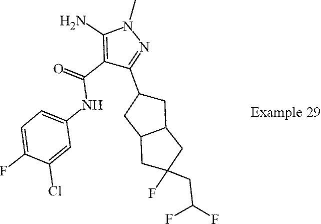 [SMILES: Cn1nc(C2CC3CC(F)(CC(F)F)CC3C2)c(C(=O)Nc2ccc(F)c(Cl)c2)c1N]

5-Amino-N-(3-chloro-4-fluorophenyl)-3-(5-(2,2-difluoroethyl)-5-fluorooctahydropentalen-2-yl)-1-methyl-1H-pyrazole-4-carboxamide. DAST (0.148 g, 0.92 mmol) was added to a stirred solution of 5-amino-N-(3-chloro-4-fluorophenyl)-3-(5-hydroxy-5-(2-oxoethyl)octahydropentalen-2-yl)-1-methyl-1H-pyrazole-4-carboxamide (0.2 g, 0.46 mmol) in DCM (3 mL) at 0° C. and the mixture stirred at room temperature for 6 h. After completion, the reaction mixture was diluted with saturated NaHCO₃ and extracted with DCM. The combined organic layer was dried over anhydrous sodium sulphate, filtered, and concentrated under reduced pressure. The crude compound was purified by prep. HPLC to afford 5-amino-N-(3-chloro-4-fluorophenyl)-3-(5-(2,2-difluoroethyl)-5-fluorooctahydropentalen-2-yl)-1-methyl-1H-pyrazole-4-carboxamide as an off white solid. MS calcd for C₂₁H₂₃ClF₄N₄O; 485.15. Found; 495.50 [M+1]⁺. ¹H NMR (400 MHz, DMSO-d₆): δ 8.95 (s, 1H), 7.91 (dd, J = 6.6, 2.0 Hz, 1H), 7.54-7.48 (m, 1H), 7.34 (t, J = 8.8 Hz, 1H), 6.35-5.94 (m, 3H), 3.68-3.55 (m, 1H), 3.48 (s, 3H), 2.72-2.60 (m, 2H), 2.50-2.30 (m, 2H, merged), 2.22-2.04 (m, 4H), 1.66-1.44 (m, 4H) ppm.

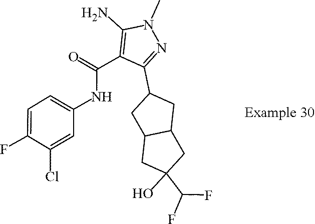 [SMILES: Cn1nc(C2CC3CC(O)(C(F)F)CC3C2)c(C(=O)Nc2ccc(F)c(Cl)c2)c1N]

5-Amino-N-(3-chloro-4-fluorophenyl)-3-(5-(difluoromethyl)-5-hydroxyoctahydropentalen-2-yl)-1-methyl-1H-pyrazole-4-carboxamide. CsF (0.015 g, 0.10 mmol), 18-Crown-6 (0.026 g, 0.10 mmol) and (difluoromethyl)-trimethylsilane (0.31 mL, 2.56 mmol) were added to a stirred suspension of 5-amino-N-(3-chloro-4-fluorophenyl)-1-methyl-3-(5-oxooctahydropentalen-2-yl)-1H-pyrazole-4-carboxamide (0.2 g, 0.51 mmol) in DME (2 mL) at 0° C., and stirring continued at room temperature for 24 h. After completion, the reaction mixture was diluted with water and extracted with ethyl acetate. The combined organic layers were dried over anhydrous sodium sulphate, filtered, and concentrated under reduced pressure. The crude compound was purified by prep. HPLC to afford 5-amino-N-(3-chloro-4-fluorophenyl)-3-(5-(difluoromethyl)-5-hydroxyoctahydropentalen-2-yl)-1-methyl-1H-pyrazole-4-carboxamide as an off-white solid. MS calcd for C₂₀H₂₂CIF₃N₄O₂; 442.14. Found; 443.05 [M + 1]⁺. ¹H NMR (400 MHz, DMSO-d₆): δ 8.92 (s, 1H), 7.91 (dd, J = 7.0, 2.4 Hz, 1H), 7.54-7.48 (m, 1H), 7.34 (t, J = 9.2 Hz, 1H), 5.97 (s, 2H), 5.73 (t, J = 56.4 Hz, 1H), 5.02 (s, 1H), 3.49 (s, 3H), 3.49-3.35 (m, 1H), 2.60-2.54 (m, 2H, merged), 2.20-2.10 (m, 2H), 1.92-1.70 (m, 4H), 1.56-1.48 (m, 2H) ppm.

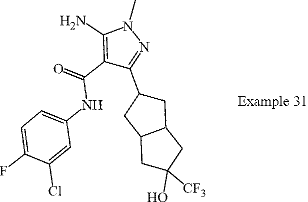 [SMILES: Cn1nc(C2CC3CC(O)(C(F)(F)F)CC3C2)c(C(=O)Nc2ccc(F)c(Cl)c2)c1N]

5-Amino-N-(3-chloro-4-fluorophenyl)-3-(5-hydroxy-5-(trifluoromethyl)octahydropentalen-2-yl)-1-methyl-1H-pyrazole-4-carboxamide. TBAF (10.24 mL THF solution, Imol/L, 10.24 mmol), and trimethyl(trifluoromethyl)silane (13 mL, >20 eq) were added slowly to a solution of 5-amino-N-(3-chloro-4-fluorophenyl)-1-methyl-3-(5-oxooctahydropentalen-2-yl)-1H-pyrazole-4-carboxamide (2 g, 5.12 mmol) in THF (50 mL), at 0° C. The mixture was stirred at room temperature for 15 mins, then at 60° C. overnight. Additional trimethyl- (trifluoromethyl)silane (3 mL) was added slowly at room temperature and the mixture stirred at 60° C. for another 3 h. The solution was quenched with H₂O, extracted with ethyl acetate. The organic layer was concentrated and the residue was purified by silica gel column chromatography (10%~20% CH₃OH/DCM), reversed phase column chromatography and then silica column chromatography (60%~80% ethyl acetate/petroleum ether) to afford 5-amino-N-(3-chloro-4-fluorophenyl)-3-(5-hydroxy-5-(trifluoromethyl)octahydropentalen-2-yl)-1-methyl-1H-pyrazole-4-carboxamide as a white solid. MS calcd for C₂₀H₂₁ClF₄N₄O₂; 460.13. Found: 461.1 [M + 1]⁺. ¹H NMR (400 MHz, DMSO-d₆): δ 8.95 (s, 1H), 7.92 (dd, J = 2.4 Hz, 4.4 Hz, 1H), 7.52 - 7.51 (m, 1H), 7.35 (t, J = 9.2 Hz, 1H), 5.99 (s, 2H), 5.70 (s, 1H), 3.49 (s, 3H), 3.41 - 3.37 (m, 1H), 2.65 - 2.63 (m, 2H), 2.16 - 2.13 (m, 2H), 2.01 - 1.95 (m, 2H), 1.82 - 1.80 (m, 2H), 1.70 - 1.67 (m, 2H) ppm.

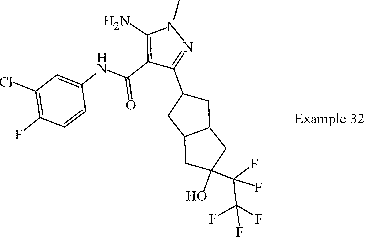 [SMILES: Cn1nc(C2CC3CC(O)(C(F)(F)C(F)(F)F)CC3C2)c(C(=O)Nc2ccc(F)c(Cl)c2)c1N]

5-Amino-N-(3-chloro-4-fluorophenyl)-3-(5-hydroxy-5-(perfluoroethyl)octahydropentalen-2-yl)-1-methyl-1H-pyrazole-4-carboxamide To solution of 1,1,1,2,2-pentafluoro-2-iodoethane (2.36 g, 5.4 eq, 9.59 mmol) in dry THF (40 mL) was added LiMe solution (1.6 M, 6 mL, 5.4 eq, 9.59 mmol) dropwise at -78° C. The reaction was stirred for 1 h at -78° C. in an Ar atmosphere. Following this, a solution of 5-amino-N-(3-chloro-4-fluorophenyl)-1-methyl-3-((2 s,3aR,6aS)-5-oxooctahydropentalen-2-yl)-1H-pyrazole-4-carboxamide (700 mg, 1.0 eq, 1.79 mmol) in dry THF (5 mL) was added dropwise at -78° C. The reaction was stirred for 2 h at -78° C. The mixture was quenched with NH₄Cl solution (40 mL) and extracted with EtOAc (20 mL x 3). The combined organic layer was dried with anhydrous Na₂SO₄ and concentrated in vacuo. The residue was purified by reserve-phase column chromatography to afford a white solid (60 mg, 6.6% yield). MS Calcd.: 510.1, MS Found: 511.1 [M + 1]⁺. ¹H-NMR (400 MHz, DMSO-d₆): δ 8.94 (s, 1H), 7.92 (dd, J = 6.8, 2.4 Hz, 1H), 7.55 - 7.49 (m, 1H), 7.35 (t, J = 9.2 Hz, 1H), 5.98 (s, 2H), 5.73 (s, 1H), 3.49 (s, 3H), 3.43 - 3.34 (m, 1H), 2.72 - 2.60 (m, 2H), 2.20 - 2.09 (m, 2H), 2.04 -1.94 (m, 2H), 1.91 - 1.80 (m, 2H), 1.76 (d, J =13.6 Hz, 2H) ppm.

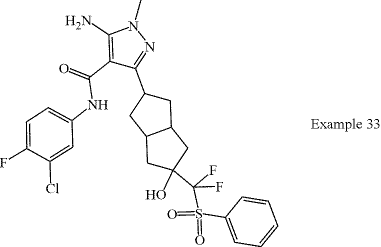 [SMILES: Cn1nc(C2CC3CC(O)(C(F)(F)S(=O)(=O)c4ccccc4)CC3C2)c(C(=O)Nc2ccc(F)c(Cl)c2)c1N]

5-Amino-N-(3-chloro-4-fluorophenyl)-3-(5-(difluoro(phenylsulfonyl)methyl)-5-hydroxyoctahydropentalen-2-yl)-1-methyl-1H-pyrazole-4-carboxamide. LiHMDS (2.3 mL, 2.3 mmol, 1 M in THF) was added to a stirred solution of 5-amino-N-(3-chloro-4-fluorophenyl)-1-methyl-3-(5-oxooctahydropentalen-2-yl)-1H-pyrazole-4-carboxamide (0.2 g, 0.51 mmol), ((difluoromethyl)-sulfonyl)benzene (0.29 g, 1.53 mmol) in THF ( 5 mL) at -78° C., and stirring continued for 1.5 h. The reaction mixture was allowed to warm to room temperature and stirred for 16 h. After completion, the mixture was diluted with water and extracted with ethyl acetate. The combined organic layers were dried over anhydrous sodium sulphate, filtered, and concentrated under reduced pressure. The crude compound was purified by prep. HPLC to afford 5-amino-N-(3-chloro-4-fluorophenyl)-3-(5-(difluoro(phenylsulfonyl)methyl)-5-hydroxyoctahydropentalen-2-yl)-1-methyl-1H-pyrazole-4-carboxamide as a white solid. MS calcd for C₂₆H₂₆ClF₃N₄O₄S; 582.13. Found; 583.10 [M + 1]⁺. ¹H NMR (400 MHz, DMSO-d₆): δ 8.92 (s, 1H), 7.98-7.80 (m, 4H), 7.71 (t, J = 7.2 Hz, 2H), 7.55-7.48 (m, 1H), 7.38-7.30 (m, 1H), 5.97 (s, 2H), 5.66 (s, 1H), 3.48 (s, 3H), 3.38-3.26 (m, 1H, merged), 2.71-2.54 (m, 2H), 2.19-2.00 (m, 4H), 1.92-1.74 (m, 4H) ppm.

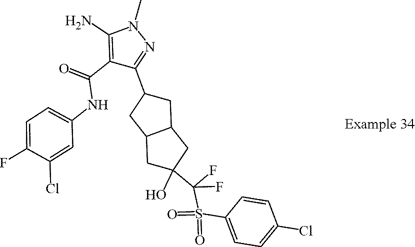 [SMILES: Cn1nc(C2CC3CC(O)(C(F)(F)S(=O)(=O)c4ccc(Cl)cc4)CC3C2)c(C(=O)Nc2ccc(F)c(Cl)c2)c1N]

5-Amino-N-(3-chloro-4-fluorophenyl)-3-(5-(((4-chlorophenyl)sulfonyl)difluoromethyl)-5-hydroxyoctahydropentalen-2-yl)-1-methyl-1H-pyrazole-4-carboxamide. To a stirred solution of compound 5-amino-N-(3-chloro-4-fluorophenyl)-1-methyl-3-(5-oxooctahydropentalen-2-yl)-1H-pyrazole-4-carboxamide (300 mg, 0.76 mmol) and 1-chloro-4-(difluoromethyl)sulfonyl)benzene (366 mg, 1.61 mmol) in THF (5 mL) at -78° C., was added LiHMDS (2.5 M in THF) (0.7 mL,1.76 mmol) and the reaction mixture stirred at room temperature for16 h. After completion, the mixture was diluted with ice cold water and extracted with ethyl acetate. The combined organic layers were collected, dried over anhydrous sodium sulphate, filtered,concentrated in vacuo and purified by prep. HPLC to give 5-amino-N-(3-chloro-4-fluorophenyl)-3-(5-(((4-chlorophenyl)sulfonyl)difluoromethyl)-5-hydroxyoctahydropentalen-2-yl)-1-methyl-1H-pyrazole-4-carboxamide as a white solid . MS calc for C₂₆H₂₅Cl₂F₃N₄O₄S; 616.09. Found; 617.03 [M + 1]⁺. ¹H NMR (400 MHz, DMSO-d₆): δ 8.92 (s, 1H), 7.96-7.90 (m, 3H), 7.77 (d, J = 8.0 Hz, 2H), 7.52-7.50 (m, 1H), 7.34 (t, J = 9.2 Hz, 1H), 5.97 (m, 2H), 5.67 (s, 1H), 3.48 (s, 3H), 3.34-3.30 (m, 1H, merged), 2.63-2.62 (m, 2H), 2.13-2.03 (m, 4H), 1.87-1.73 (m, 4H) ppm.

| | |
|---|---|
| Example | Structure and analysis |
| Example 35 | 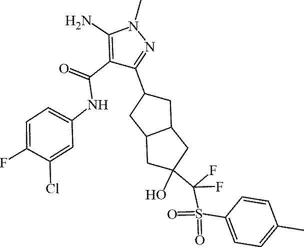 [SMILES: Cc1ccc(S(=O)(=O)C(F)(F)C2(O)CC3CC(c4nn(C)c(N)c4C(=O)Nc4ccc(F)c(Cl)c4)CC3C2)cc1] |
| | 5-Amino-N-(3-chloro-4-fluorophenyl)-3-(5-(difluoro(tosyl)methyl)-5-hydroxyoctahydropentalen-2-yl)-1-methyl-1H-pyrazole-4-carboxamide MS calcd for C₂₇H₂₈CIF₃N₄O₄S; 596.15. Found; 596.95 (M+1). ¹H NMR (400 MHz, DMSO-d₆): δ 8.92 (s, 1H), 7.92-7.90 (m, 1H), 7.81 (d, J = 8.4 Hz, 2H), 7.51 (d, J = 7.6 Hz, 3H), 7.36-7.32 (m, 1H), 5.97 (s, 2H), 5.61 (s, 1H), 3.57-3.50 (m, 1H), 3.49 (s, 3H), 3.11-2.97 (m, 4H), 2.62-2.50 (m, 1H), 2.45-2.35 (m, 1H), 2.24-2.04 (m, 5H), 1.95-1.85 (m, 2H) ppm. |
| Example 36 | 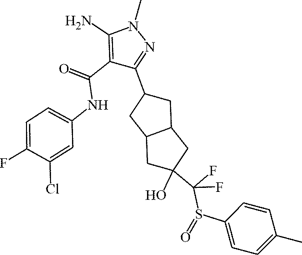 [SMILES: Cc1ccc(S(=O)C(F)(F)C2(O)CC3CC(c4nn(C)c(N)c4C(=O)Nc4ccc(F)c(Cl)c4)CC3C2)cc1] |
| | 5-Amino-N-(3-chloro-4-fluorophenyl)-3-(5-(difluoro(p-tolylsulfinyl)methyl)-5-hydroxyoctahydropentalen-2-yl)-1-methyl-1H-pyrazole-4-carboxamide MS calcd for C₂₇H₂₈ClF₃N₄O₃S; 580.15. Found; 581.05 (M+1). ¹H NMR (400 MHz, DMSO-d₆): δ 8.94 (s, 1H), 7.92 (dd, J = 6.8, 2.4 Hz, 1H), 7.58 (d, J = 8.4 Hz, 2H), 7.55-7.50 (m, 1H), 7.43 (d, J = 8.0 Hz, 2H), 7.37 (t, J = 9.2 Hz, 1H), 5.99 (s, 3H), 3.50 (s, 3H), 3.42-3.32 (m, 1H), 2.68-2.52 (m, 2H), 2.40 (s, 3H), 2.20-1.62 (m, 8H) ppm. |
| Example 37 | 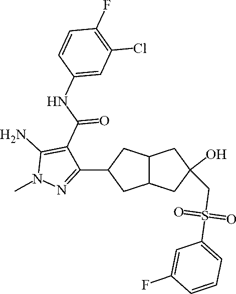 [SMILES: Cn1nc(C2CC3CC(O)(CS(=O)(=O)c4cccc(F)c4)CC3C2)c(C(=O)Nc2ccc(F)c(Cl)c2)c1N] |
| | 5-Amino-N-(3-chloro-4-fluorophenyl)-3-(5-(((3-fluorophenyl)sulfonyl)methyl)-5-hydroxyoctahydropentalen-2-yl)-1-methyl-1H-pyrazole-4-carboxamide ¹H-NMR (400 MHz, DMSO-d6): δ 8.92 (s, 1H), 7.90 (dd, J = 6.8, 2.4 Hz, 1H), 7.73-7.65 (m, 1H), 7.34 (t, J = 9.2 Hz, 1H), 5.94 (d, J = 6.4 Hz, 2H), 4.46 (s, 1H), 3.56 (s, 2H), 3.47 (s, 3H), 3.42-3.32 (m, 1H), 2.50-2.49 (m, 2H), 2.09-2.07 (m, 2H), 1.96-1.91 (m, 2H), 1.66-1.57 (m, 4H) ppm. |

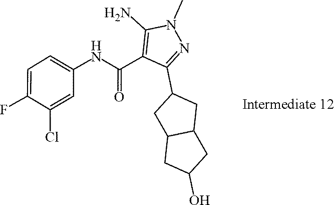 [SMILES: Cn1nc(C2CC3CC(O)CC3C2)c(C(=O)Nc2ccc(F)c(Cl)c2)c1N]

5-Amino-N-(3-chloro-4-fluorophenyl)-3-(5-hydroxyoctahydropentalen-2-yl)-1-methyl-1H-pyrazole-4-carboxamide. NaBH₄ (0.98 g, 25.70 mmol) was added to a stirred solution of 5-amino-N-(3-chloro-4-fluorophenyl)-1-methyl-3-(5-oxooctahydropentalen-2-yl)-1H-pyrazole-4-carboxamide (5 g, 12.85 mmol) in MeOH (50 mL) at 0° C. under an Ar atmosphere and the reaction stirred at 0° C. for 2 h. After completion, the reaction mixture was quenched with water and concentrated in vacuo. The residue was diluted with water and extracted with 10% MeOH/DCM. The combined organic layers were collected, dried over anhydrous sodium sulphate, filtered, concentrated in vacuo and purified by silica gel column chromatography to afford 5-amino-N-(3-chloro-4-fluorophenyl)-3-(5-hydroxyoctahydropentalen-2-yl)-1-methyl-1H-pyrazole-4-carboxamide as a white solid. TLC; 70% EtOAc/ hexanes (R_{f}: 0.1). LCMS calcd for C₁₉H₂₂ClFN₄O₂; 392.14. Found; 393.5. [M + 1]+.¹H NMR (400 MHz, DMSO-d₆,): δ 8.94 (s, 1H), 7.92-7.90 (m, 1H), 7.53-7.50 (m, 1H), 7.35 (t, J = 9.2 Hz, 1H), 5.96 (s, 2H), 4.46-4.44 (m, 1H), 4.10-4.04 (m, 1H), 3.50 (s, 3H), 3.46-3.38 (m, 1H), 2.40-2.35 (m, 2H), 2.17-2.13 (m, 2H), 1.93-1.86 (m, 2H), 1.63-1.54 (m, 2H), 1.31-1.26 (m, 2H) ppm.

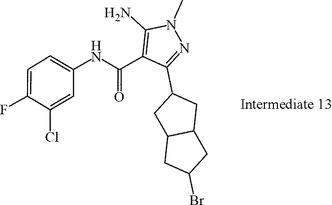 [SMILES: Cn1nc(C2CC3CC(Br)CC3C2)c(C(=O)Nc2ccc(F)c(Cl)c2)c1N]

5-Amino(5-bromooctahydropentalen-2-yl)-N-(3-chloro-4-fluorophenyl)-1-methyl-1H-pyrazole-4-carboxamide. Triphenylphosphine (1.99 g, 7.63 mmol) was added to a stirred solution of 5-amino-N-(3-chloro-4-fluorophenyl)-3-(5-hydroxyoctahydropentalen-2-yl)-1-methyl-1H-pyrazole-4-carboxamide (2 g, 5.08 mmol) in DCM (30 mL) at 0° C. under an Argon atmosphere and stirring continued for 10 min. CBr₄ (2.86 g, 8.63 mmol) was added portion-wise to this solution. The reaction mixture was stirred at room temperature for 16 h. After completion, the mixture was diluted with water and extracted with DCM. The combined organic layers were collected, dried over anhydrous sodium sulphate, filtered, concentrated in vacuo and purified by silica gel column chromatography to afford 5-amino-3-(5-bromooctahydropentalen-2-yl)-N-(3-chloro-4-fluorophenyl)-1-methyl-1H-pyrazole-4-carboxamide. TLC; EtOAc (R_{f}: 0.2). LCMS Calculated for C₁₉H₂₁BrClFN₄O— 454.06; Found; 455.3 [M + 1]⁺.

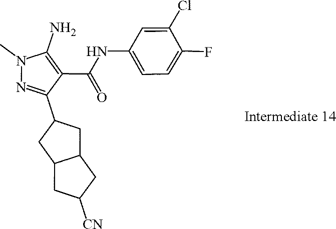 [SMILES: Cn1nc(C2CC3CC(C#N)CC3C2)c(C(=O)Nc2ccc(F)c(Cl)c2)c1N]

5-Amino-N-(3-chloro-4-fluorophenyl)-3-(5-cyanooctahydropentalen-2-yl)-1-methyl-1H-pyrazole-4-carboxamide, Isomer I. NaCN (0.719 g, 14.5 mmol) was added to a stirred solution of 5-amino-3-(5-bromooctahydropentalen-2-yl)-N-(3-chloro-4-fluorophenyl)-1-methyl-1H-pyrazole-4-carboxamide (4.4 g, 9.69 mmol) in DMSO (50 mL). The reaction mixture was stirred at 60° C. for 4 h. After completion, the reaction mixture was diluted with water and extracted with ethyl acetate. The combined organic layers were collected, dried over anhydrous sodium sulphate, filtered, concentrated in vacuo and purified by silica gel column chromatography to afford 5-amino-N-(3-chloro-4-fluorophenyl)-3-(5-cyanooctahydropentalen-2-yl)-1-methyl-1H-pyrazole-4-carboxamide, Isomer I as an off white solid. TLC; 100% EtOAc (R_{f}: 0.3). MS calcd for C₂₀H₂₁ClFN₅O; 401.4. Found; 402.50 [M + 1]⁺. ¹H NMR (400 MHz, DMSO-d₆): δ 8.97 (s, 1H), 7.91-7.89 (m, 1H), 7.59-7.49 (m, 1H), 7.35 (t, J = 8.8 Hz, 1H), 5.97 (s, 2H), 3.56-3.49 (m, 4H), 3.00-2.95 (m, 1H), 2.50-2.48 (m, 2H, merged), 2.20-2.15 (m, 4H), 1.60-1.50 (m, 4H) ppm.

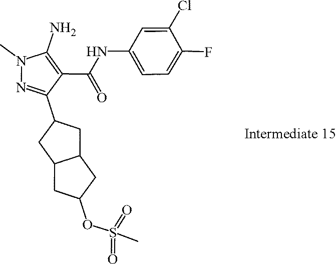 [SMILES: Cn1nc(C2CC3CC(OS(C)(=O)=O)CC3C2)c(C(=O)Nc2ccc(F)c(Cl)c2)c1N]

5-Amino-4-((3-chloro-4-fluorophenyl)carbamoyl)-1-methyl-1H-pyrazol-3-yl)octahydropentalen-2-yl methanesulfonate. DMAP (0.03 g, 0.254 mmol) and TEA (1.04 mL, 7.62 mmol) were added to a stirred solution of 5-amino-N-(3-chloro-4-fluorophenyl)-3-(5-hydroxyoctahydropentalen-2-yl)-1-methyl-1H-pyrazole-4-carboxamide (1 g, 2.54 mmol) in DCM (10 mL) at 0° C. and stirring continued for 10 min. To this solution was added MsCl (0.41 mL, 5.08 mmol). The mixture was stirred at room temperature for 16 h. After completion, the reaction mixture was diluted with water and extracted with ethyl acetate. The combined organic layers were collected, dried over anhydrous sodium sulphate, filtered, concentrated in vacuo and purified by silica gel column chromatography to afford 5-(5-amino-4-((3-chloro-4-fluorophenyl)carbamoyl)-1-methyl-1H-pyrazol-3-yl)octahydropentalen-2-yl methanesulfonate. TLC; 5% MeOH/DCM (R_{ƒ}: 0.5). LCMS calcd for C₂₀H₂₄CIFN₄O₄S; 470.12. Found; 471.20 [M + 1]⁺.

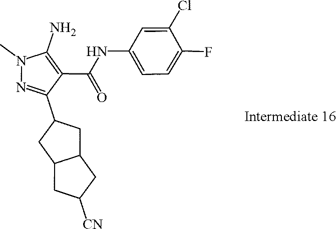 [SMILES: Cn1nc(C2CC3CC(C#N)CC3C2)c(C(=O)Nc2ccc(F)c(Cl)c2)c1N]

5-Amino-N-(3-chloro-4-fluorophenyl)-3-(5-cyanooctahydropentalen-2-yl)-1-methyl-1H-pyrazole-4-carboxamide Isomer II. NaCN (0.125 g, 2.35 mmol) was added to a stirred solution of 5-(5-amino-4-((3-chloro-4-fluorophenyl)carbamoyl)-1-methyl-1H-pyrazol-3-yl)octahydropentalen-2-yl methanesulfonate (0.6 g, 1.27 mmol) in DMSO (6 mL). The reaction mixture was stirred at 70° C. for 4 h. After completion, the reaction mixture was diluted with water and extracted with ethyl acetate. The combined organic layers were collected, dried over sodium sulphate, filtered, concentrated in vacuo and purified by silica gel column chromatography to afford 5-amino-N-(3-chloro-4-fluorophenyl)-3-(5-cyanooctahydropentalen-2-yl)-1-methyl-1H-pyrazole-4-carboxamide, Isomer II. MS calcd for C₂₀H₂₁CIFN₅O; 401.14. Found; 402.50 [M + 1]⁺. ¹H NMR (400 MHz, DMSO-d₆): δ 8.97 (s, 1H), 7.90 (dd, J = 6.8, 2.0 Hz, 1H), 7.53-7.49 (m, 1H), 7.35 (t, J = 9.2 Hz, 1H), 5.98 (s, 2H), 3.48 (s, 3H), 3.33-3.27 (m, 1H, merged), 2.94-2.88 (m, 1H), 2.60-2.57 (m, 2H), 2.16-2.12 (m, 2H), 1.86-1.73 (m, 4H), 1.30-1.22 (m, 2H) ppm.

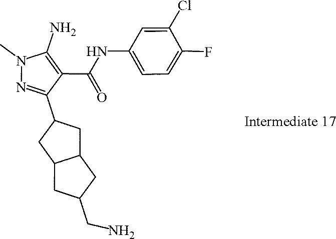 [SMILES: Cn1nc(C2CC3CC(CN)CC3C2)c(C(=O)Nc2ccc(F)c(Cl)c2)c1N]

5-Amino(5-(aminomethyl)octahydropentalen-2-yl)-N-(3-chloro-4-fluorophenyl)-1-methyl-1H-pyrazole-4-carboxamide Isomer I. A solution of 5-amino-N-(3-chloro-4-fluorophenyl)(5-cyanooctahydropentalen-2-yl)-1-methyl-1H-pyrazole-4-carboxamide, Isomer I (130.0 mg, 0.3 mmol) in BH₃-THF (1.0 M, 15 mL, 15 mmol) was stirred at 70° C. for 2 h. The reaction was quenched with MeOH. The solvent was removed in vacuo. The residue was purified by reversed phase chromatography to provide 5-amino-3-(5-(aminomethyl)octahydropentalen-2-yl)-N-(3-chloro-4-fluorophenyl)-1-methyl-1H-pyrazole-4-carboxamide Isomer I as a white solid. MS calcd; 405.2. Found; 406.3 [M + 1]⁺. ¹H NMR (400 MHz, DMSO-d₆): δ 8.96 (s, 1H), 7.92 (dd, J = 6.8, 2.8 Hz, 1H), 7.54-7.50 (m, 1H), 7.35 (t, J = 8.8 Hz, 1H), 5.97 (s, 2H), 3.59-3.49 (m, 5H), 2.43-2.33 (m, 3H), 2.16-2.12 (m, 2H), 1.96-1.94 (m, 3H), 1.45-1.37 (m, 2H), 0.93-0.86 (m, 2H) ppm. ¹H NMR (400 MHz, CD₃OD): δ 7.82 (dd, J = 6.8, 2.4 Hz, 1H), 7.45-7.41 (m, 1H), 7.21 (t, J = 8.8 Hz, 1H), 3.61-3.54 (m, 4H), 2.72 (d, J = 6.0 Hz, 2H), 2.64-2.61 (m, 2H), 2.34-2.27 (m, 2H), 2.14-2.11 (m, 3H), 1.59-1.51 (m, 2H), 1.08-1.00 (m, 2H) ppm.

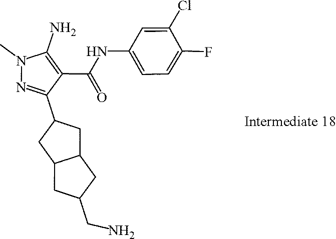 [SMILES: Cn1nc(C2CC3CC(CN)CC3C2)c(C(=O)Nc2ccc(F)c(Cl)c2)c1N]

5-Amino(5-(aminomethyl)octahydropentalen-2-yl)-N-(3-chloro-4-fluorophenyl)-1-methyl-1H-pyrazole-4-carboxamide Isomer II. A solution of 5-amino-N-(3-chloro-4-fluorophenyl)(5-cyanooctahydropentalen-2-yl)-1-methyl-1H-pyrazole-4-carboxamide Isomer II (100 mg, 0.249 mmol) in BH₃-THF (1.0 M, 10 mL, 10 mmol) was stirred at 70° C. for 2 h. The reaction was quenched with MeOH. The solvent was removed in vacuo. The residue was purified by reversed phase chromatography to provide 5-amino-3-(5-(aminomethyl)octahydropentalen-2-yl)-N-(3-chloro-4-fluorophenyl)-1-methyl-1H-pyrazole-4-carboxamide Isomer II as a white solid. MS calcd; 405.2. Found: 406.3 [M + 1]⁺. ¹H NMR (400 MHz, DMSO-d₆): δ 8.96 (s, 1H), 7.92 (dd, J = 6.8,2.4 Hz, 1H), 7.54-7.50 (m, 1H), 7.35 (t, J = 9.2 Hz, 1H), 5.97 (s, 2H), 3.49 (s, 3H), 3.32-3.24 (m, 2H), 2.48-2.42 (m, 3H), 2.17-2.11 (m, 2H), 1.96-1.88 (m, 1H), 1.74-1.65 (m, 2H), 1.49 (dd, J = 12.4, 6.0 Hz, 2H), 1.31-1.13 (m, 4H) ppm. ¹H NMR (400 MHz, CD₃OD): 7.82 (dd, J = 6.8, 2.4 Hz, 1H), 7.45-7.41 (m, 1H), 7.21 (t, J = 8.8 Hz, 1H), 3.57 (s, 3H), 3.30-3.26 (m, 1H),2.67-2.60 (m, 4H), 2.33-2.27 (m, 2H), 2.18-2.11 (m, 1H), 1.66 (dd, J = 12.4, 6.0 Hz, 2H), 1.45-1.26 (m, 4H) ppm.

| | |
|---|---|
| Example | Structure and analysis |
| Example 38 | 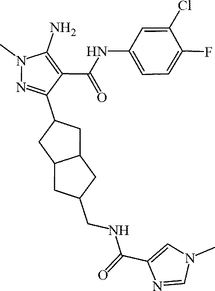 |
| | 5-Amino-N-(3-chloro-4-fluorophenyl)-1-methyl-3-(5-((1-methyl-1H-imidazole-4-carboxamido)methyl)octahydropentalen-2-yl)-1H-pyrazole-4-carboxamide MS calcd for C₂₅H₂₉ClFN₇O₂; 513.21. Found: 514.25 [M + 1]⁺. ¹H NMR (400 MHz, DMSO-d₆): δ 8.94 (s, 1H), 7.89 (dd, J = 6.8, 2.4 Hz, 1H), 7.74 (t, J = 6.4 Hz, 1H), 7.62 (s, 1H), 7.56 (s, 1H), 7.52-7.50 (m, 1H), 7.33 (t, J = 9.6 Hz, 1H), 5.95 (s, 2H), 3.66 (s, 3H), 3.57-3.53 (m, 1H), 3.49 (s, 3H), 3.19 (t, J = 6.4 Hz, 2H), 2.43-1.89 (m, 7H), 1.42-1.26 (m, 2H), 0.97-0.95 (m, 2H) ppm. |
| Example 39 | 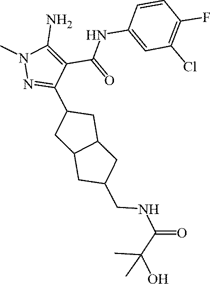 [SMILES: Cn1nc(C2CC3CC(CNC(=O)C(C)(C)O)CC3C2)c(C(=O)Nc2ccc(F)c(Cl)c2)c1N] |
| | 5-Amino-N-(3-chloro-4-fluorophenyl)-3-(5-((2-hydroxy-2-methylpropanamido)methyl)octahydropentalen-2-yl)-1-methyl-1H-pyrazole-4-carboxamide Isomer I. MS calcd for C₂₄H₃₁ClFN₅O₃; 492.21. Found 493.25 [M + 1]⁺. ¹H NMR (400 MHz, DMSO-d₆): δ 8.95 (s, 1H), 7.89 (dd, J = 6.8, 2.0 Hz, 1H), 7.54-7.49 (m, 2H), 7.34 (t, J = 8.8 Hz, 1H), 5.96 (s, 2H), 5.29 (s, 1H), 3.55-3.52 (m, 1H), 3.49 (s, 3H), 3.04 (t, J = 6.4 Hz, 2H), 2.42-2.40 (m, 2H), 2.15-2.06 (m, 3H), 1.91-1.87 (m, 2H), 1.45-1.37 (m, 2H), 1.27-1.21 (m, 6H), 0.93-0.85 (m, 2H) ppm. |
| Example 40 | 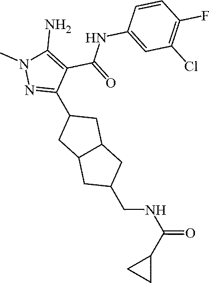 [SMILES: Cn1nc(C2CC3CC(CNC(=O)C4CC4)CC3C2)c(C(=O)Nc2ccc(F)c(Cl)c2)c1N] |
| | 5-Amino-N-(3-chloro-4-fluorophenyl)-3-(5-(cyclopropanecarboxamidomethyl)octahydropentalen-2-yl)-1-methyl-1H-pyrazole-4-carboxamide Isomer I. MS calcd for C₂₄H₂₉ClFN₅O₂; 473.20. Found; 474.10 [M + 1]⁺. ¹H NMR (400 MHz, DMSO-d₆): δ 8.95 (s, 1H), 7.97 (t, J= 6.0 Hz, 1H), 7.90 (dd, J = 6.8, 2.4 Hz, 1H), 7.53-7.44 (m, 1H), 7.34 (t, J = 9.6 Hz, 1H), 5.96 (s, 2H), 3.57-3.52 (m, 1H), 3.49 (s, 3H), 3.03 (t, J = 6.0 Hz, 2H), 2.45-2.40 (m, 2H), 2.15-1.90 (m, 5H), 1.54-1.37 (m, 3H), 0.96-0.88 (m, 2H), 0.62-0.58 (m, 4H) ppm. |
| Example 41 | 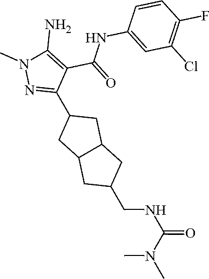 [SMILES: CN(C)C(=O)NCC1CC2CC(c3nn(C)c(N)c3C(=O)Nc3ccc(F)c(Cl)c3)CC2C1] |
| | 5-Amino-N-(3-chloro-4-fluorophenyl)-3-(5-((3,3-dimethylureido)methyl)octahydropentalen-2-yl)-1-methyl-1H-pyrazole-4-carboxamide Isomer I. MS calcd for C₂₃H₃₀ClFN₆O₂; 476.21. Found; 477.5 [M+1]⁺. ¹H NMR (400 MHz, DMSO-d₆): δ 8.94 (s, 1H), 7.89 (dd, J = 6.8, 2.4 Hz, 1H), 7.53-7.49 (m, 1H), 7.34 (t, J = 8.8 Hz, 1H), 6.15 (t, J = 5.6 Hz, 1H), 5.95 (s, 2H), 3.56-3.52 (m, 1H), 3.49 (s, 3H), 2.97 (t, J = 5.6 Hz, 2H), 2.74 (s, 6H), 2.41-2.40 (m, 2H), 2.15-1.88 (m, 5H), 1.45-1.37 (m, 2H), 0.95-0.86 (m, 2H) ppm. |
| Example 42 | 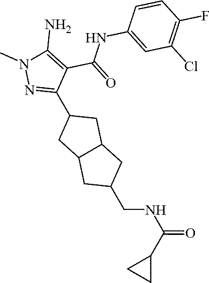 [SMILES: Cn1nc(C2CC3CC(CNC(=O)C4CC4)CC3C2)c(C(=O)Nc2ccc(F)c(Cl)c2)c1N] |
| | 5-Amino-N-(3-chloro-4-fluorophenyl)-3-(5-(cyclopropanecarboxamidomethyl)octahydropentalen-2-yl)-1-methyl-1H-pyrazole-4-carboxamide Isomer II. MS calcd for C₂₄H₂₉ClFN₅O₂; 473.20. Found; 474.10 [M+1]⁺. ¹H NMR (400 MHz, DMSO-d₆): δ 8.93 (s, 1H), 7.98 (t, J = 5.2 Hz, 1H), 7.89 (dd, J = 6.8,2.4 Hz, 1H), 7.52-7.48 (m, 1H), 7.33 (t, J = 8.8 Hz, 1H), 5.95 (s, 2H), 3.48 (s, 3H), 3.28-3.23 (m, 1H, merged), 3.03-3.00 (m, 2H), 2.48-2.44 (m, 2H, merged), 2.14-2.00 (m, 3H), 1.55-1.43 (m, 3H), 1.29-1.15 (m, 4H), 0.64-0.56 (m, 4H) ppm. |
| Example 43 | 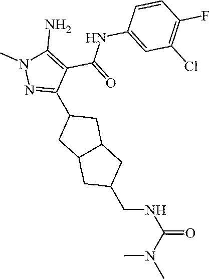 [SMILES: CN(C)C(=O)NCC1CC2CC(c3nn(C)c(N)c3C(=O)Nc3ccc(F)c(Cl)c3)CC2C1] |
| | 5-Amino-N-(3-chloro-4-fluorophenyl)-3-(5-((3,3-dimethylureido)methyl)octahydropentalen-2-yl)-1-methyl-1H-pyrazole-4-carboxamide Isomer II MS calcd for C₂₃H₃₀ClFN₆O₂; 476.21. Found; 477.15 [M+1]⁺. ¹H NMR (400 MHz, DMSO-d₆): δ 8.93 (s, 1H), 7.90-7.88 (m, 1H), 7.52-7.47 (m, 1H), 7.33 (t, J = 9.2 Hz, 1H), 6.17 (t, J = 5.6 Hz, 1H), 5.94 (s, 2H), 3.48 (s, 3H), 3.28-3.24 (m, 1H, merged), 2.97-2.93 (m, 2H), 2.74 (s, 6H), 2.50-2.40 (m, 1H, merged), 2.13-2.08 (m, 4H), 1.44-1.39 (m, 2H), 1.28-1.17 (m, 4H) ppm. |
| Example 44 | 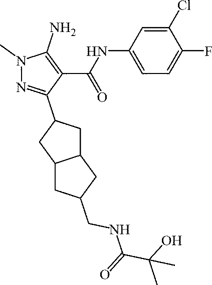 [SMILES: Cn1nc(C2CC3CC(CNC(=O)C(C)(C)O)CC3C2)c(C(=O)Nc2ccc(F)c(Cl)c2)c1N] |
| | 5-Amino-N-(3-chloro-4-fluorophenyl)-3-(5-((2-hydroxy-2-methylpropanamido)methyl)octahydropentalen-2-yl)-1-methyl-1H-pyrazole-4-carboxamide Isomer II MS calcd for C₂₄H₃₁ClFN₅O₃; 491.21. Found; 492.14 [M + 1]⁺. ¹H NMR (400 MHz, DMSO-d₆): δ 8.95 (s, 1H), 7.92-7.89 (m, 1H), 7.60-7.57 (m, 1H), 7.52-7.49 (m, 1H), 7.34 (t, J = 8.8 Hz, 1H), 5.96 (s, 2H), 5.29 (s, 1H), 3.49 (s, 3H), 3.31-3.26 (m, 1H, merged), 3.05-3.02 (m, 2H), 2.50-2.40 (m, 2H, merged), 2.15-2.05 (m, 3H), 1.44-1.39 (m, 2H), 1.28-1.17 (m, 10H). |
| Example 45 | 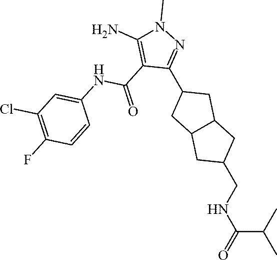 [SMILES: CC(C)C(=O)NCC1CC2CC(c3nn(C)c(N)c3C(=O)Nc3ccc(F)c(Cl)c3)CC2C1] |
| | 5-Amino-N-(3-chloro-4-fluorophenyl)-3-(5-(isobutyramidomethyl)octahydropentalen-2-yl)-1-methyl-1H-pyrazole-4-carboxamide MS calcd for C₂₄H₃₁ClFN₅O₂; 475.22. Found; 476.05 [M + 1]⁺. ¹H NMR (400 MHz, DMSO-d₆): δ 8.94 (s, 1H), 7.89 (dd, J = 6.8, 2.4 Hz, 1H), 7.64 (t, J = 5.6 Hz, 1H), 7.53-7.50 (m, 1H), 7.34 (t, J = 9.2 Hz, 1H), 5.95 (s, 2H), 3.56-3.52 (m, 1H), 3.49 (s, 3H), 3.00 (t, J = 5.6 Hz, 2H), 2.45-2.29 (m, 3H), 2.15-1.87 (m, 5H), 1.45-1.37 (m, 2H), 0.97-0.88 (m, 8H) ppm. |
| Example 46 | 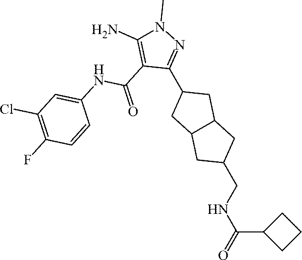 [SMILES: Cn1nc(C2CC3CC(CNC(=O)C4CCC4)CC3C2)c(C(=O)Nc2ccc(F)c(Cl)c2)c1N] |
| | 5-Amino-N-(3-chloro-4-fluorophenyl)-3-(5-(cyclobutanecarboxamidomethyl)octahydropentalen-2-yl)-1-methyl-1H-pyrazole-4-carboxamide. MS calcd for C₂₅H₃₁ClFN₅O₂; 487.22. Found; 488.05 [M + 1]⁺. ¹H NMR (400 MHz, DMSO-d₆): δ 8.94 (s, 1H), 7.89 (d, J = 5.2 Hz, 1H), 7.57-7.50 (m, 2H), 7.34 (t, J = 9.2 Hz, 1H), 5.96 (s, 2H), 3.56-3.53 (m, 1H), 3.49 (s, 3H), 3.01-2.92 (m, 3H), 2.41-2.39 (m, 2H), 2.11-1.71 (m, 11H), 1.45-1.37 (m, 2H), 0.94-0.86 (m, 2H) ppm. |
| Example 47 | 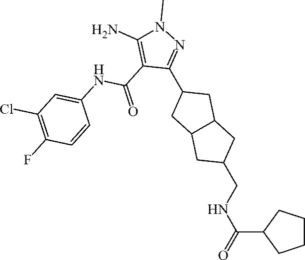 [SMILES: Cn1nc(C2CC3CC(CNC(=O)C4CCCC4)CC3C2)c(C(=O)Nc2ccc(F)c(Cl)c2)c1N] |
| | 5-Amino-N-(3-chloro-4-fluorophenyl)-3-(5-(cyclopentanecarboxamidomethyl)octahydropentalen-2-yl)-1-methyl-1H-pyrazole-4-carboxamide. MS calcd for C₂₆H₃₃ClFN₅O₂; 501.23. Found; 502.15 [M + 1]⁺. ¹H NMR (400 MHz, DMSO-d₆): δ 8.95 (s, 1H), 7.89 (d, J = 5.6 Hz, 1H), 7.66 (t, J = 6.0 Hz, 1H), 7.52-7.50 (m, 1H), 7.34 (t, J = 9.2 Hz, 1H), 5.96 (s, 2H), 3.55-3.53 (m, 1H), 3.49 (s, 3H), 3.01 (t, J = 6.0 Hz, 2H), 2.41-2.39 (m, 2H), 2.14-1.87 (m, 5H), 1.71-1.37 (m, 11H), 0.95-0.87 (m, 2H) ppm. |
| Example 48 | 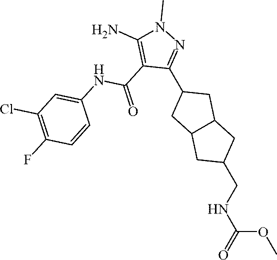 [SMILES: COC(=O)NCC1CC2CC(c3nn(C)c(N)c3C(=O)Nc3ccc(F)c(Cl)c3)CC2C1] |
| | Methyl ((5-(5-amino-4-((3-chloro-4-fluorophenyl)carbamoyl)-1-methyl-1H-pyrazol-3-yl)octahydropentalen-2-yl)methyl)carbamate. MS calcd for C₂₂H₂₇ClFN₅O₃; 463.18. Found; 464.05 [M + 1]⁺. ¹H NMR (400 MHz, DMSO-d₆): δ 8.94 (s, 1H), 7.89 (dd, J = 6.8, 2.4 Hz, 1H), 7.53-7.50 (m, 1H), 7.34 (t, J = 8.8 Hz, 1H), 7.08 (t, J = 6.0 Hz, 1H), 5.95 (s, 2H), 3.55-3.46 (m, 7H), 2.95 (t, J = 6.0 Hz, 2H), 2.42-2.41 (m, 2H), 2.16-1.88 (m, 5H), 1.45-1.36 (m, 2H), 0.95-0.87 (m, 2H) ppm. |
| Example 49 | 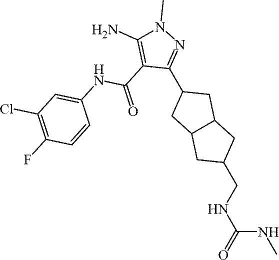 [SMILES: CNC(=O)NCC1CC2CC(c3nn(C)c(N)c3C(=O)Nc3ccc(F)c(Cl)c3)CC2C1] |
| | 5-Amino-N-(3-chloro-4-fluorophenyl)-1-methyl-3-(5-((3-methylureido)methyl)octahydropentalen-2-yl)-1H-pyrazole-4-carboxamide. MS calcd for C₂₂H₂₈ClFN₆O₂; 462.19. Found; 463.20 [M + 1]⁺. ¹H NMR (400 MHz, DMSO-d₆): δ 8.95 (s, 1H), 7.91 (d, J = 5.6 Hz, 1H), 7.52-7.50 (m, 1H), 7.35 (t, J = 8.4 Hz, 1H), 5.97 (s, 2H), 5.82-5.80 (m, 1H), 5.61-5.60 (m, 1H), 3.56-3.52 (m, 1H), 3.50 (s, 3H), 2.98-2.97 (m, 2H), 2.50-2.43 (m, 4H, merged), 2.15-1.90 (m, 5H), 1.44-0.90 (m, 5H) ppm. |
| Example 50 | 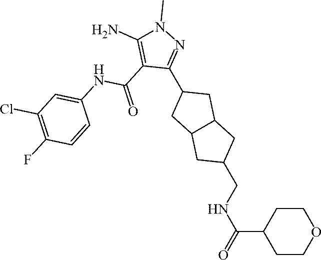 [SMILES: Cn1nc(C2CC3CC(CNC(=O)C4CCOCC4)CC3C2)c(C(=O)Nc2ccc(F)c(Cl)c2)c1N] |
| | 5-Amino-N-(3-chloro-4-fluorophenyl)-1-methyl-3-(5-((tetrahydro-2H-pyran-4-carboxamido)methyl)octahydropentalen-2-yl)-1H-pyrazole-4-carboxamide. MS calcd for C₂₆H₃₃ClFN₅O₃; 517.23. Found; 518.5 [M + 1]⁺. ¹H NMR (400 MHz, DMSO-d₆): δ 8.94 (s, 1H), 7.89 (dd, J = 6.8, 2.4 Hz, 1H), 7.71 (t, J = 6.0 Hz, 1H), 7.52-7.50 (m, 1H), 7.34 (t, J = 9.2 Hz, 1H), 5.95 (s, 2H), 3.85-3.82 (m, 2H), 3.57-3.53 (m, 1H), 3.49 (s, 3H), 3.29- |
| | 3.23 (m, 2H), 3.01 (t, J = 6.0 Hz, 2H), 2.40-2.32 (m, 3H), 2.14-1.87 (m, 5H), 1.57-1.37 (m, 6H), 0.95-0.88 (m, 2H) ppm. |
| Example 51 | 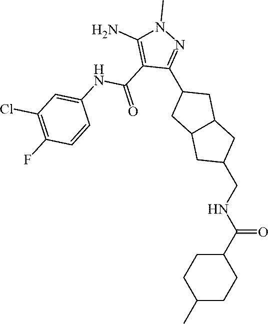 [SMILES: CC1CCC(C(=O)NCC2CC3CC(c4nn(C)c(N)c4C(=O)Nc4ccc(F)c(Cl)c4)CC3C2)CC1] |
| | 5-Amino-N-(3-chloro-4-fluorophenyl)-1-methyl-3-(5-((4-methylcyclohexane-1-carboxamido)methyl)octahydropentalen-2-yl)-1H-pyrazole-4-carboxamide. MS calcd for C₂₈H₃₇ClFN₅O₂; 529.26. Found; 530.40 [M + 1]⁺. ¹H NMR (400 MHz, DMSO-d₆): δ 8.94 (s, 1H), 7.89 (dd, J = 6.8, 2.4 Hz, 1H), 7.59-7.55 (m, 1H), 7.53-7.49 (m, 1H), 7.33 (t, J = 9.2 Hz, 1H), 5.96 (s, 2H), 3.55-3.52 (m, 1H), 3.49 (s, 3H), 3.02-2.98 (m, 2H), 2.41-1.28 (m, 18H), 0.98-0.83 (m, 6H) ppm. |
| Example 52 | 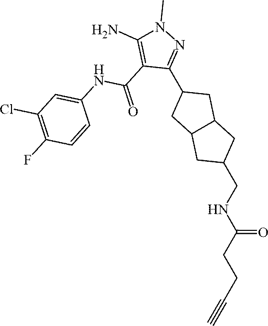 [SMILES: C#CCCC(=O)NCC1CC2CC(c3nn(C)c(N)c3C(=O)Nc3ccc(F)c(Cl)c3)CC2C1] |
| | 5-Amino-N-(3-chloro-4-fluorophenyl)-1-methyl-3-(5-(pent-4-ynamidomethyl)octahydropentalen-2-yl)-1H-pyrazole-4-carboxamide. MS calcd for C₂₅H₂₉ClFN₅O₂; 485.20. Found; 487.5 [M + 2]⁺. ¹H NMR (400 MHz, DMSO-d₆): δ 8.94 (s, 1H), 7.89 (d, J = 6.8 Hz, 1H), 7.84-7.82 (m, 1H), 7.52-7.50 (m, 1H), 7.33 (t, J = 9.2 Hz, 1H), 5.95 (s, 2H), 3.56-3.52 (m, 1H), 3.49 (s, 3H), 3.03 (t, J =6.0 Hz, 2H), 2.74 (s, 1H), 2.41-1.89 (m, 11H), 1.45-1.38 (m, 2H), 0.97-0.89 (m, 2H) ppm |
| Example 53 | 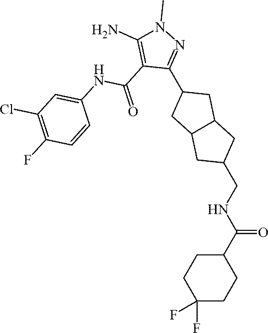 [SMILES: Cn1nc(C2CC3CC(CNC(=O)C4CCC(F)(F)CC4)CC3C2)c(C(=O)Nc2ccc(F)c(Cl)c2)c1N] |
| | 5-Amino-N-(3-chloro-4-fluorophenyl)-3-(5-((4,4-difluorocyclohexane-1-carboxamido)methyl)octahydropentalen-2-yl)-1-methyl-1H-pyrazole-4-carboxamide. MS calcd for C₂₇H₃₃ClF₃N₅O₂; 551.23. Found; 552.2 [M + 1]⁺. ¹H NMR (400 MHz, DMSO-d₆): δ 8.94 (s, 1H), 7.90-7.89 (m, 1H), 7.79-7.77 (m, 1H), 7.52-7.49 (m, 1H), 7.33 (t, J = 9.6 Hz, 1H), 5.95 (s, 2H), 3.55-3.51 (m, 1H), 3.48 (s, 3H), 3.01 (t, J = 5.6 Hz, 2H), 2.41-1.57 (m, 16H), 1.45-1.40 (m, 2H), 0.95-0.91 (m, 2H) ppm. |
| Example 54 | 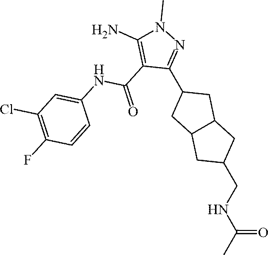 [SMILES: CC(=O)NCC1CC2CC(c3nn(C)c(N)c3C(=O)Nc3ccc(F)c(Cl)c3)CC2C1] |
| | 3-(5-(Acetamidomethyl)octahydropentalen-2-yl)-5-amino-N-(3-chloro-4-fluorophenyl)-1-methyl-1H-pyrazole-4-carboxamide. MS calcd for C₂₂H₂₇ClFN₅O₂; 447.18. Found; 448.05 [M + 1]⁺. ¹H NMR (400 MHz, DMSO-d₆): δ 8.95 (s, 1H), 7.89 (dd, J = 7.2, 2.4 Hz, 1H), 7.78-7.74 (m, 1H), 7.52-7.51 (m, 1H), 7.34 (t, J = 9.2 Hz, 1H), 5.96 (s, 2H), 3.57-3.54 (m, 1H), 3.49 (s, 3H), 3.00 (t, J = 6.0 Hz, 2H), 2.42-2.41 (m, 2H), 2.14-1.89 (m, 5H), 1.77 (s, 3H), 1.45-1.37 (m, 2H), 0.95-0.87 (m, 2H) ppm. |
| Example 55 | 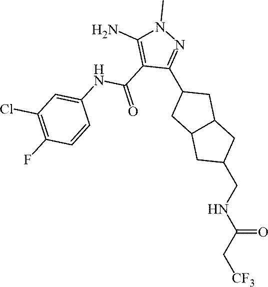 [SMILES: Cn1nc(C2CC3CC(CNC(=O)CC(F)(F)F)CC3C2)c(C(=O)Nc2ccc(F)c(Cl)c2)c1N] |
| | 5-Amino-N-(3-chloro-4-fluorophenyl)-1-methyl-3-(5-((3,3,3-trifluoropropanamido)methyl)octahydropentalen-2-yl)-1H-pyrazole-4-carboxamide. MS calcd for C₂₃H₂₆ClF₄N₅O₂; 515.17. Found; 516.10 [M + 1]⁺. ¹H NMR (400 MHz, DMSO-d₆): δ 8.95 (s, 1H), 8.19-8.18 (m, 1H), 7.92-7.89 (m, 1H), 7.52-7.50 (m, 1H), 7.34 (t, J = 9.2 Hz, 1H), 5.96 (s, 2H), 3.55-3.50 (m, 1H), 3.49 (s, 3H), 3.27-3.16 (m, 2H), 3.07 (t, J = 6.0 Hz, 2H), 2.43-2.42 (m, 2H), 2.14-1.90 (m, 5H), 1.45-1.40 (m, 2H), 0.94-0.92 (m, 2H) ppm. |
| Example 56 | 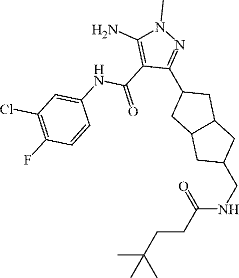 [SMILES: Cn1nc(C2CC3CC(CNC(=O)CCC(C)(C)C)CC3C2)c(C(=O)Nc2ccc(F)c(Cl)c2)c1N] |
| | 5-Amino-N-(3-chloro-4-fluorophenyl)-3-(5-((4,4-dimethylpentanamido)methyl)octahydropentalen-2-yl)-1-methyl-1H-pyrazole-4-carboxamide. MS calcd for C₂₇H₃₇ClFN₅O₂; 517.26. Found; 518.25 [M + 1]⁺. ¹H NMR (400 MHz, DMSO-d₆): δ 8.94 (s, 1H), 7.89 (d, J = 4.8 Hz, 1H), 7.73-7.72 (m, 1H), 7.52-7.50 (m, 1H), 7.34 (t, J = 9.2 Hz, 1H), 5.95 (s, 2H), 3.56-3.52 (m, 1H), 3.49 (s, 3H), 3.00 (t, J = 5.6 Hz, 2H), 2.42-2.11 (m, 2H), 2.14-1.88 (m, 7H), 1.40-1.36 (m, 4H), 0.92-0.84 (m, 11H) ppm. |
| Example 57 | 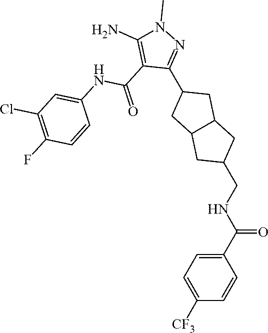 [SMILES: Cn1nc(C2CC3CC(CNC(=O)c4ccc(C(F)(F)F)cc4)CC3C2)c(C(=O)Nc2ccc(F)c(Cl)c2)c1N] |
| | 5-Amino-N-(3-chloro-4-fluorophenyl)-1-methyl-3-(5-((4-(trifluoromethyl)benzamido)methyl)octahydropentalen-2-yl)-1H-pyrazole-4-carboxamide. MS calcd for C₂₈H₂₈ClF₄N₅O₂; 577.19. Found; 578.10 [M + 1]⁺. ¹H NMR (400 MHz, DMSO-d₆): δ 8.95 (s, 1H), 8.68-8.66 (m, 1H), 8.00 (d, J = 8.4 Hz, 2H), 7.89 (dd, J = 6.8, 2.4 Hz, 1H), 7.81 (d, J = 8.4 Hz, 2H), 7.52-7.50 (m, 1H), 7.33 (t, J = 8.8 Hz, 1H), 5.96 (s, 2H), 3.55-3.52 (m, 1H), 3.48 (s, 3H), 3.26 (t, J = 6.0 Hz, 2H), 2.48-1.95 (m, 7H), 1.45-1.43 (m, 2H), 1.01-1.00 (m, 2H) ppm. |
| Example 58 | 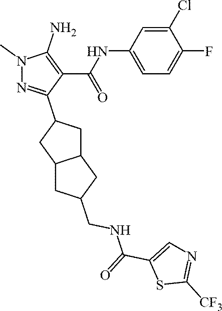 [SMILES: Cn1nc(C2CC3CC(CNC(=O)c4cnc(C(F)(F)F)s4)CC3C2)c(C(=O)Nc2ccc(F)c(Cl)c2)c1N] |
| | N-((5-(5-Amino-4-((3-chloro-4-fluorophenyl)carbamoyl)-1-methyl-1H-pyrazol-3-yl)octahydropentalen-2-yl)methyl)-2-(trifluoromethyl)thiazole-5-carboxamide. MS calcd for C₂₅H₂₅ClF₄N₆O₂S; 584.14. Found; 585.20 [M + 1]⁺. ¹H NMR (400 MHz, DMSO-d₆): δ 9.10-8.93 (m, 2H), 8.62 (s, 1H), 7.90 (d, J = 7.2 Hz, 1H), 7.55-7.48 (m, 1H), 7.33 (t, J = 8.8 Hz, 1H), 5.96 (s, 2H), 3.61-3.50 (m, 1H), 3.48 (s, 3H), 3.32-3.24 (m, 2H, merged), 2.50-2.40 (m, 1H), 2.24-2.08 (m, 4H), 2.04-1.92 (m, 2H), 1.50-1.38 (m, 2H), 1.08-0.54 (m, 2H) ppm |
| Example 59 | 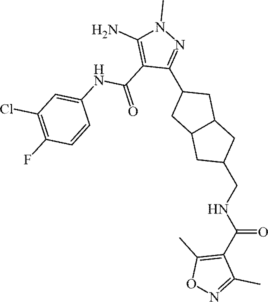 [SMILES: Cc1noc(C)c1C(=O)NCC1CC2CC(c3nn(C)c(N)c3C(=O)Nc3ccc(F)c(Cl)c3)CC2C1] |
| | N-((5-(5-Amino-4-((3-chloro-4-fluorophenyl)carbamoyl)-1-methyl-1H-pyrazol-3-yl)octahydropentalen-2-yl)methyl)-3,5-dimethylisoxazole-4-carboxamide. MS calcd for C₂₆H₃₀ClFN₆O₃; 528.21. Found; 529.15 [M + 1]⁺. ¹H NMR (400 MHz, DMSO-d₆): δ 8.95 (s, 1H), 7.98-7.96 (m, 1H), 7.89 (dd, J = 6.8, 2.4 Hz, 1H), 7.52-7.50 (m, 1H), 7.34 (t, J = 9.2 Hz, 1H), 5.96 (s, 2H), 3.55-3.52 (m, 1H), 3.48 (s, 3H), 3.19 (t, J = 6.0 Hz, 2H), 2.48-2.44 (m, 5H), 2.25 (s, 3H), 2.16-2.13 (m, 2H), 1.97-1.94 (m, 3H), 1.45-1.43 (m, 2H), 1.01-0.98 (m, 2H) ppm. |
| Example 60 | 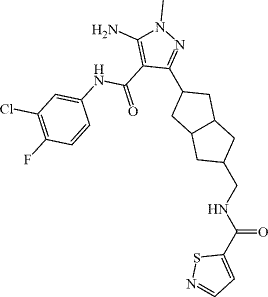 [SMILES: Cn1nc(C2CC3CC(CNC(=O)c4ccns4)CC3C2)c(C(=O)Nc2ccc(F)c(Cl)c2)c1N] |
| | N-((5-(5-Amino-4-((3-chloro-4-fluorophenyl)carbamoyl)-1-methyl-1H-pyrazol-3-yl)octahydropentalen-2-yl)methyl)isothiazole-5-carboxamide. MS calcd for C₂₄H₂₆ClFN₆O₂S; 516.15. Found; 517.16 [M + 1]⁺. ¹H NMR (400 MHz, DMSO-d₆): δ 8.95 (s, 1H), 8.85-8.84 (m, 1H), 8.64-8.63 (m, 1H), 7.91-7.89 (m, 2H), 7.52-7.50 (m, 1H), 7.34 (t, J = 9.2 Hz, 1H), 5.96 (s, 2H), 3.57-3.54 (m, 1H), 3.49 (s, 3H), 3.25-3.24 (m, 2H), 2.45-2.44 (m, 2H), 2.19-2.12 (m, 3H), 1.98-1.95 (m, 2H), 1.47-1.42 (m, 2H), 1.00-0.99 (m, 2H) ppm. |
| Example 61 | 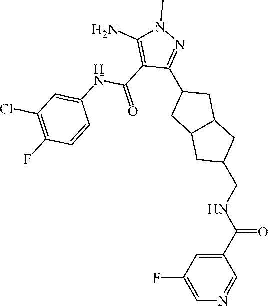 [SMILES: Cn1nc(C2CC3CC(CNC(=O)c4cncc(F)c4)CC3C2)c(C(=O)Nc2ccc(F)c(Cl)c2)c1N] |
| | N-((5-(5-Amino-4-((3-chloro-4-fluorophenyl)carbamoyl)-1-methyl-1H-pyrazol-3-yl)octahydropentalen-2-yl)methyl)-5-fluoronicotinamide. MS calcd for C₂₆H₂₇ClFN₆O₂; 528.19. Found; 529.25 [M + 1]⁺. ¹H NMR (400 MHz, DMSO-d₆): δ 8.94 (s, 1H), 8.85 (s, 1H), 8.72-8.69 (m, 2H), 8.06-8.03 (m, 1H), 7.89 (dd, J = 6.8, 2.4 Hz, 1H), 7.52-7.48 (m, 1H), 7.35 (t, J = 8.6 Hz, 1H), 5.95 (s, 2H), 3.58-3.52 (m, 1H), 3.48 (s, 3H), 3.41-3.39 (m, 2H, merged), 2.49-2.51 (m, 2H, merged), 2.22-2.12 (m, 3H), 1.99-1.96 (m, 2H), 1.48-1.40 (m, 2H), 1.05-0.96 (m, 2H) ppm. |
| Example 62 | 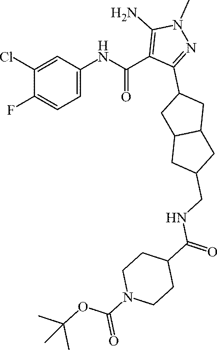 [SMILES: Cn1nc(C2CC3CC(CNC(=O)C4CCN(C(=O)OC(C)(C)C)CC4)CC3C2)c(C(=O)Nc2ccc(F)c(Cl)c2)c1N] |
| | tert-Butyl 4-(((5-(5-amino-4-((3-chloro-4-fluorophenyl)carbamoyl)-1-methyl-1H-pyrazol-3-yl)octahydropentalen-2-yl)methyl)carbamoyl)piperidine-1-carboxylate. MS calcd for C₃₁H₄₂ClFN₆O₄; 616.29. Found; 617.20 [M + 1]⁺. ¹H NMR (400 MHz, DMSO-d₆): δ 8.94 (s, 1H), 7.90 (dd, J = 6.8, 2.0 Hz, 1H), 7.75 (t, J = 8.8 Hz, 1H), 7.54-7.48 (m, 1H), 7.34 (t, J = 9.2 Hz, 1H), 5.96 (s, 2H), 3.98-3.86 (m, 2H), 3.60-3.52 (m, 1H), 3.49 (s, 3H), 3.01 (t, J = 5.6 Hz, 2H), 2.74-2.60 (m, 2H), 2.46-2.38 (m, 2H), 2.30-2.20 (m, 1H), 2.18-1.98 (m, 3H), 1.92- |
| | 1.80 (m, 2H), 1.64-1.54 (m, 2H), 1.48-1.30 (m, 13H), 0.98-0.86 (m, 2H) ppm. |
| Example 63 | 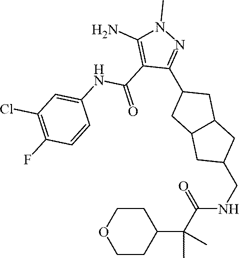 [SMILES: Cn1nc(C2CC3CC(CNC(=O)C(C)(C)C4CCOCC4)CC3C2)c(C(=O)Nc2ccc(F)c(Cl)c2)c1N] |
| | 5-Amino-N-(3-chloro-4-fluorophenyl)-1-methyl-3-(5-((2-methyl-2-(tetrahydro-2H-pyran-4-yl)propanamido)methyl)octahydropentalen-2-yl)-1H-pyrazole-4-carboxamide. MS calcd for C₂₉H₃₉ClFN₅O₃; 559.3. Found; 560.1 [M + 1]⁺. ¹H NMR (400 MHz, DMSO-d₆): δ 8.94 (s, 1H), 7.89 (dd, J = 6.8, 2.4 Hz, 1H), 7.52-7.49 (m, 1H), 7.38-7.29 (m, 2H), 5.96 (s, 2H), 3.84 (d, J = 8.0 Hz, 2H), 3.57-3.53 (m, 1H), 3.48 (s, 3H), 3.20 (t, J = 11.2 Hz, 2H), 3.02 (t, J = 5.6 Hz, 2H), 2.40-2.10 (m, 5H), 1.88-1.74 (m, 3H), 1.41-1.20 (m, 7H), 0.97-0.91 (m, 7H) ppm. |
| Example 64 | 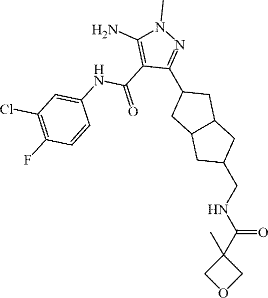 [SMILES: Cn1nc(C2CC3CC(CNC(=O)C4(C)COC4)CC3C2)c(C(=O)Nc2ccc(F)c(Cl)c2)c1N] |
| | 5-Amino-N-(3-chloro-4-fluorophenyl)-1-methyl-3-(5-((3-methyloxetane-3-carboxamido)methyl)octahydropentalen-2-yl)-1H-pyrazole-4-carboxamide. MS calcd for C₂₅H₃₁ClFN₅O₃; 503.21. Found; 504.1 [M + 1]⁺. ¹H NMR (400 MHz, DMSO-d₆): δ 8.95 (s, 1H), 7.89 (dd, J = 6.8, 2.0 Hz, 1H), 7.77 (t, J = 6.0 Hz, 1H), 7.52-7.49 (m, 1H), 7.33 (t, J = 9.2 Hz, 1H), 5.95 (s, 2H), 4.66 (d, J = 5.6 Hz, 2H), 4.20 (d, J = 6.0 Hz, 2H), 3.55-3.53 (m, 1H), 3.48 (s, 3H), 3.06 (t, J = 6.0 Hz, 2H), 2.42-2.11 (m, 2H), 2.15-1.88 (m, 5H), 1.45-1.40 (m, 5H), 0.97-0.92 (m, 2H) ppm. |
| Example 65 | 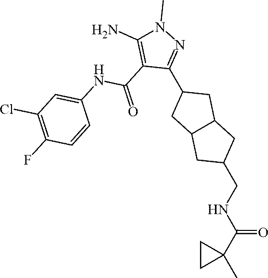 [SMILES: Cn1nc(C2CC3CC(CNC(=O)C4(C)CC4)CC3C2)c(C(=O)Nc2ccc(F)c(Cl)c2)c1N] |
| | 5-Amino-N-(3-chloro-4-fluorophenyl)-1-methyl-3-(5-((1-methylcyclopropane-1-carboxamido)methyl)octahydropentalen-2-yl)-1H-pyrazole-4-carboxamide. MS calcd for C₂₅H₃₁ClFN₅O₂; 487.22. Found; 488.0 [M + 1]⁺. ¹H NMR (400 MHz, DMSO-d₆): δ 8.96 (s, 1H), 7.89 (dd, J = 6.8, 2.4 Hz, 1H), 7.53-7.52 (m, 1H), 7.41 (t, J = 6.0 Hz, 1H), 7.34 (t, J = 8.8 Hz, 1H), 6.0 (br.s, 2H), 3.55-3.53 (m, 1H), 3.49 (s, 3H), 3.03 (t, J = 6.0 Hz, 2H), 2.41-2.40 (m, 2H), 2.13-2.11 (m, 3H), 1.91-1.87 (m, 2H), 1.42-1.40 (m, 2H), 1.22-1.19 (m, 3H), 0.92-0.89 (m, 4H), 0.45-0.44 (m, 2H) ppm. |
| Example 66 | 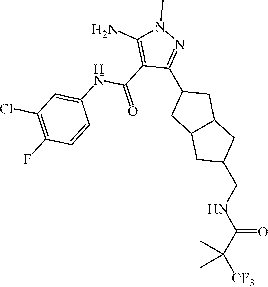 [SMILES: Cn1nc(C2CC3CC(CNC(=O)C(C)(C)C(F)(F)F)CC3C2)c(C(=O)Nc2ccc(F)c(Cl)c2)c1N] |
| | 5-Amino-N-(3-chloro-4-fluorophenyl)-1-methyl-3-(5-((3,3,3-trifluoro-2,2-dimethylpropanamido)methyl)octahydropentalen-2-yl)-1H-pyrazole-4-carboxamide. MS calcd for C₂₅H₃₀ClF₄N₅O₂; 543.20. Found; 544.1 [M + 1]⁺. ¹H NMR (400 MHz, DMSO-d6): δ 8.96 (s, 1H), 7.92-7.86 (m, 2H), 7.53-7.50 (m, 1H), 7.34 (t, J = 9.6 Hz, 1H), 6.00 (br.s, 2H), 3.55-3.53 (m, 1H), 3.49 (s, 3H), 3.07 (t, J = 5.6 Hz, 2H), 2.42-2.40 (m, 2H), 2.15-2.07 (m, 3H), 1.89-1.86 (m, 2H), 1.45-1.40 (m, 2H), 1.31 (s, 6H), 0.93-0.91 (m, 2H) ppm. |
| Example 67 | 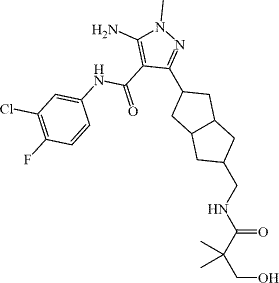 [SMILES: Cn1nc(C2CC3CC(CNC(=O)C(C)(C)CO)CC3C2)c(C(=O)Nc2ccc(F)c(Cl)c2)c1N] |
| | 5-Amino-N-(3-chloro-4-fluorophenyl)-3-(5-((3-hydroxy-2,2-dimethylpropanamido)methyl)octahydropentalen-2-yl)-1-methyl-1H-pyrazole-4-carboxamide. MS calcd for C₂₅H₃₃ClFN₅O₃; 505.23. Found; 506.1 [M + 1]⁺. ¹H NMR (400 MHz, DMSO-d6): δ 8.94 (s, 1H), 7.89 (dd, J = 6.8, 2.4 Hz, 1H), 7.53-7.49 (m, 1H), 7.41-7.31 (m, 2H), 5.95 (s, 2H), 4.85 (t, J = 5.2 Hz, 1H), 3.57-3.53 (m, 1H), 3.49 (s, 3H), 3.34-3.20 (m, 2H, merged), 3.03 (t, J = 5.6 Hz, 2H), 2.41-2.40 (m, 2H), 2.15-2.05 (m, 3H), 1.91-1.87 (m, 2H), 1.45-1.37 (m, 2H), 0.99 (s, 6H), 0.95-0.87 (m, 2H) ppm. |
| Example 68 | 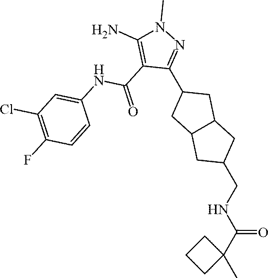 [SMILES: Cn1nc(C2CC3CC(CNC(=O)C4(C)CCC4)CC3C2)c(C(=O)Nc2ccc(F)c(Cl)c2)c1N] |
| | 5-Amino-N-(3-chloro-4-fluorophenyl)-1-methyl-3-(5-((1-methylcyclobutane-1-carboxamido)methyl)octahydropentalen-2-yl)-1H-pyrazole-4-carboxamide. MS calcd for C₂₆H₃₃ClFN₅O₂; 501.23. Found; 502.15 [M + 1]⁺. ¹H NMR (400 MHz, DMSO-d6): δ 8.94 (s, 1H), 7.89 (d, J = 7.2 Hz, 1H), 7.52-7.50 (m, 1H), 7.36 (t, J = 6.0 Hz, 1H), 7.34 (t, J = 9.2 Hz, 1H), 5.96 (s, 2H), 3.55-3.53 (m, 1H), 3.49 (s, 3H), 3.01 (t, J = 6.0 Hz, 2H), 2.41-1.40 (m, 15H), 1.27 (s, 3H), 0.92-0.91 (m, 2H) ppm. |
| Example 69 | 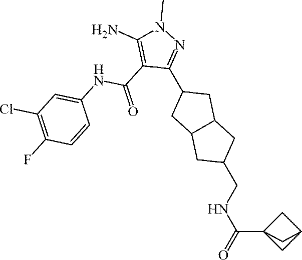 [SMILES: Cn1nc(C2CC3CC(CNC(=O)C45CC(C4)C5)CC3C2)c(C(=O)Nc2ccc(F)c(Cl)c2)c1N] |
| | 5-Amino-3-(5-((bicyclo[1.1.1]pentane-1-carboxamido)methyl)octahydropentalen-2-yl)-N-(3-chloro-4-fluorophenyl)-1-methyl-1H-pyrazole-4-carboxamide. MS calcd for C₂₆H₃₁ClFN₅O₂; 499.22. Found; 500.15 [M + 1]⁺. ¹H NMR (400 MHz, DMSO-d₆): δ 8.94 (s, 1H), 7.89 (dd, J = 6.8, 2.4 Hz, 1H), 7.61 (t, J = 6.4 Hz, 1H), 7.53-7.49 (m, 1H), 7.33 (t, J = 9.2 Hz, 1H), 5.95 (s, 2H), 3.57-3.53 (m, 1H), 3.48 (s, 3H), 2.98 (t, J = 6.4 Hz, 2H), 2.45-2.36 (m, 2H), 2.14-2.03 (m, 3H), 1.91-1.86 (m, 8H), 1.44-1.19 (m, 3H), 0.93-0.85 (m, 2H) ppm. |
| Example 70 | 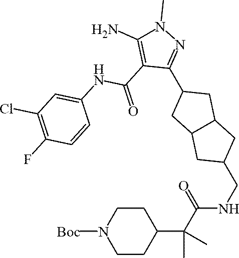 [SMILES: Cn1nc(C2CC3CC(CNC(=O)C(C)(C)C4CCN(C(=O)OC(C)(C)C)CC4)CC3C2)c(C(=O)Nc2ccc(F)c(Cl)c2)c1N] |
| | tert-Butyl 4-(1-(((5-(5-amino-4-((3-chloro-4-fluorophenyl)carbamoyl)-1-methyl-1H-pyrazol-3-yl)octahydropentalen-2-yl)methyl)amino)-2-methyl-1-oxopropan-2-yl)piperidine-1-carboxylate. MS calcd for C₃₄H₄₈ClFN₆O₄; 658.34. Found; 659.26 [M + 1]⁺. ¹H NMR (400 MHz, DMSO-d₆): δ 8.93 (s, 1H), 7.89 (dd, J = 7.2, 2.8 Hz, 1H), 7.53-7.49 (m, 1H), 7.37-7.31 (m, 2H), 5.95 (s, 2H), 3.96 (d, J = 9.2 Hz, 2H), 3.57-3.53 (m, 1H), 3.49 (s, 3H), 3.02 (t, J = 6.0 Hz, 2H), 2.41-2.40 (m, 2H), 2.14-2.07 (m, 3H), 1.88-1.85 (m, 2H), 1.67-1.61 (m, 1H), 1.45-1.36 (m, 14H), 1.02-0.87 (m, 11H) ppm. |
| Example 71 | 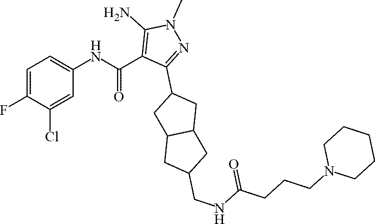 [SMILES: Cn1nc(C2CC3CC(CNC(=O)CCCN4CCCCC4)CC3C2)c(C(=O)Nc2ccc(F)c(Cl)c2)c1N] |
| | 5-Amino-N-(3-chloro-4-fluorophenyl)-1-methyl-3-(5-((4-(piperidin-1-yl)butanamido)methyl)octahydropentalen-2-yl)-1H-pyrazole-4-carboxamide. MS calcd for C₂₉H₄₀ClFN₆O₂; 558.29. Found; 559.55 [M + 1]⁺. ¹H NMR (400 MHz, DMSO-d₆): δ 8.94 (s, 1H), 7.92-7.89 (m, 1H), 7.73-7.70 (m, 1H), 7.52-7.49 (m, 1H), 7.34 (t, J = 8.8 Hz, 1H), 5.96 (s, 2H), 3.60-3.52 (m, 1H), 3.49 (s, 3H), 3.03-2.99 (m, 2H), 2.45-2.38 (m, 2H), 2.33-2.27 (m, 4H), 2.20-2.11 (m, 4H), 2.06-2.02 (m, 3H), 1.92-1.88 (m, 2H), 1.64-1.58 (m, 2H), 1.48-1.35 (m, 8H), 0.96-0.91 (m, 2H) ppm. |
| Example 72 | 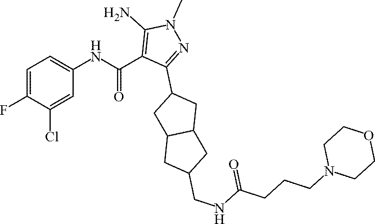 [SMILES: Cn1nc(C2CC3CC(CNC(=O)CCCN4CCOCC4)CC3C2)c(C(=O)Nc2ccc(F)c(Cl)c2)c1N] |
| | 5-Amino-N-(3-chloro-4-fluorophenyl)-1-methyl-3-(5-((4-morpholinobutanamido)methyl)octahydropentalen-2-yl)-1H-pyrazole-4-carboxamide. MS calcd for C₂₈H₃₈ClFN₆O₃; 560.27. Found; 561.1 [M + 1]⁺. ¹H NMR (400 MHz, DMSO-d6): δ 8.94 (s, 1H), 7.91 (dd, J = 6.8, 2.4 Hz, 1H), 7.73-7.70 (m, 1H), 7.52-7.49 (m, 1H), 7.34 (t, J = 8.8 Hz, 1H), 5.96 (s, 2H), 3.55-3.52 (m, 4H), 3.49 (s, 3H), 3.35-3.30 (m, 1H, merged), 3.03-2.99 (m, 2H), 2.45-2.40 (m, 2H), 2.30-2.25 (m, 4H), 2.22-2.17 (m, 2H), 2.14-2.11 (m, 2H), 2.07-2.01 (m, 3H), 1.91-1.88 (m, 2H), 1.63-1.59 (m, 2H), 1.45-1.40 (m, 2H), 0.93-0.90 (m, 2H) ppm. |
| Example 73 | 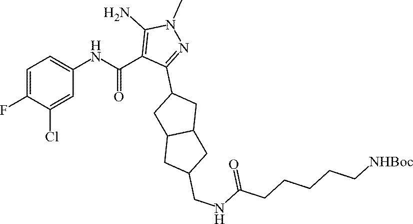 [SMILES: Cn1nc(C2CC3CC(CNC(=O)CCCCCNC(=O)OC(C)(C)C)CC3C2)c(C(=O)Nc2ccc(F)c(Cl)c2)c1N] |
| | tert-Butyl (6-(((5-(5-amino-4-((3-chloro-4-fluorophenyl)carbamoyl)-1-methyl-1H-pyrazol-3-yl)octahydropentalen-2-yl)methyl)amino)-6-oxohexyl)carbamate. MS calcd for C₃₁H₄₄ClFN₆O₄; 618.3. Found: 619.2 [M + 1]⁺. ¹H NMR (400 MHz, DMSO-d₆): δ 8.94 (s, 1H), 7.91-7.89 (m, 1H), 7.72-7.68 (m, 1H), 7.55-7.50 (m, 1H), 7.34 (t, J = 9.6 Hz, 1H), 6.75-6.70 (m, 1H), 5.96 (s, 2H), 3.55-3.47 (m, 4H), 3.32-3.28 (m, 2H), 2.90-2.84 (m, 2H), 2.45-2.40 (m, 2H), 2.15-2.10 (m, 2H), 2.05-1.96 (m, 3H), 1.95-1.90 (m, 2H), 1.50-1.30 (m, 14H), 1.20-1.15 (m, 3H), 0.95-0.89 (m, 2H) ppm. |
| Example 74 | 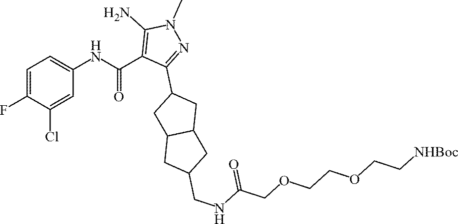 [SMILES: Cn1nc(C2CC3CC(CNC(=O)COCCOCCNC(=O)OC(C)(C)C)CC3C2)c(C(=O)Nc2ccc(F)c(Cl)c2)c1N] |
| | tert-Butyl (2-(2-(2-(((5-(5-amino-4-((3-chloro-4-fluorophenyl)carbamoyl)-1-methyl-1H-pyrazol-3-yl)octahydropentalen-2-yl)methyl)amino)-2-oxoethoxy)ethoxy)ethyl)carbamate. MS calcd for C₃₁H₄₄CIFN₆O₆; 650.30. Found; 651.5 [M + 1]⁺. ¹H NMR (400 MHz, DMSO-d₆): δ 8.95 (s, 1H), 7.89 (dd, J = 6.8, 2.4 Hz, 1H), 7.62-7.58 (m, 1H), 7.52-7.49 (m, 1H), 7.34 (t, J = 8.8 Hz, 1H), 6.80-6.70 (m, 1H), 5.96 (s, 2H), 3.84 (s, 2H), 3.60-3.49 (m, 8H), 3.39 (t, J = 5.6 Hz, 2H), 3.12-3.04 (m, 4H), 2.45-2.40 (m, 2H), 2.15-2.10 (m, 3H), 1.94-1.88 (m, 2H), 1.45-1.34 (m, 11H), 0.96-.092 (m, 2H) ppm. |
| Example 75 | 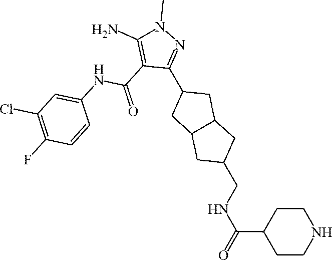 [SMILES: Cn1nc(C2CC3CC(CNC(=O)C4CCNCC4)CC3C2)c(C(=O)Nc2ccc(F)c(Cl)c2)c1N] |
| | N-((5-(5-Amino-4-((3-chloro-4-fluorophenyl)carbamoyl)-1-methyl-1H-pyrazol-3-yl)octahydropentalen-2-yl)methyl)piperidine-4-carboxamide MS calcd for C₂₆H₃₄ClFN₆O₂; 516.24. Found; 517.15 [M + 1]⁺. ¹H NMR (400 MHz, DMSO-d₆): δ 8.95 (s, 1H), 8.36 (s, 1H), 7.90 (dd, J = 6.8, 2.4 Hz, 1H), 7.75 (t, J = 5.2 Hz, 1H), 7.56-7.48 (m, 1H), 7.34 (t, J = 9.2 Hz, 1H), 5.96 (s, 2H), 3.58-3.50 (m, 1H), 3.49 (s, 3H), 3.10-3.06 (m, 2H), 3.04-2.98 (m, 2H), 2.68-2.54 (m, 2H), 2.46-2.34 (m, 2H), 2.32-2.18 (m, 1H), 2.16-1.98 (m, 3H), 1.96-1.84 (m, 2H), 1.72-1.50 (m, 4H), 1.46-1.36 (m, 2H), 0.97-0.86 (m, 2H) ppm. |
| Example 76 | 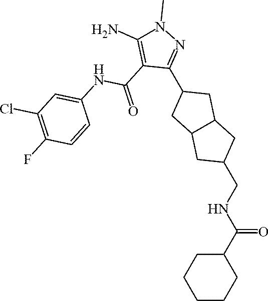 [SMILES: Cn1nc(C2CC3CC(CNC(=O)C4CCCCC4)CC3C2)c(C(=O)Nc2ccc(F)c(Cl)c2)c1N] |
| | 5-Amino-N-(3-chloro-4-fluorophenyl)-3-(5-(cyclohexanecarboxamidomethyl)octahydropentalen-2-yl)-1-methyl-1H-pyrazole-4-carboxamide MS calcd for C₂₇H₃₅ClFN₅O₂; 515.25. Found; 516.65 [M + 1]⁺. ¹H NMR (400 MHz, DMSO-d6): δ 8.94 (s, 1H), 7.93-7.88 (m, 1H), 7.60 (t, J = 5.6 Hz, 1H), 7.55-7.46 (m, 1H), 7.34 (t, J = 8.8 Hz, 1H), 5.95 (s, 2H), 3.57-3.52 (m, 1H), 3.49 (s, 3H), 2.99 (t, J = 5.6 Hz, 2H), 2.43-2.35 (m, 2H), 2.18-1.98 (m, 4H), 1.94-1.82 (m, 2H), 1.72-1.56 (m, 4H), 1.49-1.08 (m, 8H), 0.98-0.85 (m, 2H) ppm. |
| Example 77 | 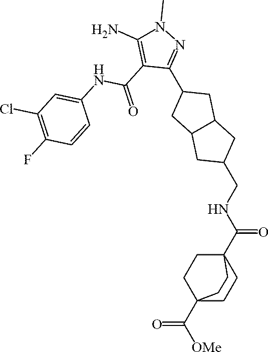 [SMILES: COC(=O)C12CCC(C(=O)NCC3CC4CC(c5nn(C)c(N)c5C(=O)Nc5ccc(F)c(Cl)c5)CC4C3)(CC1)CC2] |
| | Methyl 4-(((5-(5-amino-4-((3-chloro-4-fluorophenyl)carbamoyl)-1-methyl-1H-pyrazol-3-yl)octahydropentalen-2-yl)methyl)carbamoyl)bicyclo[2.2.2]octane-1-carboxylate. MS calcd for C₃₁H₃₉ClFN₅O₄; 599.27. Found; 600.30 [M + 1]⁺. ¹H NMR (400 MHz, DMSO-d6): δ 8.94 (s, 1H), 7.89 (dd, J = 6.8, 2.4 Hz, 1H), 7.55-7.46 (m, 1H), 7.33 (t, J = 8.8 Hz, 1H), 5.95 (s, 2H), 3.56 (s, 3H), 3.54-3.52 (m, 1H), 3.48 (s, 3H), 2.99 (t, J = 6.4 Hz, 2H), 2.45-2.34 (m, 2H), 2.18-2.02 (m, 3H), 1.92-1.80 (m, 2H), 1.64-1.58 (m, 12H), 1.48-1.34 (m, 2H), 0.96-0.84 (m, 2H) ppm; NH proton not observed. |
| Example 78 | 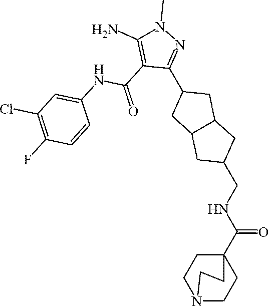 [SMILES: Cn1nc(C2CC3CC(CNC(=O)C45CCN(CC4)CC5)CC3C2)c(C(=O)Nc2ccc(F)c(Cl)c2)c1N] |
| | N-((5-(5-Amino-4-((3-chloro-4-fluorophenyl)carbamoyl)-1-methyl-1H-pyrazol-3-yl)octahydropentalen-2-yl)methyl)quinuclidine-4-carboxamide. MS calcd for C₂₈H₃₆ClFN₆O₂; 542.26. Found; 543.20 [M + 1]⁺. ¹H NMR (400 MHz, DMSO-d6): δ 8.95 (s, 1H), 8.25 (s, 1H), 7.90-7.88 (m, 1H), 7.51-7.50 (m, 1H), 7.37-7.31 (m, 2H), 5.96 (s, 2H), 3.49 (s, 3H), 3.04-3.01 (m, 3H), 2.86-2.82 (m, 6H), 2.45-2.35 (m, 2H), 2.25-2.05 (m, 3H), 1.87-1.84 (m, 2H), 1.63-1.60 (m, 6H), 1.45-1.35 (m 2H), 0.95-0.85 (m, 2H) ppm. |
| Example 79 | 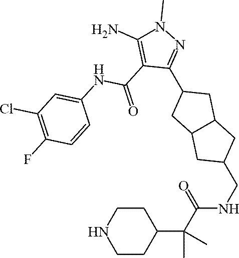 [SMILES: Cn1nc(C2CC3CC(CNC(=O)C(C)(C)C4CCNCC4)CC3C2)c(C(=O)Nc2ccc(F)c(Cl)c2)c1N] |
| | 5-Amino-N-(3-chloro-4-fluorophenyl)-1-methyl-3-(5-((2-methyl-2-(piperidin-4-yl)propanamido)methyl)octahydropentalen-2-yl)-1H-pyrazole-4-carboxamide MS calcd for C₂₉H₄₀ClFN₆O₂; 558.29. Found; 559.25 [M + 1]⁺. ¹H NMR (400 MHz, DMSO-d6): δ 8.94 (s, 1H), 7.91-7.89 (m, 1H), 7.52-7.50 (m, 1H), 7.36-7.31 (m, 2H), 5.95 (s, 2H), 3.55-3.51 (m, 1H), 3.48 (s, 3H), 3.03-3.00 (m, 2H), 2.93-2.91 (m, 2H), 2.42-2.30 (m, 4H), 2.15-2.05 (m, 3H), 1.87-1.85 (m, 2H), 1.60-1.50 (m, 1H), 1.41-1.34 (m, 4H), 1.30-1.20 (m, 1H), 1.10-1.00 (m, 2H), 1.00-0.88 (m, 7H) ppm. NH protons not observed. |
| Example 80 | 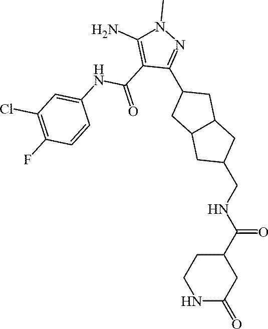 [SMILES: Cn1nc(C2CC3CC(CNC(=O)C4CCNC(=O)C4)CC3C2)c(C(=O)Nc2ccc(F)c(Cl)c2)c1N] |
| | N-((5-(5-Amino-4-((3-chloro-4-fluorophenyl)carbamoyl)-1-methyl-1H-pyrazol-3-yl)octahydropentalen-2-yl)methyl)-2-oxopiperidine-4-carboxamide MS calcd for C₂₆H₃₂ClFN₆O₃; 530.22. Found; 532.05 [M + 1]⁺. ¹H NMR (400 MHz, DMSO-d6): δ 8.94 (s, 1H), 7.91-7.89 (m, 1H), 7.85-7.82 (m, 1H), 7.52-7.49 (m, 1H), 7.42 (s, 1H), 7.36-7.31 (m, 1H), 5.95 (s, 2H), 3.57-3.50 (m, 1H), 3.49 (s, 3H), 3.11-2.97 (m, 4H), 2.62-2.50 (m, 2H, merged), 2.45-2.35 (m, 1H), 2.24-2.04 (m, 5H), 1.95-1.85 (m, 2H), 1.85-1.75 (m, 1H), 1.65-1.55 (m, 1H), 1.45-1.38 (m, 2H), 0.95-0.90 (m, 2H) ppm. |
| Example 81 | 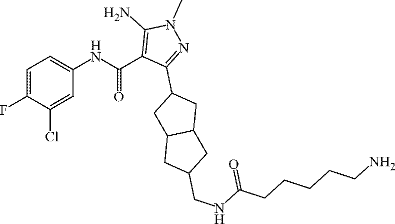 [SMILES: Cn1nc(C2CC3CC(CNC(=O)CCCCCN)CC3C2)c(C(=O)Nc2ccc(F)c(Cl)c2)c1N] |
| | 5-Amino-3-(5-((6-aminohexanamido)methyl)octahydropentalen-2-yl)-N-(3-chloro-4-fluorophenyl)-1-methyl-1H-pyrazole-4-carboxamide MS calcd for C₂₆H₃₆ClFN₆O₂; 518.26. Found; 519.35 [M + 1]⁺. ¹H NMR (400 MHz, DMSO-d6): δ 8.97 (s, 1H), 8.43 (s, 1H), 7.91-7.89 (m, 1H), 7.55-7.72 (m, 1H), 7.53-7.49 (m, 1H), 7.36-7.32 (m, 1H), 5.96 (s, 2H), 3.60-3.50 (m, 1H), 3.49 (s, 3H), 3.03-3.00 (m, 3H), 2.69-2.65 (m, 2H), 2.45-2.35 (m, 2H), 2.28-2.10 (m, 2H), 2.10-1.95 (m, 3H), 1.95-1.85 (m, 2H), 1.50-1.37 (m, 6H), 1.30-1.20 (m, 2H), 1.00-0.85 (m, 2H) ppm. |
| Example 82 | 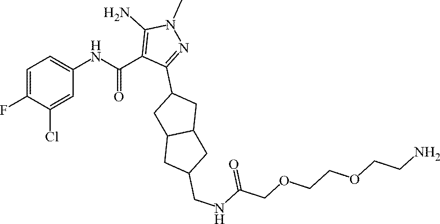 [SMILES: Cn1nc(C2CC3CC(CNC(=O)COCCOCCN)CC3C2)c(C(=O)Nc2ccc(F)c(Cl)c2)c1N] |
| | 5-Amino-3-(5-((2-(2-(2-aminoethoxy)ethoxy)acetamido)methyl)octahydropentalen-2-yl)-N-(3-chloro-4-fluorophenyl)-1-methyl-1H-pyrazole-4-carboxamide. MS calcd for C₂₆H₃₆ClFN₆O₄; 550.25. Found; 551.00 [M + 1]⁺. ¹H NMR (400 MHz, DMSO-d6): δ 9.02 (s, 1H), 7.91-7.89 (m, 1H), 7.88-7.78 (m, 3H), 7.72-7.68 (m, 1H), 7.51-7.50 (m, 1H), 7.37-7.33 (m, 1H), 3.88 (s, 2H), 3.80-3.5 (m, 7H, merged), 3.50 (s, 3H), 3.11-3.08 (m, 2H), 2.97-2.96 (m, 2H), 2.50-2.40 (m, 3H), 2.20-2.00 (m, 3H), 1.95-1.85 (m, 2H), 1.45-1.35 (m, 2H), 1.00-0.90 (m, 2H) ppm. |

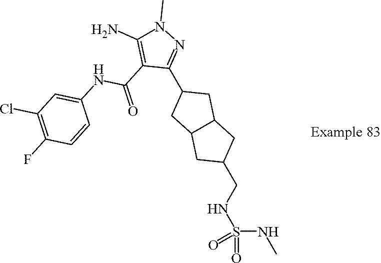 [SMILES: CNS(=O)(=O)NCC1CC2CC(c3nn(C)c(N)c3C(=O)Nc3ccc(F)c(Cl)c3)CC2C1]

5-Amino-N-(3-chloro-4-fluorophenyl)-1-methyl-3-(5-(((N-methylsulfamoyl)amino)methyl)octahydropentalen-2-yl)-1H-pyrazole-4-carboxamide. To a stirred solution of compound 5-amino-3-(5-(aminomethyl)octahydropentalen-2-yl)-N-(3-chloro-4-fluorophenyl)-1-methyl-1H-pyrazole-4-carboxamide Isomer II (0.06 g, 0.148 mmol) in DCM (3 mL) at 0° C., triethyl amine (0.022 g, 0.22 mmol) was added methylsulfamoyl chloride (0.022 g, 0.177 mmol) and stirring continued for 1 h. After completion, the reaction mixture was diluted with ice cold water and extracted with DCM. The combined organic layer was dried over dried over anhydrous sodium sulphate, filtered, and concentrated under reduced pressure. The crude compound was purified by Prep. HPLC to afford 5-amino-N-(3-chloro-4-fluorophenyl)-1-methyl-3-(5-(((N-methylsulfamoyl)amino)methyl)octahydropentalen-2-yl)-1H-pyrazole-4-carboxamide as off white solid.

### General Procedure for Sulphonamidation

To a stirred solution of 5-amino-3-(5-(aminomethyl)octahydropentalen-2-yl)-N-(3-chloro-4-fluorophenyl)-1-methyl-1H-pyrazole-4-carboxamide Isomer II (0.24 mmol, 1 eq.) in DCM/ DMF (2.5 mL) at 0° C. was added sulfonyl chloride (0.029 mmol) and TEA (0.038 mmol) and the reaction mixture stirred at room temperature for 15 min. After completion, the reaction mixture was diluted with water and extracted with DCM. The combined organic layers were collected, dried over anhydrous sodium sulphate, filtered, concentrated in vacuo and purified by prep. HPLC to afford the desired compound.

| | |
|---|---|
| Example | Structure and analysis |
| Example 84 | 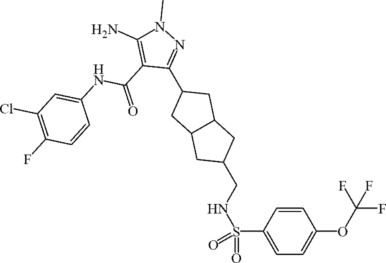 [SMILES: Cn1nc(C2CC3CC(CNS(=O)(=O)c4ccc(OC(F)(F)F)cc4)CC3C2)c(C(=O)Nc2ccc(F)c(Cl)c2)c1N] |
| | 5-Amino-N-(3-chloro-4-fluorophenyl)-1-methyl-3-(5-(((4-(trifluoromethoxy)phenyl)sulfonamido)methyl)octahydropentalen-2-yl)-1H-pyrazole-4-carboxamide MS calcd for C₂₇H₂₈CIF₄N₅O₄S; 629.15. Found; 630.10 [M + 1]⁺. ¹H NMR (400 MHz, DMSO-d₆): δ 8.92 (s, 1H), 7.94-7.88 (m, 3H), 7.75-7.68 (m, 1H), 7.58 (d, J = 8.0 Hz, 2H), 7.53-7.48 (m, 1H), 7.33 (t, J = 8.8 Hz, 1H), 5.95 (s, 2H), 3.58-3.50 (m, 1H), 3.47 (s, 3H), 2.74 (t, J = 6.4 Hz, 2H), 2.42-2.30 (m, 2H), 2.16-2.04 (m, 2H), 2.03-1.92 (m, 1H), 1.88-1.82 (m, 2H), 1.42-1.32 (m, 2H), 0.92-0.80 (m, 2H) ppm. |
| Example 85 | 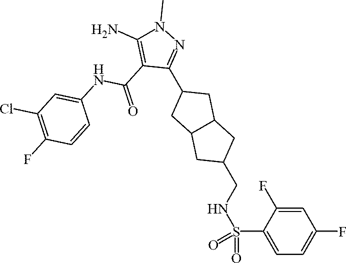 [SMILES: Cn1nc(C2CC3CC(CNS(=O)(=O)c4ccc(F)cc4F)CC3C2)c(C(=O)Nc2ccc(F)c(Cl)c2)c1N] |
| | 5-Amino-N-(3-chloro-4-fluorophenyl)-3-(5-(((2,4-difluorophenyl)sulfonamido)methyl)octahydropentalen-2-yl)-1-methyl-1H-pyrazole-4-carboxamide MS calcd for C₂₆H₂₇ClF₃N₅O₃S; 581.15. Found; 582.20 [M + 1]⁺. ¹H NMR (400 MHz, DMSO-d₆): δ 8.93(s, 1H), 7.97-7.95 (m, 1H), 7.91-7.88 (m, 1H), 7.86-7.80 (m, 1H), 7.55-7.48 (m, 2H), 7.34 (t, J = 9.6 Hz, 1H), 7.28-7.24 (m, 1H), 5.96 (s, 2H), 3.55-3.48 (m, 4H), 2.84-2.80 (m, 2H), 2.45-2.30 (m, 2H), 2.11-2.07 (m, 2H), 2.00-1.97 (m, 1H), 1.88-1.85 (m, 2H), 1.41-1.33 (m, 2H), 0.90-0.81 (m, 2H) ppm. |
| Example 86 | 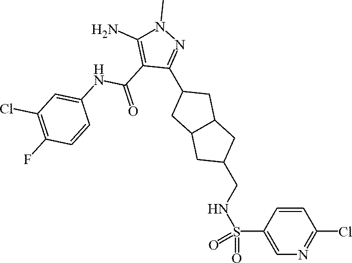 [SMILES: Cn1nc(C2CC3CC(CNS(=O)(=O)c4ccc(Cl)nc4)CC3C2)c(C(=O)Nc2ccc(F)c(Cl)c2)c1N] |
| | 5-Amino-N-(3-chloro-4-fluorophenyl)-3-(5-(((6-chloropyridine)-3-sulfonamido)methyl)octahydropentalen-2-yl)-1-methyl-1H-pyrazole-4-carboxamide MS calcd for C₂₅H₂₇Cl₂FN₆O₃S; 580.12. Found; 580.8 [M + 1]⁺. ¹H NMR (400 MHz, DMSO-d₆): δ 8.94 (s, 1H), 8.76-8.74 (m, 1H), 8.20-8.15 (m, 1H), 7.91-7.89 (m, 2H), 7.77 (d, J = 8.4 Hz, 1H), 7.53-7.48 (m, 1H), 7.34 (t, J = 9.6 Hz, 1H), 5.96 (s, 2H), 3.57-3.52 (m, 1H), 3.48 (s, 3H), 2.82-2.75 (m, 2H), 2.49-2.30 (m, 2H), 2.12-2.08 (m, 2H), 2.00-1.96 (m, 1H), 1.89-1.85 (m, 2H), 1.44-1.34 (m, 2H), 0.95-0.83 (m, 2H) ppm. |
| Example 87 | 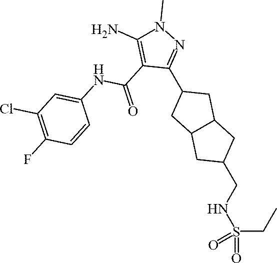 [SMILES: CCS(=O)(=O)NCC1CC2CC(c3nn(C)c(N)c3C(=O)Nc3ccc(F)c(Cl)c3)CC2C1] |
| | 5-Amino-N-(3-chloro-4-fluorophenyl)-3-(5-(ethylsulfonamidomethyl)octahydropentalen-2-yl)-1-methyl-1H-pyrazole-4-carboxamide MS calcd; 497.17. Found; 498.25 [M + 1]⁺. |
| Example 88 | 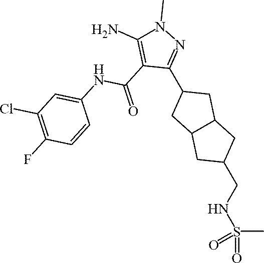 [SMILES: Cn1nc(C2CC3CC(CNS(C)(=O)=O)CC3C2)c(C(=O)Nc2ccc(F)c(Cl)c2)c1N] |
| | 5-Amino-N-(3-chloro-4-fluorophenyl)-1-methyl-3-(5-(methylsulfonamidomethyl)octahydropentalen-2-yl)-1H-pyrazole-4-carboxamide MS clalcd for C₂₁H₂₇ClFN₅O₃S; 483.15. Found; 484.00 [M + 1]⁺. ¹H NMR (400 MHz, DMSO-d₆): δ 8.94 (s, 1H), 7.90-7.87 (m, 1H), 7.51-7.49 (m, 1H), 7.33 (t, J = 9.2 Hz, 1H), 6.90 (t, J = 5.6 Hz, 1H), 5.95 (s, 2H), 3.56-3.50 (m, 1H), 3.48 (s, 3H), 2.90-2.87 (m, 2H), 2.84 (s, 3H), 2.45-2.40 (m, 2H), 2.15-1.90 (m, 5H), 1.45-1.37 (m, 2H), 1.00-0.88 (m, 2H) ppm. |
| Example 89 | 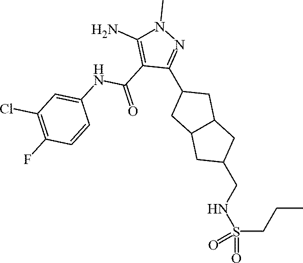 [SMILES: CCCS(=O)(=O)NCC1CC2CC(c3nn(C)c(N)c3C(=O)Nc3ccc(F)c(Cl)c3)CC2C1] |
| | 5-Amino-N-(3-chloro-4-fluorophenyl)-1-methyl-3-(5-(propylsulfonamidomethyl)octahydropentalen-2-yl)-1H-pyrazole-4-carboxamide. MS calcd for C₂₃H₃₁CIFN₅O₃S; 511.18. Found; 512.15 [M + 1]⁺. ¹H NMR (400 MHz, DMSO-d₆): δ 8.93 (s, 1H), 7.88 (dd, J = 6.8, 2.4 Hz, 1H), 7.52-7.47 (m, 1H), 7.32 (t, J = 8.8 Hz, 1H), 6.93 (t, J = 5.6 Hz, 1H), 5.94 (s, 2H), 3.58-3.51 (m, 1H), 3.47 (s, 3H), 2.92-2.85 (m, 4H), 2.45-2.35 (m, 2H), 2.15-1.90 (m, 5H), 1.67-1.57 (m, 2H), 1.45-1.36 (m, 2H), 0.96-0.89 (m, 5H) ppm. |
| Example 90 | 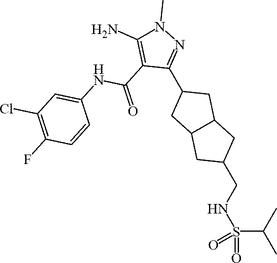 [SMILES: CC(C)S(=O)(=O)NCC1CC2CC(c3nn(C)c(N)c3C(=O)Nc3ccc(F)c(Cl)c3)CC2C1] |
| | 5-Amino-N-(3-chloro-4-fluorophenyl)-1-methyl-3-(5-(((1-methylethyl)sulfonamido)methyl)octahydropentalen-2-yl)-1H-pyrazole-4-carboxamide. MS calcd for C₂₃H₃₁ClFN₅O₃S; 511.18. |
| | Found; 512 [M + 1]⁺. ¹H NMR (400 MHz, DMSO-d₆): δ 8.96 (s, 1H), 7.91-7.89 (m, 1H), 7.52-7.50 (m, 1H), 7.34 (t, J = 8.8 Hz, 1H), 6.95-6.93 (m, 1H), 6.00-5.95 (m, 2H), 3.60-3.30 (m, 4H), 3.15-3.10 (m, 1H), 2.92-2.89 (m, 2H), 2.48-2.40 (m, 2H), 2.15-1.92 (m, 5H), 1.46-1.38 (m, 2H), 1.19 (d, J = 6.8 Hz, 6H), 1.00-0.95 (m, 2H) ppm. |
| Example 91 | 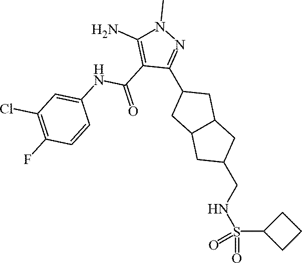 [SMILES: Cn1nc(C2CC3CC(CNS(=O)(=O)C4CCC4)CC3C2)c(C(=O)Nc2ccc(F)c(Cl)c2)c1N] |
| | 5-Amino-N-(3-chloro-4-fluorophenyl)-3-(5-(cyclobutanesulfonamidomethyl)octahydropentalen-2-yl)-1-methyl-1H-pyrazole-4-carboxamide. MS calcd for C₂₄H₃₁ClFN₅O₃S; 523.18. Found; 524.10 [M + 1]⁺. ¹H NMR (400 MHz, DMSO-d₆): δ 8.96 (s, 1H), 7.91-7.89 (m, 1H), 7.52-7.50 (m, 1H), 7.34 (t, J = 8.8 Hz, 1H), 6.95-6.92 (m, 1H), 5.96 (s, 2H), 3.90-3.78 (m, 1H), 3.60-3.49 (m, 1H), 3.49 (s, 3H), 2.92-2.89 (m, 2H), 2.48-2.40 (m, 2H), 2.35-1.80 (m, 11H), 1.46-1.38 (m, 2H), 1.05-0.95 (m, 2H) ppm. |
| Example 92 | 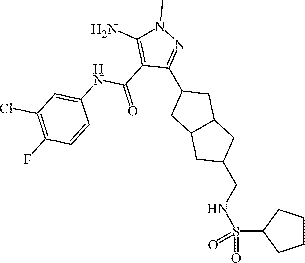 [SMILES: Cn1nc(C2CC3CC(CNS(=O)(=O)C4CCCC4)CC3C2)c(C(=O)Nc2ccc(F)c(Cl)c2)c1N] |
| | 5-Amino-N-(3-chloro-4-fluorophenyl)-3-(5-(cyclopentanesulfonamidomethyl)octahydropentalen-2-yl)-1-methyl-1H-pyrazole-4-carboxamide. MS calcd for C₂₅H₃₃ClFN₅O₃S; 537.20. Found: 538.1 [M + 1]⁺. ¹H NMR (400 MHz, DMSO-d₆): δ 8.93 (s, 1H), 7.89 (dd, J = 6.8, 2.0 Hz, 1H), 7.51-7.48 (m, 1H), 7.33 (t, J = 9.2 Hz, 1H), 6.92 (t, J = 5.6 Hz, 1H), 5.94 (s, 2H), 3.55-3.42 (m, 4H), |
| | 2.92-2.88 (m, 2H), 2.45-2.40 (m, 2H), 2.13-1.92 (m, 5H), 1.84-1.79 (m, 4H), 1.20-1.12 (m, 1H), 1.62-1.37 (m, 6H), 0.97-0.0.83 (m, 2H) ppm. |
| Example 93 | 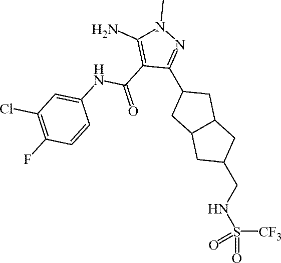 [SMILES: Cn1nc(C2CC3CC(CNS(=O)(=O)C(F)(F)F)CC3C2)c(C(=O)Nc2ccc(F)c(Cl)c2)c1N] |
| | 5-Amino-N-(3-chloro-4-fluorophenyl)-1-methyl-3-(5-(((trifluoromethyl)sulfonamido)methyl)octahydropentalen-2-yl)-1H-pyrazole-4-carboxamide MS calcd for C₂₁H₂₄CIF₄N₅O₃S; 537.12. Found; 538.30 [M + 1]⁺. ¹H NMR (400 MHz, DMSO-d₆): δ 8.95 (s, 1H), 7.93-7.88 (m, 1H), 7.54-7.48 (m, 1H), 7.34 (t, J = 9.6 Hz, 1H), 5.96 (s, 2H), 3.61-3.48 (m, 1H), 3.49 (s, 3H), 3.13-3.04 (m, 2H), 2.48-2.39 (m, 2H), 2.20-2.02 (m, 3H), 2.00-1.89 (m, 2H), 1.49-1.37 (m, 2H), 1.04-0.94 (m, 2H) ppm; NH proton not observed. |
| Example 94 | 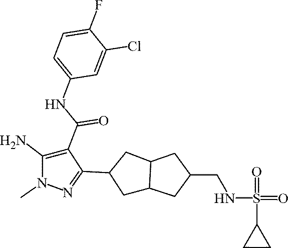 [SMILES: Cn1nc(C2CC3CC(CNS(=O)(=O)C4CC4)CC3C2)c(C(=O)Nc2ccc(F)c(Cl)c2)c1N] |
| | 5-Amino-N-(3-chloro-4-fluorophenyl)-3-(5-(cyclopropanesulfonamidomethyl)octahydropentalen-2-yl)-1-methyl-1H-pyrazole-4-carboxamide MS calcd for C₂₃H₂₉CIFN₅O₃S; 509.17. Found; 510.10 [M + 1]⁺. ¹H NMR (400 MHz, DMSO-d₆): δ 8.96 (s, 1H), 7.91 (dd, J = 6.4, 2.4 Hz, 1H), 7.54-7.50 (m, 1H), 7.34 (t, J = 9.2 Hz, 1H), 7.00-6.97 (m, 1H), 5.97 (s, 2H), 3.56-3.52 (m, 1H), 3.49 (s, 3H), 2.96-2.93 (m, 2H), 2.43 (br.s, 2H), 2.14-2.04 (m, 3H), 1.97-1.94 (m, 2H), 1.46-1.38 (m, 2H), 0.99-0.86 (m, 7H) ppm. |
| Example 95 | 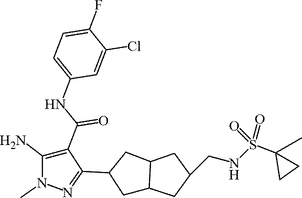 |
| | 5-Amino-N-(3-chloro-4-fluorophenyl)-1-methyl-3-(5-(((1-methylcyclopropane)-l-sulfonamido)methyl)octahydropentalen-2-yl)-1H-pyrazole-4-carboxamide MS calcd for C₂₄H₃₁ClFN₅O₃S; 523.18. Found; 524.15 [M + 1]⁺. ¹H NMR (400 MHz, DMSO-d₆): δ 8.96 (s, 1H), 7.90 (dd, J = 6.4, 2.4 Hz, 1H),7.52-7.49 (m, 1H), 7.34 (t, J = 9.2 Hz, 1H), 7.08 (t, J = 6.0 Hz, 1H), 5.96 (s, 2H), 3.54-3.52 (m, 1H), 3.49 (s, 3H), 2.92 (t, J = 6.0 Hz, 2H), 2.50-2.43 (m, 2H), 2.14-1.94 (m, 5H), 1.46-1.41 (m, 2H), 1.38 (s, 3H), 1.10-1.08 (m, 2H), 0.96-0.94 (m, 2H), 0.76-0.74 (m, 2H) ppm. |
| Example 96 | 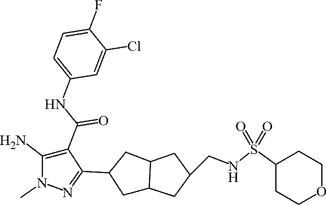 [SMILES: Cn1nc(C2CC3CC(CNS(=O)(=O)C4CCOCC4)CC3C2)c(C(=O)Nc2ccc(F)c(Cl)c2)c1N] |
| | 5-Amino-N-(3-chloro-4-fluorophenyl)-1-methyl-3-(5-(((tetrahydro-2H-pyran)-4-sulfonamido)methyl)octahydropentalen-2-yl)-1H-pyrazole-4-carboxamide MS calcd for C₂₅H₃₃ClFN₅O₄S; 553.19. Found; 554.00 [M + 1]⁺. ¹H NMR (400 MHz, DMSO-d₆): δ 8.95 (s, 1H), 7.89 (d, J = 4.8 Hz, 1H), 7.52-7.50 (m, 1H), 7.34 (t, J = 8.8 Hz, 1H), 7.05 (m, 1H), 5.96 (s, 2H), 3.91 (d, J = 7.6 Hz, 2H), 3.56-3.52 (m, 3H), 3.27-3.16 (m, 2H), 2.92 (m, 2H), 2.49-2.43 (m, 4H), 2.14-1.94 (m, 5H), 1.84-1.81(m, 2H), 1.61-1.41 (m, 4H), 0.95-0.94 (m, 2H) ppm |
| Example 97 | 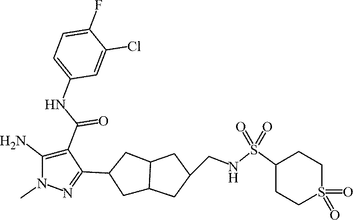 [SMILES: Cn1nc(C2CC3CC(CNS(=O)(=O)C4CCS(=O)(=O)CC4)CC3C2)c(C(=O)Nc2ccc(F)c(Cl)c2)c1N] |
| | 5-Amino-N-(3-chloro-4-fluorophenyl)-3-(5-(((1,1-dioxidotetrahydro-2H-thiopyran)-4-sulfonamido)methyl)octahydropentalen-2-yl)-1-methyl-1H-pyrazole-4-carboxamide MS calcd for C₂₅H₃₃CIFN₅O₅S₂; 601.16. Found; 602.05 [M + 1]⁺. ¹H NMR (400 MHz, DMSO-d₆): δ 8.95 (s, 1H), 7.89 (d, J = 6.8 Hz, 1H), 7.50 (m, 1H), 7.36-7.32 (m, 2H), 5.96 (s, 2H), 3.64 (m, 1H), 3.48 (s, 3H), 3.32-3.14 (m, 4H), 2.92 (m, 2H), 2.50-2.33 (m, 5H), 2.15-1.96 (m, 7H), 1.43-1.41 (m, 2H), 0.96-0.94 (m, 2H) ppm. |
| Example 98 | 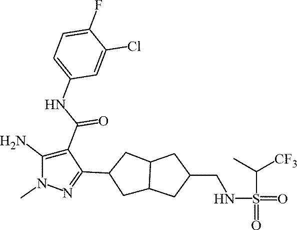 [SMILES: CC(C(F)(F)F)S(=O)(=O)NCC1CC2CC(c3nn(C)c(N)c3C(=O)Nc3ccc(F)c(Cl)c3)CC2C1] |
| | 5-Amino-N-(3-chloro-4-fluorophenyl)-1-methyl-3-(5-(((2,2,2-trifluoro-1-methylethyl)sulfonamido)methyl)octahydropentalen-2-yl)-1H-pyrazole-4-carboxamide MS calcd for C₂₅H₃₃ClFN₅O₅S₂; 601.16. Found; 602.05 [M + 1]⁺. ¹H NMR (400 MHz, DMSO-d₆): δ 8.95 (s, 1H), 7.91 (dd, J = 6.4, 2.4 Hz, 1H), 7.77-7.74 (m, 1H), 7.52-7.49 (m, 1H), 7.34 (t, J = 9.2 Hz, 1H), 5.96 (s, 2H), 4.35-4.31 (m, 1H), 3.56-3.52 (m, 1H), 3.48 (s, 3H), 2.97-2.94 (m, 2H), 2.50 (m, 2H, merged), 2.48-2.43 (m, 2H), 2.15-2.07 (m, 2H), 1.97-1.94 (m, 2H), 1.42-1.40 (m, 4H), 0.96-0.94 (m, 2H) ppm. |

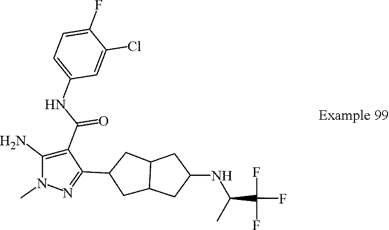 [SMILES: C[C@@H](NC1CC2CC(c3nn(C)c(N)c3C(=O)Nc3ccc(F)c(Cl)c3)CC2C1)C(F)(F)F]

5-Amino-N-(3-chloro-4-fluorophenyl)-1-methyl-3-(5-(((R)-1,1,1-trifluoropropan-2-yl)amino)octahydropentalen-2-yl)-1H-pyrazole-4-carboxamide. To a solution of 5-amino-N-(3-chloro-4-fluorophenyl)-1-methyl-3-(5-oxooctahydropentalen-2-yl)-1H-pyrazole-4-carboxamide (100 mg, 0.26 mmol) and (R)-1,1,1-trifluoropropan-2-amine hydrochloride (114 mg, 0.77 mmol) in DCE (2 mL) was added acetic acid (0.1 mL) and the mixture was stirred at 40° C. for 5 h. Na(OAc)₃BH (108 mg, 0.52 mmol) was added at r.t., and the reaction mixture stirred at 40° C. for 16 h. The reaction was quenched with 10 mL of water and extracted with ethyl acetate (10 mL x 3). The organic layers were concentrated and purified by Prep-TLC (petroleum ether/ethyl acetate=1/1) to provide the crude product. The crude product was purified by Prep-HPLC to afford 5-amino-N-(3-chloro-4-fluorophenyl)-1-methyl-3-(5-(((R)-1, 1, 1-trifluoropropan-2-yl)amino)octahydropentalen-2-yl)- 1H-pyrazole-4-carboxamide. MS Calcd.: 487.2, MS Found: 488.2 [M + 1]⁺. ¹H-NMR (400 MHz, CDCl₃): δ 7.70 (dd, J = 2.8, 6.4 Hz, 1H), 7.32-7.27 (m, 2H), 7.10 (t, J = 8.8 Hz, 1H), 5.25 (brs, 2H), 3.58 (s, 3H), 3.40-3.28 (m, 2H), 3.25-3.14 (m, 2H), 2.60-2.48 (m, 2 H), 2.40-2.30 (m, 2H), 2.28-2.14 (m, 2H), 1.86-1.75 (m, 2H), 1.26-1.19 (m, 5H) ppm.

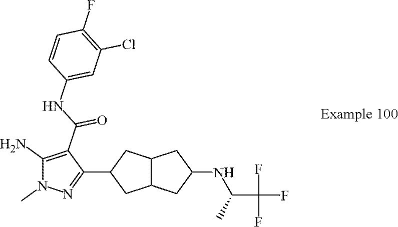 [SMILES: C[C@H](NC1CC2CC(c3nn(C)c(N)c3C(=O)Nc3ccc(F)c(Cl)c3)CC2C1)C(F)(F)F]

5-Amino-N-(3-chloro-4-fluorophenyl)-1-methyl-3-(5-(((S)-1,1,1-trifluoropropan-2-yl)amino)octahydropentalen-2-yl)-1H-pyrazole-4-carboxamide. To a solution of 5-amino-N-(3-chloro-4-fluorophenyl)-1-methyl-3-(5-oxooctahydropentalen-2-yl)-1H-pyrazole-4-carboxamide (100 mg, 0.26 mmol), (S)-1.1,1-trifluoropropan-2-amine (114 mg, 0.77 mmol) in DCE (2 mL) was added acetic acid (0.1 mL) and the mixture was stirred at 40° C. for 5 h. Na(OAc)₃BH (108 mg, 0.52 mmol) was added and then the reaction mixture was stirred at 40° C. for 16 h. The reaction was quenched with 10 mL of water and extracted with ethyl acetate (10 mL x 3). The organic layers were concentrated and purified by Prep-TLC (petroleum ether/ethyl acetate =1/1) to the crude product. The crude product was purified by Prep-HPLC to afford 5-amino-N-(3-chloro-4-fluorophenyl)-1-methyl-3-(5-(((S)-1,1,1-trifluoropropan-2-yl)amino)octahydropentalen-2-yl)-1H-pyrazole-4-carboxamide. MS Calcd.: 487.2, Found: 488.2 [M + 1]⁺. ¹H-NMR (400 MHz, CDCl₃): δ 7.70 (dd, J = 2.8, 6.4 Hz, 1H), 7.35-7.27 (m, 2H), 7.10 (t, J = 8.8 Hz, 1H), 5.26 (brs, 2H), 3.57 (s, 3H), 3.40-3.28 (m, 1H), 3.25-3.14 (m, 2H), 2.62-2.46 (m, 2 H), 2.42-2.29 (m, 2H), 2.28-2.15 (m, 2H), 1.86-1.74 (m, 2H), 1.30-1.11 (m, 5H) ppm.

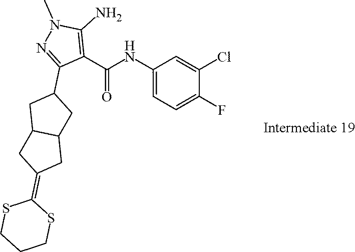 [SMILES: Cn1nc(C2CC3CC(=C4SCCCS4)CC3C2)c(C(=O)Nc2ccc(F)c(Cl)c2)c1N]

3-(1,3-Dithian-2-ylidene)octahydropentalen-2-yl)-5-amino-N-(3-chloro-4-fluorophenyl)-1-methyl-1H-pyrazole-4-carboxamide. A solution of (1,3-dithian-2-yl)trimethylsilane (7.4 g, 38.5 mmol) in THF (70 mL) was cooled at -78° C. under a nitrogen atmosphere to which was added n-BuLi (15.4 mL, 38.5 mmol). The reaction was stirred at -78° C. for 1 hour. 5-Amino-N-(3-chloro-4-fluorophenyl)-1-methyl-3-(5-oxooctahydropentalen-2-yl)-1H-pyrazole-4-carboxamide (2.0 g, 5.1 mmol) was added in one portion. The reaction was stirred at -78° C. for 3 hours then quenched with sat. NH₄Cl. The mixture was extracted with ethyl acetate.The organic phase was washed with brine, dried over Na₂SO₄, filtered, and concentrated to give the crude product which was purified by silica gel column (eluted with petroleum ether/ethyl acetate = ½) to afford 3-(5-(1,3-dithian-2-ylidene)octahydropentalen-2-yl)-5-amino-N-(3-chloro-4-fluorophenyl)-1-methyl-1H-pyrazole-4-carboxamide (1.1 g, 43%) as white solid. TLC: 67% petroleum ether/ethyl acetate (Rf: 0.3). MS Calcd.: 492.1. Found: 493.3 [M + 1] ⁺.

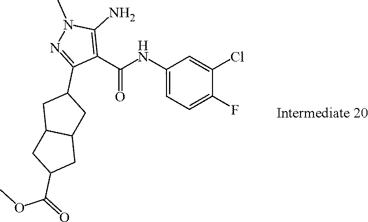 [SMILES: COC(=O)C1CC2CC(c3nn(C)c(N)c3C(=O)Nc3ccc(F)c(Cl)c3)CC2C1]

Methyl 5-(5-amino-4-(3-chloro-4-fluorophenylcarbamoyl)-1-methyl-1H-pyrazol-3-yl)octahydropentalene-2-carboxylate. To a solution of 3-(5-(1,3-dithian-2-ylidene)octahydropentalen-2-yl)-5-amino-N-(3-chloro-4-fluorophenyl)-1-methyl-1H-pyrazole-4-carboxamide (1.1 g, 2.2 mmol) in MeOH (25 mL) was added successively, HCl (6N, 1.1 mL, 6.7 mmol), HgCl₂ (1.4 g, 5.0 mmol) and trifluoroacetate (637 mg, 5.6 mmol). The reaction was stirred at room temperature for 4 hours. The milky mixture was filtered through Celite® and the filter cake washed with methanol. The solvent was removed and the residue purified by silica gel column chromatography (eluted with petroleum ether/ethyl acetate = ⅓) to afford methyl 5-(5-amino-4-(3-chloro-4-fluorophenylcarbamoyl)-1-methyl-1H-pyrazol-3-yl)octahydropentalene-2-carboxylate as a white solid. TLC: 67% petroleum ether/ ethyl acetate (Rf: 0.2). MS Calcd.: 434.2. Found: 435.3 [M + 1]⁺.

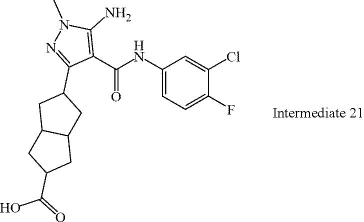 [SMILES: Cn1nc(C2CC3CC(C(=O)O)CC3C2)c(C(=O)Nc2ccc(F)c(Cl)c2)c1N]

5-Amino-4-(3-chloro-4-fluorophenylcarbamoyl)-1-methyl-1H-pyrazol-3-yl)octahydro pentalene-2-carboxylic acid. Methyl 5-amino-4-(3-chloro-4-fluorophenylcarbamoyl)-1-methyl-1H-pyrazol-3-yl)octahydropentalene-2-carboxylate (130 mg, 0.3 mmol) was dissolved in methanol (10 mL) and water (5 mL), then LiOH.H₂O (50 mg, 1.2 mmol) was added in one portion. The reaction was stirred at room temperature for 4 hours. After the starting material was consumed, the reaction was acidified with dilute HCl and extracted with ethyl acetate. The organic layer was wash with brine, dried over Na₂SO₄, filtered and concentrated to afford crude 5-(5-amino-4-(3-chloro-4-fluorophenylcarbamoyl)-1-methyl-1H-pyrazol-3-yl)octahydro pentalene-2-carboxylic acid as a white solid. TLC: 10% MeOH/ CH₂Cl₂ (Rf— 0.4). MS Calcd.: 420.1; MS Found: 421.2 [M + 1]⁺.

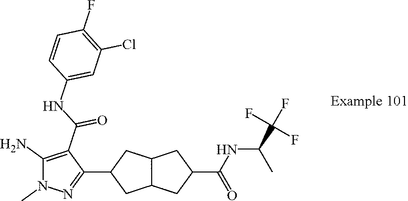 [SMILES: C[C@@H](NC(=O)C1CC2CC(c3nn(C)c(N)c3C(=O)Nc3ccc(F)c(Cl)c3)CC2C1)C(F)(F)F]

5-Amino-N-(3-chloro-4-fluorophenyl)-1-methyl-3-(5-(((R)-1,1,1-trifluoropropan-2-yl)carbamoyl)octahydropentalen-2-yl)-1H-pyrazole-4-carboxamide Isomer I. To a solution of compound 5-(5-amino-4-(3-chloro-4-fluorophenylcarbamoyl)-1-methyl-1H-pyrazol-3-yl)octahydro pentalene-2-carboxylic acid (50 mg, 0.1 mmol) and (R)-1,1,1-trifluoropropan-2-amine hydrochloride (23 mg, 0.15 mmol) in DMF (2 mL) was added HATU (136 mg, 0.36 mmol) and Et₃N (36 mg, 0.36 mmol). The reaction was stirred at room temperature for 2 h. After the starting material was consumed, the reaction was diluted with ethyl acetate, washed with H₂O, then brine. The organic layer was dried over anhydrous Na₂SO₄, filtered, and concentrated under reduced pressure. The residue was purified by pre-HPLC to afford 5-amino-N-(3-chloro-4-fluorophenyl)-1-methyl-3-(5-(((R)-1,1,1-trifluoropropan-2-yl)carbamoyl)octahydropentalen-2-yl)-1H-pyrazole-4-carboxamide as a mixture of isomers. Chiral-separation to afforded 5-amino-N-(3-chloro-4-fluorophenyl)-1-methyl-3-(5-(((R)-1,1,1 -trifluoropropan-2-yl)carbamoyl)octahydropentalen-2-yl)-1H-pyrazole-4-carboxamide Isomer I and 5-amino-N-(3-chloro-4-fluorophenyl)-1-methyl-3-(5-(((R)-1,1,1-trifluoropropan-2-yl)carbamoyl)octahydropentalen-2-yl)-1H-pyrazole-4-carboxamide Isomer II. TLC: 67% ethyl acetate / petroleum ether (R_{f}: 0.3). MS Calcd.: 515.2; MS Found: 516.3 [M + 1]⁺; ¹H NMR (400 MHz, CD₃OD): δ 7.80 (dd, J = 6.8, 2.4 Hz, 1H), 7.43-7.39 (m, 1H), 7.19 (t, J = 8.8 Hz, 1H), 4.66-4.58 (m, 1H), 3.56 (s, 3H), 3.31-3.25 (m, 1H), 2.83-2.75 (m, 1H), 2.72-2.67 (m, 2H), 2.33-2.26 (m, 2H), 1.87-1.78 (m, 2H), 1.70-1.63 (m, 2H), 1.43-1.35 (m, 2H), 1.29 (d, J = 7.2 Hz, 3H) ppm.

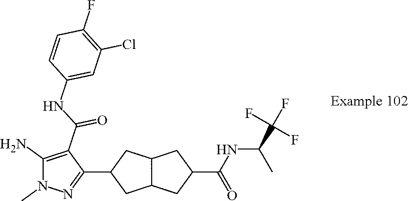 [SMILES: C[C@@H](NC(=O)C1CC2CC(c3nn(C)c(N)c3C(=O)Nc3ccc(F)c(Cl)c3)CC2C1)C(F)(F)F]

5-Amino-N-(3-chloro-4-fluorophenyl)-1-methyl-3-(5-(((R)-1.1,1-trifluoropropan-2-yl)carbamoyl)octahydropentalen-2-yl)-1H-pyrazole-4-carboxamide Isomer II. ¹H NMR (400 MHz, CD₃OD): δ 7.79 (dd, J = 6.4, 2.4 Hz, 1H), 7.43-7.39 (m, 1H), 7.19 (t, J = 9.2 Hz, 1H), 4.64-4.57 (m, 1H), 3.63-3.58 (m, 1H), 3.56 (s, 3H), 2.82-2.77 (m, 1H), 2.62-2.58 (m, 2H), 2.32-2.26 (m, 2H), 2.14-2.03 (m, 2H), 1.64-1.54 (m, 4H), 1.28 (d, J = 7.2 Hz, 3H) ppm.

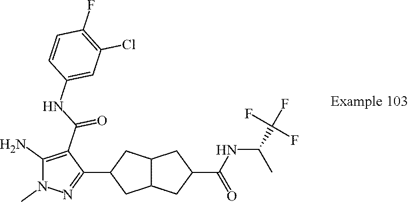 [SMILES: C[C@H](NC(=O)C1CC2CC(c3nn(C)c(N)c3C(=O)Nc3ccc(F)c(Cl)c3)CC2C1)C(F)(F)F]

5-Amino-N-(3-chloro-4-fluorophenyl)-1-methyl-3-(5-(((S)-1,1,1-trifluoropropan-2-yl)carbamoyl)octahydropentalen-2-yl)-1H-pyrazole-4-carboxamide Isomer I. To a solution of 5-(5-amino-4-(3-chloro-4-fluorophenylcarbamoyl)-1-methyl-1H-pyrazol-3-yl)octahydro pentalene-2-carboxylic acid (80 mg, 0.19 mmol) and (S)-1,1,1-trifluoropropan-2-amine hydrochloride (37 mg, 0.25 mmol) in DMF (2 mL) was added HATU (217 mg, 0.57 mmol) and Et₃N (58 mg, 0.57 mmol). The reaction was stirred at room temperature for 2 h. After the starting material was consumed, the reaction was diluted with ethyl acetate, washed with H₂O, then brine and dried over anhydrous Na₂SO₄. After filtration, the filtrate was concentrated under reduced pressure and the residue was purified by pre-HPLC to afford 5-amino-N-(3-chloro-4-fluorophenyl)-1-methyl-3-(5-(((S)-1,1,1-trifluoropropan-2-yl)carbamoyl)octahydropentalen-2-yl)-1H-pyrazole-4-carboxamide as a mixture of diastereomers Isomer I and Isomer II. Chiral-separation to afforded 5-amino-N-(3-chloro-4-fluorophenyl)- 1-methyl-3-(5-(((S)-1,1,1-trifluoropropan-2-yl)carbamoyl)octahydropentalen-2-yl)-1H-pyrazole-4-carboxamide as a mixture of diastereomers Isomer I and 5-amino-N-(3-chloro-4-fluorophenyl)-1-methyl-3-(5-(((S)-1,1,1 -trifluoropropan-2-yl)carbamoyl)octahydropentalen-2-yl)-1H-pyrazole-4-carboxamide as a mixture of diastereomers Isomer II. TLC: 67% ethyl acetate / petroleum ether (Rf: 0.3). MS Calcd.: 515.2. Found: 516.3 [M + 1]⁺. ¹H NMR (400 MHz, CD₃OD): δ 7.80 (dd, J = 6.8, 2.8 Hz, 1H), 7.43-7.39 (m, 1H), 7.19 (t, J = 8.8 Hz, 1H), 4.64-4.60 (m, 1H), 3.56 (s, 3H), 3.31-3.26 (m, 1H), 2.81-2.77 (m, 1H), 2.72-2.67 (m, 2H), 2.33-2.26 (m, 2H), 1.85-1.77 (m, 2H), 1.70-1.63 (m, 2H), 1.43-1.35 (m, 2H), 1.29 (d, J = 7.2 Hz, 3H) ppm.

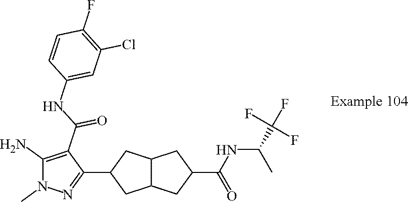 [SMILES: C[C@H](NC(=O)C1CC2CC(c3nn(C)c(N)c3C(=O)Nc3ccc(F)c(Cl)c3)CC2C1)C(F)(F)F]

5-Amino-N-(3-chloro-4-fluorophenyl)-1-methyl-3-(5-(((S)-1,1,1-trifluoropropan-2-yl)carbamoyl)octahydropentalen-2-yl)-1H-pyrazole-4-carboxamide Isomer II. ¹H NMR (400 MHz, CD₃OD): δ 7.79 (dd, J = 6.8, 2.8 Hz, 1H), 7.43-7.39 (m, 1H), 7.19 (t, J = 9.2 Hz, 1H), 4.64-4.57 (m, 1H), 3.63-3.58 (m, 1H), 3.56 (s, 3H), 2.82-2.77 (m, 1H), 2.61-2.59 (m, 2H), 2.32-2.26 (m, 2H), 2.14-2.03 (m, 2H), 1.64-1.54 (m, 4H), 1.28 (d, J = 7.2 Hz, 3H) ppm.

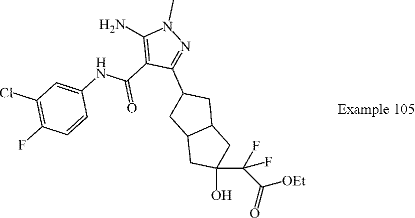 [SMILES: CCOC(=O)C(F)(F)C1(O)CC2CC(c3nn(C)c(N)c3C(=O)Nc3ccc(F)c(Cl)c3)CC2C1]

Ethyl 2-(5-(5-amino-4-((3-chloro-4-fluorophenyl)carbamoyl)-1-methyl-1H-pyrazol-3-yl)-2-hydroxyoctahydropentalen-2-yl)-2,2-difluoroacetate. To a mixture of Zn powder (12 eq, 18.46 mmol) in dry THF (40 mL) was added ethyl 2-bromo-2,2-difluoroacetate (11 eq, 16.92 mmol) at 60° C., the mixture was stirred for 0.5 h at 60° C. in an Ar atmosphere. The mixture was cooled to 45° C., a solution of 5-amino-N-(3-chloro-4-fluorophenyl)-1-methyl-3-(5-oxooctahydropentalen-2-yl)-1H-pyrazole-4-carboxamide (600 mg, 1 eq, 1.54 mmol) in dry THF (5 mL) was added. The mixture was cooled to room temperature, and Et₂AlCl (1.0 M, 1.1 eq, 1.69 mmol) was added. The reaction mixture was stirred for 6 h at room temperature. The mixture was quenched with sat. aq. NH₄Cl (40 mL) and extracted with EtOAc (10 mL x 3). The combined organic layers were dried over anhydrous Na₂SO₄ and concentrated in vacuo. The crude product was purified by reserved column chromatography to afford ethyl 2-(5-(5-amino-4-((3-chloro-4-fluorophenyl)carbamoyl)-1-methyl-1H-pyrazol-3-yl)-2-hydroxyoctahydropentalen-2-yl)-2,2-difluoroacetate as a white solid. MS Calcd; 514.1. Found: 515.2 [M + 1]⁺. ¹H NMR (400 MHz, DMSO-d₆): δ 8.93 (s, 1H), 7.92 (dd, J = 7.0, 2.6 Hz, 1H), 7.55-7.47 (m, 1H), 7.35 (t, J = 9.0 Hz, 1H), 5.98 (s, 2H), 5.47 (s, 1H), 4.26 (q, J = 7.2 Hz, 1H), 3.48 (s, 3H), 3.42-3.30 (m, 1H), 2.65-2.55 (m, 2H), 2.17-2.07 (m, 2H), 2.06-1.96 (m, 2H), 1.85-1.73 (m, 2H), 1.67 (d, J =14.0 Hz, 2H), 1.25 (t, J =7.2 Hz, 3H) ppm.

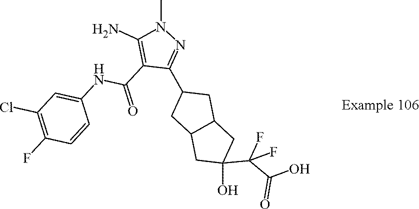 [SMILES: Cn1nc(C2CC3CC(O)(C(F)(F)C(=O)O)CC3C2)c(C(=O)Nc2ccc(F)c(Cl)c2)c1N]

2-Amino-4-((3-chloro-4-fluorophenyl)carbamoyl)-1-methyl-1H-pyrazol-3-yl)-2-hydroxyoctahydropentalen-2-yl)-2,2-difluoroacetic acid. To a solution of ethyl 2-amino-4-((3-chloro-4-fluorophenyl)carbamoyl)-1-methyl-1H-pyrazol-3-yl)-2-hydroxyoctahydropentalen-2-yl)-2,2-difluoroacetate (130 mg, 1 eq, 0.25 mmol) in MeOH (5 mL) was added LiOH (10 eq, 2.53 mmol). The reaction was stirred for 1 h at room temperature. The reaction mixture was adjusted to pH 3 with 1N HCl and extracted with EtOAc (10 mL x 3). The combined organic layers were dried over anhydrous Na₂SO₄ and concentrated in vacuo. The residue was purified by reserved column chromatography to afford 2-(5-(5-amino-4-((3-chloro-4-fluorophenyl)carbamoyl)-1-methyl-1H-pyrazol-3-yl)-2-hydroxyoctahydropentalen-2-yl)-2,2-difluoroacetic acid a a white solid. MS Calcd.: 486.1. Found: 487.1 [M + 1]⁺.¹H NMR (400 MHz, CD₃OD): δ 7.81 (dd, J = 6.6, 2.6 Hz, 1H), 7.44-7.37 (m, 1H), 7.19 (t, J = 9.0 Hz, 1H), 3.59 (s, 3H), 3.44-3.32 (m, 1H), 2.80-2.66 (m, 2H), 2.36-2.16 (m, 4H), 1.96-1.83 (m, 2H), 1.76 (d, J =14.0 Hz, 2H) ppm.

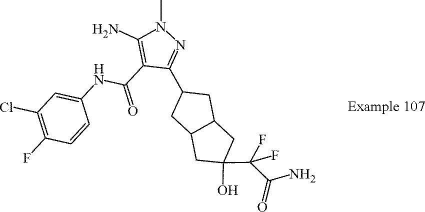 [SMILES: Cn1nc(C2CC3CC(O)(C(F)(F)C(N)=O)CC3C2)c(C(=O)Nc2ccc(F)c(Cl)c2)c1N]

5-Amino(5-(2-amino-1,1-difluoro-2-oxoethyl)-5-hydroxyoctahydropentalen-2-yl)-N-(3-chloro-4-fluorophenyl)-1-methyl-1H-pyrazole-4-carboxamide. To a solution of ethyl 2-(5-(5-amino-4-((3-chloro-4-fluorophenyl)carbamoyl)- 1-methyl-1H-pyrazolyl)-2-hydroxyoctahydropentalen-2-yl)-2,2-difluoroacetate (100 mg, 1 eq, 0.19 mmol) in MeOH (2 mL) was added NH₄OH (2 mL) and the reaction mixture stirred for 2 h at 70° C. in a sealed tube. The reaction mixture was concentrated in vacuo. The residue was purified by reserved column chromatography to afford a white solid 5-amino-3-(5-(2-amino-1,1-difluoro-2-oxoethyl)-5-hydroxyoctahydropentalen-2-yl)-N-(3-chloro-4-fluorophenyl)-1-methyl-1H-pyrazole-4-carboxamide. MS Calcd.: 485.1. Found: 486.3 [M+H]⁺. ¹H NMR (400 MHz, d₆-DMSO): δ 8.93 (s, 1H), 7.92 (dd, J = 7.0, 2.6 Hz, 1H), 7.79 (brs, 1H), 7.74 (brs, 1H), 7.54-7.48 (m, 1H), 7.35 (t, J = 9.2 Hz, 1H), 5.98 (s, 2H), 5.19 (s, 1H), 3.48 (s, 3H), 3.42-3.34 (m, 1H), 2.62-2.52 (m, 2H), 2.17-2.00 (m, 4H), 1.86-1.72 (m, 2H), 1.62 (d, J=13.6 Hz, 2H) ppm.

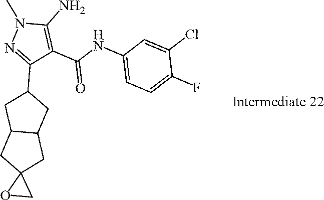 [SMILES: Cn1nc(C2CC3CC4(CO4)CC3C2)c(C(=O)Nc2ccc(F)c(Cl)c2)c1N]

5-Amino-N-(3-chloro-4-fluorophenyl)-3-(hexahydro-1′H-spiro[oxirane-2,2′-pentalene]-5′-yl)-1-methyl-1H-pyrazole-4-carboxamide. To a solution of potassium 2-methylpropan-2-olate (230 mg, 2.05 mmol) in THF (30 mL) was added trimethylsulfoxonium iodide (450 mg, 2.05 mmol). The mixture was stirred at RT for 1h under N₂. 5-Amino-N-(3-chloro-4-fluorophenyl)-1-methyl-3-(5-oxooctahydropentalen-2-yl)-1H-pyrazole-4-carboxamide (200 mg, 0.53 mmol) was then added to the mixture and stirring continued at 60° C. for 5 h under an N₂ atmosphere. The solvent was removed under vacuum and the product purified by silica gel column chromatography using 1:1 ethyl acetate/petroleum ether to afford 5-amino-N-(3-chloro-4-fluorophenyl)-3-(hexahydro-1′H-spiro[oxirane-2,2′-pentalene]-5′-yl)-1-methyl-1H-pyrazole-4-carboxamide. (200 mg, 96.6%) as yellow solid. MS Calcd.: 404.1; MS Found: 405.2 [M+1] ⁺.

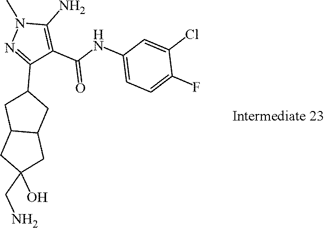 [SMILES: Cn1nc(C2CC3CC(O)(CN)CC3C2)c(C(=O)Nc2ccc(F)c(Cl)c2)c1N]

5-Amino(5-(aminomethyl)-5-hydroxyoctahydropentalen-2-yl)-N-(3-chloro-4-fluorophenyl)-1-methyl-1H-pyrazole-4-carboxamide. To a solution of 5-amino-N-(3-chloro-4-fluorophenyl)(hexahydro-1′H-spiro[oxirane-2,2′-pentalen]-5′-yl)-1-methyl-1H-pyrazole-4-carboxamide (200 mg, 0.495 mmol) in THF (5 mL) was added NH₄OH (5 mL). The mixture was stirred at room temperature overnight. The solvent was removed and the product purified by silica gel column chromatography using ethyl acetate to afford 5-amino-3-(5-(aminomethyl)-5-hydroxyoctahydropentalen-2-yl)-N-(3-chloro-4-fluorophenyl)-1-methyl-1H-pyrazole-4-carboxamide as a white solid. MS Calcd.: 421.1; MS Found: 422.3 [M + 1]⁺.

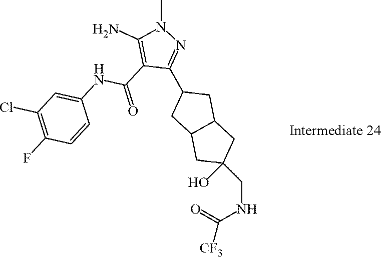 [SMILES: Cn1nc(C2CC3CC(O)(CNC(=O)C(F)(F)F)CC3C2)c(C(=O)Nc2ccc(F)c(Cl)c2)c1N]

5-Amino-N-(3-chloro-4-fluorophenyl)-3-(5-hydroxy-5-((2,2,2-trifluoroacetamido)methyl)octahydropentalen-2-yl)-1-methyl-1H-pyrazole-4-carboxamide. To a solution of 5-amino-3-(5-(aminomethyl)-5-hydroxyoctahydropentalen-2-yl)-N-(3-chloro-4-fluorophenyl)-1-methyl-1H-pyrazole-4-carboxamide (150 mg, 0.38 mmol), Et₃N (76 mg, 0.76 mmol) in THF (2 mL) and DCM (2 mL), at 0° C. was added trifluoroacetic anhydride (88 mg, 0.42 mmol) and the reaction stirred at r.t for 2 h. The reaction was quenched with 30 mL of water and extracted with ethyl acetate (30 mL x 2). The organic layers were concentrated. The residue was purified by Prep-TLC to afford 5-amino-N-(3-chloro-4-fluorophenyl)-3-(5-hydroxy-5-((2,2,2-trifluoroacetamido)methyl)octahydropentalen-2-yl)-1-methyl-1H-pyrazole-4-carboxamide. MS Calcd.: 517.2. MS Found: 518.2 [M + 1]⁺.

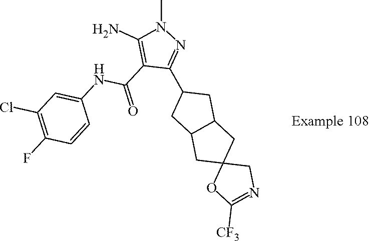 [SMILES: Cn1nc(C2CC3CC4(CN=C(C(F)(F)F)O4)CC3C2)c(C(=O)Nc2ccc(F)c(Cl)c2)c1N]

5-Amino-N-(3-chloro-4-fluorophenyl)-1-methyl-3-(2-(trifluoromethyl)-3′,3a′,4′,5′,6′,6a′-hexahydro-1′H,4H-spiro[oxazole-5,2′-pentalen]-5′-yl)-1H-pyrazole-4-carboxamide. To a solution of 5-amino-N-(3-chloro-4-fluorophenyl)-3-(5-hydroxy-5-((2,2,2-trifluoroacetamido)methyl)octahydropentalen-2-yl)-1-methyl-1H-pyrazole-4-carboxamide (60 mg, 0.12 mmol) in THF (1 mL) was added P₂O₅ (50 mg, 0.35 mmol). The reaction mixture was irradiated with microwave radiation for 1 h at 90° C. The reaction was poured into 30 mL of aq. NaHCO₃ and extracted with ethyl acetate (30 mL x 2). The organic layers were concentrated, and the residue was purified by Prep-HPLC to afford 5-amino-N-(3-chloro-4-fluorophenyl)-1-methyl-3-(2-(trifluoromethyl)-3′,3a′,4′,5′,6′,6a′-hexahydro-1′H,4H-spiro[oxazole-5,2′-pentalen]-5′-yl)-1H-pyrazole-4-carboxamide. MS Calcd.: 499.1. Found: 500.2 [M + 1]⁺. ¹H NMR (400 MHz, CDCl₃): δ 7.71 (dd, J = 2.4, 6.4 Hz, 1H), 7.32-7.27 (m, 2H), 7.12 (t, J = 8.8 Hz, 1H), 6.34 (brs, 1H), 5.55 (s, 1H), 5.25 (s, 2H), 4.00 (s, 2H), 3.58 (s, 3 H), 3.35-3.25 (m, 1H), 3.15-3.05 (m, 1H), 2.90-2.80 (m, 1H), 2.64-2.55 (m, 1H), 2.45-2.33 (m, 2H), 2.18-2.11 (m, 1H), 1.73-1.64 (m, 2H) ppm.

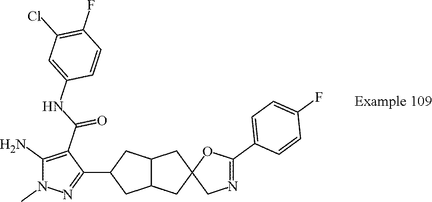 [SMILES: Cn1nc(C2CC3CC4(CN=C(c5ccc(F)cc5)O4)CC3C2)c(C(=O)Nc2ccc(F)c(Cl)c2)c1N]

5-Amino-N-(3-chloro-4-fluorophenyl)-3-(2-(4-fluorophenyl)-3′,3a′,4′,5′,6′,6a′-hexahydro-1′H,4H-spiro[oxazole-5,2′-pentalen]-5′-yl)-1-methyl-1H-pyrazole-4-carboxamide. ¹H NMR (400 MHz, DMSO-d₆ + D₂O): δ 9.01 (s, 1H), 7.98-7.91 (m, 3H), 7.55-7.51 (m, 1H), 7.38-7.27 (m, 3H), 6.01 (s, 2H), 3.83 (s, 2H), 3.60 (s, 3H), 3.45-3.39 (m, 1H), 2.67 (s, 2H), 2.22-2.16 (m, 2H), 1.95-1.84 (m, 6H) ppm.

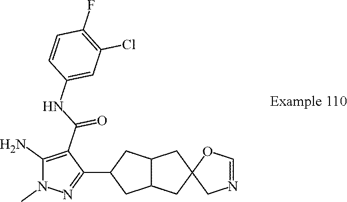 [SMILES: Cn1nc(C2CC3CC4(CN=CO4)CC3C2)c(C(=O)Nc2ccc(F)c(Cl)c2)c1N]

5-Amino-N-(3-chloro-4-fluorophenyl)-3-(3′,3a′,4′,5′,6′,6a′-hexahydro-1′H,4H-spiro[oxazole-5,2′-pentalen]-5′-yl)-1-methyl-1H-pyrazole-4-carboxamide. ¹H-NMR (400 MHz, DMSO-d₆): δ 7.93-7.90 (dd, J = 7.2, 2.4 Hz, 1H), 7.53-7.51 (m, 1H), 7.35 (t, J = 8.8 Hz, 1H), 5.97 (s, 2H), 5.41 (s, 1H), 3.70 (d, J = 4.8, 2H), 3.42 (s, 3H), 3.39-3.30 (m, 1H), 3.10-3.08 (m, 1H), 2.67-2.63 (m, 1H), 2.45-2.38 (m, 1H), 2.22-2.14 (m, 2H), 1.99-1.95 (m, 1H), 1.39-1.31 (m, 2H) ppm.

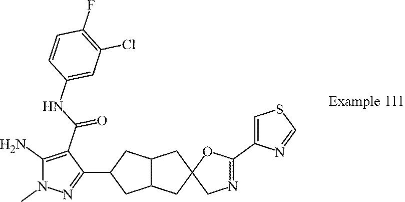 [SMILES: Cn1nc(C2CC3CC4(CN=C(c5cscn5)O4)CC3C2)c(C(=O)Nc2ccc(F)c(Cl)c2)c1N]

5-Amino-N-(3-chloro-4-fluorophenyl)-1-methyl-3-(2-(thiazol-4-yl)-3′,3a′,4′,5′,6′,6a′-hexahydro-1′H,4H-spiro[oxazole-5,2′-pentalen]-5′-yl)-1H-pyrazole-4-carboxamide. ¹H-NMR (400 MHz, DMSO-d₆): δ 7.92-7.90 (m, 1H), 7.53-7.49 (m, 1H), 7.35 (t, J = 9.2 Hz, 1H), 5.96 (s, 2H), 5.37 (s, 1H), 3.88-3.87 (m, 2H), 3.48 (s, 3H), 3.40-3.37 (m, 1H), 3.10-3.08 (m, 1H), 2.68-2.66 (m, 1H), 2.49-2.44 (m, 1H), 2.22-2.14 (m, 2H), 2.02-1.98 (d, J = 16 Hz, 1H), 1.37-1.33 (m, 2H) ppm.

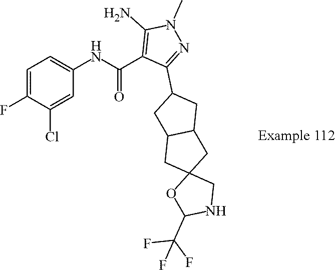 [SMILES: Cn1nc(C2CC3CC4(CNC(C(F)(F)F)O4)CC3C2)c(C(=O)Nc2ccc(F)c(Cl)c2)c1N]

5-Amino-N-(3-chloro-4-fluorophenyl)-1-methyl-3-(2-(trifluoromethyl)hexahydro-1′H-spiro[oxazolidine-5,2′-pentalen]-5′-yl)-1H-pyrazole-4-carboxamide Isomer I. To a solution of 5-amino-3-(5-(aminomethyl)-5-hydroxyoctahydropentalen-2-yl)-N-(3-chloro-4-fluorophenyl)-1-methyl-1H-pyrazole-4-carboxamide (150 mg, 0.36 mmol) in toluene (2 mL) was added 1-ethoxy-2,2,2-trifluoroethanal (71 mg (81% purity), 0.39 mmol) and PPTS (9 mg, 0.036 mmol). The reaction solution was stirred at 90° C. for 1 hour. After the starting material was consumed, the volatiles were removed in vacuo, and the product purified by pre-HPLC to afford 5-amino-N-(3-chloro-4-fluorophenyl)-1-methyl-3-(2-(trifluoromethyl)hexahydro-1′H-spiro[oxazolidine-5,2′-pentalen]-5′-yl)-1H-pyrazole-4-carboxamide as a mixture of Isomer I and Isomer II. Chiral-separation afforded 5-amino-N-(3-chloro-4-fluorophenyl)-1-methyl-3-(2-(trifluoromethyl)hexahydro-1′H-spiro[oxazolidine-5,2′-pentalen]-5′-yl)-1H-pyrazole-4-carboxamide Isomer I and 5-amino-N-(3-chloro-4-fluorophenyl)-1-methyl-3-(2-(trifluoromethyl)hexahydro-1′H-spiro[oxazolidine-5,2′-pentalen]-5′-yl)-1H-pyrazole-4-carboxamide Isomer II. TLC: 9% MeOH / DCM (R_{f}: 0.3). MS Calcd.: 501.2. MS Found: 502.2 [M + 1]⁺. ¹H NMR (400 MHz, CD₃OD): δ 7.80 (dd, J = 6.4, 2.4 Hz, 1H), 7.43-7.39 (m, 1H), 7.19 (t, J = 9.2 Hz, 1H), 4.92-4.88 (m, 1H), 3.56 (s, 3H), 3.44-3.40 (m, 1H), 2.93 (dd, J = 116, 11.6 Hz, 2H), 2.55-2.53 (m, 2H), 2.33-2.28 (m, 2H), 1.93-1.61 (m, 6H) ppm.

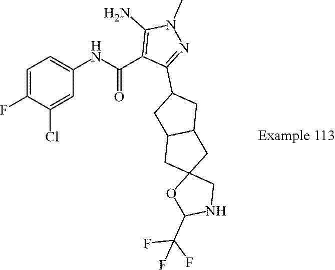 [SMILES: Cn1nc(C2CC3CC4(CNC(C(F)(F)F)O4)CC3C2)c(C(=O)Nc2ccc(F)c(Cl)c2)c1N]

5-Amino-N-(3-chloro-4-fluorophenyl)-1-methyl-3-(2-(trifluoromethyl)hexahydro-1′H-spiro[oxazolidine-5,2′-pentalen]-5′-yl)-1H-pyrazole-4-carboxamide Isomer II. ¹H NMR (400 MHz, CD₃OD): δ 7.80 (dd, J = 6.8, 2.4 Hz, 1H), 7.43-7.39 (m, 1H), 7.19 (t, J = 8.8 Hz, 1H), 4.92-4.88 (m, 1H), 3.56 (s, 3H), 3.44-3.40 (m, 1H), 2.94 (dd, J = 116, 11.6 Hz, 2H), 2.55-2.54 (m, 2H), 2.33-2.28 (m, 2H), 1.94-1.61 (m, 6H) ppm.

| | |
|---|---|
| Example | Structure and analysis |
| Example 114 | 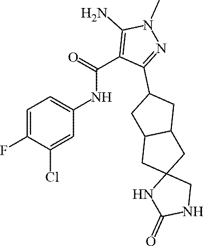 [SMILES: Cn1nc(C2CC3CC4(CNC(=O)N4)CC3C2)c(C(=O)Nc2ccc(F)c(Cl)c2)c1N] |
| | 5-Amino-N-(3-chloro-4-fluorophenyl)-1-methyl-3-(2-oxohexahydro-1′H-spiro[imidazolidine-4,2′-pentalen]-5′-yl)-1H-pyrazole-4-carboxamide MS calcd. for C₂₁H₂₄ClFN₆O₂— 446.2; Found: 447.2 [M + 1]⁺. ¹H NMR (400 MHz, DMSO-d₆): δ 9.00 (s, 1H), 7.89 (dd, J = 6.8, 2.4 Hz, 1H), 7.54-7.48 (m, 1H), 7.34 (t, J = 8.8 Hz, 1H), 6.61 (s, 1H), 6.01 (s, 1H), 5.94 (s, 2H), 3.54-3.50 (m, 1H), 3.48 (s, 3H), 3.21 (s, 2H), 2.64-2.56 (m, 2H), 2.20-2.12 (m, 2H), 1.90-1.81 (m, 2H), 1.48-1.34 (m, 4H). |
| Example 115 | 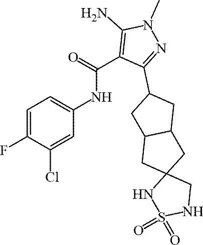 [SMILES: Cn1nc(C2CC3CC4(CNS(=O)(=O)N4)CC3C2)c(C(=O)Nc2ccc(F)c(Cl)c2)c1N] |
| | 5-Amino-N-(3-chloro-4-fluorophenyl)-3-(1′,1′-dioxidohexahydro-1H-spiro[pentalene-2,3′-[1,2,5]-thiadiazolidin]-5-yl)-1-methyl-1H-pyrazole-4-carboxamide MS calcd. for C₂₀H₂₄ClFN₆O₃S: 482.1; Found; 483.0 [M + 1]⁺. ¹H NMR (400 MHz, DMSO-d₆): δ 8.94 (s, 1H), 7.96-7.88 (m, 2H), 7.56-7.48 (m, 1H), 7.34 (t, J = 8.8 Hz, 1H), 6.86 (t, J = 6.4 Hz, 1H), 6.80-5.20 (m, 2H), 3.62-3.32 (m, 4H, merged), 3.21 (s, 2H), 2.60-2.52 (m, 2H, merged), 2.20-2.08 (m, 4H), 1.48-1.36 (m, 4H) ppm. |
| Example 116 | 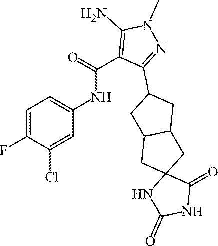 [SMILES: Cn1nc(C2CC3CC4(CC3C2)NC(=O)NC4=O)c(C(=O)Nc2ccc(F)c(Cl)c2)c1N] |
| | 5-Amino-N-(3-chloro-4-fluorophenyl)-3-(2,5-dioxohexahydro-1′H-spiro[imidazolidine-4,2′-pentalen]-5′-yl)-1-methyl-1H-pyrazole-4-carboxamide MS calcd. for C₂₁H₂₂ClFN₆O₃— 460.1; Found; 461.2 [M + 1]⁺. ¹H NMR (400 MHz, DMSO-d₆): δ 10.52 (s, 1H), 9.00 (s, 1H), 8.36 (s, 1H), 7.89 (dd, J = 6.8, 2.4 Hz, 1H), 7.55-7.49 (m, 1H), 7.34 (t, J = 8.8 Hz, 1H), 5.96 (s, 2H), 3.68-3.58 (m, 1H), 3.50 (s, 3H), 2.73-2.62 (m, 2H), 2.22-2.12 (m, 2H), 1.91-1.82 (m, 2H), 1.77-1.68 (m, 2H), 1.56-1.44 (m, 2H) ppm. |

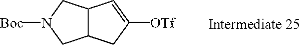 [SMILES: CC(C)(C)OC(=O)N1CC2C=C(OS(=O)(=O)C(F)(F)F)CC2C1]

tert-Butyl 5-(trifluoromethylsulfonyloxy)-3,3a,6,6a-tetrahydrocyclopenta[c]pyrrole-2(1H)-carboxylate. LiHMDS (60 mL, 60 mmol, 1.0 M in THF) was added slowly to a solution of tert-butyl 5-oxohexahydrocyclopenta[c]pyrrole-2(1H)-carboxylate (4.5 g, 20 mmol) in anhydrous THF (50 mL) at -78° C. for 30 min. 1,1,1-Trifluoro-N-phenyl-N-(trifluoromethylsulfonyl)methanesulfonamide (14.3 g, 40 mmol) in THF (30 mL) was added slowly and the mixture stirred for 2 h. The reaction mixture was warmed to room temperature and quenched with NH₄Cl (aq.). The solution was extracted with ethyl acetate (50 mL x 3). The combined organic layers were dried over Na₂SO₄, concentrated and purified by silica gel column chromatography using 5 - 10% ethyl acetate / petroleum ether (v/v) to afford compound tert-butyl 5-(trifluoromethylsulfonyloxy)-3,3a,6,6a-tetrahydrocyclopenta[c]pyrrole-2(1H)-carboxylate (4.5 g, 63%) as a pale-yellow solid. TLC: 30% ethyl acetate / petroleum ether (R_{f}: 0.3).

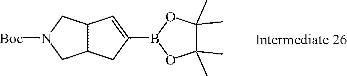 [SMILES: CC(C)(C)OC(=O)N1CC2C=C(B3OC(C)(C)C(C)(C)O3)CC2C1]

tert-Butyl 5-(4,4,5,5-tetramethyl-1,3,2-dioxaborolan-2-yl)-3,3a,6,6a-tetrahydrocyclopenta[c]pyrrole-2(1H)-carboxylate. A solution of tert-butyl 5-(((trifluoromethyl)sulfonyl)oxy)-3,3a,6,6a-tetrahydrocyclopenta[c]pyrrole-2(1H)-carboxylate (4.5 g, 12.6 mmol), 4,4,5,5-tetramethyl-2-(4,4,5,5-tetramethyl-1,3,2-dioxaborolan -2-yl)-1,3,2-dioxaborolane (4.2 g, 16.4 mmol), Pd(dppf)Cl₂ (182.9 mg, 0.25 mmol) and potassium phosphate (3.5 g, 16.4 mmol) in dioxane (80 mL) was stirred at 80° C. for 16 h under N₂ atmosphere. The reaction was then filtered through a pad of Celite and the cake washed with ethyl acetate (20 mL x 2). The filtrate was concentrated in vacuum and the crude product, tert-butyl 5-(4,4,5,5-tetramethyl-1,3,2-dioxaborolan-2-yl)-3,3a,6,6a-tetrahydrocyclopenta[c]pyrrole-2(1H)-carboxylate was used in the next step without further purification.

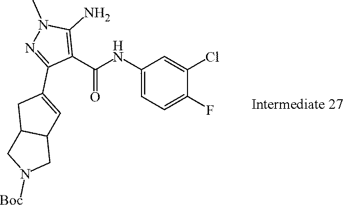 [SMILES: Cn1nc(C2=CC3CN(C(=O)OC(C)(C)C)CC3C2)c(C(=O)Nc2ccc(F)c(Cl)c2)c1N]

tert-Butyl 5-(5-amino-4-(3-chloro-4-fluorophenylcarbamoyl)-1-methyl-1H-pyrazol-3-yl)-3,3a,6,6a-tetrahydrocyclopenta[c]pyrrole-2(1H)-carboxylate. To a solution of crude tert-butyl 5-(4,4,5,5-tetramethyl-1,3,2-dioxaborolan-2-yl)-3,3a,6,6a-tetrahydrocyclopenta[c]pyrrole-2(1H)-carboxylate (12.6 mmol) in dioxane/H₂O (80 mL/16 mL) was added potassium phosphate (3.5 g, 16.4 mmol), Pd(dppf)Cl₂ ((182.9 mg, 0.25 mmol) and ethyl 5-amino-3-bromo-1-methyl-pyrazole-4-carboxylate (4.4 g, 12.6 mmol). The reaction was stirred at 80° C. for 4 h under N₂ atmosphere. The mixture was quenched with H₂O and extracted with ethyl acetate (50 mL x 2). The organic layer was concentrated in vacuo and the residue purified by silica gel column chromatography to afford tert-butyl 5-(5-amino-4-(3-chloro-4-fluorophenylcarbamoyl)-1-methyl-1H-pyrazol-3-yl)-3,3a,6,6a-tetrahydrocyclopenta[c]pyrrole-2(1H)-carboxylate (3.2 g, 53.4 % for two steps). MS Calcd.: 475.2; Found: 420.2 [M⁺-55].

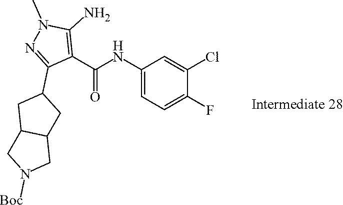 [SMILES: Cn1nc(C2CC3CN(C(=O)OC(C)(C)C)CC3C2)c(C(=O)Nc2ccc(F)c(Cl)c2)c1N]

tert-Butyl 5-(5-amino-4-((3-chloro-4-fluorophenyl)carbamoyl)-1-methyl-1H-pyrazol-3-yl)hexahydrocyclopenta[c]pyrrole-2(1H)-carboxylate. To a solution of tert-butyl 5-(5-amino-4-(3-chloro-4-fluorophenylcarbamoyl)-1-methyl-1H-pyrazol-3-yl)-3,3a,6,6atetrahydrocyclopenta [c]pyrrole-2(1H)-carboxylate (3.1 g, 6.5 mmol) in THF (90 mL) was added 10% Pd/C (1 g, containing 67% H₂O). The reaction was stirred at 15° C. for 15 min under a H₂ atmosphere (1 atm.) then filtered. The filtrate was concentrated, and the residue purified by silica gel column chromatography to give tert-butyl 5-(5-amino-4-((3-chloro-4-fluorophenyl)carbamoyl)-1-methyl-1H-pyrazol-3-yl)hexahydrocyclopenta[c]pyrrole-2(1H)-carboxylate as a white solid. TLC; 50% ethyl acetate / petroleum ether (R_{f.} 0.3). MS Calcd.: 477.2; Found: 422.3 [M⁺ - 55]. ¹H NMR (400 MHz, DMSO-d₆): δ 8.98 (s, 1H), 7.91 (dd, J = 6.8, 2.4 Hz, 1H), 7.54-7.50 (m, 1H), 7.34 (t, J = 9.6 Hz, 1H), 5.98 (s, 2H), 3.59-3.52 (m, 1H), 3.49 (s, 3H), 3.32 (s, 2H), 3.14-3.10 (m, 2H), 2.61-2.57 (m, 2H), 2.21-2.14 (m, 2H), 1.56-1.48 (m, 2H), 1.38 (s, 9H) ppm.

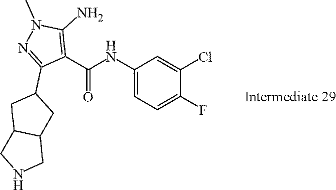 [SMILES: Cn1nc(C2CC3CNCC3C2)c(C(=O)Nc2ccc(F)c(Cl)c2)c1N]

5-Amino-N-(3-chloro-4-fluorophenyl)-1-methyl-3-(octahydrocyclopenta[c]pyrrol-5-yl)-1H-pyrazole-4-carboxamide. A solution of tert-butyl 5-(5-amino-4-(3-chloro-4-fluorophenylcarbamoyl)-1-methyl-1H-pyrazol-3-yl)hexahydrocyclopenta[c]pyrrole-2(1H)-carboxylate (3 g, 6.3 mmol) in 2 M HCl/dioxane (30 mL) was stirred at room temperature for 1 h. The reaction was concentrated and diluted with DCM. The DCM solution was adjusted to pH 8 ~ 9 with NaHCO₃ and extracted with DCM (30 mL x 3). The organic layer was dried and concentrated. The residue was purified by reversed column chromatography to afford 5-amino-N-(3-chloro-4-fluorophenyl)-1-methyl-3-(octahydrocyclopenta[c]pyrrol-5-yl)-1H-pyrazole-4-carboxamide (2.1 g, 84%) as a white solid. TLC: 10% MeOH / DCM (R_{f:} 0.4); MS Calcd.: 377.2; Found: 378.2 [M⁺ + 1]⁺. ¹H NMR (400 MHz, DMSO-d₆): δ 8.97 (s, 1H), 7.91 (dd, J = 6.8, 2.8 Hz, 1H), 7.53-7.51 (m, 1H), 7.34 (t, J = 9.2 Hz, 1H), 5.97 (s, 2H), 3.55-3.53 (m, 1H), 3.49 (s, 3H), 3.44-3.41 (m, 3H), 3.22-3.14 (m, 1H), 2.57 (s, 3H), 2.19-2.13 (m, 2H), 1.52-1.50 (m, 1H), 1.34-1.32 (m, 1H) ppm.

| | |
|---|---|
| Example | Structure and analysis |
| Example 117 | 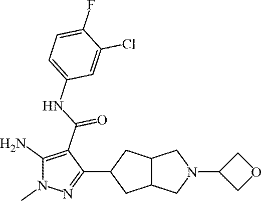 [SMILES: Cn1nc(C2CC3CN(C4COC4)CC3C2)c(C(=O)Nc2ccc(F)c(Cl)c2)c1N] |
| | 5-Amino-N-(3-chloro-4-fluorophenyl)-1-methyl-3-(2-(oxetan-3-yl)octahydrocyclopenta[c]pyrrol-5-yl)-1H-pyrazole-4-carboxamide. |
| | ¹H NMR (400 MHz, DMSO-d₆): δ 8.97 (s, 1H), 7.92 (dd, J = 6.8, 2.4 Hz, 1H), 7.54-7.50 (m, 1H), 7.37 (t, J = 9.2 Hz, 1H), 5.98 (d, J = 6.4 Hz, 2H), 3.62-3.53 (m, 1H), 3.49 (s, 3H), 3.40-3.31 (m, 2H), 3.23-3.20 (m, 2H), 3.05-2.95 (m, 2H), 2.63-2.50 (m, 4H), 2.23-2.07 (m, 2H), 1.57-1.50 (m, 2H) ppm. |
| Example 118 | 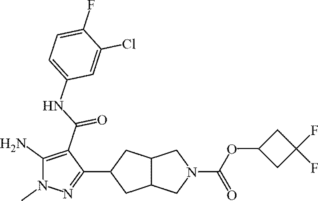 [SMILES: Cn1nc(C2CC3CN(C(=O)OC4CC(F)(F)C4)CC3C2)c(C(=O)Nc2ccc(F)c(Cl)c2)c1N] |
| | 3,3-Difluorocyclobutyl 5-(5-amino-4-((3-chloro-4-fluorophenyl)carbamoyl)-1-methyl-1H-pyrazol-3-yl)hexahydrocyclopenta[c]pyrrole-2(1H)-carboxylate. ¹H NMR (400 MHz, DMSO-d₆): δ 8.99 (s, 1H), 7.92 (dd, J = 7.2, 2.4 Hz, 1H), 7.55-7.51 (m, 1H), 7.37 (t, J = 9.2 Hz, 1H), 5.99 (s, 2H), 4.81-4.75 (m, 1H), 4.55 (t, J = 6.4 Hz, 2H), 4.41 (t, J = 6 Hz, 2H), 3.50 (s, 3H), 3.45-3.42 (m, 1H), 3.37 (m, 1H), 2.50-2.49 (m, 4H), 2.18-2.13 (m, 2H), 2.03-2.00 (m, 2H), 1.50-1.48 (m, 2H) ppm. |
| Example 119 | 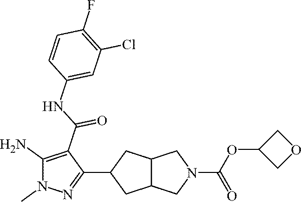 [SMILES: Cn1nc(C2CC3CN(C(=O)OC4COC4)CC3C2)c(C(=O)Nc2ccc(F)c(Cl)c2)c1N] |
| | Oxetan-3-yl 5-(5-amino-4-((3-chloro-4-fluorophenyl)carbamoyl)-1-methyl-1H-pyrazol-3-yl)hexahydrocyclopenta[c]pyrrole-2(1H)-carboxylate. ¹H NMR (400 MHz, DMSO-d₆): δ 9.00 (s, 1H), 7.92 (dd, J = 6.8, 2.8 Hz, 1H), 7.54-7.52 (m, 1H), 7.37 (t, J = 9.2 Hz, 1H), 5.99 (s, 2H), 5.29-5.25 (m, 1H), 4.75-4.72 (m, 2H), 4.50-4.46 (m, 2H), 3.61-3.54 (m, 1H), 3.42-3.33 (m, 5H), 3.31-3.14 (m, 2H), 2.70-2.66 (m, 2H), 2.21-2.18 (m, 2H), 1.58-1.5- (m, 2H) ppm |
| Example 120 | 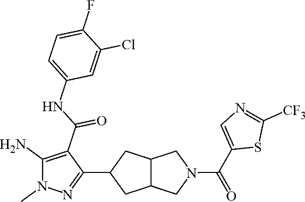 [SMILES: Cn1nc(C2CC3CN(C(=O)c4cnc(C(F)(F)F)s4)CC3C2)c(C(=O)Nc2ccc(F)c(Cl)c2)c1N] |
| | 5-Amino-N-(3-chloro-4-fluorophenyl)-1-methyl-3-(2-(2-(trifluoromethyl)thiazole-5-carbonyl)octahydrocyclopenta[c]pyrrol-5-yl)-1H-pyrazole-4-carboxamide. ¹H NMR (400 MHz, MeOD): δ 8.39 (d, J = 1.2 Hz, 1H), 7.80 (dd, J = 2.8, 6.8 Hz, 1H), 7.46-7.40 (m, 1H), 7.19 (t, J = 9 Hz, 1H), 3.95-4.02 (m, 1H), 3.78-3.60 (m, 4H), 3.55 (s, 3H), 2.96-2.78 (m, 2H), 2.41-2.36 (m, 2H), 1.82-1.75 (m, 2H) ppm. |
| Example 121 | 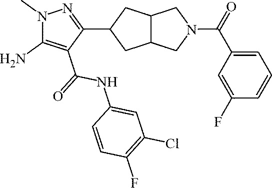 [SMILES: Cn1nc(C2CC3CN(C(=O)c4cccc(F)c4)CC3C2)c(C(=O)Nc2ccc(F)c(Cl)c2)c1N] |
| | 5-Amino-N-(3-chloro-4-fluorophenyl)-3-(2-(3-fluorobenzoyl)octahydrocyclopenta[c]pyrrol-5-yl)-1-methyl-1H-pyrazole-4-carboxamide. ¹H NMR (400 MHz, DMSO-d₆): δ 9.00 (s, 1H), 7.92-7.89 (m, 1H), 7.55-7.50 (s, 1H), 7.49-7.45(m, 1H), 7.37-7.27 (m, 3H), 6.00 (s, 2H), 3.60-3.55 (m, 4H), 3.51 (s, 3H), 3.23-3.21 (m, 1H), 2.67 (s, 2H), 2.26-2.11 (m, 2H), 1.64-1.45 (m, 2H) ppm. |
| Example 122 | 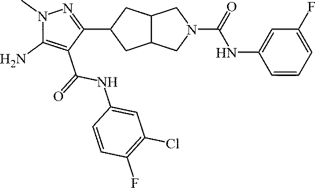 [SMILES: Cn1nc(C2CC3CN(C(=O)Nc4cccc(F)c4)CC3C2)c(C(=O)Nc2ccc(F)c(Cl)c2)c1N] |
| | 5-(5-Amino-4-((3-chloro-4-fluorophenyl)carbamoyl)-1-methyl-1H-pyrazol-3-yl)-N-(3-fluorophenyl)hexahydrocyclopenta[c]pyrrole-2(1H)-carboxamide. |
| | ¹H NMR (400 MHz, DMSO-d₆): δ 9.01 (s, 1H), 8.34 (s, 1H), 7.93-7.91 (m, 1H), 7.56-7.47 (m, 2H), 7.38-7.33 (m, 1H), 7.29-7.20 (m, 2H), 6.73-6.69 (m, 1H), 5.99 (s, 2H), 3.59 (t, J =7.6 Hz, 1H), 3.49 (m, 2H), 3.36-3.32 (m, 2H), 2.69-2.67 (m, 2H), 2.26-2.20 (m, 2H), 1.63-1.56 (m, 2H) ppm. |
| Example 123 | 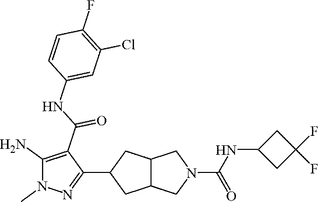 [SMILES: Cn1nc(C2CC3CN(C(=O)NC4CC(F)(F)C4)CC3C2)c(C(=O)Nc2ccc(F)c(Cl)c2)c1N] |
| | 5-(5-Amino-4-((3-chloro-4-fluorophenyl)carbamoyl)-1-methyl-1H-pyrazol-3-yl)-N-(3,3-difluorocyclobutyl)hexahydrocyclopenta[c]-pyrrole-2(1H)-carboxamide. ¹H NMR (400 MHz, DMSO-d₆): δ 9.00 (s, 1H), 7.91 (dd, J = 6.8, 2.4 Hz, 1H), 7.55-7.51 (m, 1H),7.35 (t, J = 9.2 Hz, 1H), 6.50 (d, J = 6.8 Hz, 1H), 6.00 (s, 2H), 3.97-3.94 (m, 1H), 3.59-3.54 (m, 1H), 3.49 (s, 3H), 3.32-3.26 (m, 2H), 3.17-3.14 (m, 2H),2.81-2.73 (m, 2H), 2.66-2.56 (m, 4H), 2.22-2.16 (m, 2H),1.56-1.48 (m, 2H) ppm. |
| Example 124 | 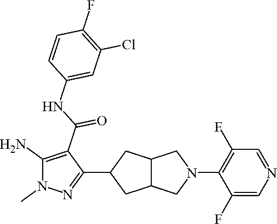 [SMILES: Cn1nc(C2CC3CN(c4c(F)cncc4F)CC3C2)c(C(=O)Nc2ccc(F)c(Cl)c2)c1N] |
| | 5-Amino-N-(3-chloro-4-fluorophenyl)-3-(2-(3,5-difluoropyridin-4-yl)octahydrocyclopenta[c]pyrrol-5-yl)-1-methyl-1H-pyrazole-4-carboxamide. ¹H NMR (400 MHz, CD₃OD): δ 7.97 (q, J = 1.6 Hz, 2H), 7.82 (dd, J = 2.4, 6.8 Hz, 1H), 7.45-7.41 (m, 1H), 7.21(t, J = 8.8 Hz, 1H), 3.74-3.64 (m, 4H), 3.58-3.54 (m, 4H), 2.80-2.78 (m, 2H), 2.42-2.35 (m, 2H), 1.75-1.67 (m, 2H) ppm. |
| | 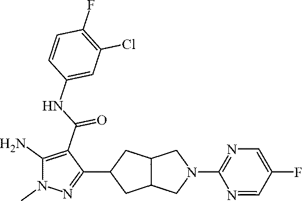 [SMILES: Cn1nc(C2CC3CN(c4ncc(F)cn4)CC3C2)c(C(=O)Nc2ccc(F)c(Cl)c2)c1N] |
| Example 125 | 5-Amino-N-(3-chloro-4-fluorophenyl)-3-(2-(5-fluoropyrimidin-2-yl)octahydrocyclopenta[c]pyrrol-5-yl)-1-methyl-1H-pyrazole-4-carboxamide. ¹H NMR (400 MHz, DMSO-d₆): δ 9.00(s, 1H), 8.41 (s, 2H), 7.90-7.93 (m, 1H), 7.52-7.56 (m, 1H), 7.35(t, J=8.8 Hz, 1H), 5.99 (S, 2H), 3.52-3.63 (m, 3H), 3.40-3.44(m, 5H), 2.76-2.78 (m, 2H), 2.24-2.50 (m, 2H), 1.57-1.64 (m, 2H) ppm |
| | 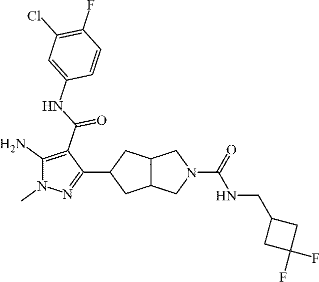 [SMILES: Cn1nc(C2CC3CN(C(=O)NCC4CC(F)(F)C4)CC3C2)c(C(=O)Nc2ccc(F)c(Cl)c2)c1N] |
| Example 126 | 5-(5-Amino-4-((3-chloro-4-fluorophenyl)carbamoyl)-1-methyl-1H-pyrazol-3-yl)-N-((3,3-difluorocyclobutyl)methyl)-hexahydrocyclopenta[c]pyrrole-2(1H)-carboxamide. ¹H NMR (400 MHz, DMSO-d₆): δ 8.99 (s, 1H), 7.91 (dd, J = 6.8, 2.4 Hz, 1H), 7.55-7.51 (m, 1H), 7.35 (t, J = 9.2 Hz, 1H), 6.30 (t, J = 5.6 Hz, 1H), 6.00 (s, 2H), 3.56-3.53 (m, 1H), 3.49 (s, 3H), 3.29-3.25 (m, 2H), 3.16-3.09 (m, 4H), 2.62-2.61 (m, 2H), 2.58-2.54 (m, 2H), 2.33-2.15 (m, 5H),1.55-1.48 (m, 2H) ppm. |
| Example 127 | 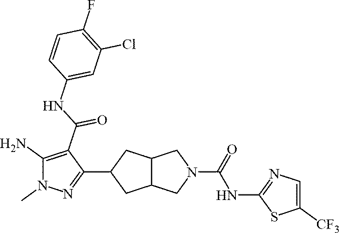 [SMILES: Cn1nc(C2CC3CN(C(=O)Nc4ncc(C(F)(F)F)s4)CC3C2)c(C(=O)Nc2ccc(F)c(Cl)c2)c1N] |
| | 5-(5-Amino-4-((3-chloro-4-fluorophenyl)carbamoyl)-1-methyl-1H-pyrazol-3-yl)-N-(5-(trifluoromethyl)thiazol-2-yl)hexahydrocyclopenta[c]pyrrole-2(1H)-carboxamide. ¹H NMR (400 MHz, DMSO-d₆): δ 11.29 (s, 1H), 9.01 (s, 1H), 7.97 (d, J = 1.6 Hz, 1H), 7.92 (dd, J = 6.8, 2.4 Hz, 1H), 7.56-7.52 (m, 1H),7.35 (t, J = 8.8 Hz, 1H), 6.00 (s, 2H), 3.63-3.60 (m, 1H), 3.58-3.48 (m, 5H), 3.42-3.39 (m, 2H), 2.73-2.71 (m, 2H), 2.26-2.19 (m, 2H), 1.61-1.23 (m, 2H) ppm. |
| Example 128 | 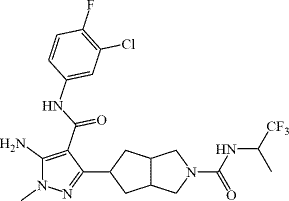 [SMILES: CC(NC(=O)N1CC2CC(c3nn(C)c(N)c3C(=O)Nc3ccc(F)c(Cl)c3)CC2C1)C(F)(F)F] |
| | 5-(5-Amino-4-((3-chloro-4-fluorophenyl)carbamoyl)-1-methyl-1H-pyrazol-3-yl)-N-(1,1,1-trifluoropropan-2-yl)hexahydrocyclopenta[c]pyrrole-2(1H)-carboxamide Isomer I. ¹H NMR (400 MHz, DMSO-d₆): δ 8.99 (s, 1H), 7.91 (dd, J = 7.2, 2.4 Hz, 1H), 7.54-7.52 (m, 1H), 7.35 (t, J = 9.2 Hz, 1H), 6.48 (d, J = 8.8 Hz, 1H), 6.00 (s, 2H), 4.53-4.42 (m, 1H), 3.58-3.50 (m, 1H), 3.49 (s, 3H), 3.37-3.36 (m, 1H), 3.28-3.15 (m, 3H), 2.64-2.63 (m, 2H), 2.21-2.18 (m, 2H), 1.55-1.52 (m, 2H), 1.24 (d, J = 7.2 Hz, 3H) ppm. |
| Example 129 | 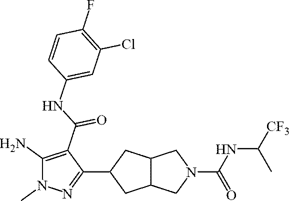 [SMILES: CC(NC(=O)N1CC2CC(c3nn(C)c(N)c3C(=O)Nc3ccc(F)c(Cl)c3)CC2C1)C(F)(F)F] |
| | 5-(5-Amino-4-((3-chloro-4-fluorophenyl)carbamoyl)-1-methyl-1H-pyrazol-3-yl)-N-(1,1,1-trifluoropropan-2-yl)hexahydrocyclopenta[c]pyrrole-2(1H)-carboxamide Isomer II ¹H NMR (400 MHz, DMSO-d₆): δ 8.99 (s, 1H), 7.91 (dd, J = 7.2, 2.4 Hz, 1H), 7.54-7.52 (m, 1H), 7.35 (t, J = 9.2 Hz, 1H), 6.48 (d, J = 8.8 Hz, 1H), 6.00 (s, 2H), 4.52-4.44 (m, 1H), 3.60-3.53 (m, 1H), 3.49 (s, 3H), |
| | 3.37-3.36 (m, 1H), 3.28-3.15 (m, 3H), 2.66-2.64 (m, 2H), 2.21-2.18 (m, 2H), 1.55-1.52 (m, 2H), 1.24 (d, J = 7.2 Hz, 3H) ppm. |
| Example 130 | 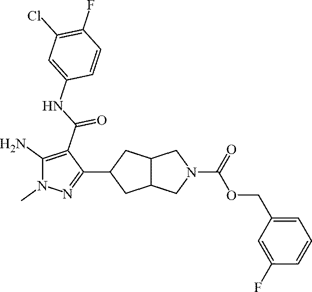 [SMILES: Cn1nc(C2CC3CN(C(=O)OCc4cccc(F)c4)CC3C2)c(C(=O)Nc2ccc(F)c(Cl)c2)c1N] |
| | 3-fluorobenzyl 5-(5-amino-4-((3-chloro-4-fluorophenyl)carbamoyl)-1-methyl-1H-pyrazol-3-yl)hexahydrocyclopenta[c]pyrrole-2(1H)-carboxylate. ¹H NMR Data (400 MHz, CD₃OD): δ 7.79 (dd, J = 2.8 Hz, 6.8 Hz, 1H), 7.44 - 7.40 (m, 1H), 7.37 - 7.32 (m, 1H), 7.16 (t, J = 9.2 Hz, 2H), 7.09 (d, J = 9.6 Hz, 1H), 7.79 (td, J = 2.4 Hz, 8.4 Hz, 1H), 5.10 (s, 2H), 3.61 -3.47 (m, 6H), 3.39 (t, J = 12.4 Hz, 2H), 2.76 (s, 2H), 2.37 - 2.30 (m, 2H), 1.70 - 1.62 (m, 2H) ppm. |
| Example 131 | 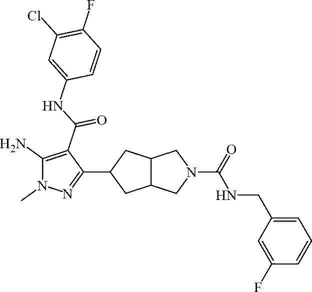 [SMILES: Cn1nc(C2CC3CN(C(=O)NCc4cccc(F)c4)CC3C2)c(C(=O)Nc2ccc(F)c(Cl)c2)c1N] |
| | 5-(5-Amino-4-((3-chloro-4-fluorophenyl)carbamoyl)-1-methyl-1H-pyrazol-3-yl)-N-(3-fluorobenzyl)hexahydrocyclopenta[c]pyrrole-2(1H)-carboxamide. ¹H NMR (400 MHz, CD₃OD): δ 7.80 (dd, J = 2.8 Hz, 6.8 Hz, 1H), 7.45 - 7.41 (m, 1H), 7.32 - 7.26 (m, 1H), 7.19 (t, J = 8.8 Hz, 1H), 7.09 (d, J = 8.0 Hz, 1H), 7.02 (d, J = 10.0 Hz, 1H), 6.93 (td, J = 2.4 Hz, 8.4 Hz, 1H), 4.34 (s, 2H), 3.66 - 3.57 (m, 1H), 3.55 (s, 3H), 3.51 - 3.47 (m, 2H), 3.36 |
| | - 3.33 (m, 2H), 2.83 - 2.77 (m, 2H), 2.39 - 2.32 (m, 2H), 1.74 - 1.67 (m, 2H) ppm. |
| Example 132 | 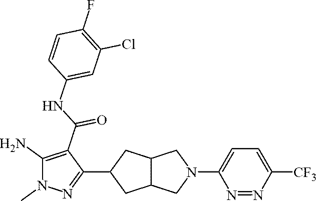 [SMILES: Cn1nc(C2CC3CN(c4ccc(C(F)(F)F)nn4)CC3C2)c(C(=O)Nc2ccc(F)c(Cl)c2)c1N] |
| | 5-Amino-N-(3-chloro-4-fluorophenyl)-1-methyl-3-(2-(6-(trifluoromethyl)pyridazin-3-yl)octahydrocyclopenta[c]pyrrol-5-yl)-1H-pyrazole-4-carboxamide. ¹H NMR (400 MHz, DMSO-d₆): δ 9.03 (s, 1H), 7.91 (dd, J = 6.8, 2.4 Hz, 1H), 7.76 (d, J = 9.6 Hz, 1H), 7.57-7.53 (m, 1H), 7.36 (t, J = 9.2 Hz, 1H), 7.03 (d, J = 9.6 Hz, 1H), 5.99 (s, J = 6.4 Hz, 2H), 3.70-3.63 (m, 3H), 3.50-3.45 (m, 5H), 2.87-2.85 (m, 2H), 2.32-2.25 (m, 2H), 1.71-1.64 (m, 2H) ppm. |
| Example 133 | 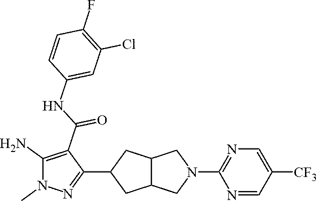 [SMILES: Cn1nc(C2CC3CN(c4ncc(C(F)(F)F)cn4)CC3C2)c(C(=O)Nc2ccc(F)c(Cl)c2)c1N] |
| | 5-Amino-N-(3-chloro-4-fluorophenyl)-1-methyl-3-(2-(5-(trifluoromethyl)pyrimidin-2-yl)octahydrocyclopenta[c]pyrrol-5-yl)-1H-pyrazole-4-carboxamide. ¹H NMR (400 MHz, DMSO-d₆): δ 9.01 (s, 1H), 8.67-8.66 (d, J = 0.8 Hz, 2H), 7.93-7.91 (dd, J = 6.8, 2.8 Hz, 1H), 7.56-7.52 (m, 1H), 7.38-7.33 (m, 1H), 5.99 (s, 2H), 3.70-3.65 (m, 3H), 3.54-3.50 (m, 2H), 3.45 (s, 3H), 2.82-2.78 (m, 2H), 2.30-2.23 (m, 2H), 1.68-1.60 (m, 2H) ppm |
| Example 134 | 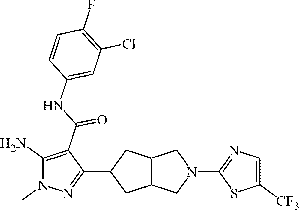 [SMILES: Cn1nc(C2CC3CN(c4ncc(C(F)(F)F)s4)CC3C2)c(C(=O)Nc2ccc(F)c(Cl)c2)c1N] |
| | 5-Amino-N-(3-chloro-4-fluorophenyl)-1-methyl-3-(2-(5-(trifluoromethyl)thiazol-2-yl)octahydrocyclopenta[c]pyrrol-5-yl)-1H-pyrazole-4-carboxamide. ¹H NMR (400 MHz, DMSO-d₆): δ 9.02 (s, 1H), 7.91 (dd, J = 7.2, 2.8 Hz, 1H), 7.70 (d, J = 1.2 Hz, 1H), 7.56-7.52 (m, 1H), 7.35 (t, J = 9.2 Hz, 1H), 5.99 (s, 2H), 3.67-3.54 (m, 3H), 3.45 (s, 3H), 3.34-3.31 (m, 2H), 2.89-2.85 (m, 2H), 2.30-2.23 (m, 2H), 1.68-1.61 (m, 2H) ppm |
| Example 135 | 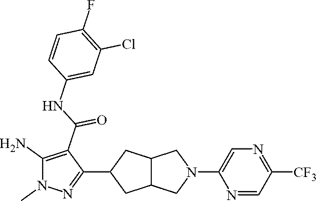 [SMILES: Cn1nc(C2CC3CN(c4cnc(C(F)(F)F)cn4)CC3C2)c(C(=O)Nc2ccc(F)c(Cl)c2)c1N] |
| | 5-Amino-N-(3-chloro-4-fluorophenyl)-1-methyl-3-(2-(5-(trifluoromethyl)pyrazin-2-yl)octahydrocyclopenta[c]pyrrol-5-yl)-1H-pyrazole-4-carboxamide. ¹H NMR (400 MHz, DMSO-d₆): δ 9.02 (s, 1H), 8.45 (s, 1H), 8.06 (d, J = 0.8 Hz, 1H), 7.91 (dd, J = 6.8, 2.4 Hz, 1H), 7.57-7.53 (m, 1H), 7.38-7.33 (m, 1H), 5.99 (s, 2H), 3.68-3.62 (m, 3H), 3.50-3.45 (m, 5H), 2.84 (s, 2H), 2.29-2.24 (m, 2H), 1.68-1.65 (m, 2H) ppm. |
| Example 136 | 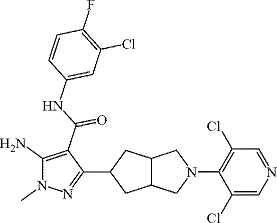 [SMILES: Cn1nc(C2CC3CN(c4c(Cl)cncc4Cl)CC3C2)c(C(=O)Nc2ccc(F)c(Cl)c2)c1N] |
| | 5-Amino-N-(3-chloro-4-fluorophenyl)-3-(2-(3,5-dichloropyridin-4-yl)octahydrocyclopenta[c]pyrrol-5-yl)-1-methyl-1H-pyrazole-4-carboxamide. ¹H NMR (400 MHz, DMSO-d₆): δ 9.00 (s, 1H), 8.36 (s, 2H), 7.91 (dd, J = 2.4, 6.8 Hz, 1H), 7.56-7.52 (m, 1H), 7.36 (t, J = 9.2 Hz, 1H), 5.99 (s, 2H), 3.58-3.54 (m, 2H), 3.49 (s, 3H), 3.39-3.37 (m, 2H), 2.72-2.67 (m, 2H), 2.25-2.19 (m, 2H), 1.71-1.63 (m, 2H) ppm. |

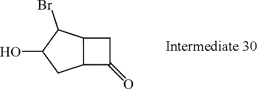

2-Bromohydroxybicyclo[3.2.0]heptan-6-one. To a solution of 3-hydroxybicyclo[3.2.0]heptan-6-one (11.0 g, 101 mmol) in acetone (100 mL) and water (30 mL) was added NBS (23.0 g, 130 mmol) in portions. The reaction mixture was stirred at room temperature for 20 h, and the aqueous sodium metabisulfite (80 mL; 10% w/w) was added to the solution until the initial yellow color had faded. The acetone was removed under reduced pressure. The residue (white precipitate in water) was re-dissolved in EtOAc (500 mL) and washed twice with water (50 mL) and brine (50 mL). The organic layer was dried (MgSO₄), concentrated under reduced pressure and the residue was purified by crystallization in EtOAc (50 mL) and PE (100 mL) to give 2-bromo-3-hydroxybicyclo[3.2.0]heptan-6-one as white crystals. ¹H NMR (400 MHz, CDCl₃): δ 4.64 (d, J = 4.0 Hz, 1H), 4.32 (s, 1H), 3.78-3.83 (m, 1H), 3.19-3.34 (m, 3H), 2.65 (brs, 1H), 2.48-2.55 (m, 1H), 2.26 (d, J = 14.8 Hz, 1H) ppm.

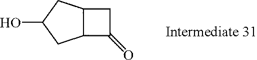

3-Hydroxybicyclo[3.2.0]heptan-6-one. To a solution of 2-bromo-3-hydroxybicyclo[3.2.0]heptan-6-one (11.0 g, 54 mmol) in dry toluene (100 mL) was added under nitrogen n-tributyltinhydride (23.0 g, 81 mmol) and AIBN (30 mg, 0.2 mmol). The reaction mixture was heated to 80° C. for 1 h, allowed to cool to room temperature and concentrated under reduced pressure providing a yellow liquid. The tin residues were removed by partitioning between acetonitrile (200 mL) and hexane (150 mL) and extracting the acetonitrile layer with hexane (4 × 150 mL). The combined hexane layers were evaporated under reduced pressure and the crude product was purified by chromatography on silica gel with PE/EtOAc (2:1, v/v) to give 3-hydroxybicyclo[3.2.0]heptan-6-one as white crystals. ¹H NMR (400 MHz, CDCl₃): δ 4.54 (brs,1H), 3.58-3.64 (m, 1H), 3.17-3.25 (m, 1H), 3.01-3.07 (m, 1H), 2.89-2.93 (m, 1H), 2.13-2.28 (m, 2H), 1.83-1.98 (m, 3H) ppm.

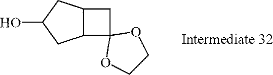 [SMILES: OC1CC2CC3(OCCO3)C2C1]

Spiro[bicyclo[3.2.0]heptane-6,2′-[1,3]dioxolan]-3-ol. To a solution of 3-hydroxybicyclo[3.2.0]heptan-6-one (6.5 g, 51 mmol) in toluene (150 mL) was added ethanediol (5 mL) and TsOH (30 mg). The mixture was then refluxed and stirred overnight. After cooling to room temperature, the mixture was quenched with saturated sodium bicarbonate solution (50 ml). The toluene phase was separated and concentrated to a residue. The residue was purified by chromatography on silica gel with PE/EtOAc (3:1, v/v) to give spiro[bicyclo[3.2.0]heptane-6,2′-[1,3]dioxolan]-3-ol as a clear oil. ¹H NMR (400 MHz, CDCl₃): δ 4.38-4.40 (m, 1H), 3.87-3.95 (m, 4H), 3.55 (d, J = 8.0 Hz, 1H), 2.98-3.03 (m, 1H), 2.56-2.66 (m, 2H), 2.32-2.37 (m, 1H), 2.01-2.05 (m, 1H), 1.82-1.83 (m, 2H) ppm.

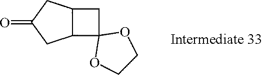 [SMILES: O=C1CC2CC3(OCCO3)C2C1]

Spiro[bicyclo[3.2.0]heptane-6,2′-[1,3]dioxolan]-3-one. To a solution of spiro[bicyclo[3.2.0]heptane-6,2′-[1,3]dioxolan]-3-ol (2.0 g, 11 mmol) in DCM (100 mL) was added PCC (9.5 g, 44 mmol) and NaOAc (1.8 g, 22 mmol). The mixture was then refluxed and stirred overnight. The solvent was evaporated, and the residue was purified by chromatography on silica gel with PE/EtOAc (5:1, v/v) to give spiro[bicyclo[3.2.0]heptane-6,2′-[1,3]dioxolan]-3-one (2.1 g, 67%) as a white solid. ¹H NMR (400 MHz, CDCl₃)— δ 3.79-3.94 (m, 4H), 3.17-3.22(m, 1H), 2.68-2.79 (m, 2H), 2.43-2.54 (m, 2H), 2.17-2.35 (m, 2H), 2.02-2.08 (m, 1H) ppm.

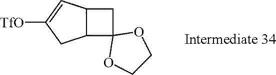 [SMILES: O=S(=O)(OC1=CC2CC3(OCCO3)C2C1)C(F)(F)F]

Spiro[bicyclo[3.2.0]hept[2]ene-6,2′-[1,3]dioxolane]-3-yltrifluoro methanesulfonate. To a solution of spiro[bicyclo[3.2.0]heptane-6,2′-[1,3]dioxolan]-3-one (2.0 g, 12 mmol) in THF (100 mL) was added LiHMDS (15 mmol, 1 M, 15 mL) at -78° C. The reaction mixture was stirred at -78° C. for 1h and then a solution of 1,1,1-trifluoro-N-phenyl-N-(trifluoromethylsulfonyl)methane sulfonamide (5.4 g, 15 mmol) in THF (25 mL) was added dropwise. The reaction mixture was warmed to room temperature and stirred overnight. The solvent was evaporated, and the residue was purified by column chromatography in silica gel using PE/EA = 5/1 (v/v) to afford spiro[bicyclo[3.2.0]hept[2]ene-6,2′-[1,3]dioxolane]-3-yltrifluoro methanesulfonate (2.7 g, 75%) as a yellow solid. ¹H NMR (400 MHz, CDCl₃): δ 5.66-5.69 (m, 1H), 3.80-3.97 (m, 4H), 3.47-3.50 (m, 1H), 2.59-2.87 (m, 3H), 2.23-2.43 (m, 2H) ppm.

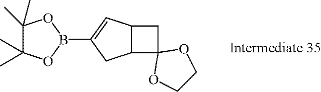 [SMILES: CC1(C)OB(C2=CC3CC4(OCCO4)C3C2)OC1(C)C]

4,4,5,5-Tetramethyl-2-(spiro[bicyclo[3.2.0]hept[2]ene-6,2′-[1,3]dioxolane]-3-yl)-1,3,2-dioxaborolane. A mixture of spiro[bicyclo[3.2.0]hept[2]ene-6,2′-[1,3]dioxolane]-3-yltrifluoro methanesulfonate (2.7 g, 9 mmol),4,4,5,5-tetramethyl-2-(4,4,5,5-tetramethyl-1,3,2-dioxaborolan-2-yl)-1,3,2-dioxaborolane (2.5 g, 10 mmol), Pd(dppf)Cl₂ (292 mg, 0.4 mmol) and potassium acetate (2.0 g, 20 mmol) in dioxane (50 mL) was stirred at 80° C. under N₂ atmosphere for 4 h. The reaction was filtered through a pad of celite, the cake was washed with EtOAc (10 mL x 3). The filtrate was concentrated in vacuum and the residue was purified through silica gel column chromatography using PE/EA = 5/1 (v/v) to afford compound 4,4,5,5-tetramethyl-2-(spiro[bicyclo[3.2.0]hept[2]ene-6,2′-[1,3]dioxolane]-3-yl)-1,3,2-dioxaborolane as a yellow solid. ¹H NMR (400 MHz, CDCl₃): δ 6.40-6.46 (m, 1H), 3.83-3.96 (m, 4H), 3.55-3.57 (m, 1H), 2.35-2.69 (m, 4H), 2.05-2.13 (m, 1H), 1.24-1.30 (m, 12H) ppm.

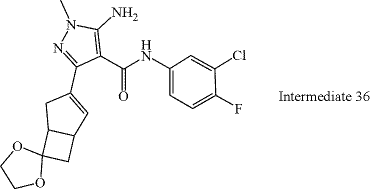 [SMILES: Cn1nc(C2=CC3CC4(OCCO4)C3C2)c(C(=O)Nc2ccc(F)c(Cl)c2)c1N]

5-Amino-N-(3-chloro-4-fluorophenyl)-1-methyl-3-(spiro[bicyclo[3.2.0] hept[2]ene-6,2′-[1,3]dioxolane]-3-yl)-1H-pyrazole-4-carboxamide. A mixture of 4,4,5,5-tetramethyl-2-(spiro[bicyclo[3.2.0]hept[2]ene-6,2′-[1,3]dioxolane]-3-yl)-1,3,2-dioxaborolane (2.2 g, 7.9 mmol), 5-amino-3-bromo-N-(3-chloro-4-fluoro-phenyl)-1-methyl-pyrazole-4-carboxamide (2.7 g, 7.9 mmol), K₃PO₄ (3.1 g, 14.5 mmol) and Pd(dppf)Cl₂ (530 mg, 0.7 mmol) in dioxane (60 mL) and H₂O (3 mL) were stirred at 100° C. overnight under N₂ atmosphere. The solvent was removed in vacuum and the residue was purified by silica gel column chromatography using PE/EA = 3/1 (v/v) to afford compound 5-amino-N-(3-chloro-4-fluorophenyl)-1-methyl-3-(spiro[bicyclo[3.2.0]hept[2]ene-6,2′-[1,3]dioxolane]-3-yl)-1H-pyrazole-4-carboxamide (1.7 g, 51 % yield) as a yellow solid. TLC: 50% PE/EA (v/v) (R_{f}: 0.4), MS calcd.: 418.2; Found: 419.2 [M + 1]⁺.

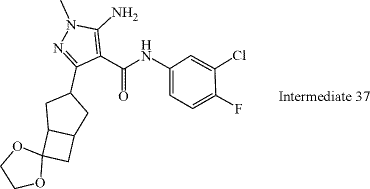 [SMILES: Cn1nc(C2CC3CC4(OCCO4)C3C2)c(C(=O)Nc2ccc(F)c(Cl)c2)c1N]

5-Amino-N-(3-chloro-4-fluorophenyl)-l-methyl-3-(spiro[bicyclo[3.2.0] heptane-6,2′-[1,3]dioxolane]-3-yl)-1H-pyrazole-4-carboxamide. A mixture of 5-amino-N-(3-chloro-4-fluorophenyl)-1-methyl-3-(spiro[bicyclo[3.2.0] hept[2]ene-6,2′-[1,3]dioxolane]-3-yl)-1H-pyrazole-4-carboxamide (850 mg, 2.0 mmol) and Pt/C (100 mg) in methanol (100 mL) was stirred under H₂ at room temperature for two days. The mixture was filtered with a pad of celite, the filtrate was concentrated to afford compound 5-amino-N-(3-chloro-4-fluorophenyl)-1-methyl-3-(spiro[bicyclo[3.2.0] heptane-6,2′-[1,3]dioxolane]-3-yl)-1H-pyrazole-4-carboxamide as a yellow solid which was directly used for next step without further purification. TLC: 50% PE/EA (v/v) (R_{f}: 0.4), MS calcd.: 420.8; Found: 421.2 [M + 1]⁺.

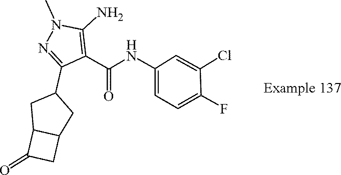 [SMILES: Cn1nc(C2CC3CC(=O)C3C2)c(C(=O)Nc2ccc(F)c(Cl)c2)c1N]

5-Amino-N-(3-chloro-4-fluorophenyl)-l-methyl-3-(6-oxobicyclo[3.2.0] heptan-3-yl)-1H-pyrazole-4-carboxamide. To a solution of 5-amino-N-(3-chloro-4-fluorophenyl)-1-methyl-3-(spiro[bicyclo[3.2.0] heptane-6,2′-[1,3]dioxolane]-3-yl)-1H-pyrazole-4-carboxamide (850 mg, 2.0 mmol) in acetone (20 mL) was added water (1.0 mL) and TsOH (172 mg, 1.0 mmol). The reaction mixture was stirred at room temperature for 2 h. The mixture was quenched with saturated sodium bicarbonate solution (5 mL) and concentrated under reduced pressure to a residue. The residue was purified through silica gel column chromatography using PE/EA = 1/1 (v/v) to afford compound 5-amino-N-(3-chloro-4-fluorophenyl)-1-methyl-3-(6-oxobicyclo[3.2.0] heptan-3-yl)-1H-pyrazole-4-carboxamide as a white solid. MS calcd.: 376.8; Found: 377.2 [M+1]⁺. ¹H-NMR (400 MHz, DMSO-d₆): δ 9.00 (s, 1H), 7.89 (dd, J = 2.8, 6.8 Hz, 1H), 7.51-7.55 (m, 1H), 7.35 (t, J = 9.2 Hz, 1H), 6.00 (s, 2H), 3.77-3.81 (m, 1H), 3.61-3.67 (m, 1H), 3.47 (s, 3H), 3.05-3.12 (m, 1H), 2.78-2.83 (m, 1H), 2.58-2.65(m, 1H), 2.30-2.37 (m, 1H), 2.02-2.11 (m, 2H), 1.77-1.84 (m, 1H) ppm.

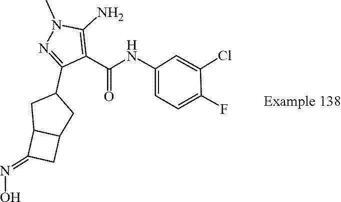 [SMILES: Cn1nc(C2CC3C/C(=N\O)C3C2)c(C(=O)Nc2ccc(F)c(Cl)c2)c1N]

(E)Amino-N-(3-chloro-4-fluorophenyl)-3-(6-(hydroxyimino) bicyclo[3.2.0]heptan-3-yl)-1-methyl-1H-pyrazole-4-carboxamide. A mixture of 5-amino-N-(3-chloro-4-fluorophenyl)-1-methyl-3-(6-oxobicyclo[3.2.0] heptan-3-yl)-1H-pyrazole-4-carboxamide (350 mg, 0.9 mmol), NH₂OH.HCl (67 mg, 1.0 mmol), NaHCO₃ (168 mg, 2.0 mmol) in methanol (25 mL) was stirred at 50° C. overnight. The solvent was evaporated, and the residue was purified by chromatography on silica gel using DCM/MeOH = 10/1 (v/v) to afford compound (E)-5-amino-N-(3-chloro-4-fluorophenyl)-3-(6-(hydroxyimino) bicyclo[3.2.0]heptan-3-yl)-1-methyl-1H-pyrazole-4-carboxamide as a white solid. MS calcd.: 391.8; Found: 392.2 [M+1]⁺. ¹H-NMR (400 MHz, DMSO-d₆): δ 10.05 (brs, 1H), 9.00 (s, 1H), 7.89-7.91 (m, 1H), 7.50-7.54 (m, 1H), 7.35 (t, J = 5.2 Hz, 1H), 5.98 (s, 2H), 3.56-3.65 (m, 1H), 3.32-3.44 (m, 4H), 2.79-2.90 (m, 1H), 2.66-2.70 (m, 1H), 2.41-2.48 (m, 1H), 2.15-2.33(m, 2H), 1.89-2.00 (m, 1H), 1.66-1.73 (m, 1H) ppm.

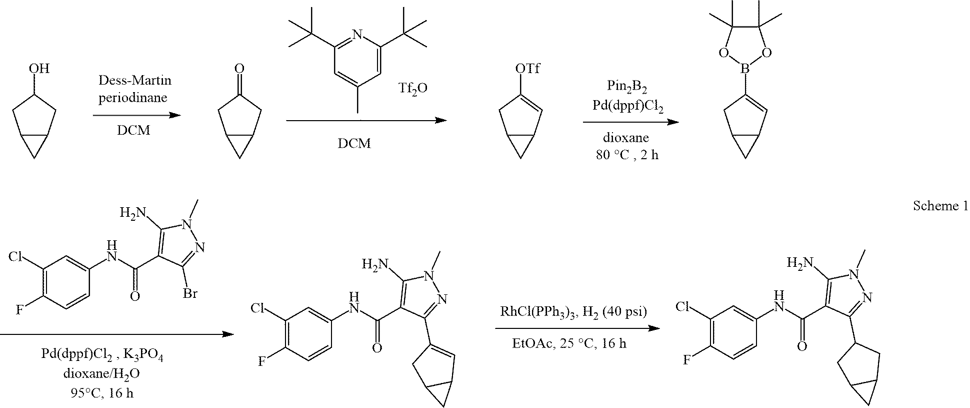

| | |
|---|---|
| Example | Structure and analysis |
| Example 139 | 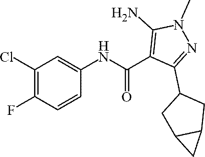 [SMILES: Cn1nc(C2CC3CC3C2)c(C(=O)Nc2ccc(F)c(Cl)c2)c1N] |
| | 5-Amino-3-(bicyclo[3.1.0]hexan-3-yl)-N-(3-chloro-4-fluorophenyl)-1-methyl-1H-pyrazole-4-carboxamide. MS calcd. for C₁₇H₁₈ClFN₄O— 348.1; Found: 349.2 [M + 1]⁺. ¹H NMR (400 MHz, DMSO-d₆): δ 8.80-8.76 (m, 1H), 7.92 (d, J=6.69 Hz, 1H), 7.54 (ddd, J=9.02, 4.31, 2.57 Hz, 1H), 7.35 (t, J=9.11 Hz, 1H), 6.00 (s, 2 H), 3.96-3.88 (m, 1H), 3.48 (s, 3H), 2.22-2.13 (m, 2H), 2.08-1.99 (m, 2H), 1.28-1.21 (m, 2H), 0.32 (td, J=8.01, 4.28 Hz, 1H), 0.24 (q, J=4.03 Hz, 1H) ppm. |

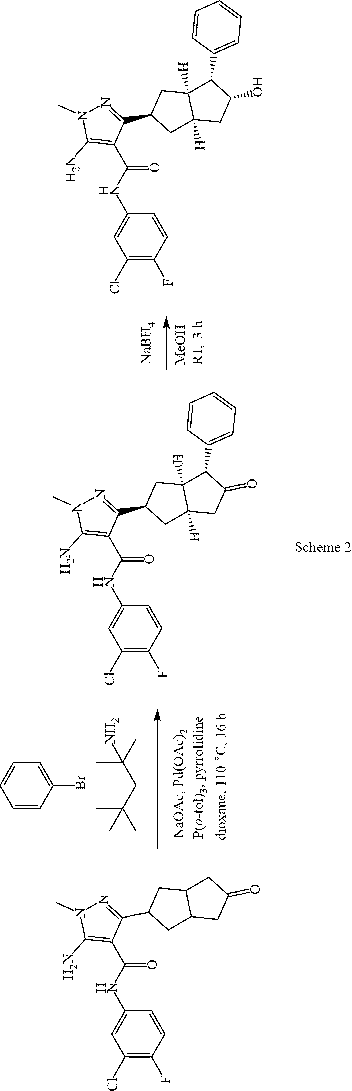

| | |
|---|---|
| Example | Structure and analysis |
| Example 140 | 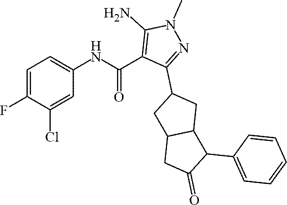 [SMILES: Cn1nc(C2CC3CC(=O)C(c4ccccc4)C3C2)c(C(=O)Nc2ccc(F)c(Cl)c2)c1N] |
| | 5-Amino-N-(3-chloro-4-fluorophenyl)-1-methyl-3-(5-oxo-4-phenyloctahydropentalen-2-yl)-1H-pyrazole-4-carboxamide MS calcd. for C₂₅H₂₄ClFN₄O₂— 466.2; Found: 467.0 [M + 1]⁺. 1H NMR (400 MHz, DMSO-d₆): δ 9.01 (s, 1H), 7.90 (dd, J = 7.2, 2.4 Hz 1H), 7.55-7.51 (m, 1H), 7.36-7.21 (m, 4H), 7.14-7.12 (m, 2H), 5.99 (s, 2H), 3.75-3.70 (m, 1H), 3.52 (s, 3H), 3.36-3.31 (m, 1H), 2.77 (br.s, 2H), 2.62-2.58 (m, 1H), 2.34-2.55 (m, 3H), 2.86 -1.70 (m, 2H) ppm. |
| Example 141 | 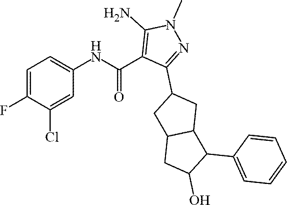 [SMILES: Cn1nc(C2CC3CC(O)C(c4ccccc4)C3C2)c(C(=O)Nc2ccc(F)c(Cl)c2)c1N] |
| | 5-Amino-N-(3-chloro-4-fluorophenyl)-3-(5-hydroxy-4-phenyloctahydropentalen-2-yl)-1-methyl-1H-pyrazole-4-carboxamide Isomer I. MS calcd. for C₂₅H₂₆ClFN₄O₂— 468.2; Found: 469.1 [M + 1]⁺. ¹H NMR (400 MHz, DMSO-d₆): δ 8.95 (s, 1H), 7.88 (dd, J = 6.8, 2.4 Hz 1H), 7.51-7.47 (m, 1H), 7.34-7.22 (m, 5H), 7.17-7.13 (m, 1H), 5.96 (s, 2H), 4.67 (d, J = 6.4 Hz, 1H), 4.05 (m, 1H), 3.51 (s, 3H), 3.49-3.44 (m, 1H), 2.56-2.53 (m, 1H), 2.50-2.38 (m, 2H), 2.24-2.18 (m, 2H), 2.11-2.04 (m, 1H), 1.64-1.52 (m, 2H), 1.39-1.32 (m, 1H) ppm. |
| Example 142 | 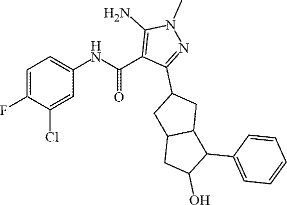 [SMILES: Cn1nc(C2CC3CC(O)C(c4ccccc4)C3C2)c(C(=O)Nc2ccc(F)c(Cl)c2)c1N] |
| | 5-Amino-N-(3-chloro-4-fluorophenyl)-3-(5-hydroxy-4-phenyloctahydropentalen-2-yl)-1-methyl-1H-pyrazole-4-carboxamide Isomer II. |
| | MS calcd. for C₂₅H₂₇FN₄O₂: 434.2; Found: 435.3 [M + 1]⁺. ¹H NMR (400 MHz, DMSO-d₆): δ 8.79 (s, 1H), 7.56-7.53 (m, 1H), 7.26-7.18 (m, 4H), 7.15-7.05 (m, 3H), 6.92 (s, 1H), 5.89 (br.s, 2H), 4.03-4.02 (m, 1H), 3.49 (s, 3H), 2.54-2.48 (br.s, 2H, merged), 2.42-2.38 (m, 2H), 2.22-2.21 (m, 2H), 2.07-2.05 (m, 2H), 1.60-1.53 (m, 2H), 1.35-1.33 (m, 1H) ppm. |

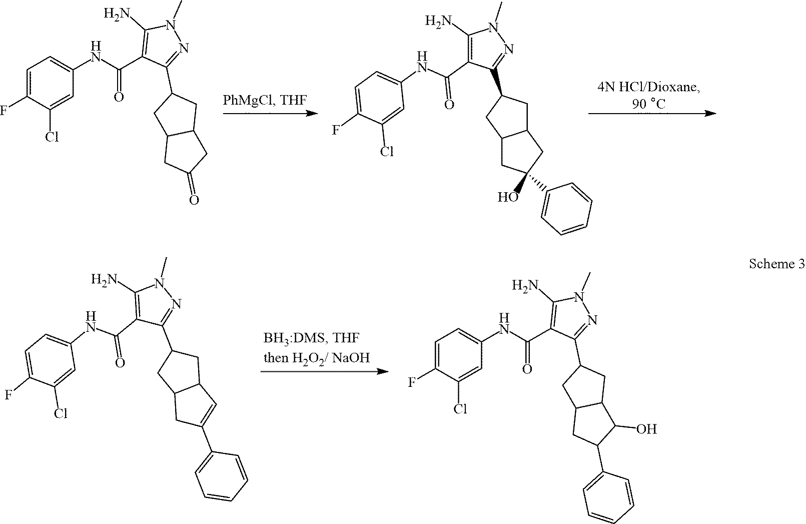

| | |
|---|---|
| Example | Structure and analysis |
| Example 143 | 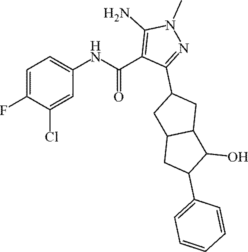 [SMILES: Cn1nc(C2CC3CC(c4ccccc4)C(O)C3C2)c(C(=O)Nc2ccc(F)c(Cl)c2)c1N] |
| | 5-Amino-N-(3-chloro-4-fluorophenyl)-3-(4-hydroxy-5-phenyloctahydropentalen-2-yl)-1-methyl-1H-pyrazole-4-carboxamide Isomer I MS calcd. for C₂₅H₂₆CIFN₄O₂— 468.2; Found: 469.1 [M + 1]⁺. ¹H NMR (400 MHz, DMSO-d₆): δ 8.96 (s, 1H), 7.92 (dd, J= 6.8, 2.4 Hz 1H), 7.55-7.51 (m, 1H), 7.35 (t, J = 9.2 Hz, 1H), 7.27-7.21 (m, 4H), 7.17-7.13 (m, 1H), 5.99 (s, 2H), 4.66 (d, J = 4 Hz, 1H), 3.72-3.54 (m, 2H), 3.52 (s, 3H), |
| | 3.32-2.61 (m, 1H), 2.58-2.49 (m, 1H), 2.35-2.08 (m, 4H), 1.74-1.67 (m, 1H), 1.58-1.45 (m, 2H) ppm. |
| Example 144 | 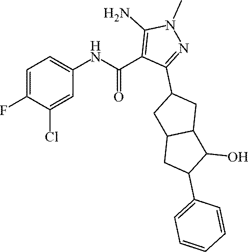 [SMILES: Cn1nc(C2CC3CC(c4ccccc4)C(O)C3C2)c(C(=O)Nc2ccc(F)c(Cl)c2)c1N] |
| | 5-Amino-N-(3-chloro-4-fluorophenyl)-3-(4-hydroxy-5-phenyloctahydropentalen-2-yl)-1-methyl-1H-pyrazole-4-carboxamide Isomer II MS calcd. for C₂₅H₂₆ClFN₄O₂— 468.2; Found: 469.2 [M + 1]⁺. ¹H NMR (400 MHz, DMSO-d₆): δ 8.96 (s, 1H), 7.92 (dd, J= 6.8, 2.4 Hz 1H), 7.54-7.52 (m, 1H), 7.35 (t, J = 9.2 Hz, 1H), 7.27-7.21 (m, 4H), 7.17-7.13 (m, 1H), 6.00 (s, 2H), 4.66 (d, J = 4 Hz, 1H), 3.71-3.61 (m, 2H), 3.52 (s, 3H), 2.92-2.88 (m, 1H), 2.58-2.54 (m, 1H), 2.35-2.31 (m, 1H), 2.25-2.07 (m, 3H), 1.74-1.66 (m, 1H), 1.61-1.48 (m, 2H) ppm. |
| Example 145 | 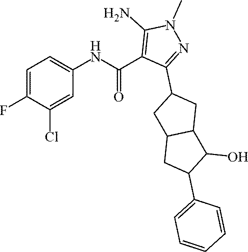 [SMILES: Cn1nc(C2CC3CC(c4ccccc4)C(O)C3C2)c(C(=O)Nc2ccc(F)c(Cl)c2)c1N] |
| | 5-Amino-N-(3-chloro-4-fluorophenyl)-3-(4-hydroxy-5-phenyloctahydropentalen-2-yl)-1-methyl-1H-pyrazole-4-carboxamide Isomer III MS calcd. for C₂₅H₂₆CIFN₄O₂— 468.2; Found: 469.1 [M + 1]⁺. ¹H NMR (400 MHz, DMSO-d₆): δ 8.96 (s, 1H), 7.92 (dd, J = 6.8, 2.4 Hz 1H),7.55-7.51 (m, 1H), 7.35 (t, J = 9.2 Hz, 1H), 7.27-7.22 (m, 4H), 7.17-7.13 (m, 1H), 6.00 (s, 2H), 4.66 (br.s, 1H), 3.71-3.59 (m, 2H), 3.52 (s, 3H), 2.95-2.88 (m, 1H), 2.58-2.54 (m, 1H), 2.35-2.31 (m, 1H), 2.26-2.07 (m, 3H), 1.74-1.69 (m, 1H), 1.61-1.45 (m, 2H) ppm. |

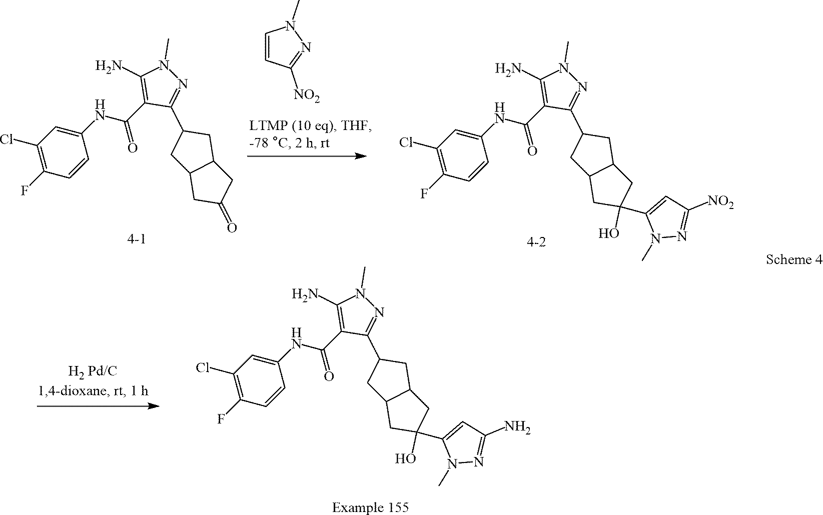

Step 1. Synthesis of 5-amino-N-(3-chloro-4-fluorophenyl)-3-((2 s,3aR,5r,6aS)-5-hydroxy-5-(1-methyl-3-nitro-1H-pyrazol-5-yl)octahydropentalen-2-yl)-1-methyl-1H-pyrazole-4-carboxamide: To a solution of 1-methyl-3-nitro-1H-pyrazole (0.488 g, 3.8 mmol) in dry THF (8 mL), LTMP (2.0 mL, 3.8 mmol) was added in one portion at -78° C. under Ar. After the mixture was stirred at -78° C. for 0.5 h, Compd. 4-1 (0.15 g, 0.38 mmol) in THF (1 mL) was added and stirred at rt for 2 h. The solution was quenched by NH₄Cl aq. and extracted with EA (20 mL x 2), washed with brine (20 mL) then concentrated and purified by prep-HPLC to afford Compd. 4-2 (148 mg, 74%) as a white solid. TLC: 10% MeOH/DCM (R_{f}: 0.4); MS Calcd.: 517.2; Found: 518.2 [M+ 1] ⁺. ¹H NMR (400 Hz, DMSO-d₆): δ 8.93 (s, 1H), 7.93-7.90 (dd, J = 6.8 Hz, 2.8 Hz, 1H), 7.53-7.49 (m, 1H), 7.34 (t, J = 9.2 Hz, 1H), 6.93 (s, 1H), 5.98 (s, 2H), 5.53 (s, 1H), 4.03 (s, 3H), 3.50 (s, 3H), 3.44-3.22 (m, 1H), 2.51-2.49 (m, 2H), 2.27-219 (m, 4H), 2.07-1.89 (m, 2H),1.89-1.76 (m, 2H) ppm.

Step 2. Synthesis of 5-amino-3-((2 s,3aR,5r,6aS)-5-(3-amino-1-methyl-1H-pyrazol-5-yl)-5-hydroxyoctahydropentalen-2-yl)-N-(3-chloro-4-fluorophenyl)-1-methyl-1H-pyrazole-4-carboxamide (Example 155): To a solution of Compd. 4-2 (148 mg, 0.28 mmol) in 1,4-dioxane (5 mL) was added Pd/C (70 mg). The flask was then evacuated and backfilled with H₂. The solution was stirred at rt for 1 h. Then the mixture was filtered and concentrated. And the residue was purified by prep-HPLC to afford Example 155 (36 mg, 25%) as a white solid, TLC: 65% EA/PE (v/v) (R_{f}: 0.3); MS Calcd.: 487.2; Found: 488.19 [M + 1] ⁺. ¹H NMR (400 MHz, DMSO-d₆): δ 8.91 (s, 1H), 7.93-7.91 (m, 1H), 7.52-7.49 (m, 1H), 7.34 (t, J = 9.0 Hz, 1H), 5.99 (s, 2H), 5.24 (s, 1H), 5.03 (s, 1H), 4.33 (s, 2H), 3.62 (s, 3H), 3.49 (s, 3H), 3.46-3.40 (m, 1H), 2.50-2.49 (m, 2H), 2.15-2.06 (m, 4H), 1.81-1.71 (m, 4H) ppm.

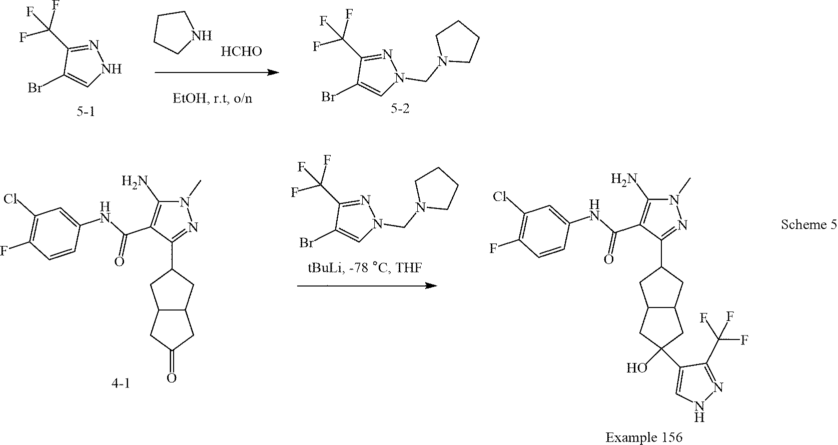

Step 1. Synthesis of 4-bromo-1-(pyrrolidin-1-ylmethyl)-3-(trifluoromethyl)-1H-pyrazole (5-2): To a solution of Compd. 5-1 (15.0 g, 70.0 mmol) in EtOH (200 mL) was added pyrrolidine (4.98 g, 70.0 mmol), the reaction was stirred at room temperature and HCHO (11.4 mL, 140.0 mmol, 37% in H₂O) was added. The reaction mixture was stirred at room temperature overnight. After concentration in vacuo, the residue was to give crude Compd. 5-2 (20.5 g, 98% yield) as a yellow oil. ¹H NMR (400 MHz, CDCl₃): δ 7.58 (s, 1H), 5.04 (s, 2H), 2.69 (m, 4H), 1.77 (m, 4H) ppm.

Step 2. Synthesis of 5-amino-N-(3-chloro-4-fluorophenyl)-3-(5-hydroxy-5-(3-(trifluoromethyl)-1H-pyrazol-4-yl)octahydropentalen-2-yl)-1-methyl-1H-pyrazole-4-carboxamide (Example 156): To a solution of Compd. 5-2 (5.96 g, 20.0 mmol) in Ether (30 mL) was added t-BuLi (15.4 mL, 20.0 mmol, 1.3 M) dropwise (about 1 min) at -78° C. The reaction mixture was stirred at -78° C. for 5 min, then Compd. 4-1 (391 mg, 1.0 mmol) was added. The mixture was stirred at -78° C. for 2 h, sat. aq. NH₄Cl solution was added, concentrated, and purified by flash (MeOH in DCM, 0~15%) to give the crude product. The crude was purified by prep-HPLC to give Example 156 (156 mg, 30% yield) as a white solid. MS Calcd.: 526.2; Found: 528.0 [M + 2] ⁺. ¹H NMR (400 MHz, CD₃OD): δ 7.80 (dd, J = 6.8, 2.4 Hz, 1H), 7.71 (s, 1H), 7.43 - 7.36 (m, 1H), 7.18 (t, J = 9.2 Hz, 1H), 3.55 (s, 3H), 3.47 - 3.38 (m, 1H), 2.69 - 2.57 (m, 2H), 2.39 -2.25 (m, 4H), 1.97 - 1.83 (m, 4H) ppm.

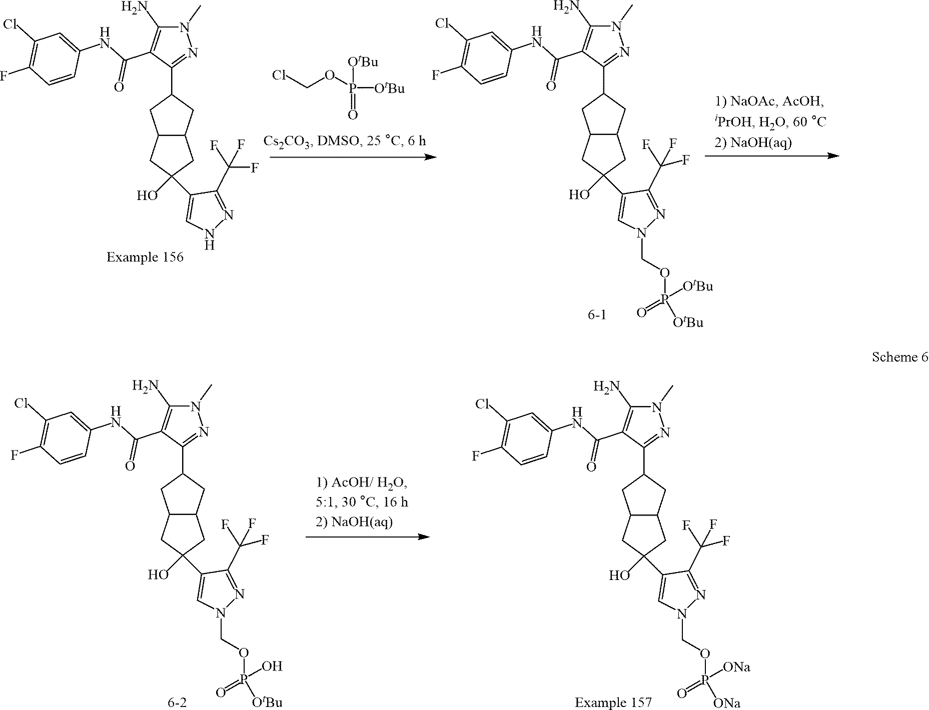

Step 1. Synthesis of (4-(5-(5-amino-4-((3-chloro-4-fluorophenyl)carbamoyl)-1-methyl-1H-pyrazol-3-yl)-2-hydroxyoctahydropentalen-2-yl)-3-(trifluoromethyl)-1H-pyrazol-1-yl)methyl di-tert-butyl phosphate (6-1): To a solution of Example 156 (80 mg, 0.15 mmol) in DMSO (1.5 mL) was added di-tert-butyl (chloromethyl) phosphate (58 mg, 0.225 mmol) and Cs₂CO₃ (54 mg, 0.165 mmol), the reaction was stirred at 25° C. for 6 h. The mixture was purified by prep-HPLC to give Compd. 6-1 (75 mg, 67% yield) as a white solid. MS Calcd.: 748.3; Found: 730.8 [M - H₂O]. ¹H NMR (400 MHz, DMSO-d₆): δ 8.98 (s, 1H), 7.97 (s, 1H), 7.90 (dd, J = 6.8, 2.4 Hz, 1H), 7.55 - 7.48 (m, 1H), 7.34 (t, J = 9.2 Hz, 1H), 5.98 (s, 2H), 5.92 - 5.75 (m, 2H), 4.97 (s, 1H), 3.50 (s, 3H), 3.44 - 3.37 (m, 1H), 2.57 - 2.47 (m, 2H), 2.20 - 2.01 (m, 4H), 1.87 - 1.71 (m, 4H), 1.37 (s, 18H) ppm.

Step 2. Synthesis of (4-(5-(5-amino-4-((3-chloro-4-fluorophenyl)carbamoyl)-1-methyl-1H-pyrazol-3-yl)-2-hydroxyoctahydropentalen-2-yl)-3-(trifluoromethyl)-1H-pyrazol-1-yl)methyl tert-butyl hydrogen phosphate (6-2): To a solution of Compd. 6-1 (115 mg, 0.154 mmol) in ⁱPrOH (4 mL) was added NaOAc (101 mg, 1.23 mmol) in H₂O (2 mL) and AcOH (230 mg, 3.85 mmol). The reaction mixture was stirred at 60° C. for 4 h, then the mixture was cooled to 0° C., and the resulting mixture was adjusted to pH 8 ~ 9 with 2 M aq. NaOH. The residue was concentrated and purified by prep-HPLC to give Compd. 6-2 (70 mg, 66% yield) as a white solid. MS Calcd.: 692.2; Found: 694.0 [M + 2]⁺. ¹H NMR (400 MHz, CD₃OD): δ 7.92 (s, 1H), 7.80 (dd, J = 6.8, 2.4 Hz, 1H), 7.45 - 7.35 (m, 1H), 7.19 (t, J = 9.2 Hz, 1H), 5.71 (d, J = 10.8 Hz, 2H), 3.55 (s, 3H), 3.49 -3.40 (m, 1H), 2.70 - 2.58 (m, 2H), 2.37 - 2.25 (m, 4H), 1.97 - 1.79 (m, 4H), 1.35 (s, 9H) ppm.

Step 3. Synthesis of sodium sodium (4-(5-(5-amino-4-((3-chloro-4-fluorophenyl)carbamoyl)-1-methyl-1H-pyrazol-3-yl)-2-hydroxyoctahydropentalen-2-yl)-3-(trifluoromethyl)-1H-pyrazol-1-yl)methyl phosphate (Example 157): To a solution of Compd. 6-2 (70 mg, 0.10 mmol) in AcOH/H₂O (1.2 mL, v/v, 5:1), the reaction mixture was stirred at 30° C. for 10 h, then added ice water (2 mL). The resulting mixture was adjusted to pH 8 ~ 9 with 6 N aq. NaOH solution. The residue was concentrated and purified by prep-HPLC to give Example 157 (20 mg, 29% yield) as a white solid. MS Calcd.: 680.1; Found: 636.8 [M - 2Na + 2]⁺. ¹H NMR (400 MHz, CD₃OD): δ 7.97 (s, 1H), 7.82 (dd, J = 6.8, 2.4 Hz, 1H), 7.42 - 7.36 (m, 1H), 7.19 (t, J = 9.2 Hz, 1H), 5.72 (d, J = 9.6 Hz, 2H), 3.55 (s, 3H), 3.50 - 3.42 (m, 1H), 2.69 - 2.58 (m, 2H), 2.39 - 2.25 (m, 4H), 1.95 - 1.80 (m, 4H) ppm.

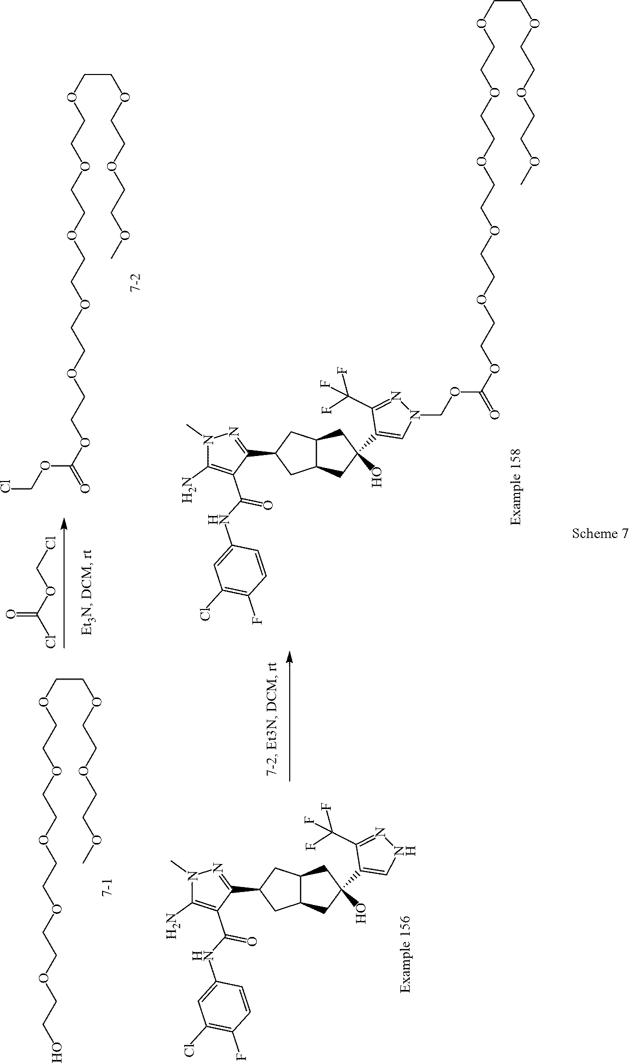

Step 1. Synthesis of chloromethyl (2,5,8,11,14,17,20,23-octaoxapentacosan-25-yl) carbonate (7-2): To a solution of Compd. 7-1 (768 mg, 2 mmol) and Et₃N (404 mg, 4 mmol) in DCM (15 mL), chloromethyl carbonochloridate (384 mg, 3 mmol) was added. The solution was stirred at rt for overnight. The mixture was quenched by water (20 ml) and extracted with DCM (15 mL x 3). The organic solvent was concentrated in vacuum and the residue was purified by chromatography (20 g silica gel), eluted with EA in PE from 10-55% (v/v) to afford Compd. 7-2 (280 mg, 29%) as a colorless liquid. TLC: 50% EA/PE (R_{f}: 0.25).

Step 2. Synthesis of (4-((2r,3aR,5 s,6aS)-5-(5-amino-4-((3-chloro-4-fluorophenyl)carbamoyl)-1-methyl-1H-pyrazol-3-yl)-2-hydroxyoctahydropentalen-2-yl)-3-(trifluoromethyl)-1H-pyrazol-1-yl)methyl (2,5,8,11,14,17,20,23-octaoxapentacosan-25-yl) carbonate (Example 158): To a solution of Example 156 (105 mg, 0.2 mmol) and Et₃N (40.4 mg, 0.4 mmol) in DCM (5 mL), Compd. 7-2 (143 mg, 0.3 mmol) was added. The solution was stirred at rt overnight. The mixture was quenched by water (10 ml) and extracted with DCM (10 mL × 3). The organic solvent was concentrated in vacuum and the residue was purified by prep-HPLC to afford Example 158 (20 mg, 10%) as a yellow oil. TLC: 10% MeOH/DCM (v/v) (R_{f}: 0.35); MS Calcd.: 966.3; Found: 966.8 [M + 1]⁺.

The following examples were readily synthesized by following similar synthetic routes described above with the corresponding starting materials:

| Compd. | STRUCTURE | MS [M + 1]⁺ | ¹H NMR |
|---|---|---|---|
| Example 146 | 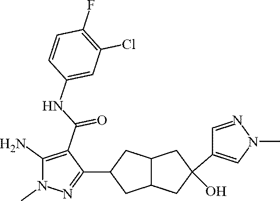 | 473.3 | ¹H NMR (DMSO-d6, 400 MHz): δ 8.92 (s, 1H), 7.92 (dd, J₁ = 3.6 Hz, J₂ = 6.8 Hz, 1H), 7.52 - 7.49 (m, 2H), 7.35 (t, J = 8.8 Hz, 1H), 7.29 (s, 1H), 5.99 (s, 2H), 4.68 (s, 1H), 3.75 (m, 3H), 3.41 (s, 1H), 3.35 (m, 1H), 2.50 (m, 2H), 2.14 - 2.12 (m, 2H), 2.04 - 2.00 (m, 2H), 1.78 - 1.70 (m, 4H) ppm. |
| Example 147 | 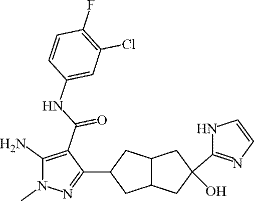 [SMILES: Cn1nc(C2CC3CC(O)(c4ncc[nH]4)CC3C2)c(C(=O)Nc2ccc(F)c(Cl)c2)c1N] | 459.3 | ¹H NMR (DMSO-d6, 400 MHz): δ 11.70 (s, 1H), 8.90 (s, 1H), 7.93 (dd, J₁ = 2.8 Hz, J₂ = 4.4 Hz, 1H), 7.51 (m, 1H), 7.35 (t, J = 9.2 Hz, 1H), 6.85 (s, 2H), 6.00 (m, 2H), 5.11 (s, 1H), 3.50 (s, 3H), 3.41 (m, 1H), 2.56 -2.51 (m, 2H), 2.30 - 2.25 (m, 2H), 2.17 - 2.11 (m, 2H), 1.85 - 1.65 (m, 4H) ppm. |
| Example 148 | 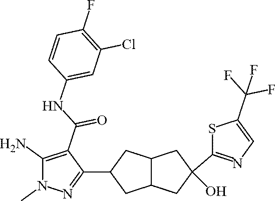 [SMILES: Cn1nc(C2CC3CC(O)(c4ncc(C(F)(F)F)s4)CC3C2)c(C(=O)Nc2ccc(F)c(Cl)c2)c1N] | 544.2 | ¹H NMR (DMSO-d6, 400 MHz): δ 8.95 (s, 1H), 8.30 (d, J = 1.2 Hz, 1H), 7.93 (dd, J₁ = 2.4 Hz, J₂ = 6.4 Hz, 1H), 7.53 (m, 1H), 7.35 (t, J = 5.2 Hz, 1H), 6.33 (s, 1H), 6.00 (s, 2H), 3.50 (s, 3H), 3.42 (m, 1H), 2.80 - 2.60 (m, 2H), 2.33 - 2.16 (m, 4H), 1.92 - 1.81 (m, 4H) ppm. |
| Example 149 | 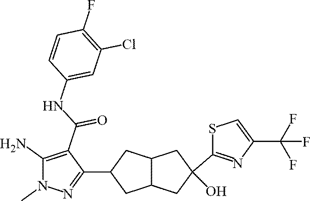 [SMILES: Cn1nc(C2CC3CC(O)(c4nc(C(F)(F)F)cs4)CC3C2)c(C(=O)Nc2ccc(F)c(Cl)c2)c1N] | 544.2 | ¹H NMR (DMSO-d6, 400 MHz): δ 8.94 (s, 1H), 8.33 (d, J = 0.8 Hz, 1H), 7.93 (dd, J₁ = 2.4 Hz, J₂ = 6.8 Hz, 1H), 7.53 (m, 1H), 7.35 (t, J = 8.8 Hz, 1H), 6.17 (s, 1H), 6.00 (s, 2H), 3.50 (s, 3H), 3.43 (m, 1H), 2.80 - 2.73 (m, 2H), 2.27 - 2.16 (m, 4H), 1.91 - 1.86 (m, 4H) ppm. |
| Example 150 | 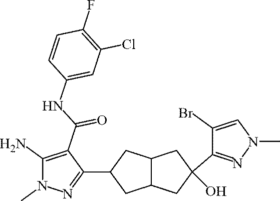 | 533.2 [M-H₂O+1]⁺ | ¹H NMR (DMSO-d6, 400 MHz): δ 8.89 (s, 1H), 7.92 (dd, J₁ = 2.8 Hz, J₂ = 6.8 Hz, 1H), 7.80 (s, 1H), 7.51 (m, 1H), 7.37 (t, J = 9.2 Hz, 1H), 5.99 (br s, 2H), 4.78 (s, 1H), 3.75 (s, 3H), 3.50 (s, 3H), 3.40 (m, 1H), 2.42 - 2.36 (m, 4H), 2.15 - 2.12 (m, 2H), 1.78 - 1.72 (m, 4H) ppm |
| Example 151 | 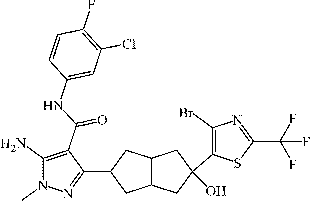 [SMILES: Cn1nc(C2CC3CC(O)(c4sc(C(F)(F)F)nc4Br)CC3C2)c(C(=O)Nc2ccc(F)c(Cl)c2)c1N] | 622.0 | ¹H NMR (DMSO-d6, 400 MHz): δ 8.96 (s, 1H), 7.93 (dd, J₁ = 2.8 Hz, J₂ = 6.8 Hz, 1H), 7.52 (m, 1H), 7.35 (t, J = 5.2 Hz, 1H), 6.35 (s, 1H), 5.99 (s, 2H), 3.50 95, 3H), 3.40 (m, 1H), 2.80 - 2.70 (m, 2H), 2.54 - 2.50 (m, 2H), 2.20 - 2.10 (m, 2H), 1.94 - 1.89 (m, 4H) ppm. |
| Example 152 | 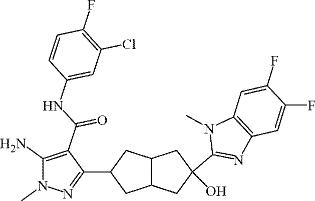 [SMILES: Cn1nc(C2CC3CC(O)(c4nc5cc(F)c(F)cc5n4C)CC3C2)c(C(=O)Nc2ccc(F)c(Cl)c2)c1N] | 559.3 | ¹H NMR (DMSO-d6, 400 MHz): δ 8.96 (s, 1H), 7.92 (dd, J₁ = 1.6 Hz, J₂ = 5.2 Hz, 1H), 7.71 - 7.62 (m, 2H), 7.53 - 7.50 (m, 1H), 7.34 (t, J = 9.2 Hz, 1H), 6.00 (br s, 2H), 5.61 (s, 1H), 3.91 (s, 3H), 3.51 (s, 3H), 3.47 (m, 1H), 2.55 - 2.46 (m, 4H), 2.20 - 2.17 (m, 2H), 1.93 - 1.90 (m, 2H), 1.82 - 1.77 (m, 2H) ppm |
| Example 153 | 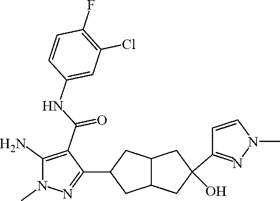 | 455.3 [M-H₂O+1]⁺ | ¹H NMR (DMSO-d6, 400 MHz): δ 8.90 (s, 1H), 7.92 (dd, J₁ = 2.4 Hz, J₂ = 5.6 Hz, 1H), 7.53 - 7.49 (m, 2H), 7.35 (t, J = 9.2 Hz, 1H), 6.14 (d, 1H), 5.99 (s, 1H), 4.71 (s, 1H), 3.75 (s, 3H), 3.50 (s, 3H), 3.36 (m, 1H), 2.51 -2.48 (m, 2H), 2.22 - 2.10 (m, 4H), 1.82 - 1.78 (m, 2H), 1.72 - 1.68 (m, 2H) ppm |
| Example 154 | 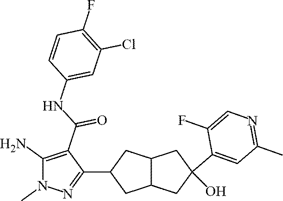 | 502.1 | ¹H NMR (DMSO-d6, 400 MHz): δ 8.94 (s, 1H), 8.24 (d, J = 3.2 Hz, 1H), 7.93 (dd, J₁ = 2.8 Hz, J₂ = 6.8 Hz, 1H), 7.52 (m, 1H), 7.37 - 7.35 (m, 2H), 5.99 (br s, 2H), 5.15 (s, 1H), 3.50 (s, 3H), 3.40 (m, 1H), 2.69 - 2.64 (m, 2H), 2.44 (s, 3H), 2.25 - 2.13 (m, 2H), 2.00 - 1.92 (m, 2H), 1.84 -1.80 (m, 2H) ppm |
| Compd. 4-2 | 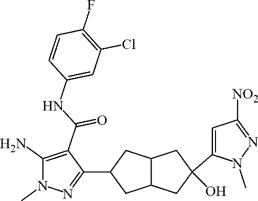 [SMILES: Cn1nc([N+](=O)[O-])cc1C1(O)CC2CC(c3nn(C)c(N)c3C(=O)Nc3ccc(F)c(Cl)c3)CC2C1] | 518.9 | ¹H NMR (DMSO-d₆, 400 MHz): δ 8.93 (s, 1H), 7.93-7.90 (dd, J = 6.8 Hz, 2.8 Hz, 1H), 7.53-7.49 (m, 1H), 7.34 (t, J = 9.2 Hz, 1H), 6.93 (s, 1H), 5.98 (s, 2H), 5.53 (s, 1H), 4.03 (s, 3H), 3.50 (s, 3H), 3.44-3.22 (m, 1H), 2.51-2.49 (m, 2H), 2.27-219 (m, 4H), 2.07-1.89 (m, 2H),1.89-1.76 (m, 2H) ppm |
| Example 155 | 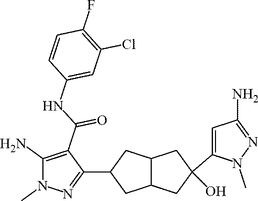 [SMILES: Cn1nc(N)cc1C1(O)CC2CC(c3nn(C)c(N)c3C(=O)Nc3ccc(F)c(Cl)c3)CC2C1] | 488.9 | ¹H NMR (DMSO-d₆, 400 MHz): δ 8.91 (s, 1H), 7.93-7.91 (m, 1H), 7.52-7.49 (m, 1H), 7.34 (t, J = 9.0 Hz, 1H), 5.99 (s, 2H), 5.24 (s, 1H), 5.03 (s, 1H), 4.33 (s, 2H), 3.62 (s, 3H), 3.49 (s, 3H), 3.46-3.40 (m, 1H), 2.50-2.49 (m, 2H), 2.15-2.06 (m, 4H), 1.81-1.71 (m, 4H) ppm |
| Example 156 | 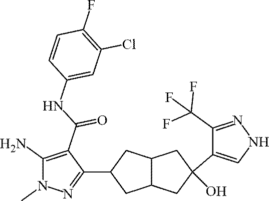 [SMILES: Cn1nc(C2CC3CC(O)(c4c[nH]nc4C(F)(F)F)CC3C2)c(C(=O)Nc2ccc(F)c(Cl)c2)c1N] | 526.9 | ¹H NMR (400 MHz, MeOD-d4) δ 7.80 (dd, J = 6.8, 2.4 Hz, 1H), 7.71 (s, 1H), 7.43 - 7.36 (m, 1H), 7.18 (t, J = 9.2 Hz, 1H), 3.55 (s, 3H), 3.47 - 3.38 (m, 1H), 2.69 - 2.57 (m, 2H), 2.39 - 2.25 (m, 4H), 1.97 -1.83 (m, 4H) ppm |
| Compd. 6-1 | 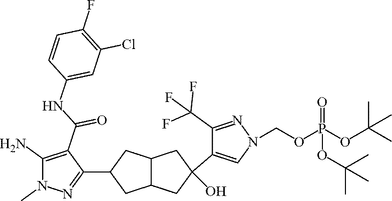 [SMILES: Cn1nc(C2CC3CC(O)(c4cn(COP(=O)(OC(C)(C)C)OC(C)(C)C)nc4C(F)(F)F)CC3C2)c(C(=O)Nc2ccc(F)c(Cl)c2)c1N] | 730.8 [M+H-18]⁺ | ¹H NMR (400 MHz, DMSO-d6) δ 8.98 (s, 1H), 7.97 (s, 1H), 7.90 (dd, J = 6.8, 2.4 Hz, 1H), 7.55 - 7.48 (m, 1H), 7.34 (t, J = 9.2 Hz, 1H), 5.98 (s, 2H), 5.92 - 5.75 (m, 2H), 4.97 (s, 1H), 3.50 (s, 3H), 3.44 - 3.37 (m, 1H), 2.57 - 2.47 (m, 2H), 2.20 - 2.01 (m, 4H), 1.87 -1.71 (m, 4H), 1.37 (s, 18H) ppm |
| Cpmpd. 6-2 | 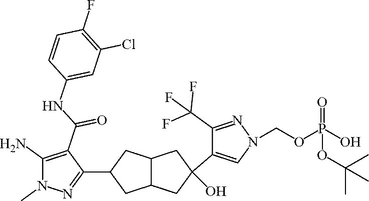 [SMILES: Cn1nc(C2CC3CC(O)(c4cn(COP(=O)(O)OC(C)(C)C)nc4C(F)(F)F)CC3C2)c(C(=O)Nc2ccc(F)c(Cl)c2)c1N] | 691.2 [M-H] | ¹H NMR (400 MHz, MeOD-d4) δ 7.92 (s, 1H), 7.80 (dd, J = 6.8, 2.4 Hz, 1H), 7.45 - 7.35 (m, 1H), 7.19 (t, J = 9.2 Hz, 1H), 5.71 (d, J = 10.8 Hz, 2H), 3.55 (s, 3H), 3.49 - 3.40 (m, 1H), 2.70 - 2.58 (m, 2H), 2.37 -2.25 (m, 4H), 1.97 - 1.79 (m, 4H), 1.35 (s, 9H) ppm |
| Example 157 | 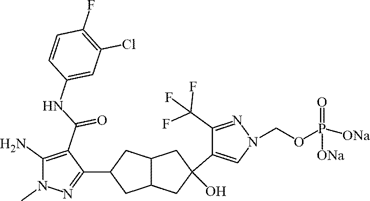 | 635.2 [M-H]⁻ | ¹H NMR (400 MHz, MeOD-d4) δ 7.97 (s, 1H), 7.82 (dd, J = 6.8, 2.4 Hz, 1H), 7.42 - 7.36 (m, 1H), 7.19 (t, J = 9.2 Hz, 1H), 5.72 (d, J = 9.6 Hz, 2H), 3.55 (s, 3H), 3.50 - 3.42 (m, 1H), 2.69 - 2.58 (m, 2H), 2.39 -2.25 (m, 4H), 1.95 - 1.80 (m, 4H) ppm |
| Example 158 | 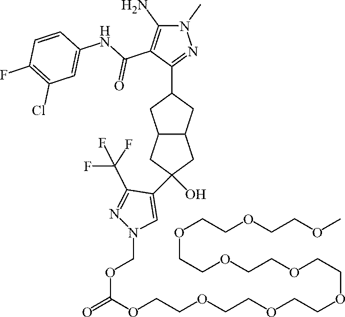 [SMILES: COCCOCCOCCOCCOCCOCCOCCOCCOC(=O)OCn1cc(C2(O)CC3CC(c4nn(C)c(N)c4C(=O)Nc4ccc(F)c(Cl)c4)CC3C2)c(C(F)(F)F)n1] | 967.3 | ¹H NMR (DMSO-d6, 400 MHz): δ 8.94 (s, 1H), 8.00 (s, 1H), 7.92 (dd, J = 2.4, 2.4 Hz, 1H), 7.52-7.49 (m, 1H), 7.34 (t, J = 8.8 Hz, 1H), 6.07 (s, 2H), 5.98 (s, 2H), 4.95 (s, 1H), 4.25-4.22 (m, 2H), 3.62-3.60 (m, 2H), 3.54-3.49 (m, 32H), 3.22 (s, 3H), 2.50-2.49 (m, 2H), 2.15-2.03 (m, 4H), 1.85-1.79 (m, 4H) ppm |
| Example 159 | 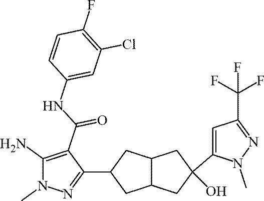 [SMILES: Cn1nc(C(F)(F)F)cc1C1(O)CC2CC(c3nn(C)c(N)c3C(=O)Nc3ccc(F)c(Cl)c3)CC2C1] | 541.2 | ¹H NMR (DMSO-d₆, 400 MHz): δ 8.92 (brs, 1H), 7.92 (dd, J = 7.0, 2.6 Hz, 1H), 7.54-7.49 (m, 1H), 7.35 (t, J = 9.2 Hz, 1H), 6.59 (s, 1H), 6.00 (brs, 2H), 5.39 (s, 1H), 3.99 (s, 3H), 3.50 (s, 3H), 3.48-3.39 (m, 1H), 2.58-2.52 (m, 2H), 2.26-2.12 (m, 4H), 1.90 (dd, J =12.8, 2.8 Hz, 2H), 1.84-1.72 (m, 2H) ppm |
| Example 160 | 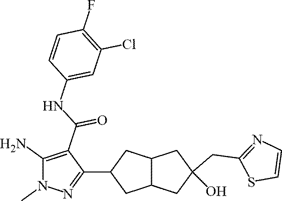 [SMILES: Cn1nc(C2CC3CC(O)(Cc4nccs4)CC3C2)c(C(=O)Nc2ccc(F)c(Cl)c2)c1N] | 490.2 | ¹H NMR (DMSO-d₆, 400 MHz): δ 8.92 (s, 1H), 7.91 (dd, J = 6.8, 2.4 Hz, 1H), 7.67 (d, J = 3.2 Hz, 1H), 7.54 (d, J = 3.6 Hz, 1H), 7.53-7.48 (m, 1H), 7.35 (dd, J = 9.2, 9.2 Hz, 1H), 5.98 (s, 2H), 4.78 (s, 1H), 3.49 (s, 3H), 3.47-3.38 (m, 1H), 3.10 (s, 2H), 2.46-2.39 (m, 2H), 2.19-2.06 (m, 2H), 1.84-1.74 (m, 2H), 1.67-1.55 (m, 2H), 1.48-1.35 (m, 2H). |
| Example 161 | 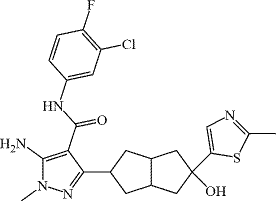 | 490.3 | ¹H NMR (DMSO-d₆, 400 MHz): δ 8.92 (s, 1H), 7.92 (dd, J = 7.0, 2.6 Hz, 1H), 7.56-7.47 (m, 1H), 7.39 (s, 1H), 7.35 (dd, J = 9.2, 9.2 Hz, 1H), 5.99 (s, 2H), 5.41 (s, 1H), 3.49 (s, 3H), 3.46-3.36 (m, 1H), 2.57 (s, 5H), 2.18-2.05 (m, 4H), 1.90-1.73 (m, 4H) ppm |
| Example 162 | 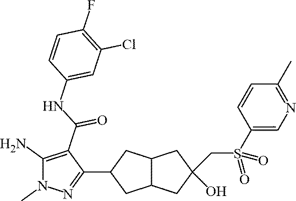 | 562.3 | ¹H NMR (DMSO-d₆, 400 MHz): δ 8.92 (s, 1H), 8.83 (d, J = 2.0 Hz, 1H), 8.10 (dd, J = 8.4, 2.4 Hz, 1H), 7.91-7.89 (m, 1H), 7.50-7.47 (m, 2H), 7.34 (t, J = 9.2 Hz, 1H), 5.95 (s, 2H), 4.41 (s, 1H), 3.55 (s, 2H), 3.47 (s, 3H), 3.33-3.32 (m, 1H), 2.56 (s, 3H), 2.52-2.50 (m, 2H), 2.09-2.06 (m, 2H), 1.93-1.92 (m, 2H), 1.65-1.57 (m, 4H) ppm |
| Example 163 | 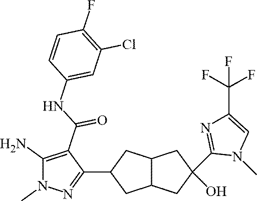 | 541.3 | ¹H NMR (DMSO-d₆, 400 MHz): δ 8.89 (s, 1H), 7.92 (dd, J = 7.2, 2.4 Hz, 1H), 7.71 (d, J = 1.2 Hz, 1H), 7.53-7.49 (m, 1H), 7.35 (t, J = 9.2 Hz, 1H), 6.00 (s, 2H), 5.40 (s, 1H), 3.79 (s, 3H), 3.50 (s, 3H), 3.48 -3.40 (m, 1H), 2.58 - 2.53 (m, 2H), 2.41 - 2.36 (m, 2H), 2.19-2.13 (m, 2H), 1.88 - 1.69 (m, 4H) ppm |
| Example 164 | 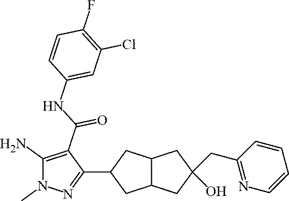 [SMILES: Cn1nc(C2CC3CC(O)(Cc4ccccn4)CC3C2)c(C(=O)Nc2ccc(F)c(Cl)c2)c1N] | 484.2 | ¹H NMR (DMSO-d₆, 400 MHz): δ 8.93 (brs, 1H), 8.46 (d, J = 4.0 Hz, 1H), 7.91 (dd, J = 7.0, 2.6 Hz, 1H), 7.71-7.66 (m, 1H), 7.54-7.48 (m, 1H), 7.35 (t, J = 9.2 Hz, 1H), 7.30 (d, J = 7.6 Hz, 1H), 7.24-7.19 (m, 1H), 5.97 (brs, 2H), 4.85 (s, 1H), 3.48 (s, 3H), 3.47-3.36 (m, 1H), 2.86 (s, 2H), 2.47-2.38 (m, 2H), 2.16-2.08 (m, 2H), 1.82-1.75 (m, 2H), 1.66-1.56 (m, 2H), 1.37 (dd, J = 12.8, 4.8 Hz, 2H) ppm |
| Example 165 | 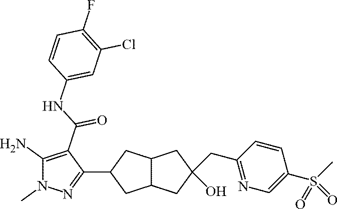 [SMILES: Cn1nc(C2CC3CC(O)(Cc4ccc(S(C)(=O)=O)cn4)CC3C2)c(C(=O)Nc2ccc(F)c(Cl)c2)c1N] | 562.0 | ¹H NMR (DMSO-d₆, 400 MHz): δ 8.94 (d, J=2 Hz, 1H), 8.93 (s, 1H), 8.20-8.18 (dd, J=8.4, 2.4 Hz, 1H), 7.92-7.90 (dd, J = 6.8,2.8 Hz, 1H), 7.56 (d, J=8.0 Hz,1H), 7.50 (t, J=2.4 Hz, 1H), 7.35 (t, J = 9.2 Hz,1H), 5.96 (s, 2H), 4.66 (s,1H), 3.47 (s, 3H), 3.45-3.37 (m,1H), 3.29(s, 3H), 2.98 (s, 2H), 2.51-2.49 (m,2H), 2.14-2.07 (m, 2H), 1.85-1.80 (m,2H),1.65-1.57 (m, 2H), 1.41-1.37 (dd, J=12.8, 4.4 Hz, 2H) ppm |
| Example 166 | 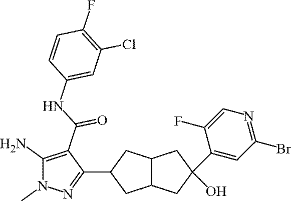 [SMILES: Cn1nc(C2CC3CC(O)(c4cc(Br)ncc4F)CC3C2)c(C(=O)Nc2ccc(F)c(Cl)c2)c1N] | 567.8 [M+2]⁺ | ¹H NMR (DMSO-d₆, 400 MHz): δ 8.94 (s, 1H), 8.32 (d, J=2.8 Hz, 1H), 7.93-9-7.91 (dd, J = 6.8, 2.4 Hz, 1H), 7.66 (d, J = 6.0 Hz, 1H), 7.54-7.50 (m, 1H), 7.32 (t, J = 9.2 Hz, 1H), 5.99 (s, 2H), 5.36 (s,1H), 3.50 (s, 3H), 3.47-3.36 (m, 1H), 2.72-2.66 (m, 2H), 2.25-2.13 (m, 4H), 1.99-1.91 (m, 2H), 1.82 (d, J=13.2 Hz, 1H) ppm |
| Example 167 | 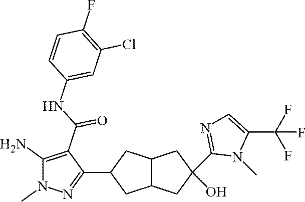 [SMILES: Cn1nc(C2CC3CC(O)(c4ncc(C(F)(F)F)n4C)CC3C2)c(C(=O)Nc2ccc(F)c(Cl)c2)c1N] | 541.3 | ¹H NMR (DMSO-d₆, 400 MHz): δ 8.91 (s, 1H), 7.92 (dd, J = 6.8, 2.4 Hz, 1H), 7.53 - 7.49 (m, 1H), 7.39 - 7.29 (m, 2H), 6.00 (s, 2H), 5.55 (s, 1H), 3.84 (s, 3H), 3.50 (s, 3H), 3.50 - 3.42 (m, 1H), 2.50 - 2.40 (m, 4H), 2.20 - 2.12 (m, 2H), 1.87 - 1.81 (m, 2H), 1.79 - 1.69 (m, 2H) ppm |
| Example 168 | 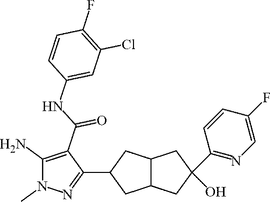 [SMILES: Cn1nc(C2CC3CC(O)(c4ccc(F)cn4)CC3C2)c(C(=O)Nc2ccc(F)c(Cl)c2)c1N] | 488.3 | ¹H NMR (DMSO-d₆, 400 MHz): δ 8.91 (s, 1H), 8.32-8.31 (m, 1H), 7.91 (dd, J = 7.2, 2.8 Hz, 1H), 7.66-7.61 (m, 1H), 7.52-7.49 (m, 1H), 7.40-7.36 (m, 1H), 7.34 (t, J = 8.8 Hz, 1H), 5.99 (s, 2H), 5.18 (s, 1H), 3.50 (s, 3H), 3.43-3.42 (m, 1H), 2.51-2.43 (m, 4H), 2.16-2.14 (m, 2H), 1.85-1.82 (m, 4H) ppm |
| Example 169 | 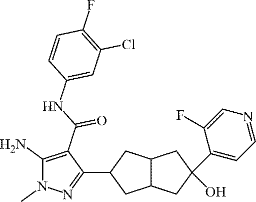 [SMILES: Cn1nc(C2CC3CC(O)(c4ccncc4F)CC3C2)c(C(=O)Nc2ccc(F)c(Cl)c2)c1N] | 488.3 | ¹H NMR (DMSO-d₆, 400 MHz): δ 8.93 (s, 1H), 8.41-8.36 (m, 2H), 7.92 (dd, J = 7.2, 2.4 Hz, 1H), 7.55-7.51 (m, 3H), 7.35 (t, J = 9.2 Hz, 1H), 5.98 (s, 2H), 5.23 (s, 1H), 3.50 (s, 3H), 3.42-3.37 (m, 1H), 2.71-2.69 (m, 2H), 2.23-2.14 (m, 4H), 1.98-1.97 (m, 2H), 1.85-1.82 (m, 2H) ppm |
| Example 170 | 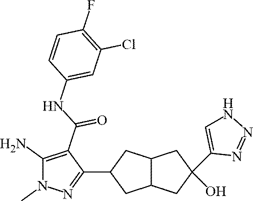 [SMILES: Cn1nc(C2CC3CC(O)(c4c[nH]nn4)CC3C2)c(C(=O)Nc2ccc(F)c(Cl)c2)c1N] | 460.2 | ¹H NMR (DMSO-d₆, 400 MHz): δ = 8.89 (s, 1H), 7.91-7.89 (dd, J = 6.8, 2.8 Hz, 1H), 7.64 (s, 1H), 7.51-7.47 (m, 1H), 7.35-7.30 (t, J = 9.2 Hz, 1H), 5.97 (s, 2H), 5.04 (s, 1H), 3.48 (s, 3H), 3.41-3.38 (m, 1H), 2.49-2.47 (m, 2H), 2.16-2.05 (m, 4H), 1.82-1.74 (m, 4H) ppm |
| Example 171 | 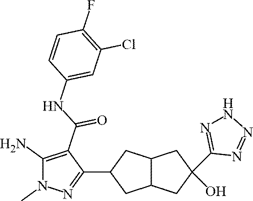 [SMILES: Cn1nc(C2CC3CC(O)(c4nn[nH]n4)CC3C2)c(C(=O)Nc2ccc(F)c(Cl)c2)c1N] | 461.2 | ¹H NMR (CD₃OD, 400 MHz): δ 7.81 (dd, J = 6.8, 2.4 Hz, 1H), 7.44-7.38 (m, 1H), 7.19 (t, J = 8.8 Hz, 1H), 3.56 (s, 3H), 3.47-3.40 (m, 1H), 2.80-2.70 (m, 2H), 2.50-2.30 (m, 4H), 2.06-1.80 (m, 4H) ppm |
| Example 172 | 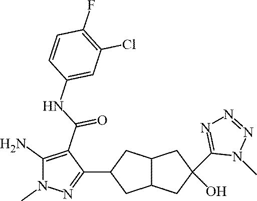 | 475.3 | ¹H NMR (CD₃OD, 400 MHz): δ 7.81 (dd, J = 6.8, 2.4 Hz, 1H), 7.44-7.38 (m, 1H), 7.19 (t, J = 8.8 Hz, 1H), 4.20 (s, 3H), 3.56 (s, 3H), 3.50-3.40 (m, 1H), 2.80-2.70 (m, 2H), 2.45-2.53 (m, 2H), 2.33-2.40 (m, 2H),2.09-1.95 (m, 2H), 1.90-1.80 (m, 2H) ppm |
| Example 173 | 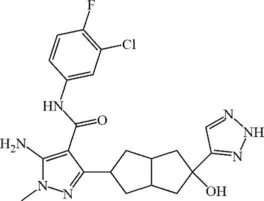 [SMILES: Cn1nc(C2CC3CC(O)(c4cn[nH]n4)CC3C2)c(C(=O)Nc2ccc(F)c(Cl)c2)c1N] | 460.3 | ¹H NMR (DMSO-d₆, 400 MHz): δ 13.63 (s, 1H), 8.91 (s, 1H), 7.93 (dd, J = 6.8, 2.4 Hz, 1H), 7.77 (s, 1H), 7.58 - 7.45 (m, 1H), 7.35 (t, J = 9.2 Hz, 1H), 6.00 (s, 2H), 5.43 (s, 1H), 3.50 (s, 3H), 3.47 - 3.39 (m, 1H), 2.70 - 2.54 (m, 2H), 2.36-2.10 (m, 4H), 1.91 - 1.69 (m, 4H) ppm |
| Example 174 | 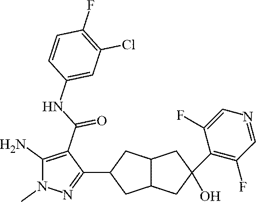 [SMILES: Cn1nc(C2CC3CC(O)(c4c(F)cncc4F)CC3C2)c(C(=O)Nc2ccc(F)c(Cl)c2)c1N] | 506.2 | ¹H NMR (DMSO-d₆, 400 MHz): δ 8.94 (s, 1H), 8.41 (d, J = 1.2, 2H), 7.93 (dd, J = 6.8, 2.4 Hz, 1H), 7.52-7.51 (m, 1H), 7.35 (t, J = 9.2 Hz, 1H), 5.98 (s, 2H), 5.41 (s, 1H), 3.49(s, 3H), 2.67-2.60 (m, 2H), 2.33-2.26 (m, 3H), 2.17-2.14 (m, 4H), 1.94-1.91 (m, 2H) ppm |
| Example 175 | 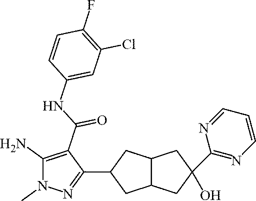 [SMILES: Cn1nc(C2CC3CC(O)(c4ncccn4)CC3C2)c(C(=O)Nc2ccc(F)c(Cl)c2)c1N] | 471.9 | ¹H NMR (DMSO-d₆, 400 MHz): δ 8.92 (s, 1H), 8.79 (d, J = 4.8 Hz, 2H), 7.94-7.91 (m, 1H), 7.53-7.49 (m, 1H), 7.38-7.32 (m, 2H), 5.98 (s, 2H), 5.05 (s, 1H), 3.51 (s, 3H), 3.46-3.35 (m, 1H), 2.67-2.62 (m, 2H), 2.65-2.38 (m, 2H), 2.18-2.12 (m, 2H), 1.92-1.84 (m, 2H), 1.74 (d,J = 12.4 Hz, 2H) ppm |
| Example 176 | 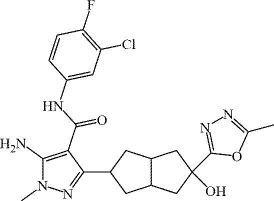 | 475.3 | ¹H NMR (DMSO-d₆, 400 MHz): δ 8.92 (s, 1H), 7.92 (dd, J = 6.8, 2.4 Hz, 1H), 7.51-7.49 (m, 1H), 7.34 (t, J = 9.2 Hz, 1H), 5.99 (s, 2H), 5.76 (s, 1H), 3.49 (s, 3H), 3.43-3.38 (m, 1H), 2.54-2.50 (m, 2H), 2.47 (s, 3H), 2.32-2.27 (m, 2H), 2.17-2.15 (m, 2H), 1.87-1.82 (m, 2H), 1.75-1.73 (m, 2H) ppm |
| Example 177 | 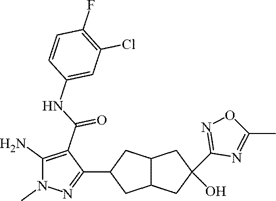 | 475.2 | ¹H NMR (DMSO-d₆, 400 MHz): δ 8.92 (s, 1H), 7.92 (dd, J = 7.2, 2.8 Hz, 1H), 7.53-7.49 (m, 1H), 7.35 (t, J = 9.2 Hz, 1H), 5.99 (s, 2H), 5.45 (s, 1H), 3.50 (s, 3H), 3.45-3.40 (m, 1H), 3.33-3.23 (m, 2H), 2.55 (s, 3H), 2.54-2.51 (m, 1H), 2.27-2.22 (m, 2H), 2.18-2.12 (m, 2H), 1.82-1.74 (m, 4H) ppm |
| Example 178 | 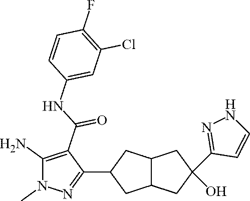 [SMILES: Cn1nc(C2CC3CC(O)(c4cc[nH]n4)CC3C2)c(C(=O)Nc2ccc(F)c(Cl)c2)c1N] | 459.1 | ¹H NMR (CD3OD, 400 MHz): δ 8.19 (d, J = 2.8 Hz, 1H), 7.91 (dd, J = 6.8, 2.8 Hz, 1H), 7.56-7.52 (m,, 1H), 7.24 (t, J = 9.2 Hz, 1H), 6.72 (d, J = 2.8 Hz, 1H), 3.80 (s, 3H), 3.65 - 3.54 (m, 1H), 2.97 - 2.85 (m, 2H), 2.51-2.40 (m, 2H), 2.29-2.24 (m, 2H), 2.14-2.11 (m,, 2H), 2.06 - 1.93 (m, 2H) ppm |
| Example 179 | 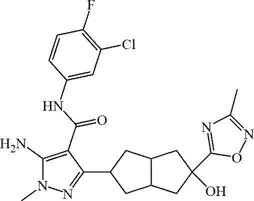 [SMILES: Cc1noc(C2(O)CC3CC(c4nn(C)c(N)c4C(=O)Nc4ccc(F)c(Cl)c4)CC3C2)n1] | 475.2 | ¹H NMR (DMSO-d₆, 400 MHz): δ 8.93 (s, 1H), 7.92 (dd, J = 7.2, 2.8 Hz, 1H), 7.54-7.49 (m, 1H), 7.35 (t, J = 9.2 Hz, 1H), 6.00-5.98 (m, 2H), 5.88 (s, 1H), 3.50 (s, 3H), 3.45-3.40 (m, 1H), 2.62-2.61 (m, 2H), 2.31 (s, 3H), 2.29-2.15 (m, 4H), 1.92-1.89 (m, 2H), 1.81-1.79 (m, 2H) ppm |
| Example 180 | 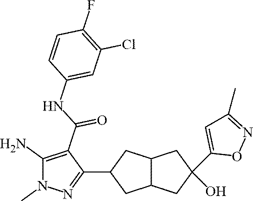 [SMILES: Cc1cc(C2(O)CC3CC(c4nn(C)c(N)c4C(=O)Nc4ccc(F)c(Cl)c4)CC3C2)on1] | 474.3 | ¹H NMR (DMSO-d₆, 400 MHz): δ = 8.93 (s, 1H), 7.94-7.91 (dd, J = 7.2, 2.8 Hz, 1H), 7.54-7.50 (m, 1H), 7.37-7.33 (t, J = 9.2 Hz, 1H), 6.15 (s, 1H), 6.00 (s, 2H), 5.45 (s, 1H), 3.50 (s, 3H), 3.46-3.39 (m, 3H), 2.58 (s, 2H), 2.18-1.85 (m, 7H), 1.81-1.78 (d, J = 11.6 Hz, 4H) ppm |
| Example 181 | 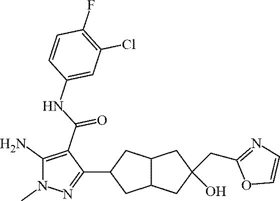 [SMILES: Cn1nc(C2CC3CC(O)(Cc4ncco4)CC3C2)c(C(=O)Nc2ccc(F)c(Cl)c2)c1N] | 474.3 | ¹H NMR (DMSO-d₆, 400 MHz): δ 8.93 (s, 1H), 7.97 (d, J = 1.2 Hz, 1H), 7.91 (dd, J = 7.0, 2.6 Hz, 1H), 7.53-7.48 (m, 1H), 7.35 (t, J = 9.0 Hz, 1H), 7.09 (d, J = 0.4 Hz, 1H) 5.97 (s, 2H), 4.58 (s, 1H), 3.48 (s, 3H), 3.44-3.56 (m, 1H), 2.87 (s, 2H), 2.46-2.41 (m, 2H), 2.17-2.07 (m, 2H), 1.92-1.82 (m, 2H), 1.72-1.60 (m, 2H), 1.48 (dd, J =13.0, 3.8 Hz, 2H) ppm |
| Example 182 | 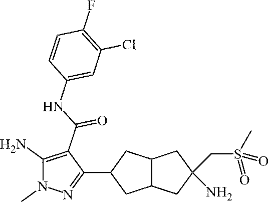 [SMILES: Cn1nc(C2CC3CC(N)(CS(C)(=O)=O)CC3C2)c(C(=O)Nc2ccc(F)c(Cl)c2)c1N] | 484.1 | ¹H NMR (CD3OD, 400 MHz): δ 7.78 (dd, J = 6.8, 2.8 Hz, 1H), 7.44-7.40 (m, 1H), 7.19 (t, J = 8.8 Hz, 1H), 3.56-3.45 (4H, m), 3.35 (s, 2H), 3.01 (s, 3H), 2.68-2.60 (m, 2H), 2.37-2.29 (m, 4H), 1.66-1.45 (m, 4H) ppm |
| Example 183 | 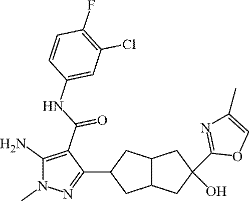 [SMILES: Cc1coc(C2(O)CC3CC(c4nn(C)c(N)c4C(=O)Nc4ccc(F)c(Cl)c4)CC3C2)n1] | 474.3 | ¹H NMR (DMSO-d₆, 400 MHz): δ 8.90 (s, 1H), 7.92 (dd, J = 7.2, 2.8 Hz, 1H), 7.68 (s, 1H), 7.52-7.50 (m, 1H), 7.34 (t, J = 9.2 Hz, 1H), 6.00 (s, 2H), 4.42 (s, 1H), 3.49 (s, 3H), 3.41-3.39 (m, 1H), 2.50-2.48 (m, 2H), 2.31-2.26 (m, 2H), 2.16-2.13 (m, 2H), 2.04 (s, 3H), 1.78-1.73 (m, 4H) ppm |
| Example 184 | 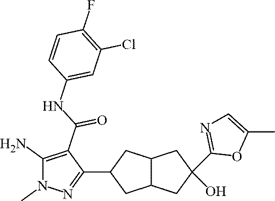 | 474.3 | ¹H NMR (CD₃OD, 400 MHz): δ 7.81 (dd, J = 7.2, 2.4 Hz, 1H), 7.41-7.38 (m, 1H), 7.18 (t, J = 8.8 Hz, 1H), 3.55 (s, 3H), 3.43-3.39 (m, 1H), 2.66-2.64 (m, 2H), 2.49-2.44 (m, 2H), 2.33-2.30 (m, 5H), 1.93-1.83 (m, 4H) ppm |
| Example 185 | 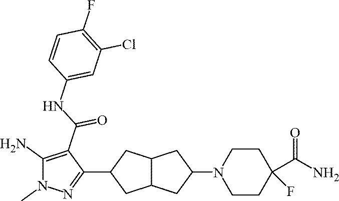 [SMILES: Cn1nc(C2CC3CC(N4CCC(F)(C(N)=O)CC4)CC3C2)c(C(=O)Nc2ccc(F)c(Cl)c2)c1N] | 521.3 | ¹H NMR (DMSO-d₆, 400 MHz): δ = 8.97 (s, 1H), 7.92-7.89 (dd, J = 6.8, 2.8 Hz, 1H), 7.54-7.50 (m, 2H), 7.37-7.32 (t, J = 9.2 Hz, 2H), 5.97 (s, 2H), 3.50 (s, 4H), 2.81 (s, 2H), 2.59 (s, 1H), 2.38 (s, 2H), 2.19-2.13 (m, 3H), 2.11-2.01 (m, 4H), 1.96-1.88 (m, 1H), 1.72-1.66 (t, J = 12.4 Hz, 2H), 1.49-1.45 (m, 2H), 1.15-1.13 (m, 2H) ppm |
| Example 186 | 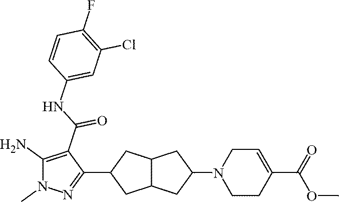 | 516.3 | ¹H NMR (CD3OD, 400 MHz): δ 7.80 (dd, J = 6.8, 2.8 Hz, 1H), 7.44 -7.39 (m, 1H), 7.19 (t, J = 9.2 Hz, 1H), 6.91 - 6.85 (m, 1H), 3.73 (s, 3H), 3.55 (s, 3H), 3.54 - 3.46 (m, 1H), 3.29 - 3.21 (m, 4H), 2.86 -2.77 (m, 1H), 2.75 - 2.68 (m, 2H), 2.60 - 2.51 (m, 2H), 2.41 (s, 2H), 2.38 - 2.23 (m, 4H), 1.63 - 1.54 (m, 2H) ppm |
| Example 187 | 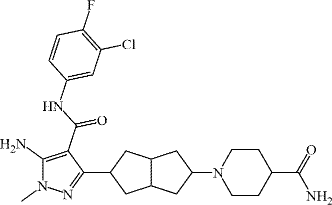 [SMILES: Cn1nc(C2CC3CC(N4CCC(C(N)=O)CC4)CC3C2)c(C(=O)Nc2ccc(F)c(Cl)c2)c1N] | 503.3 | ¹H NMR (CD3OD, 400 MHz): δ 7.79 (dd, J = 6.8, 2.8 Hz, 1H), 7.44 -7.38 (m, 1H), 7.19 (t, J = 9.0 Hz, 1H), 4.62 (s, 2H), 3.55 (S, 3H), 3.55 - 3.47 (m, 1H), 3.11 (d, J = 12.8 Hz, 2H), 2.77 - 2.62 (m, 1H), 2.58 -2.44 (m, 2H), 2.36 - 2.28 (m, 2H), 2.27 - 2.18 (m, 3H), 2.17 - 2.07 (m, 2H), 1.86 - 1.68 (m, 4H), 1.63 - 1.52 (m, 2H) ppm |
| Example 188 | 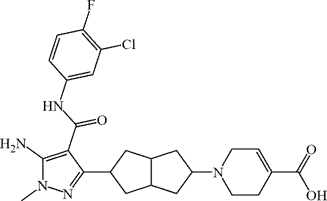 [SMILES: Cn1nc(C2CC3CC(N4CC=C(C(=O)O)CC4)CC3C2)c(C(=O)Nc2ccc(F)c(Cl)c2)c1N] | 502.3 | ¹H NMR (CD3OD, 400 MHz): δ 7.79 (dd, J = 6.8, 2.8 Hz, 1H), 7.46 -7.38 (m, 1H), 7.19 (t, J = 9.2 Hz, 1H), 6.54 (s, 1H), 3.78 (s, 2H), 3.63 - 3.50 (m, 5H), 3.30 - 3.27 (m, 2H), 2.68 - 2.56 (m, 4H), 2.51 - 2.42 (m, 2H), 2.41 - 2.32 (m, 2H), 1.70 - 1.60 (m, 2H), 1.56 - 1.46 (m, 2H) ppm |
| Example 189 | 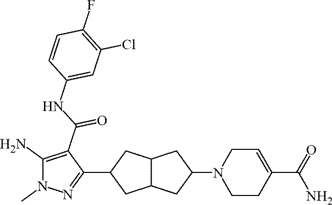 [SMILES: Cn1nc(C2CC3CC(N4CC=C(C(N)=O)CC4)CC3C2)c(C(=O)Nc2ccc(F)c(Cl)c2)c1N] | 501.3 | ¹H NMR (DMSO-d₆, 400 MHz): δ 8.97 (s, 1H), 7.91 (dd, J = 6.8, 2.8 Hz, 1H), 7.55 - 7.48 (m, 1H), 7.35 (t, J = 9.2 Hz, 1H), 7.26 (s, 1H), 6.88 (s, 1H), 6.47 (s, 1H), 5.97 (s, 2H), 3.59 - 3.51 (m, 1H), 3.49 (s, 3H), 3.02 (d, J = 2.4 Hz, 2H), 2.67 - 2.57 (m, 1H), 2.46 - 2.29 (m, 3H), 2.24 - 2.13 (m, 4H), 2.12 - 2.04 (m, 2H), 2.03 - 1.93 (m, 1H), 1.51 - 1.41 (m, 2H), 1.19 - 1.09 (m, 2H) ppm |
| Example 190 | 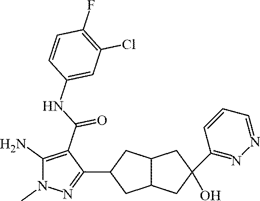 [SMILES: Cn1nc(C2CC3CC(O)(c4cccnn4)CC3C2)c(C(=O)Nc2ccc(F)c(Cl)c2)c1N] | 471.3 | ¹H NMR (CD₃OD, 400 MHz): δ9.03-9.01 (m, 1H), 7.99-7.96 (m, 1H), 7.82 (dd, J = 6.8, 2.8 Hz, 1H), 7.70-7.67 (m, 1H), 7.44-7.40 (m, 1H), 7.19 (t, J = 9.2 Hz, 1H), 3.56 (s, 3H), 3.46-3.44 (m, 1H), 2.85-2.84 (m, 2H), 2.57-2.52 (m, 2H), 2.36-2.31 (m, 2H), 2.04-1.93 (m, 4H) ppm |
| Example 191 | 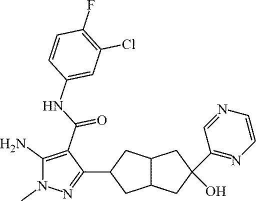 [SMILES: Cn1nc(C2CC3CC(O)(c4cnccn4)CC3C2)c(C(=O)Nc2ccc(F)c(Cl)c2)c1N] | 471.9 | ¹H NMR (DMSO-d₆, 400 MHz): δ 8.93 (s, 1H), 8.88 (s, 1H), 8.87-8.52 (m, 1H), 8.87 (d, J = 2.8 Hz, 1H), 7.94-7.91 (dd, J = 6.8 Hz, 2.8 Hz,1H), 7.54-7.50 (m, 1H), 7.35 (t, J = 9.0 Hz, 1H), 5.99 (s, 2H), 5.28 (s, 1H), 3.51 (s, 3H), 3.49-3.38 (m, 1H), 2.68 (d, J = 5.6 Hz, 2H), 2.32-2.24 (m, 2H), 2.19-2.13 (m, 2H), 1.98-1.90 (m, 2H), 1.77 (t, J = 11.0 Hz, 2H) ppm |
| Example 192 | 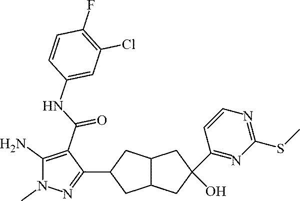 | 518.0 | ¹H NMR (DMSO-d₆, 400 MHz): δ 8.94 (s, 1H), 8.57 (t, J = 2.0 Hz, 1H), 7.92 (d, J = 4 Hz, 1H), 7.52 (t, J = 4.6 Hz, 1H), 7.38-7.33 (m, 2H), 5.99 (s, 2H), 5.25 (s, 1H), 3.50 (s, 3H), 3.43-3.40 (m, 1H), 3.33-3.26 (m, 3H), 2.75 (d, J = 4.4 Hz, 2H), 2.26-2.23 (m, 2H), 2.21-2.15 (m, 2H), 1.96-1.91 (m, 2H), 1.72 (d, J = 13.2 Hz, 2H) ppm |
| Example 193 | 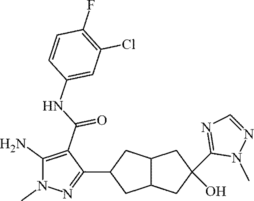 | 474.3 | ¹H NMR (DMSO-d₆, 400 MHz): δ 8.92 (brs, 1H), 7.92 (dd, J = 7.0, 2.6 Hz, 1H), 7.71 (s, 1H), 7.55-7.48 (m, 1H), 7.35 (t, J = 9.2 Hz, 1H), 6.00 (s, 2H), 5.54 (s, 1H), 3.93 (s, 3H), 3.50 (s, 3H), 3.49-3.39 (m, 1H), 2.60-2.53 (m, 2H), 2.40-2.30 (m, 2H), 2.21-2.11 (m, 2H), 1.86 (dd, J = 13.2, 3.2 Hz, 2H), 1.82-1.71 (m, 2H) ppm |
| Example 194 | 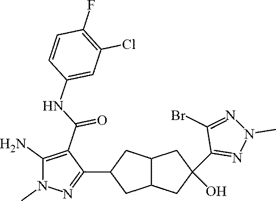 | 552.2 | ¹H NMR (DMSO-d₆, 400 MHz): δ 8.91 (brs, 1H), 7.92 (dd, J = 6.8, 2.8 Hz, 1H), 7.54-7.47 (m, 1H), 7.35 (t, J = 9.2 Hz, 1H), 6.00 (brs, 2H), 5.12 (s, 1H),4.07 (s, 3H), 3.50 (s, 3H), 3.47-3.37 (m, 1H), 2.48-2.41 (m, 2H), 2.37-2.28 (m, 2H), 2.20-2.10 (m, 2H) 1.88-1.69 (m, 4H) ppm |
| Example 195 | 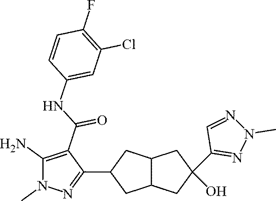 | 474.2 | ¹H NMR (DMSO-d₆, 400 MHz): δ 8.91 (brs, 1H), 7.92 (dd, J = 6.8, 2.8 Hz, 1H), 7.57 (s, 1H), 7.54-7.47 (m, 1H), 7.35 (t, J = 9.2 Hz, 1H), 6.00 (brs, 2H), 5.08 (s, 1H),4.06 (s, 3H), 3.50 (s, 3H), 3.46-3.39 (m, 1H), 2.46-2.40 (m, 2H),2.21-2.09 (m, 4H), 1.85-1.69 (m, 4H) ppm |
| Example 196 | 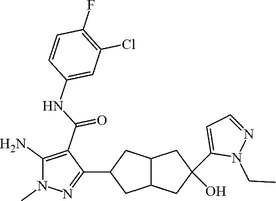 [SMILES: CCn1nccc1C1(O)CC2CC(c3nn(C)c(N)c3C(=O)Nc3ccc(F)c(Cl)c3)CC2C1] | 486.9 | ¹H NMR (DMSO-d₆, 400 MHz): 88.92 (s, 1H), 7.92 (dd, J = 6.8, 2.4 Hz, 1H), 7.53-7.49 (m, 1H), 7.34 (t, J = 9.2 Hz, 1H), 7.25 (s, 1H), 6.03 (s, 1H), 5.99 (s, 2H), 5.19 (s, 1H), 4.30-4.24 (m, 2H), 3.50 (s, 3H), 3.44-3.39 (m, 1H), 3.33-3.30 (m, 2H), 2.18-2.13 (m, 4H), 1.90-1.86 (m, 2H), 1.82-1.77 (m, 2H), 1.32 (t, J = 7.6 Hz, 3H) ppm |
| Example 197 | 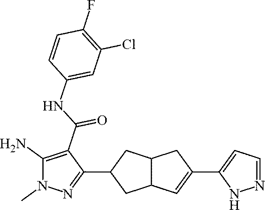 [SMILES: Cn1nc(C2CC3C=C(c4ccn[nH]4)CC3C2)c(C(=O)Nc2ccc(F)c(Cl)c2)c1N] | 440.2 | ¹H NMR (CD3OD, 400 MHz): δ 7.91-7.89 (dd, J = 7.2, 2.4 Hz, 1H), 7.62 (s, 1H), 7.54-7.50 (m, 1H), 7.33 (s, 1H), 7.24 (t, J = 8.8 Hz, 1H), 6.03 (d, J = 1.6 Hz, 1H), 3.77 (s, 1H), 3.44-3.42 (m, 2H), 3.01-2.83 (m, 2H), 2.64-2.60 (m, 1H), 2.42-2.26 (m, 2H), 2.16 (s, 3H), 1.75-1.63 (m, 2H) ppm |
| Example 198 | 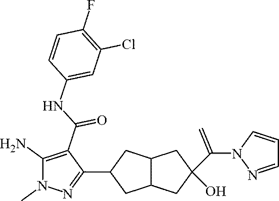 [SMILES: C=C(n1cccn1)C1(O)CC2CC(c3nn(C)c(N)c3C(=O)Nc3ccc(F)c(Cl)c3)CC2C1] | 484.8 | ¹H NMR (DMSO-d₆, 400 MHz):δ 8.93 (s, 1H), 8.11 (d, J = 1.2 Hz, 1H), 7.91 (dd, J = 6.8, 2.4 Hz, 1H), 7.58 (d, J =0.4 Hz, 1H), 7.53-7.49 (m, 1H), 7.34 (t, J = 9.2 Hz, 1H), 6.35 (t, J = 2.0 Hz, 1H), 5.98 (d, J = 3.2 Hz, 2H), 5.41 (s, 1H), 5.34 (s, 1H), 5.15 (s, 1H), 3.50 (s, 3H), 3.47-3.38 (m, 1H), 2.50-2.52 (m, 2H), 2.15-2.08 (m, 4H), 1.79-1.65 (m, 4H) ppm |
| Example 199 | 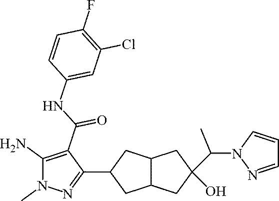 [SMILES: CC(n1cccn1)C1(O)CC2CC(c3nn(C)c(N)c3C(=O)Nc3ccc(F)c(Cl)c3)CC2C1] | 486.9 | ¹H NMR (CD3OD, 400 MHz): δ 7.79 (dd, J = 6.4, 2.4 Hz, 1H),7.68 (d, J =1.2 Hz, 1H), 7.44-7.38 (m, 2H), 7.18 (t, J = 9.2 Hz, 1H), 6.27 (t, J = 2.0 Hz, 1H), 4.30 (dd, J =14.0, 7.2 Hz, 1H),3.54 (s, 3H), 3.41-3.33 (m, 1H), 2.65-2.62 (m, 2H), 2.31-2.18 (m, 2H), 1.97-1.91 (m, 1H), 1.85-1.76 (m, 2H), 1.71-1.65 (m, 2H), 1.49 (d, J =3.6 Hz, 3H), 1.23-1.19 (m, 1H) ppm |
| Example 200 | 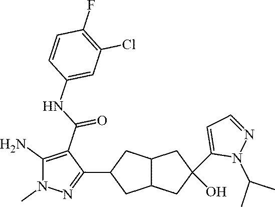 [SMILES: CC(C)n1nccc1C1(O)CC2CC(c3nn(C)c(N)c3C(=O)Nc3ccc(F)c(Cl)c3)CC2C1] | 501.2 | ¹H NMR (DMSO-d₆, 400 MHz): δ 8.93 (s, 1H), 7.93-7.91 (m, 1H), 7.53-7.49 (m, 1H), 7.34 (t, J = 9.2 Hz, 1H), 7.26 (d, J = 1.6 Hz, 1H), 6.00-5.99 (m, 3H), 5.22 (s, 1H), 5.09-5.06 (m, 1H), 3.50 (s, 3H), 3.44-3.40 (m, 1H), 2.51-2.50 (m, 2H), 2.18-2.14 (m, 4H), 1.91-1.87 (m, 2H), 1.83-1.75 (m, 2H), 1.35-1.33 (m, 6H) ppm |
| Example 201 | 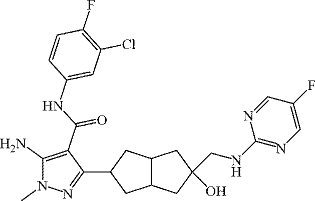 [SMILES: Cn1nc(C2CC3CC(O)(CNc4ncc(F)cn4)CC3C2)c(C(=O)Nc2ccc(F)c(Cl)c2)c1N] | 518.9 | ¹H NMR (DMSO-d₆, 400 MHz): δ 8.92 (s, 1H), 8.32 (s, 2H), 7.91 (dd, J = 6.8 Hz, 1.6 Hz, 1H), 7.53-7.49 (m, 1H), 7.35 (t, J = 9.2 Hz, 1H), 6.80 (t, J = 6.0 Hz, 1H), 5.98 (s, 2H), 4.56 (s, 1H), 3.49 (s, 3H), 3.42-3.38 (m, 1H), 2.51-2.44 (m, 4H), 2.15-2.10 (m, 2H), 1.84-1.79 (m, 2H), 1.68-1.60 (m, 2H), 1.42-1.38 (m, 2H) ppm |
| Example 202A | 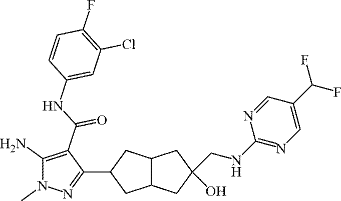 [SMILES: Cn1nc(C2CC3CC(O)(CNc4ncc(C(F)F)cn4)CC3C2)c(C(=O)Nc2ccc(F)c(Cl)c2)c1N] | 550.0 | Isomer A: ¹H NMR (CD3OD-d₄, 400 MHz): δ 8.30 (s, 1H), 7.69 (dd, J = 6.8, 2.4 Hz, 1H), 7.32-7.29 (m, 1H), 7.06 (t, J = 9.2 Hz, 1H), 6.74 (s, 1H), 6.61 (s, 1H),6.47(s, 1H),4.51 (s, 2H), 3.49 (s, 2H), 3.47 (s, 1H),3.45 (s, 3H), 3.21-3.20 (m, 2H), 2.74-2.69 (m, 2H),2.23-2.19 (m, 2H),1.87-1.82 (m,2H),1.46-1.38 (m,4H) ppm |
| Example 202B | 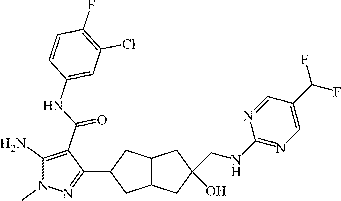 [SMILES: Cn1nc(C2CC3CC(O)(CNc4ncc(C(F)F)cn4)CC3C2)c(C(=O)Nc2ccc(F)c(Cl)c2)c1N] | 550.0 | Isomer B: ¹H NMR (CD3OD-d₄, 400 MHz): δ 8.30 (s, 1H), 7.70 (dd, J = 6.8, 2.4 Hz, 1H), 7.33-7.29 (m, 1H), 7.06 (t, J = 9.2 Hz, 1H), 6.74 (s, 1H), 6.60 (s, 1H), 6.46 (s, 1H),4.51 (s, 2H), 3.45 (s, 2H), 3.43 (s, 3H), 3.38-3.32 (m, 1H), 3.21-3.20 (m, 2H), 2.55-2.50 (m, 2H),2.24-2.18 (m, 2H),1.93-1.88 (m,2H), 1.65-1.57 (m,2H), 1.49-1.44 (m,2H) ppm |
| Example 203A | 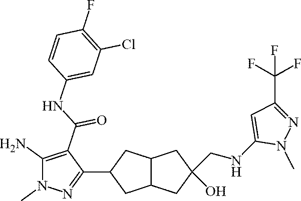 [SMILES: Cn1nc(C(F)(F)F)cc1NCC1(O)CC2CC(c3nn(C)c(N)c3C(=O)Nc3ccc(F)c(Cl)c3)CC2C1] | 570.2 | Isomer A: ¹H NMR (DMSO-d6, 400 MHz): δ 8.94 (s, 1H), 7.92 - 7.90 (m, 1H), 7.53 - 7.49 (m, 1H), 7.35 (t, J = 9.2 Hz, 1H), 5.98 (s, 2H), 5.75 (s, 1H), 5.48 (t, J = 6.0 Hz, 1H), 4.50 (s, 1H), 3.60 (s, 3H), 3.49 (s, 3H), 3.46 - 3.37 (m, 1H), 2.98 (d, J = 6.0 Hz, 2H), 2.47 - 2.42 (m, 2H), 2.17 - 2.12 (m, 2H), 1.89 - 1.84 (m, 2H), 1.73 - 1.65 (m, 2H), 1.45 (dd, J = 12.8, 4.0 Hz, 2H) ppm |
| Example 203B | 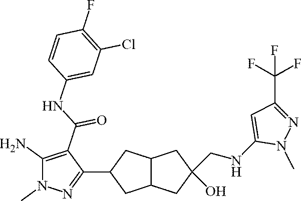 [SMILES: Cn1nc(C(F)(F)F)cc1NCC1(O)CC2CC(c3nn(C)c(N)c3C(=O)Nc3ccc(F)c(Cl)c3)CC2C1] | 570.0 | Isomer B: ¹H NMR (DMSO-d6, 400 MHz): δ 8.95 (s, 1H), 7.92 - 7.90 (m, 1H), 7.54 - 7.50 (m, 1H), 7.35 (t, J = 9.2 Hz, 1H), 5.98 (s, 2H), 5.73 (s, 1H), 5.53 (t, J = 6.0 Hz, 1H), 4.35 (s, 1H), 3.60 (s, 3H), 3.57 -3.51 (m, 1H), 3.48 (s, 3H), 3.06 (d, J = 6.0 Hz, 2H), 2.71 -2.61 (m, 2H), 2.20 - 2.09 (m, 2H), 1.84 - 1.79 (m, 2H), 1.44 - 1.34 (m, 4H) ppm |
| Example 20 | 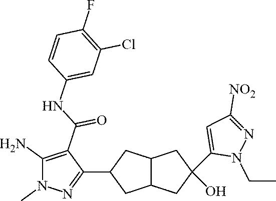 [SMILES: CCn1nc([N+](=O)[O-])cc1C1(O)CC2CC(c3nn(C)c(N)c3C(=O)Nc3ccc(F)c(Cl)c3)CC2C1] | 532.1 | ¹H-NMR (DMSO-d₆, 400 MHz): δ 8.93 (s, 1H), 7.93-7.90 (m, 1H), 7.53-7.49 (m, 1 H), 7.34 (t, J = 9.2 Hz, 1H), 6.90 (s, 1H), 5.98 (s, 2H), 5.58 (s, 1 H), 4.44-4.38 (m, 2H), 3.49 (s, 3H), 3.47-3.43 (m, 1H), 2.55-2.49 (m, 2H), 2.25-2.14 (m, 4H), 1.94-1.90 (m, 2H), 1.83-1.75 (m, 2H), 1.41-1.37 (t, J = 7.0 Hz, 3H) ppm |
| Example 205A | 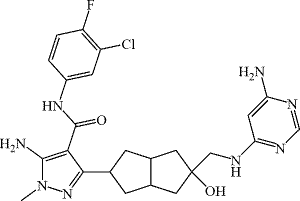 [SMILES: Cn1nc(C2CC3CC(O)(CNc4cc(N)ncn4)CC3C2)c(C(=O)Nc2ccc(F)c(Cl)c2)c1N] | 515.0 | Isomer A: ¹H NMR (DMSO-d₆, 400 MHz): δ 8.93 (s, 1H), 7.89 (dd, J = 6.8, 2.4 Hz, 1H), 7.82 (s, 1H), 7.53-7.49 (m, 1H),7.32 (t, J = 9.2 Hz, 1H), 6.35 (s, 1H), 6.01(s, 2H),5.97 (s, 2H), 5.40(s, 1H), 4.52 (s, 1H),3.57-3.51 (m, 1H), 3.48 (s, 3H), 3.21(s,2H), 2.64(s,2H), 2.11(t,J = 11.6 Hz, 2H), 1.78-1.73 (m, 2H),1.38-1.29 (m, 4H) ppm |
| Example 205B | 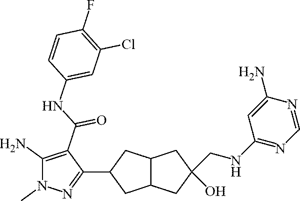 [SMILES: Cn1nc(C2CC3CC(O)(CNc4cc(N)ncn4)CC3C2)c(C(=O)Nc2ccc(F)c(Cl)c2)c1N] | 515.0 | Isomer B: ¹H NMR (DMSO-d₆, 400 MHz): δ 8.92 (s, 1H), 7.90 (dd, J = 6.8, 2.4 Hz, 1H), 7.82 (s, 1H), 7.53-7.49 (m, 1H),7.32 (t, J = 9.2 Hz, 1H), 6.41 (s, 1H),6.02(s, 2H),5.98 (s, 2H), 5.43(s, 1H), 4.82 (s, 1H), 3.49 (s, 3H),3.46-3.39 (m, 1H),3.18(s,2H),2.43(s,2H),2.16-2.11(m,2H), 1.84-1.78 (m, 2H),1.66-1.58 (m, 2H),1.40-1.36 (m, 2H) ppm |
| Example 206 | 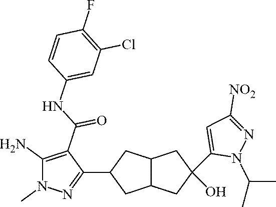 [SMILES: CC(C)n1nc([N+](=O)[O-])cc1C1(O)CC2CC(c3nn(C)c(N)c3C(=O)Nc3ccc(F)c(Cl)c3)CC2C1] | 546.2 | ¹H NMR (DMSO-d₆, 400 MHz): δ 8.93 (s, 1H), 7.93-7.90 (m, 1H), 7.53-7.49 (m, 1H), 7.35 (t, J = 9.0 Hz, 1H), 6.87 (s, 1H), 5.99 (s, 2H), 5.62 (s, 1H), 5.24-5.22 (m, 1H), 3.49 (s, 3H), 3.44-3.31 (m, 1H), 2.55-2.49 (m, 2H), 2.24-2.16 (m, 4 H), 1.92 (d, J =11.2 Hz, 2H), 1.81-1.78 (m, 2H), 1.40 (d, J = 6.8 Hz, 6H) ppm |
| Example 207 | 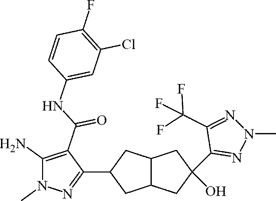 | 541.9 | ¹H NMR (DMSO-d₆, 400 MHz) δ 8.93 (s, 1H), 7.93 - 7.91 (m, 1H), 7.54 - 7.50 (m, 1H), 7.35 (t, J = 9.2 Hz, 1H), 6.00 (s, 2H), 5.29 (s, 1H), 4.17 (s, 3H), 3.50 (s, 3H), 3.46 - 3.38 (m, 1H), 2.62 - 2.53 (m, 2H), 2.25 - 2.12 (m, 4H), 1.88 - 1.75 (m, 4H) ppm |
| Example 208 | 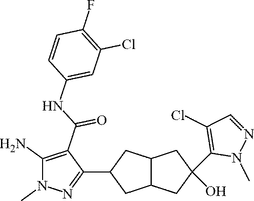 | 507.1 | ¹HNMR(MeOD-d4, 400 MHz) δ 7.71 (dd, J = 6.8, 2.8 Hz, 1H), 7.35 - 7.29 (m, 1H), 7.19 (s, 1H), 7.10 (t, J = 9.2 Hz, 1H), 3.92 (s, 3H), 3.46 (s, 3H), 3.39 - 3.29 (m, 1H), 2.72 - 2.60 (m, 2H), 2.46 (dd, J = 13.6, 8.8 Hz, 2H), 2.27 - 2.18 (m, 2H), 2.02 (d, J = 12.8 Hz, 2H), 1.95 - 1.83 (m, 2H) ppm |
| Example 209 | 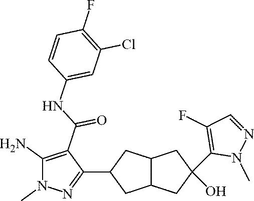 | 491.0 | ¹H NMR (MeOD-d₄, 400 MHz) δ 7.71 (dd, J = 6.8, 2.8 Hz, 1H), 7.38 -7.27 (m, 1H), 7.16 - 7.05 (m, 2H), 3.84 (s, 3H), 3.46 (s, 3H), 3.40 -3.30 (m, 1H), 2.65 - 2.53 (m, 2H), 2.38 - 2.27 (m, 2H), 2.27 - 2.17 (m, 2H), 1.98 - 1.88 (m, 2H), 1.88 - 1.76 (m, 2H) ppm |
| Example 210 | 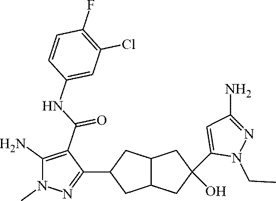 [SMILES: CCn1nc(N)cc1C1(O)CC2CC(c3nn(C)c(N)c3C(=O)Nc3ccc(F)c(Cl)c3)CC2C1] | 502.9 | ¹H NMR (DMSO-d₆, 400 MHz): δ 8.91 (s, 1H), 7.93-7.91 (m, 1H), 7.53-7.49 (m, 1H), 7.34 (t, J = 9.2 Hz, 1H), 5.97 (s, 2H), 5.20 (s, 1H), 5.04 (s, 1H), 4.36 (s, 2H), 4.03-3.98 (m, 2H), 3.49 (s, 3H), 3.44-3.38 (m, 1H), 2.51-2.49 (m, 2H), 2.16-2.05 (m, 4H), 1.82-1.76 (m, 4H), 1.23 (t, J = 7.0 Hz, 3H) ppm |
| Example 211 | 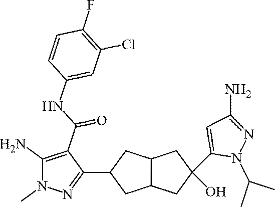 [SMILES: CC(C)n1nc(N)cc1C1(O)CC2CC(c3nn(C)c(N)c3C(=O)Nc3ccc(F)c(Cl)c3)CC2C1] | 517.0 | ¹H NMR (DMSO-d₆, 400 MHz): δ 8.92 (s, 1H), 7.93-7.91 (m, 1H), 7.53-7.49 (m, 1H), 7.34 (t, J = 9.2 Hz, 1H), 5.97 (s,2H), 5.17 (s, 1H), 5.06 (s, 1H), 4.85-4.79 (m, 1H), 4.41-4.34 (m, 2H), 3.49 (s, 3H), 3.46-3.30 (m, 1H), 2.51-2.49 (m, 2H), 2.15-2.03 (m, 4H), 1.83-1.74 (m, 4H), 1.25 (d, J = 6.4 Hz, 6H) ppm |
| Example 212 | 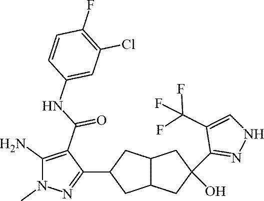 [SMILES: Cn1nc(C2CC3CC(O)(c4n[nH]cc4C(F)(F)F)CC3C2)c(C(=O)Nc2ccc(F)c(Cl)c2)c1N] | 527.1 | ¹H NMR (MeOH-d₄, 400 MHz): δ 7.88-7.62 (m, 2H), 7.33-7.29 (m, 1H), 7.09 (t, J = 8.8 Hz, 1H), 3.46 (s, 3H), 3.36-3.27 (m, 1H), 2.63-2.61 (m, 2H), 2.44-2.17 (m, 4H), 1.85-1.84 (m, 4H) ppm |
| Example 213 | 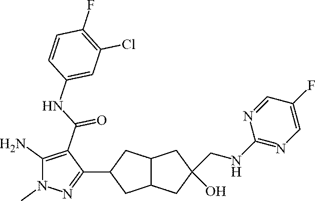 [SMILES: Cn1nc(C2CC3CC(O)(CNc4ncc(F)cn4)CC3C2)c(C(=O)Nc2ccc(F)c(Cl)c2)c1N] | 518.9 | ¹H NMR (DMSO-d₆, 400 MHz): δ 8.93 (s, 1H), 8.32 (d, J = 0.8 Hz, 2H), 7.91 (dd, J1 = 6.8 Hz, 2.4 Hz, 1H), 7.53-7.49 (m, 1H), 7.34 (t, J = 9.2 Hz, 1H), 6.90 (t, J = 6.0 Hz, 1H), 5.97 (s, 2H), 4.37 (s, 1H), 3.56-3.51 (m, 1H), 3.48 (s, 3H), 3.36-3.31 (m, 2H), 2.68-2.63 (m, 2H), 2.16-2.11 (m, 2H), 1.78-1.73 (m, 2H), 1.40-1.32 (m, 4H) ppm |
| Example 214 | 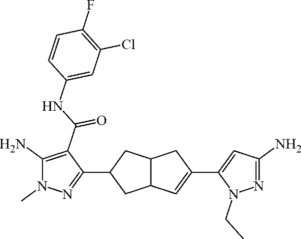 [SMILES: CCn1nc(N)cc1C1=CC2CC(c3nn(C)c(N)c3C(=O)Nc3ccc(F)c(Cl)c3)CC2C1] | 484.9 | ¹H NMR (DMSO-d₆, 400 MHz): δ: 9.01 (s, 1H), 7.93-7.91 (m, 1H), 7.55-7.51 (m, 1H), 7.35 (t, J = 9.0 Hz, 1H), 5.96 (s, 2H), 5.78 (s, 1H), 5.35 (s, 1H), 4.45 (s, 2H), 3.94-3.89 (m, 2H), 3.46-3.42 (m, 4H), 2.79-2.66 (m, 2H), 2.39-2.27 (m, 3H), 2.23-2.19 (m, 1H), 1.50-1.40 (m, 2H), 1.23-1.61 (m, 3H) ppm |
| Example 215 | 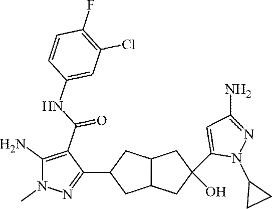 [SMILES: Cn1nc(C2CC3CC(O)(c4cc(N)nn4C4CC4)CC3C2)c(C(=O)Nc2ccc(F)c(Cl)c2)c1N] | 514.9 | ¹H NMR (DMSO-d₆, 400 MHz): δ 8.92 (s, 1H), 7.93-7.91 (dd, J = 2.8 Hz, 6.8 Hz, 1H), 7.53-7.49 (m, 1H), 7.34 (t, J = 9.2 Hz, 1H), 5.99 (s, 2H), 5.26 (s, 1H), 5.09 (s, 1H), 4.37 (s, 2H), 3.94-3.90 (m, 1H), 3.49 (s, 3H), 3.46-3.39 (m, 1H), 2.51-2.49 (m, 2H), 2.20-2.07 (m, 4H), 1.91-1.82 (m, 2H), 1.79-1.74 (m, 2H), 1.01-0.97 (m, 2H), 0.77-0.72 (m, 2H) ppm |
| Example 216 | 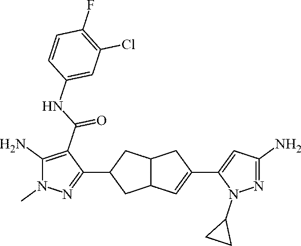 [SMILES: Cn1nc(C2CC3C=C(c4cc(N)nn4C4CC4)CC3C2)c(C(=O)Nc2ccc(F)c(Cl)c2)c1N] | 496.9 | ¹H NMR (DMSO-d₆, 400 MHz): δ 9.02 (s, 1H), 7.93-7.93 (dd, J = 7.2 Hz, 2.8 Hz, 1H), 7.55-7.51 (m, 1H), 7.35 (t, J = 9.2 Hz, 1H), 6.16 (s, 1H), 5.97 (s, 2H), 5.36 (s, 1H), 4.48 (s, 2H), 3.49 (s, 4H), 3.47-3.36 (m, 1H), 2.83-2.66 (m, 2H), 2.51-2.49 (m, 1H), 2.33-2.25 (m, 2H), 2.24-2.19 (m, 1H), 2.15-2.13 (m, 2H), 0.93-0.89 (m, 4H) ppm |
| Example 217 | 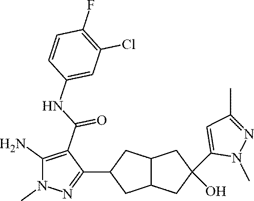 | 487.2 | ¹H NMR (CD₃OD, 400 MHz): δ 7.71 ( dd, J = 6.4, 2.4 Hz, 1H), 7.32-7.29 (m, 1H), 7.09 (t, J = 9.2 Hz, 1H), 5.87 (s, 1H), 3.79 (s, 3H), 3.47 (s, 3H), 3.40-3.33 (m, 1H), 2.52 (s, 2H), 2.29-2.20 (m, 4H), 2.05 (s, 3H), 1.86-1.81 (m, 2H), 1.78-1.69 (m, 2H) ppm |
| Example 218 | 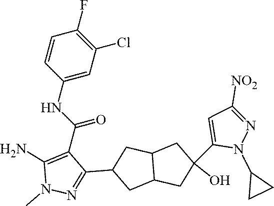 [SMILES: Cn1nc(C2CC3CC(O)(c4cc([N+](=O)[O-])nn4C4CC4)CC3C2)c(C(=O)Nc2ccc(F)c(Cl)c2)c1N] | 544.9 | ¹H NMR (DMSO-d₆400 MHz): δ 8.93 (s, 1H), 7.93-7.90 (m, 1H), 7.53-7.49 (m, 1H), 7.34 (t, J = 9.2 Hz, 1H), 6.95 (s, 1H), 5.99 (s, 2H), 5.62 (s, 1H), 4.40-4.36 (m, 1H), 3.49 (s, 3H), 3.46-3.41 (m, 1H), 2.53-2.50 (m, 2H), 2.34-2.29 (m, 2H), 2.19-2.16 (m, 2H), 2.00 (d, J = 11.2 Hz, 2H), 1.84-1.76 (m, 2H),1.24-1.20 (m, 2H), 1.09-1.04 (m, 2H) ppm |
| Example 219 | 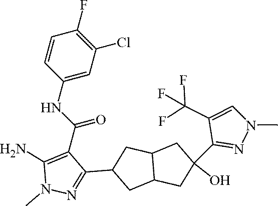 [SMILES: Cn1cc(C(F)(F)F)c(C2(O)CC3CC(c4nn(C)c(N)c4C(=O)Nc4ccc(F)c(Cl)c4)CC3C2)n1] | 540.8 | ¹H NMR (MeOH-d₄, 400 MHz): δ 7.80 (s, 1H), 7.71 (dd, J = 6.8, 2.4 Hz, 1H), 7.62 (s, 1H), 7.33-7.29 (m, 1H), 7.09 (t, J =9.2 Hz, 1H), 3.74 (s, 3H), 3.46 (s, 3H), 3.35-3.32 (m, 1H), 2.58-2.56 (m, 2H), 2.39-2.34 (m, 2H), 2.22-2.18 (m, 2H), 1.82-1.76 (m, 4H) ppm |
| Example 220 | 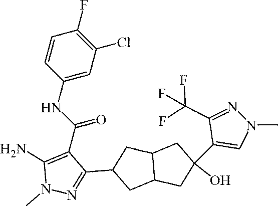 [SMILES: Cn1cc(C2(O)CC3CC(c4nn(C)c(N)c4C(=O)Nc4ccc(F)c(Cl)c4)CC3C2)c(C(F)(F)F)n1] | 541.0 | ¹H NMR (400 MHz, DMSO-d₆): δ 8.95 (s, 1H), 7.93 - 7.91 (m, 1H), 7.78 (s, 1H), 7.52 - 7.50 (m, 1H), 7.35 (t, J = 9.2 Hz, 1H), 5.99 (s, 2H), 4.81 (s, 1H), 3.83 (s, 3H), 3.50 (s, 3H), 3.45 - 3.39 (m, 1H), 2.54 - 2.46 (m, 2H),2.20 - 2.04 (m, 4H), 1.83 - 1.75 (m, 4H) ppm |
| Example 221 | 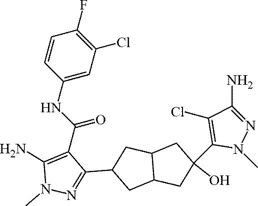 [SMILES: Cn1nc(C2CC3CC(O)(c4c(Cl)c(N)nn4C)CC3C2)c(C(=O)Nc2ccc(F)c(Cl)c2)c1N] | 523.4 [M + 2]⁺ | ¹H NMR (DMSO-d₆, 400 MHz): δ 8.93 (s, 1H), 7.93-7.91 (m, 1H), 7.53-7.49 (m, 1H), 7.35 (t, J = 9.0 Hz, 1H), 5.97 (s, 2H), 5.19 (s, 1H), 4.55 (d, J = 9.0 Hz, 2H), 3.71 (s, 3H), 3.49 (s, 3H), 3.34-3.37 (m, 1H), 2.59-2.50 (m, 2H), 2.34-2.28 (m, 2H), 2.17-2.12 (m, 2H), 1.96 (d, J = 12.8 Hz, 2H), 1.92-1.84 (m, 2H) ppm |
| Example 222 | 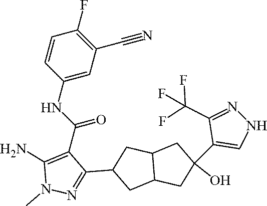 [SMILES: Cn1nc(C2CC3CC(O)(c4c[nH]nc4C(F)(F)F)CC3C2)c(C(=O)Nc2ccc(F)c(C#N)c2)c1N] | 518.0 | ¹H NMR (400 MHz, MeOD-d4): δ 8.03 (dd, J = 2.8, 5.6 Hz, 1H), 7.82-7.78 (m, 1H), 7.72 (s, 1H), 7.31 (t, J = 8.8 Hz, 1H), 3.55 (s, 3H), 3.48-3.39 (m, 1H), 2.65-2.61 (m, 2H), 2.34-2.28 (m, 4H), 1.96-1.84 (m, 4H) ppm |
| Example 223 | 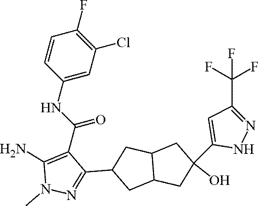 [SMILES: Cn1nc(C2CC3CC(O)(c4cc(C(F)(F)F)n[nH]4)CC3C2)c(C(=O)Nc2ccc(F)c(Cl)c2)c1N] | 527.0 | ¹H NMR (DMSO-d₆, 400 MHz): δ 13.37 (s, 1H), 8.92 (s, 1H), 7.93 (dd, J = 6.8, 2.8 Hz, 1H), 7.53-7.49 (m, 1H), 7.35 (t, J = 9.2, 1H), 6.50 (s, 1H), 6.00 (s, 2H), 5.32 (s, 1H), 3.49 (s, 3H), 3.42-3.34 (m, 1H), 2.57-2.52 (m, 2H), 2.16-2.10 (m, 4H), 1.86-1.78 (m, 4H) ppm |
| Example 224 | 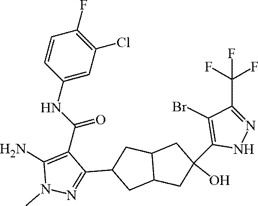 [SMILES: Cn1nc(C2CC3CC(O)(c4[nH]nc(C(F)(F)F)c4Br)CC3C2)c(C(=O)Nc2ccc(F)c(Cl)c2)c1N] | 606.8 [M + 2]⁺ | ¹H NMR (DMSO-d6, 400 MHz): δ 13.65 (s, 1H), 8.94 (s, 1H), 7.93 (dd, J = 7.2, 2.8 Hz, 1H), 7.54-7.50 (m, 1H), 7.35 (t, J = 8.8, 1H), 5.99 (s, 2H), 5.46 (s, 1H), 3.49 (s, 3H), 3.45-3.39 (m, 1H), 2.67-2.66 (m, 2H), 2.34-2.29 (m, 2H), 2.16-2.13 (m, 2H), 1.95 -1.89 (m, 4H) ppm |
| Example 225 | 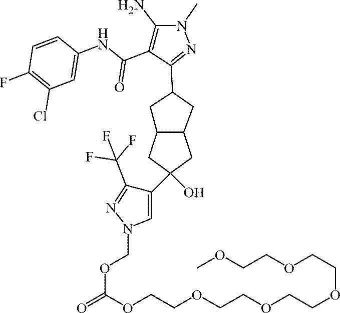 [SMILES: COCCOCCOCCOCCOCCOC(=O)OCn1cc(C2(O)CC3CC(c4nn(C)c(N)c4C(=O)Nc4ccc(F)c(Cl)c4)CC3C2)c(C(F)(F)F)n1] | 791.2 | ¹H NMR (DMSO-d6, 400 MHz): δ 8.94 (s, 1H), 8.00 (s, 1H), 7.91 (dd, J = 6.8, 2.8 Hz, 1H), 7.53-7.49 (m, 1H), 7.34 (t, J = 9.2, 1H), 6.06 (s, 2H), 5.97 (s, 2H), 4.95 (s, 1H), 4.24-4.22 (m, 2H), 3.62-3.60 (m, 2H), 3.52-3.51 (m, 13H), 3.50-3.47 (m, 3H), 3.22 (s, 3H), 2.51-2.50 (m, 2H), 2.15-2.03 (m, 4H), 1.85-1.79 (m, 4H) ppm |
| Example 226 | 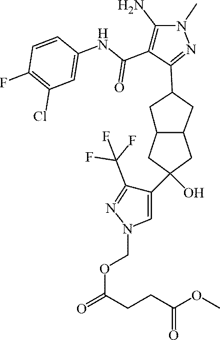 [SMILES: COC(=O)CCC(=O)OCn1cc(C2(O)CC3CC(c4nn(C)c(N)c4C(=O)Nc4ccc(F)c(Cl)c4)CC3C2)c(C(F)(F)F)n1] | 671.2 | ¹H NMR (DMSO-d6, 400 MHz): δ 8.95 (s, 1H), 7.97 (s, 1H), 7.92 (dd, J = 2.4, 2.8 Hz, 1H), 7.53-7.49 (m, 1H), 7.34 (t, J = 8.8 Hz, 1H), 6.03 (s, 2H), 5.98 (s, 2H ), 4.94 (s, 1H), 3.56 (s, 3H), 3.53 (s, 3H), 3.49-3.33 (m, 1H), 2.67-2.53 (m, 6H), 2.15-2.03 (m, 4H), 1.85-1.79 (m, 4H) ppm |
| Example 227 | 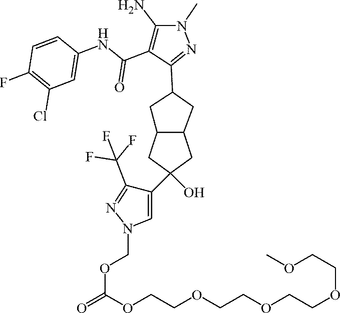 [SMILES: COCCOCCOCCOCCOC(=O)OCn1cc(C2(O)CC3CC(c4nn(C)c(N)c4C(=O)Nc4ccc(F)c(Cl)c4)CC3C2)c(C(F)(F)F)n1] | 835.3 | ¹H NMR (DMSO-d6, 400 MHz): δ 8.95 (s, 1H), 8.00 (s, 1H), 7.92 (dd, J = 6.4, 2.0 Hz, 1H), 7.52-7.50 (m, 1H), 7.34 (t, J = 9.6, 1H), 6.07 (s, 2H), 5.98 (s, 2H), 4.96 (s, 1H), 4.23-4.22 (m, 2H), 3.62-3.61 (m, 2H), 3.57-3.39 (m, 20H), 3.22 (s, 3H), 2.67-2.50 (m, 2H), 2.08-2.03 (m, 4H), 1.85-1.75 (m, 4H) ppm |
| Example 228 | 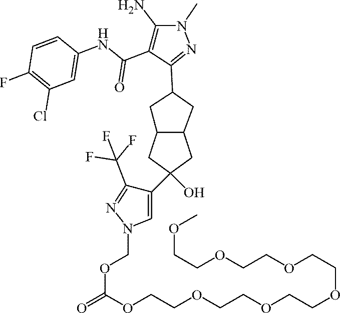 [SMILES: COCCOCCOCCOCCOCCOCCOC(=O)OCn1cc(C2(O)CC3CC(c4nn(C)c(N)c4C(=O)Nc4ccc(F)c(Cl)c4)CC3C2)c(C(F)(F)F)n1] | 879.3 | ¹H NMR (DMSO-d6, 400 MHz): δ 8.93 (s, 1H), 8.00 (s, 1H), 7.92 (dd, J = 2.4, 2.0 Hz, 1H), 7.53-7.49 (m, 1H), 7.34 (t, J = 8.8 Hz, 1H), 6.07 (s, 2H), 5.97 (s, 2H ), 4.94 (s, 1H), 4.24-4.22 (m, 2H), 3.62-3.60 (m, 2H), 3.54-3.49 (m, 21H), 3.42-3.38 (m, 3H), 3.22 (s, 3H), 2.50-2.49 (m, 2H), 2.15-2.03 (m, 4H), 1.85-1.75 (m, 4H) ppm |
| Example 229 | 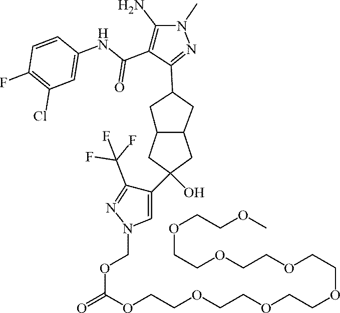 [SMILES: COCCOCCOCCOCCOCCOCCOCCOC(=O)OCn1cc(C2(O)CC3CC(c4nn(C)c(N)c4C(=O)Nc4ccc(F)c(Cl)c4)CC3C2)c(C(F)(F)F)n1] | 923.3 | ¹H NMR (DMSO-d6, 400 MHz): δ 8.94 (s, 1H), 8.00 (s, 1H), 7.92 (dd, J = 2.4, 2.4 Hz, 1H), 7.52-7.49 (m, 1H), 7.34 (t, J = 8.8 Hz, 1H), 6.07 (s, 2H), 5.98 (s, 2H ), 4.95 (s, 1H), 4.24-4.22 (m, 2H), 3.62-3.60 (m, 2H), 3.52-3.49 (m, 25H), 3.44-3.37 (m, 3H), 3.22 (s, 3H), 2.50-2.49 (m, 2H), 2.15-2.03 (m, 4H), 1.85-1.75 (m, 4H) ppm |
| Example 230 | 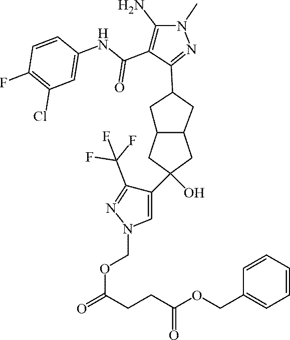 [SMILES: Cn1nc(C2CC3CC(O)(c4cn(COC(=O)CCC(=O)OCc5ccccc5)nc4C(F)(F)F)CC3C2)c(C(=O)Nc2ccc(F)c(Cl)c2)c1N] | 747.2 | ¹H NMR (DMSO-d6, 400 MHz): δ 8.95 (s, 1H), 7.96 (s, 1H), 7.92 (dd, J = 2.8, 2.8 Hz, 1H), 7.53-7.49 (m, 1H), 7.38-7.31 (m, 6H), 6.03 (s, 2H), 5.98 (s, 2H), 5.07 (s, 2H), 4.94 (s, 1H), 3.49 (s, 3H), 3.42-3.36 (m, 1H), 2.67-2.64 (m, 4H), 2.51-2.49 (m, 2H), 2.16-2.02 (m, 4H), 1.88-1.77 (m, 4H) ppm |
| Example 231 | 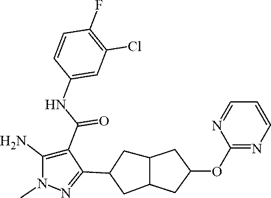 [SMILES: Cn1nc(C2CC3CC(Oc4ncccn4)CC3C2)c(C(=O)Nc2ccc(F)c(Cl)c2)c1N] | 471.2 | ¹H NMR (400 MHz, Chloroform-d): δ 8.51 (d, J = 4.7 Hz, 2H), 7.74 (dd, J = 6.5, 2.5 Hz, 1H), 7.53 (d, J = 3.1 Hz, 1H), 7.32 (ddd, J = 9.0, 4.1, 2.6 Hz, 1H), 7.12 (q, J = 8.4 Hz, 1H), 6.92 (t, J = 4.7 Hz, 1H), 5.44 (p, J = 5.8 Hz, 1H), 5.25 (s, 1H), 3.62 (s, 3H), 3.21 (dq, J = 11.7, 5.9 Hz, 1H), 3.08 (s, 1H), 2.66 (d, J = 27.3 Hz, 2H), 2.42 (dd, J = 12.3, 6.5 Hz, 2H), 2.33 (ddd, J = 13.5, 7.6, 5.5 Hz, 2H), 2.12 - 1.94 (m, 2H), 1.87 (dt, J = 13.8, 5.1 Hz, 2H) ppm |

### VI. Biological Data

### Assay Measuring Activity of Test Compounds on Viral Production From HepAD38 Cells

HepAD38 cells grown in a T-150 flask (Corning, cat#: 430825) with Growth Medium (DMEM/F12 (1:1) (Hyclone, cat#: SH30023.02), 1X Pen/Strep (Invitrogen, cat#: 15140-122), 10% FBS (Tissue Culture Biologics, cat#: 101), 250 µg/mL G418 (Alfa Aesar, cat#: J62671), 1 µg/mL Tetracycline (Teknova, cat#: T3320)) were detached with 0.25% trypsin-EDTA (Invitrogen, cat#: 25200-056). Tetracycline-free treatment medium (15 mL DMEM/F12 (1:1) 1× Pen/step, with 2% FBS, Tet-system approved (Clontech, cat#: 631106) were then added to mix, transferred into a 50 ml conical tube (Falcon, cat#: 21008-918,) and spun at 1300 rpm for 5 min. Pelleted cells were then re-suspended/washed with 50 mL of 1X DPBS (Invitrogen, cat#: 14190-136) 2 times and 50 mL treatment medium twice. HepAD38 cells were then re-suspended with 10 mL of treatment medium, syringed and counted. Wells of 96-well clear bottom TC plate (Corning, cat#: 3904,) were seeded at 50.000 cells/well in 180 µL of treatment medium, and 20 µL of either 10% DMSO (Sigma, cat#: D4540) as controls or a 10X solution of test compounds in 10% DMSO in treatment media was added for a final compound concentration starting at 10 µM, and plates were incubated in 5% CO₂ incubator at 37° C. for 5 days.

Subsequently viral load production was assayed by quantitative PCR (qPCR) of the HBV core sequence. PCR reaction mixture containing forward primers HBV-f 5′-CTGTGCCTTGGGTGGCTTT-3′ (IDT DNA), Reverse primers HBV-r 5′-AAGGAAAGAAGTCAGAAGGCAAAA-3′ (IDT DNA), Fluorescent TaqMan^{tm} Probes HBV-probe 5′-FAM/AGCTCCAAA/ZEN/TTCTTTATAAGGGTCGATGTC/3IABkFQ-3′ (IDT DNA), 10 µL/well of PerfeCTa® qPCR ToughMix® (Quanta Biosciences, Cat#: 95114-05 K), and 6 µL/well of DEPC water (Alfa Aesar, cat#: J62087) was prepared. Four µL of supernatant was added to 16 µL of the reaction mixture in a qPCR plate (Applied Biosytems, Cat#: 4309849), sealed with a film (Applied Biosystems, Cat#: 4311971), centrifuged for a few seconds, and subsequently run on an Applied Biosystems VIIA7. The PCR mixture was incubated at 45° C. for 5 min, then 95° C. for 10 min, followed by 40 cycles of 10 seconds at 95° C. and 20 seconds at 60° C. Viral load was quantified against known HBV DNA standards by using ViiA™ 7 Software. Viral load in the supernatant from wells with treated cells were compared against viral load in supernatant from DMSO control wells (≥ 3 per plate). Cell viability assay was performed with CellTiter-Glo Luminescent Cell Viability Assay (Promega, cat#: G7573) with modification. Mixed appropriate amount of CellTiter-Glo (CTG) 1X DPBS in a 1:1 ratio, added 100 uL of the mixture to each well followed completely removal of all supernatant in each well without touching cell surface. Incubated the plate at room temperature for 10 min on an orbital shaker, and then read the plate with a plate reader (TECAN M1000 or Envision). EC₅₀ or CC₅₀ values were calculated through curve-fitting of the four-parameter nonlinear-logistic-regression model (GraphPad Prism or Dotmatics). CC₅₀ values were all >10 µM.

Table 1 gives the viral load lowering EC₅₀ values for exemplified compounds of the invention grouped in the following ranges: A indicates EC₅₀ ≤ 0.010 µM; B indicates EC₅₀ of > 0.010 and ≤ 0.050 µM; C indicates EC₅₀ of > 0.050 and ≤ 0.500 µM; and D indicates > 0.500 µM

TABLE 1

Viral load lowering for exemplified compounds of the invention

| | |
|---|---|
| Example # | Activity |
| Example 1 | A |
| Example 2 | A |
| Example 3 | B |
| Example 4 | B |
| Example 5 | A |
| Example 6 | A |
| Example 7 | A |
| Example 8 | A |
| Example 9 | A |
| Example 10 | A |
| Example 11 | B |
| Example 12 | B |
| Example 13 | B |
| Example 14 | A |
| Example 15 | A |
| Example 16 | A |
| Example 17 | A |
| Example 18 | A |
| Example 19 | A |
| Example 20 | A |
| Example 21 | A |
| Example 22 | A |
| Example 23 | A |
| Example 24 | A |
| Example 25 | A |
| Example 26 | A |
| Example 27 | B |
| Example 28 | B |
| Example 29 | B |
| Example 30 | B |
| Example 31 | B |
| Example 32 | B |
| Example 33 | B |
| Example 34 | B |
| Example 35 | B |
| Example 36 | B |
| Example 37 | A |
| Example 38 | A |
| Example 39 | A |
| Example 40 | A |
| Example 41 | A |
| Example 42 | A |
| Example 43 | A |
| Example 44 | A |
| Example 45 | A |
| Example 46 | A |
| Example 47 | A |
| Example 48 | A |
| Example 49 | A |
| Example 50 | A |
| Example 51 | B |
| Example 52 | A |
| Example 53 | A |
| Example 54 | A |
| Example 55 | A |
| Example 56 | A |
| Example 57 | B |
| Example 58 | B |
| Example 59 | A |
| Example 60 | A |
| Example 61 | A |
| Example 62 | A |
| Example 63 | B |
| Example 64 | A |
| Example 65 | A |
| Example 66 | B |
| Example 67 | A |
| Example 68 | A |
| Example 69 | A |
| Example 70 | B |
| Example 71 | B |
| Example 72 | A |
| Example 73 | B |
| Example 74 | A |
| Example 75 | B |
| Example 76 | B |
| Example 77 | C |
| Example 78 | C |
| Example 79 | C |
| Example 80 | C |
| Example 81 | C |
| Example 82 | C |
| Example 83 | A |
| Example 84 | C |
| Example 85 | A |
| Example 86 | A |
| Example 87 | A |
| Example 88 | A |
| Example 89 | A |
| Example 90 | A |
| Example 91 | B |
| Example 92 | B |
| Example 93 | B |
| Example 94 | A |
| Example 95 | A |
| Example 96 | A |
| Example 97 | B |
| Example 98 | B |
| Example 99 | B |
| Example 100 | B |
| Example 101 | B |
| Example 102 | B |
| Example 103 | B |
| Example 104 | B |
| Example 105 | C |
| Example 106 | C |
| Example 107 | A |
| Example 108 | B |
| Example 109 | B |
| Example 110 | B |
| Example 111 | A |
| Example 112 | A |
| Example 113 | B |
| Example 114 | C |
| Example 116 | C |
| Example 117 | C |
| Example 118 | C |
| Example 119 | B |
| Example 120 | C |
| Example 121 | B |
| Example 122 | B |
| Example 123 | C |
| Example 124 | B |
| Example 125 | B |
| Example 126 | C |
| Example 127 | C |
| Example 128 | C |
| Example 129 | C |
| Example 130 | B |
| Example 131 | B |
| Example 132 | B |
| Example 133 | C |
| Example 134 | C |
| Example 135 | C |
| Example 136 | C |
| Example 146 | A |
| Example 147 | B |
| Example 148 | B |
| Example 149 | B |
| Example 150 | A |
| Example 151 | C |
| Example 152 | B |
| Example 153 | A |
| Example 154 | A |
| Compd. 4-2 | A |
| Example 155 | A |
| Example 156 | A |
| Compd. 6-1 | B |
| Cpmpd. 6-2 | B |
| Example 157 | A |
| Example 158 | A |
| Example 159 | B |
| Example 160 | A |
| Example 161 | A |
| Example 162 | A |
| Example 163 | B |
| Example 164 | B |
| Exmaple 165 | A |
| Example 166 | B |
| Example 167 | B |
| Example 168 | A |
| Example 169 | A |
| Example 170 | A |
| Example 171 | D |
| Example 172 | B |
| Example 173 | B |
| Example 174 | A |
| Example 175 | A |
| Example 176 | B |
| Example 177 | A |
| Example 178 | A |
| Example 179 | A |
| Example 180 | A |
| Example 181 | A |
| Example 182 | D |
| Example 183 | A |
| Example 184 | A |
| Example 185 | B |
| Example 186 | C |
| Example 187 | C |
| Example 188 | D |
| Example 189 | B |
| Example 190 | A |
| Example 191 | A |
| Example 192 | B |
| Example 193 | A |
| Example 194 | A |
| Example 195 | A |
| Example 196 | A |
| Example 197 | A |
| Example 198 | A |
| Example 199 | A |
| Example 200 | A |
| Example 201 | A |
| Example 202A | B |
| Example 202B | C |
| Example 203A | A |
| Example 203B | B |
| Example 20 | A |
| Example 205A | A |
| Example 205B | C |
| Example 206 | A |
| Example 207 | A |
| Example 208 | A |
| Example 209 | A |
| Example 210 | A |
| Example 211 | A |
| Example 212 | A |
| Example 213 | B |
| Example 214 | A |
| Example 215 | A |
| Example 216 | B |
| Example 217 | A |
| Example 218 | A |
| Example 219 | A |
| Example 220 | A |
| Example 221 | A |
| Example 222 | A |
| Example 223 | A |
| Example 224 | B |
| Example 225 | A |
| Example 226 | A |
| Example 227 | A |
| Example 228 | A |
| Example 229 | A |
| Example 230 | A |
| Example 231 | B |

Table 2 gives the viral load lowering EC₅₀ values for exemplified compounds of the invention grouped in the following ranges: A indicates EC₅₀ < 0.1 µM; B indicates EC₅₀ of ≥ 0.1 to <1.0 µM; C indicates EC₅₀ of ≥1.0 to <10 µM.

TABLE 2

Viral load lowering for exemplified compounds of the invention

| | |
|---|---|
| | VL HepAD38 EC₅₀ range |
| Example 137 | A |
| Example 138 | A |
| Example 139 | A |
| Example 140 | A |
| Example 141 | A |
| Example 142 | A |
| Example 143 | A |
| Example 144 | A |
| Example 145 | A |

### Stereochemistry of Example

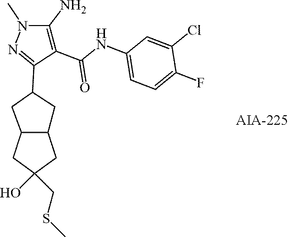 [SMILES: CSCC1(O)CC2CC(c3nn(C)c(N)c3C(=O)Nc3ccc(F)c(Cl)c3)CC2C1]

5-Amino-N-(3-chloro-4-fluorophenyl)-3-(5-hydroxy-5-(methylthiomethyl)octahydropentalen-2-yl)-1-methyl-1H-pyrazole-4-carboxamide. To a solution of 5-amino-N-(3-chloro-4-fluorophenyl)-3-(hexahydro-1′H-spiro[oxirane-2,2′-pentalene]-5′-yl)-1-methyl-1H-pyrazole-4-carboxamide (200 mg, 0.495 mmol) in THF/H₂O (6 mL/2 mL ) was added NaSMe (138.6 mg, 1.98 mmol). The mixture was stirred at rt overnight. The solvent was removed and the crude product purified by silica gel column chromatography using 3:1 petroleum ether/ethyl acetate to afford 5-amino-N-(3-chloro-4-fluorophenyl)-3-(5-hydroxy-5-(methylthiomethyl)octahydropentalen-2-yl)-1-methyl-1H-pyrazole-4-carboxamide (100 mg, 44.7%) as a yellow solid. MS (m/z): Calcd.: 452.1, Found: 452.2 [M+1]⁺.

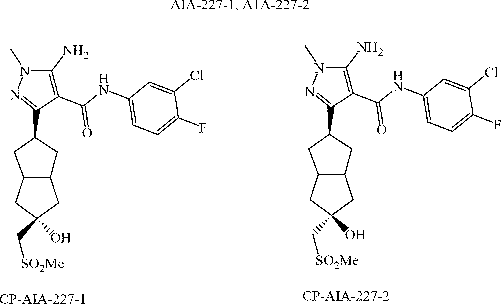

5-Amino-N-(3-chloro-4-fluorophenyl)-3-((2r,5r)-5-hydroxy-5-(methylsulfonylmethyl)octahydropentalen-2-yl)-1-methyl-1H-pyrazole-4-carboxamide (AIA-227-1) and 5-Amino-N-(3-chloro-4-fluorophenyl)-3-((2 s,5 s)-5-hydroxy-5-(methylsulfonylmethyl)octahydropentalen-2-yl)-1-methyl-1H-pyrazole-4-carboxamide (AIA-227-2). To a solution of 5-amino-N-(3-chloro-4-fluorophenyl)-3-(5-hydroxy-5-(methylthiomethyl)octahydropentalen-2-yl)-1-methyl-1H-pyrazole-4-carboxamide (100 mg, 0.22 mmol) in DCM (5 mL) was added m-CPBA (114.8 mg, 0.66 mmol). The mixture was stirred at rt overnight. The solvent was removed, and the crude material purified by silica gel column chromatography using 3:1 (v/v) DCM/MeOH to afford AIA-227 (40 mg, 37.3%) as a white solid. MS (m/z): Calcd.: 484.1, Found: 484.3 [M+1] ⁺. AIA-227 was separated by SFC to give AIA-227-1 (4 mg) as a white solid and AIA-227-2 (4 mg) as a white solid. AIA-227-1: ¹H NMR (400 MHz, DMSO-d₆): δ 8.95 (s, 1H), 7.91 (dd, J = 6.8, 2.4 Hz, 1H), 7.54-7.50 (m, 1H), 7.35 (t, J = 9.2 Hz, 1H), 5.97 (s, 2H), 4.79 (s, 1H), 3.59-3.53 (m, 1H), 3.49 (s, 3H), 3.35 (s, 2H), 2.97 (s, 3H), 2.67-2.60 (m, 2H), 2.18-2.12 (m, 2H), 2.07-2.02 (m, 2H), 1.45-1.36 (m, 4H) ppm. AIA-227-2: ¹H NMR (400 MHz, DMSO-d₆): δ 8.94 (s, 1H), 7.91 (dd, J = 2.8, 2.4 Hz, 1H), 7.53 - 7.49 (m, 1H), 7.34 (t, J = 9.2 Hz, 1H), 5.97 (s, 2H), 4.87 (s, 1H), 3.49 (s, 3H), 3.43 - 3.35 (m, 1H), 3.25 (s, 2H), 2.97 (s, 3H), 2.49 (s, 2H), 2.15 - 2.09 (m, 2H), 2.02 - 1.97 (m, 2H), 1.73 - 1.60 (m, 4H) ppm.

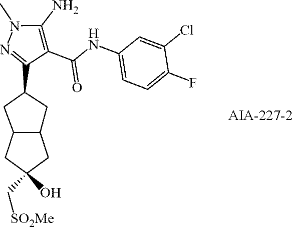 [SMILES: Cn1nc([C@H]2CC3C[C@](O)(CS(C)(=O)=O)CC3C2)c(C(=O)Nc2ccc(F)c(Cl)c2)c1N]

Alternative synthesis of 5-amino-N-(3-chloro-4-fluorophenyl)-3-((2 s,5 s)-5-hydroxy-5-(methylsulfonylmethyl)octahydropentalen-2-yl)-1-methyl-1H-pyrazole-4-carboxamide. To a solution of dimethylsulfone (77.0 g, 818.7 mmol) in THF (800 mL) was added n-BuLi (327.5 mL, 818.7 mmol, 2.5 M) dropwise at -78° C. The resulting solution was allowed to warm to -20° C. and stirred for 1 hr. The reaction was cooled to -78° C., and a solution of AIA-002 (40.0 g, 102.3 mmol) in anhydrous tetrahydrofuran (1200 mL) was added over 2 hr. The mixture was warmed to rt and stirred for an additional 4 hr. The reaction mixture was quenched with saturated aqueous ammonium chloride solution (200 mL). The solvent was removed, followed by dilution with water, extraction with ethyl acetate (3 × 200 mL), drying over Na₂SO₄, filtration, and concentration to give the crude product. The crude product was purified by column chromatography using 0-5% (v/v) methanol in DCM and basic prep-HPLC to afford 5-amino-N-(3-chloro-4-fluorophenyl)-3-((2 s,5 s)-5-hydroxy-5-(methylsulfonylmethyl)octahydropentalen-2-yl)-1-methyl-1H-pyrazole-4-carboxamide (26.0 g, 52.4%) as a white solid. MS (m/z): Calcd.: 484.1, Found: 485.2 [M+ 1] ⁺; ¹H NMR (400 MHz, DMSO-d₆): δ 8.96 (s, 1H), 7.92 (dd, J = 6.8, 2.8 Hz, 1H), 7.54 - 7.50 (m, 1H), 7.35 (t, J = 8.8 Hz, 1H), 5.98 (s, 2H), 4.88 (s, 1H), 3.49 (s, 3H), 3.42 - 3.37 (m, 1H), 3.25 (s, 2H), 2.97 (s, 3H), 2.15 - 2.10 (m, 2H), 2.03 - 1.97 (m, 2H), 1.73 - 1.60 (m, 4H) ppm.

A crystal with size of 0.08 × 0.10 × 0.20 mm of compound AIA-227-2 was obtained from EtOH after 20 days of volatilization and was used for X-ray diffraction data collection. The data were collected on a Bruker SMART CCD area-detector diffractometer at room temperature using CuKα radiation by ω/φ scan mode. 10846 reflections were collected, of which 3754 reflections were unique (Rint = 0.0507).

The crystal belongs to monoclinic crystal system, with a space group P2₁/c. The unit cell parameters were as follows: a= 6.6143(3), b=14.0381(8), c=23.6870(14)Å, α=γ=90.0°, β=97.702(3)°, V= 2179.5(2)Å³, Z=4.

The structure was solved by direct methods and all of the non-H atoms were refined against F² by full-matrix least-squares methods using the SHELXTL program. All H atoms were placed in geometrically idealized positions and constrained to ride on their parent atoms. Multi-scans absorption correction method was used, and the maximum and minimum transmission parameters were 0.7531 and 0.6017, respectively. The final R, wR₂, GOF are 0.0457, 0.1293 and 1.024, respectively.

There is one C₂₁H₂₆FClN₄O₄S molecule in the asymmetric unit and hydrogen bonds can be found between them, which play an important role for the stable packing of the crystal structure.

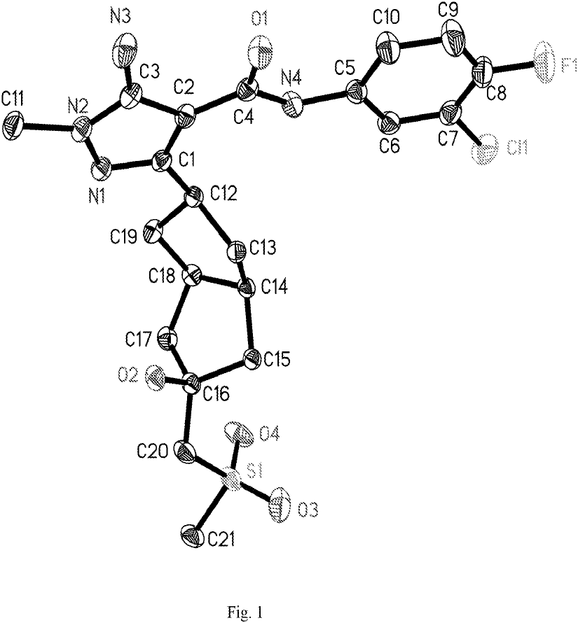

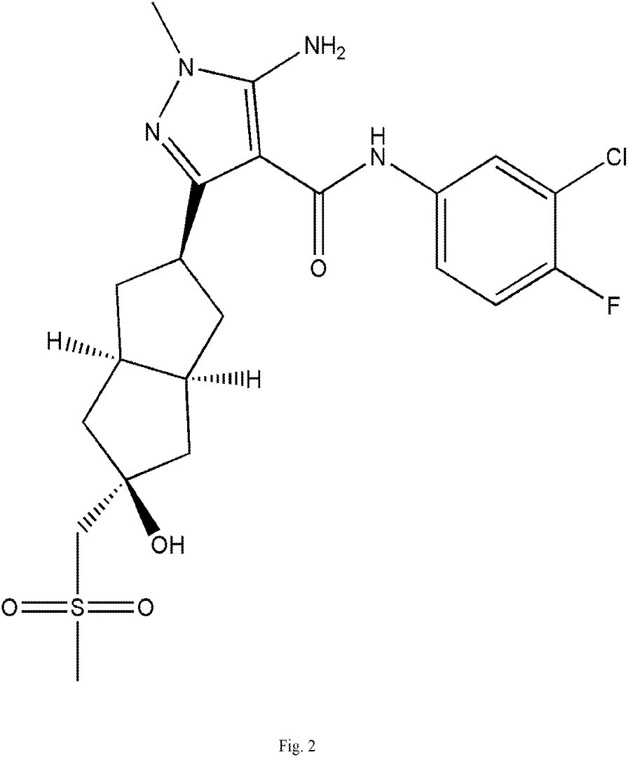

The ORTEP plot for compound AIA-227-2 is present in FIG. 1. The relative stereochemistry scheme of compound AIA-227-2 is shown in FIG. 2. The depictions of stereochemistry in the chemical structures of related examples are based on this assignment.

### INCORPORATION BY REFERENCE

All publications and patents mentioned herein, including those items listed below, are hereby incorporated by reference in their entirety for all purposes as if each individual publication or patent was specifically and individually incorporated by reference. In case of conflict, the present application, including any definitions herein, will control.

### EQUIVALENTS

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure.

1. A compound of Formula I 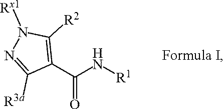
or a pharmaceutically acceptable salt thereof, wherein:
L is C₁₋₄alkylene or haloC₁₋₄alkylene;
L¹ is a bond, C₁₋₆alkylene, O, NR^{c}, C(O), C(O)O, C(O)NR^{c}, S(O)t or S(O)ₜNR^{c};
X³ is NR⁴ or CR⁴R⁸;
X⁴ is O or S;
X⁵ is O, S or NR°;
R^{a}, R^{b} and R^{c} are independently selected for each occurrence from the group consisting of hydrogen, C₁₋₆ alkyl, haloC₁₋₆ alkyl and C₃₋₆ monocycloalkyl;
R^{d} is hydrogen, OH, C₁₋₆ alkyl or C₁₋₆ alkoxy;
R^{x1} is hydrogen, C₁₋₄ alkyl, C₁₋₄ alkenyl, C₁₋₄ alkynyl, haloC₁₋₄ alkyl, or C₃₋₆ monocycloalkyl; or R^{x1} and R² together form a —CH₂CH₂CH₂—, —CH₂CH₂CH₂CH₂—, —CH₂CH₂O—, —CH₂OCH₂—, —CH₂CH₂CH₂O— —CH₂CH₂OCH₂—, —CH₂CH₂—NH— —CH₂NHCH₂—, —CH₂CH₂CH₂NH— or —CH₂CH₂NHCH₂— group;
R^{0a} is independently selected for each occurrence from the group consisting of halogen, OH, CN, NO₂, R^{a}R^{b}N—, C₁₋₄alkyl and haloC₁₋₄alkyl;
R^{4a} and R^{6b} are independently hydrogen or C₁₋₄ alkyl;
R⁰, R⁶ and R¹¹ are independently selected for each occurrence from the group consisting of halogen, OH, CN, NO₂, oxo, R^{d}N═, hydrazino, formyl, azido, silyl, siloxy, HOC(O)—, R^{a}R^{b}N—, R^{a}R^{b}NS(O)ₜ—, C₁₋₆alkyl, C₂₋₆alkenyl, C₂₋₆alkynyl, haloC₁₋₆alkyl, hydroxyC₁₋₆alkyl-, R^{a}R^{b}NC₁₋₆alkyl-, HOC(O)C₁₋₆alkyl-, R^{a}R^{b}NC₁₋₆alkylNR^{c}, C₁₋₆alkylNR^{a}C₁₋₆alkylNR^{c-}, C₁₋₆alkoxy, haloC₁₋₆alkoxy, hydroxyC₁₋₆alkoxy-, R^{a}R^{b}NC₁₋₆alkoxy-, C₁-₆alkoxyC₁-₆alkyl-, haloC₁₋₆alkoxyC₁₋₆alkyl-, R^{a}R^{b}NC(O)—, C₁₋₆alkylC(O)-, C₁-₆alkoxyC(O)-, C₁₋₆alkylC(O)O-, C₁₋₆alkylS(O)_{q}-, C₁₋₆alkylS(O)ₜNR^{c}-, C₁₋₆alkylS(O)tC₁₋₆alkyl-, C₁₋₆alkylS(O)tNR^{a}C₁₋₆alkyl-, C₃₋₆cycloalkylS(O)ₜC₁₋₆alkyl-, C₁₋₆alkylC(O)C₁₋₆alkyl-, and C₁₋₆alkylC(O)OC₁₋₆alkyl-;
R¹ is a phenyl or 5-6 membered monocyclic heteroaryl, wherein the phenyl or 5-6 membered monocyclic heteroaryl is optionally substituted with one, two, or three independently selected R¹¹ groups;
R² and R⁸ are independently selected from the group consisting of hydrogen, halo, CN, OH, R^{a}R^{b}N, C₁₋₄alkyl, haloC₁₋₄alkyl, C₃₋₅monocycloalkyl, C₁₋₄alkoxy, and haloC₁₋₄alkoxy;
R^{3a} is 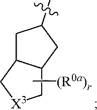
R⁴ is R⁵—L¹— or R⁹;
R⁵ is 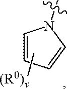 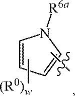 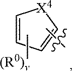 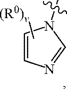 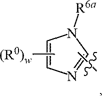 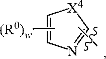 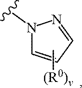 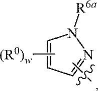 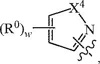 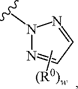 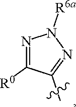 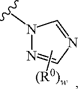 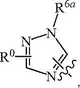 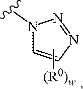 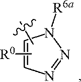 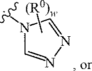 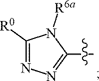
R⁹ is R¹⁴S(O)_{q}—L, R¹⁴S(O)_{q}NH—L— or R¹⁴C(O)NH—L;
R¹⁴is R^{a}R^{b}N—, C₁₋₆alkyl, C₂₋₆alkenyl, C₂₋₆alkynyl, C₁₋₆haloalkyl, C₁₋₆alkoxy, C₁₋₆haloalkyl, C₁₋₆haloalkoxy, or R⁵—L¹—;
q, r, t, and w are independently selected for each occurrence from 0, 1 and 2; and
v is independently selected for each occurrence from 0, 1, 2 and 3.

2. The compound of claim 1, or a pharmaceutically acceptable salt thereof, wherein R^{x1} is hydrogen or methyl.

3. The compound of claim 1, or a pharmaceutically acceptable salt thereof, wherein R^{x1} is methyl.

4. The compound according to claim 1, or a pharmaceutically acceptable salt thereof, wherein r is 0.

5. The compound according to claim 1, or a pharmaceutically acceptable salt thereof, wherein R² is R^{a}R^{b}N.

6. The compound of claim 5, or a pharmaceutically acceptable salt thereof, wherein R² is NH₂.

7. The compound according to claim 1, or a pharmaceutically acceptable salt thereof, wherein: R¹ is 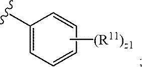
R
¹¹ is independently selected for each occurrence from the group consisting of halogen, CN, C₁₋₆ alkyl and haloC₁₋₆ alkyl; and z1 is 0, 1, 2 or 3.

8. The compound of claim 7, or a pharmaceutically acceptable salt thereof, wherein for each occurrence R¹¹ is independently selected from the group consisting of CN, F, Cl, Br and I.

9. The compound of claim 8, or a pharmaceutically acceptable salt thereof, wherein R¹ is 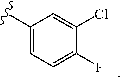
.

10. The compound according to claim 1, or a pharmaceutically acceptable salt thereof, wherein X³ is CR⁴R⁸.

11. The compound of claim 10, or a pharmaceutically acceptable salt thereof, wherein R⁴ is R⁹.

12. The compound of claim 10, or a pharmaceutically acceptable salt thereof, wherein R⁴ is R⁵—L¹—.

13. The compound of 12, or a pharmaceutically acceptable salt thereof, wherein L¹ is a bond.

14. The compound according to claim 1, or a pharmaceutically acceptable salt thereof, wherein R⁸ is hydrogen, OH or C₁₋₆ alkoxy.

15. The compound of claim 14, or a pharmaceutically acceptable salt thereof, wherein R⁸ is OH.

16. The compound of claim 14, or a pharmaceutically acceptable salt thereof, wherein R⁸ is hydrogen.

17. A pharmaceutical composition comprising the compound according to claim 1, or a pharmaceutically acceptable salt thereof, and a pharmaceutically acceptable excipient.

18. A method of treating Hepatitis B (HBV) infection in a subject in need thereof, the method comprising: administering to the subject a therapeutically effective amount of a compound according to claim 1, or a pharmaceutically acceptable salt thereof.

19. A method of treating Hepatitis B (HBV) infection in a subject in need thereof, the method comprising: administering to the subject a therapeutically effective amount of pharmaceutical composition of claim 17.
